(19) United States
(12) Patent Application Publication
(10) Pub. No.: US 2024/0309038 A1
(43) Pub. Date: Sep. 19, 2024

(54) NOVEL GALACTOSIDE INHIBITOR OF GALECTINS

(71) Applicant: GALECTO BIOTECH AB, Copenhagen N (DK)

(72) Inventors: Fredrik ZETTERBERG, Copenhagen N (DK); Kristoffer PETERSON, Copenhagen N (DK); Ulf NILSSON, Copenhagen N (DK)

(73) Assignee: GALECTO BIOTECH AB, Copenhagen N (DK)

(21) Appl. No.: 18/259,146

(22) PCT Filed: Dec. 22, 2021
(86) PCT No.: PCT/EP2021/087295

§ 371 Date: Jun. 23, 2023

(30) Foreign Application Priority Data
Dec. 28, 2020 (EP) 20217433.0
Jun. 29, 2021 (EP) 21182401.6

(51) Int. Cl. C07H 19/04; A61K 31/7056; A61K 31/706

(52) U.S. Cl. CPC C07H 19/04; A61K 31/7056; A61K 31/706

(57) ABSTRACT

A D-galactopyranose compound of formula (1)

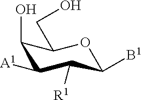

wherein
the pyranose ring is beta-D-galactopyranose, and these compounds are high affinity galectin-3 inhibitors for use in treatment of disorders relating to the binding of a galectin-3 to a ligand in a mammal. The disorders include inflammation; fibrosis; endometriosis; scarring; keloid formation; aberrant scar formation; surgical adhesions; scleroderma; systemic sclerosis; septic shock; cancers; metastasising cancers; systemic lupus erythematosus; metabolic disorders; coagulopathies; cardiovascular disorders; heart disease; heart failure; and eye diseases; atherosclerosis; endocrine disorders; metabolic diseases; insulin resistance; obesity; Diastolic HF; atrophic diseases in the brain; disorders related to transplantation in organs; acute burn; acute inflammatory reaction; chronic acute skin graft rejection; chronic scarring; asthma and other interstitial lung diseases; Otosclerosis, mesothelioma; post-surgery disorders; toxin exposure disorders; Tissue injury; congenital hepatic fibrosis; hereditary fibrosing poikiloderma; liver disorders; and neurodegenerative disorders.

### TECHNICAL FIELD

The present invention relates to novel compounds, the use of said compounds as medicament and for the manufacture of a medicament for the treatment of diseases or disorders such as but not limited to cancers; fibrosis; scarring; keloid formation; aberrant scar formation; surgical adhesions; pathological angiogenesis; eye diseases; HIV-1 diseases; inflammation or transplant rejection in mammals. The invention also relates to pharmaceutical compositions comprising said novel compounds.

### BACKGROUND ART

Galectins are proteins with a characteristic carbohydrate recognition domain (CRD). This is a tightly folded β-sandwich of about 130 amino acids (about 15 kDa) with the two defining features 1) a β-galactose binding site and 2) sufficient similarity in a sequence motif of about seven amino acids, most of which (about six residues) make up the β-galactose binding site. Galectins are synthesized as cytosolic proteins from where they can be targeted to the nucleus, specific cytososlic sites, or secreted to engage in mechanisms effecting physiological functions such as inflammation, immune responses, cell-migration and autophagy. (Johannes et. al 2018) There are now over 9319 publications on galectins in PubMed, with most, as mentioned above, about galectins-1 (>1989) and -3 (>4791). Evidence from literature suggests roles for galectins in e.g. fibrosis, inflammation and cancer (Dings et. al., Dubé-Delarosbil et. al 2017)

Galectin-3 is widely expressed in many cell types and tissues (www.proteinatlas.org) being involved in mechanisms such as apoptosis, adhesion and migration, cell transformation, invasion and metastasis immune escape and angiogenesis. Upregulation of galectin 3 has also been associated with cancer, inflammation, neurodegenerative disease, fibrotic disease and diabetes (Dings et. al. 2018, Slack et. al. 2020, Li et. al. 2016) Example of small molecule ligands including β-D-galactopyranoside were recently reviewed and examplified in Blanchard et. al 2014 and Sethi et. al 2021.

### SUMMARY OF THE INVENTION

The compounds of the present invention are novel β-D-galactopyranose compounds that unexpectedly have shown high affinity for galectin-3 and are considered novel potent drug candidates. In addition, it is seen that with these compounds the advantage is that the pyrazole moiety A¹ results in an improved uptake over CACO-2 cells predicting high oral bioavailability after oral administration and this is supported by disclosed ADME data herein. Further compared to compounds where A¹ is a triazole moiety this improvement is significant.

In a broad aspect the present invention concerns a β-D-galactopyranose compound of formula (I)

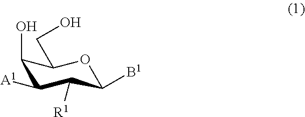

wherein
the pyranose ring is β-D-galactopyranose,
A1 is

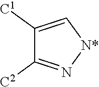

wherein the asterix * indicates the nitrogen atom of the triazole ring that is covalently attached to the galactopyranose;
C¹ is selected from a) an aryl, such as phenyl or naphthyl, optionally substituted with a group selected from a halogen; CN; a C₂₋₄ alkenyl; an ethynyl; a spiro heterocycle; OH; —COOH; —CONR²R³, wherein R² and R³ are independently selected from H, C₁₋₃ alkyl, and cyclopropyl, or R² and R³ together with the nitrogen may form a heterocycloalkyl; C₁₋₃ alkyl, optionally substituted with a F; C₁₋₃ alkyl, optionally substituted with an ethynyl; cyclopropyl, optionally substituted with a F; OC₁₋₃ alkyl, optionally substituted with a F; O-cyclopropyl, optionally substituted with a F; NR⁴R⁵, wherein R⁴ and R⁵ are independently selected from H, C₁₋₃ alkyl and cyclopropyl; C(═O)—R^{11a}, wherein R^{11a} is selected from H and C₁₋₃ alkyl; and R⁶—CONH— wherein R⁶ is selected from C₁₋₃ alkyl and cyclopropyl; or b) a heterocycle, such as heteroaryl or heterocycloalkyl, optionally substituted with a group selected from a halogen; a spiro heterocycle; CN; OH; —COOH; —CONR⁷R⁸, wherein R⁷ and R⁸ are independently selected from H, C₁₋₃ alkyl, and cyclopropyl, or R⁷ and R⁸ together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR⁹R¹⁰, wherein R⁹ and R¹⁰ are independently selected from H, C₁₋₃ alkyl, and cyclopropyl; C(═O)—R¹¹, wherein R¹¹ is selected from H and C₁₋₃ alkyl; and R¹²—CONH— wherein R¹² is selected from C₁₋₃ alkyl and cyclopropyl; C² is selected from the group consisting of
a) H; C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{4a}R^{5a}, wherein R^{4a} and R^{5a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{2a}R^{3a}, wherein R^{2a} and R^{3a} are independently selected from H, C₁₋₃ alkyl optionally substituted with a halogen, and cyclopropyl optionally substituted with a halogen; and S(O₂)NR^{6a}R^{7a} wherein R^{6a} and R^{7a} are independently selected from H, C₁₋₆ alkyl optionally substituted with a halogen, and cyclopropyl optionally substituted with a halogen;
b) phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{4b}R^{5b}, wherein R^{4b} and R^{5b} are independently selected from H, C₁₋₆ alkyl optionally substituted with a halogen, C₃_6 cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{2b}R^{3b}, wherein R^{2b} and R^{3b} are independently selected from H, C₁₋₃ alkyl optionally substituted with a halogen, and cyclopropyl optionally substituted with a halogen; and S(O₂)NR^{6b}R^{7b} wherein R^{6b} and R^{7b} are independently selected from H, C₁₋₆ alkyl optionally substituted with a halogen, and cyclopropyl optionally substituted with a halogen;
c) Y—Z wherein
Y is linked to the pyrazole of A¹ and is selected from the group consisting of S, Se, SO, SO₂, O, C═O, and CR^{2c}R^{3c} wherein R^{2c} and R^{3c} are independently selected from hydrogen, OH, or halogen; and
Z is selected from the group consisting of phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{4c}R^{5c}, wherein R^{4c} and R^{5c} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{2d}R^{3d}, wherein R^{2d} and R^{3d} are independently selected from H, C₁₋₃ alkyl optionally substituted with a halogen, and cyclopropyl optionally substituted with a halogen; and S(O₂)NR^{6c}R^{7c} wherein R^{6c} and R^{7c} are independently selected from H, C₁₋₆ alkyl optionally substituted with a halogen, and cyclopropyl optionally substituted with a halogen;
B¹ is selected from the group consisting of a heteroaryl, an aryl and a heterocycloalkyl, optionally substituted with one or more groups selected from a) C₁₋₆ alkyl optionally substituted with one or more of C₁₋₆ alkyl, amino, CN, halogen, hydroxy, C₁₋₆ alkoxy, carboxy, alkoxycarbonyl, H₂NCO, b) R¹³—C₁₋₆ alkyl, c) C₃₋₆ cycloalkyl optionally substituted with one or more of C₁₋₆ alkyl, amino, CN, halogen, or hydroxy, d) C₁₋₆ alkoxy, e) C₁₋₆ alkylthio, f) C₁₋₆ alkylsulfonyl, g) carbonyl substituted with any one of hydroxy, C₁₋₆ alkoxy, C₁₋₆ alkylNH, ((R¹⁴)(R¹⁵)N)C₁₋₆ alkylNH, or (pyridinyl)C₁₋₆ alkylNH, h) (R¹⁶)(R¹⁷)N, i) C₂-alkynyl, j) R¹³, and k) C₁₋₆ alkenyl,
wherein R¹³ is selected from any one of a) phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thiazolo[4,5-b]pyridinyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of cyano, nitro, OH, C₂-alkynyl, halogen, C₁₋₆ alkyl, halo-C₁₋₆ alkyl, C₃₋₆ cycloalkyl, C₁₋₆ alkoxy, halo-C₁₋₆ alkoxy, C₁₋₆ alkylthio, carboxy, C₁₋₆ alkoxycarbonyl, CONH₂, and (R¹⁸)(R¹⁹)N; or b) (C₁₋₆ alkyl-SO₂)phenyl, (C₁₋₆ alkyl SO₂)(halo)phenyl, (aminoSO₂)phenyl, (di-C₁₋₆ alkylaminoSO₂)phenyl, ((C₁₋₆ alkyl-NHSO₂)—C₁₋₆ alkyl)phenyl, (pyrrolyl)phenyl, (imidazolyl)phenyl, (oxazolyl)phenyl, (tetrazolyl)phenyl, ((pyridinyl)methyl)phenyl, phenoxyphenyl, (benzyloxy)phenyl, ((methyl)thiazolyl)-phenyl, (thiazolyl)-benzenesulfamido, ((methyl)thiadiazolyl)benzenesulfamido, (methyl)-benzothiazolonyl, or fluoropyrazolopyrimidinyl;
wherein
R¹⁴ is hydrogen or C₁₋₆ alkyl;
R¹⁵ is hydrogen or C₁₋₆ alkyl; or
(R¹⁴)(R¹⁵)N taken together is any one of azetidinyl, pyrrolidinyl, piperidinyl, piperazinyl, or morpholinyl, optionally substituted with one or more substituents selected from halogen, C₁₋₆ alkyl, and hydroxy;
R¹⁶ is hydrogen, C₁₋₆ alkyl, C₁₋₆ alkylcarbonyl, or C₁₋₆ alkylsulfonyl;
R¹⁷ is hydrogen or C₁₋₆ alkyl; or
(R¹⁶)(R¹⁷)N taken together is any one of azetidinyl, pyrrolidinyl, piperidinyl, piperazinyl, or morpholinyl, optionally substituted with one or more substituents selected from halogen, C₁₋₆ alkyl, and C₁₋₆ alkylcarbonyl;
R¹⁸ is hydrogen, C₁₋₆ alkyl, C₁₋₆ alkylcarbonyl, or C₁₋₆ alkylsulfonyl;
R¹⁹ is hydrogen or C₁₋₆ alkyl; or
(R¹⁸)(R¹⁹)N taken together is any one of azetidinyl, pyrrolidinyl, piperidinyl, piperazinyl, or morpholinyl, optionally substituted with one or more substituents selected from halogen, C₁₋₆ alkyl, and C₁₋₆ alkylcarbonyl;
R¹ is selected from the group consisting of a) H, b) OH, c) OC₁₋₆ alkyl optionally substituted with one or more halogen, phenyl, phenyl substituted with one or more groups selected form OH and halogen, CN, OR²⁰, NR²¹R²², and CONH₂, wherein R²⁰ is selected from the group consisting of H, CN, a halogen, methyl optionally substituted with a F, OCH₃ optionally substituted with a F, OCH₂CH₃ optionally substituted with a F, OH, and R²³—CONH— wherein R²³ is selected from C₁₋₃ alkyl and cyclopropyl, R²¹ is selected from the group consisting of H, CN, a halogen, methyl optionally substituted with a F, OCH₃ optionally substituted with a F, OCH₂CH₃ optionally substituted with a F, OH, and R²⁴—CONH— wherein R²⁴ is selected from C₁₋₃ alkyl and cyclopropyl, and R²² is selected from the group consisting of H, CN, a halogen, methyl optionally substituted with a F, OCH₃ optionally substituted with a F, OCH₂CH₃ optionally substituted with a F, OH, and R²⁵—CONH— wherein R²⁵ is selected from C₁₋₃ alkyl and cyclopropyl, d) branched OC₃₋₆ alkyl optionally substituted with one or more halogen, CN, OR²⁶, NR²⁷R²⁸, and CONH₂, wherein R²⁶ is selected from the group consisting of H, CN, a halogen, methyl optionally substituted with a F, OCH₃ optionally substituted with a F, OCH₂CH₃ optionally substituted with a F, OH, and R²⁹—CONH— wherein R²⁹ is selected from C₁₋₃ alkyl and cyclopropyl, R²⁷ is selected from the group consisting of H, CN, a halogen, methyl optionally substituted with a F, OCH₃ optionally substituted with a F, OCH₂CH₃ optionally substituted with a F, OH, and R³⁰—CONH— wherein R^{3c} is selected from C₁₋₃ alkyl and cyclopropyl, and R²⁸ is selected from the group consisting of H, CN, a halogen, methyl optionally substituted with a F, OCH₃ optionally substituted with a F, OCH₂CH₃ optionally substituted with a F, OH, and R³¹—CONH— wherein R³¹ is selected from C₁₋₃ alkyl and cyclopropyl, e) cyclic OC₃₋₆ alkyl optionally substituted with one or more halogen, CN, OR³², NR³³R³⁴, and CONH₂, wherein R³² is selected from the group consisting of H, CN, a halogen, methyl optionally substituted with a F, OCH₃ optionally substituted with a F, OCH₂CH₃ optionally substituted with a F, OH, and R³⁵—CONH— wherein R³⁵ is selected from C₁₋₃ alkyl and cyclopropyl, R³³ is selected from the group consisting of H, CN, a halogen, methyl optionally substituted with a F, OCH₃ optionally substituted with a F, OCH₂CH₃ optionally substituted with a F, OH, and R³⁶—CONH— wherein R³⁶ is selected from C₁₋₃ alkyl and cyclopropyl, and R³⁴ is selected from the group consisting of H, CN, a halogen, methyl optionally substituted with a F, OCH₃ optionally substituted with a F, OCH₂CH₃ optionally substituted with a F, OH, and R³⁷—CONH— wherein R³⁷ is selected from C₁₋₃ alkyl and cyclopropyl, and f) a fluorine (F); or a pharmaceutically acceptable salt or solvate thereof.

In a still further aspect the present invention concerns a β-D-galactopyranose compound of formula (I)

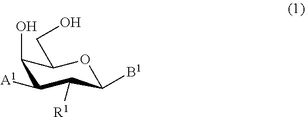

wherein
the pyranose ring is β-D-galactopyranose,
A1 is

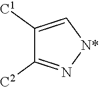

wherein the asterix * indicates the nitrogen atom of the triazole ring that is covalently attached to the galactopyranose;
C¹ is selected from a) an aryl, such as phenyl or naphthyl, optionally substituted with a group selected from a halogen; CN; an ethynyl; a spiro heterocycle; OH; —COOH; —CONR²R³, wherein R² and R³ are independently selected from H, C₁₋₃ alkyl, and cyclopropyl, or R² and R³ together with the nitrogen may form a heterocycloalkyl; C₁₋₃ alkyl, optionally substituted with a F; C₁₋₃ alkyl, optionally substituted with an ethynyl; cyclopropyl, optionally substituted with a F; OC₁₋₃ alkyl, optionally substituted with a F; O-cyclopropyl, optionally substituted with a F; NR⁴R⁵, wherein R⁴ and R⁵ are independently selected from H, C₁₋₃ alkyl and cyclopropyl; C(═O)—R^{11a}, wherein R^{11a} is selected from H and C₁₋₃ alkyl; and R⁶—CONH— wherein R⁶ is selected from C₁₋₃ alkyl and cyclopropyl; or b) a heterocycle, such as heteroaryl or heterocycloalkyl, optionally substituted with a group selected from a halogen; a spiro heterocycle; CN; OH; —COOH; —CONR⁷R⁸, wherein R⁷ and R⁸ are independently selected from H, C₁₋₃ alkyl, and cyclopropyl, or R⁷ and R⁸ together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR⁹R¹⁰, wherein R⁹ and R¹⁰ are independently selected from H, C₁₋₃ alkyl, and cyclopropyl; C(═O)—R¹¹, wherein R¹¹ is selected from H and C₁₋₃ alkyl; and R¹²—CONH— wherein R¹² is selected from C₁₋₃ alkyl and cyclopropyl;
C² is selected from the group consisting of
a) H; C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{4a}R^{5a}, wherein R^{4a} and R^{5a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{2a}R^{3a}, wherein R^{2a} and R^{3a} are independently selected from H, C₁₋₃ alkyl optionally substituted with a halogen, and cyclopropyl optionally substituted with a halogen; and S(O₂)NR^{6a}R^{7a} wherein R^{6a} and R^{7a} are independently selected from H, C₁₋₆ alkyl optionally substituted with a halogen, and cyclopropyl optionally substituted with a halogen;
b) phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{4b}R^{5b}, wherein R^{4b} and R^{5b} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃_6 cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{2b}R^{3b}, wherein R^{2b} and R^{3b} are independently selected from H, C₁₋₃ alkyl optionally substituted with a halogen, and cyclopropyl optionally substituted with a halogen; and S(O₂)NR^{6b}R^{7b} wherein R^{6b} and R^{7b} are independently selected from H, C₁₋₆ alkyl optionally substituted with a halogen, and cyclopropyl optionally substituted with a halogen;
c) Y—Z wherein
Y is linked to the pyrazole of A¹ and is selected from the group consisting of S, Se, SO, SO₂, O, C═O, and CR^{2c}R^{3c} wherein R^{2c} and R^{3c} are independently selected from hydrogen, OH, or halogen; and
Z is selected from the group consisting of phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{4c}R^{5c}, wherein R^{4c} and R^{5c} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{2d}R^{3d}, wherein R^{2d} and R^{3d} are independently selected from H, C₁₋₃ alkyl optionally substituted with a halogen, and cyclopropyl optionally substituted with a halogen; and S(O₂)NR^{6c}R^{7c} wherein R^{6c} and R^{7c} are independently selected from H, C₁₋₆ alkyl optionally substituted with a halogen, and cyclopropyl optionally substituted with a halogen;
B¹ is selected from the group consisting of a heteroaryl, an aryl and a heterocycloalkyl, optionally substituted with one or more groups selected from a) C₁₋₆ alkyl optionally substituted with one or more of C₁₋₆ alkyl, amino, CN, halogen, hydroxy, C₁₋₆ alkoxy, carboxy, alkoxycarbonyl, H₂NCO, b) R¹³—C₁₋₆ alkyl, c) C₃₋₆ cycloalkyl optionally substituted with one or more of C₁₋₆ alkyl, amino, CN, halogen, or hydroxy, d) C₁₋₆ alkoxy, e) C₁₋₆ alkylthio, f) C₁₋₆ alkylsulfonyl, g) carbonyl substituted with any one of hydroxy, C₁₋₆ alkoxy, C₁₋₆ alkylNH, ((R¹⁴)(R¹⁵)N)C₁₋₆ alkylNH, or (pyridinyl)C₁₋₆ alkylNH, h) (R¹⁶)(R¹⁷)N, i) C₂-alkynyl, j) R¹³, and k) C₁₋₆ alkenyl,
wherein R¹³ is selected from any one of a) phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of cyano, nitro, OH, C₂-alkynyl, halogen, C₁₋₆ alkyl, halo-C₁₋₆ alkyl, C₃₋₆ cycloalkyl, C₁₋₆ alkoxy, halo-C₁₋₆ alkoxy, C₁₋₆ alkylthio, carboxy, C₁₋₆ alkoxycarbonyl, CONH₂, and (R¹⁸)(R¹⁹)N; or b) (C₁₋₆ alkyl-S02)phenyl, (C₁₋₆ alkyl SO₂)(halo)phenyl, (aminoSO₂)phenyl, (di-C₁₋₆ alkylaminoSO₂)phenyl, ((C₁₋₆ alkyl-NHSO₂)—C₁₋₆ alkyl)phenyl, (pyrrolyl)phenyl, (imidazolyl)phenyl, (oxazolyl)phenyl, (tetrazolyl)phenyl, ((pyridinyl)methyl)phenyl, phenoxyphenyl, (benzyloxy)phenyl, ((methyl)thiazolyl)-phenyl, (thiazolyl)-benzenesulfamido, ((methyl)thiadiazolyl)benzenesulfamido, (methyl)-benzothiazolonyl, or fluoropyrazolopyrimidinyl;
wherein
R¹⁴ is hydrogen or C₁₋₆ alkyl;
R¹⁵ is hydrogen or C₁₋₆ alkyl; or
(R¹⁴)(R¹⁵)N taken together is any one of azetidinyl, pyrrolidinyl, piperidinyl, piperazinyl, or morpholinyl, optionally substituted with one or more substituents selected from halogen, C₁₋₆ alkyl, and hydroxy;
R¹⁶ is hydrogen, C₁₋₆ alkyl, C₁₋₆ alkylcarbonyl, or C₁₋₆ alkylsulfonyl;
R¹⁷ is hydrogen or C₁₋₆ alkyl; or
(R¹⁶)(R¹⁷)N taken together is any one of azetidinyl, pyrrolidinyl, piperidinyl, piperazinyl, or morpholinyl, optionally substituted with one or more substituents selected from halogen, C₁₋₆ alkyl, and C₁₋₆ alkylcarbonyl;
R¹⁸ is hydrogen, C₁₋₆ alkyl, C₁₋₆ alkylcarbonyl, or C₁₋₆ alkylsulfonyl;
R¹⁹ is hydrogen or C₁₋₆ alkyl; or
(R¹⁸)(R¹⁹)N taken together is any one of azetidinyl, pyrrolidinyl, piperidinyl, piperazinyl, or morpholinyl, optionally substituted with one or more substituents selected from halogen, C₁₋₆ alkyl, and C₁₋₆ alkylcarbonyl;
R¹ is selected from the group consisting of a) H, b) OH, c) OC₁₋₆ alkyl optionally substituted with one or more halogen, phenyl, phenyl substituted with one or more groups selected form OH and halogen, CN, OR²⁰, NR²¹R²², and CONH₂, wherein R²⁰ is selected from the group consisting of H, CN, a halogen, methyl optionally substituted with a F, OCH₃ optionally substituted with a F, OCH₂CH₃ optionally substituted with a F, OH, and R²³—CONH— wherein R²³ is selected from C₁₋₃ alkyl and cyclopropyl, R²¹ is selected from the group consisting of H, CN, a halogen, methyl optionally substituted with a F, OCH₃ optionally substituted with a F, OCH₂CH₃ optionally substituted with a F, OH, and R²⁴—CONH— wherein R²⁴ is selected from C₁₋₃ alkyl and cyclopropyl, and R²² is selected from the group consisting of H, CN, a halogen, methyl optionally substituted with a F, OCH₃ optionally substituted with a F, OCH₂CH₃ optionally substituted with a F, OH, and R²⁵—CONH— wherein R²⁵ is selected from C₁₋₃ alkyl and cyclopropyl, d) branched OC₃₋₆ alkyl optionally substituted with one or more halogen, CN, OR²⁶, NR²⁷R²⁸, and CONH₂, wherein R²⁶ is selected from the group consisting of H, CN, a halogen, methyl optionally substituted with a F, OCH₃ optionally substituted with a F, OCH₂CH₃ optionally substituted with a F, OH, and R²⁹—CONH— wherein R²⁹ is selected from C₁₋₃ alkyl and cyclopropyl, R²⁷ is selected from the group consisting of H, CN, a halogen, methyl optionally substituted with a F, OCH₃ optionally substituted with a F, OCH₂CH₃ optionally substituted with a F, OH, and R³⁰—CONH— wherein R^{3c} is selected from C₁₋₃ alkyl and cyclopropyl, and R²⁸ is selected from the group consisting of H, CN, a halogen, methyl optionally substituted with a F, OCH₃ optionally substituted with a F, OCH₂CH₃ optionally substituted with a F, OH, and R³¹—CONH— wherein R³¹ is selected from C₁₋₃ alkyl and cyclopropyl, e) cyclic OC₃₋₆ alkyl optionally substituted with one or more halogen, CN, OR³², NR³³R³⁴, and CONH₂, wherein R³² is selected from the group consisting of H, CN, a halogen, methyl optionally substituted with a F, OCH₃ optionally substituted with a F, OCH₂CH₃ optionally substituted with a F, OH, and R³⁵—CONH— wherein R³⁵ is selected from C₁₋₃ alkyl and cyclopropyl, R³³ is selected from the group consisting of H, CN, a halogen, methyl optionally substituted with a F, OCH₃ optionally substituted with a F, OCH₂CH₃ optionally substituted with a F, OH, and R³⁶—CONH— wherein R³⁶ is selected from C₁₋₃ alkyl and cyclopropyl, and R³⁴ is selected from the group consisting of H, CN, a halogen, methyl optionally substituted with a F, OCH₃ optionally substituted with a F, OCH₂CH₃ optionally substituted with a F, OH, and R³⁷—CONH— wherein R³⁷ is selected from C₁₋₃ alkyl and cyclopropyl, and f) a fluorine (F); or a pharmaceutically acceptable salt or solvate thereof.

In a further aspect the present invention concerns a β-D-galactopyranose compound of formula (I)

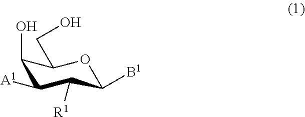

wherein
the pyranose ring is β-D-galactopyranose,
A1 is

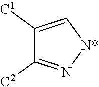

wherein the asterix * indicates the nitrogen atom of the triazole ring that is covalently attached to the galactopyranose;
C¹ is selected from a) an aryl, such as phenyl or naphthyl, optionally substituted with a group selected from a halogen; CN; OH; a spiro heterocycle; —COOH; —CONR²R³, wherein R² and R³ are independently selected from H, C₁₋₃ alkyl, and cyclopropyl, or R² and R³ together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR⁴R⁵, wherein R⁴ and R⁵ are independently selected from H, C₁₋₃ alkyl and cyclopropyl; C(═O)—R^{11a}, wherein R^{11a} is selected from H and C₁₋₃ alkyl; and R⁶—CONH— wherein R⁶ is selected from C₁₋₃ alkyl and cyclopropyl; or b) a heterocycle, such as heteroaryl or heterocycloalkyl, optionally substituted with a group selected from a halogen; a spiro heterocycle; CN; OH; —COOH; —CONR⁷R⁸, wherein R⁷ and R⁸ are independently selected from H, C₁₋₃ alkyl, and cyclopropyl, or R⁷ and R⁸ together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR⁹R¹⁰, wherein R⁹ and R¹⁰ are independently selected from H, C₁₋₃ alkyl, and cyclopropyl; C(═O)—R¹¹, wherein
R¹¹ is selected from H and C₁₋₃ alkyl; and R¹²—CONH— wherein R¹² is selected from C₁₋₃ alkyl and cyclopropyl;
C² is selected from the group consisting of
a) H; C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{4a}R^{5a}, wherein R^{4a} and R^{5a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{2a}R^{3a}, wherein R^{2a} and R^{3a} are independently selected from H, C₁₋₃ alkyl optionally substituted with a halogen, and cyclopropyl optionally substituted with a halogen; and S(O₂)NR^{6a}R^{7a} wherein R^{6a} and R^{7a} are independently selected from H, C₁₋₆ alkyl optionally substituted with a halogen, and cyclopropyl optionally substituted with a halogen;
b) phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{4b}R^{5b}, wherein R^{4b} and R^{5b} are independently selected from H, C₁₋₆ alkyl optionally substituted with a halogen, C₃_6 cycloalkyl optionally substituted with a halogen, C(O)C₁₋₆ alkyl optionally substituted with a halogen, and S(O₂)C₁₋₆ alkyl optionally substituted with a halogen; C(O)C₃_6 cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{2b}R^{3b}, wherein R^{2b} and R^{3b} are independently selected from H, C₁₋₃ alkyl optionally substituted with a halogen, and cyclopropyl optionally substituted with a halogen; and S(O₂)NR^{6b}R^{7b} wherein R^{6b} and R^{7b} are independently selected from H, C₁₋₆ alkyl optionally substituted with a halogen, and cyclopropyl optionally substituted with a halogen;
c) Y—Z wherein
Y is linked to the pyrazole of A¹ and is selected from the group consisting of S, Se, SO, SO₂, O, C═O, and CR^{2c}R^{3c} wherein R^{2c} and R^{3c} are independently selected from hydrogen, OH, or halogen; and
Z is selected from the group consisting of phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{4c}R^{5c}, wherein R^{4c} and R^{5c} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{2d}R^{3d}, wherein R^{2d} and R^{3d} are independently selected from H, C₁₋₃ alkyl optionally substituted with a halogen, and cyclopropyl optionally substituted with a halogen; and S(O₂)NR^{6c}R^{7c} wherein R^{6c} and R^{7c} are independently selected from H, C₁₋₆ alkyl optionally substituted with a halogen, and cyclopropyl optionally substituted with a halogen;
B¹ is selected from the group consisting of a heteroaryl, an aryl and a heterocycloalkyl, optionally substituted with one or more groups selected from a) C₁₋₆ alkyl optionally substituted with one or more of C₁₋₆ alkyl, amino, CN, halogen, hydroxy, C₁₋₆ alkoxy, carboxy, alkoxycarbonyl, H₂NCO, b) R¹³—C₁₋₆ alkyl, c) C₃₋₆ cycloalkyl optionally substituted with one or more of C₁₋₆ alkyl, amino, CN, halogen, or hydroxy, d) C₁₋₆ alkoxy, e) C₁₋₆ alkylthio, f) C₁₋₆ alkylsulfonyl, g) carbonyl substituted with any one of hydroxy, C₁₋₆ alkoxy, C₁₋₆ alkylNH, ((R¹⁴)(R¹⁵)N)C₁₋₆ alkylNH, or (pyridinyl)C₁₋₆ alkylNH, h) (R¹⁶)(R¹⁷)N, i) C₂-alkynyl, j) R¹³, and k) C₁₋₆ alkenyl.
wherein R¹³ is selected from any one of a) phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of cyano, OH, C₂-alkynyl, halogen, C₁₋₆ alkyl, halo-C₁₋₆ alkyl, C₃_6 cycloalkyl, C₁₋₆ alkoxy, halo-C₁₋₆ alkoxy, C₁₋₆ alkylthio, carboxy, C₁₋₆ alkoxycarbonyl, CONH₂, and (R¹⁸)(R¹⁹)N; or b) (pyrrolyl)phenyl, (imidazolyl)phenyl, (oxazolyl)phenyl, (tetrazolyl)phenyl, ((pyridinyl)methyl)phenyl, phenoxyphenyl, (benzyloxy)phenyl, ((methyl)thiazolyl)-phenyl, (thiazolyl)-benzenesulfamido, ((methyl)thiadiazolyl)benzenesulfamido, (methyl)-benzothiazolonyl, or fluoropyrazolopyrimidinyl;
wherein
R¹⁴ is hydrogen or C₁₋₆ alkyl;
R¹⁵ is hydrogen or C₁₋₆ alkyl; or
(R¹⁴)(R¹⁵)N taken together is any one of azetidinyl, pyrrolidinyl, piperidinyl, piperazinyl, or morpholinyl, optionally substituted with one or more substituents selected from halogen, C₁₋₆ alkyl, and hydroxy;
R¹⁶ is hydrogen, C₁₋₆ alkyl, C₁₋₆ alkylcarbonyl, or C₁₋₆ alkylsulfonyl;
R¹⁷ is hydrogen or C₁₋₆ alkyl; or
(R¹⁶)(R¹⁷)N taken together is any one of azetidinyl, pyrrolidinyl, piperidinyl, piperazinyl, or morpholinyl, optionally substituted with one or more substituents selected from halogen, C₁₋₆ alkyl, and C₁₋₆ alkylcarbonyl;
R¹⁸ is hydrogen, C₁₋₆ alkyl, C₁₋₆ alkylcarbonyl, or C₁₋₆ alkylsulfonyl;
R¹⁹ is hydrogen or C₁₋₆ alkyl; or
(R¹⁸)(R¹⁹)N taken together is any one of azetidinyl, pyrrolidinyl, piperidinyl, piperazinyl, or morpholinyl, optionally substituted with one or more substituents selected from halogen, C₁₋₆ alkyl, and C₁₋₆ alkylcarbonyl;
R¹ is selected from the group consisting of a) H, b) OH, c) OC₁₋₆ alkyl optionally substituted with one or more halogen, phenyl, phenyl substituted with one or more groups selected form OH and halogen, CN, OR²⁰, NR²¹R²², and CONH₂, wherein R²⁰ is selected from the group consisting of H, CN, a halogen, methyl optionally substituted with a F, OCH₃ optionally substituted with a F, OCH₂CH₃ optionally substituted with a F, OH, and R²³—CONH— wherein R²³ is selected from C₁₋₃ alkyl and cyclopropyl, R²¹ is selected from the group consisting of H, CN, a halogen, methyl optionally substituted with a F, OCH₃ optionally substituted with a F, OCH₂CH₃ optionally substituted with a F, OH, and R²⁴—CONH— wherein R²⁴ is selected from C₁₋₃ alkyl and cyclopropyl, and R²² is selected from the group consisting of H, CN, a halogen, methyl optionally substituted with a F, OCH₃ optionally substituted with a F, OCH₂CH₃ optionally substituted with a F, OH, and R²⁵—CONH— wherein R²⁵ is selected from C₁₋₃ alkyl and cyclopropyl, d) branched OC₃₋₆ alkyl optionally substituted with one or more halogen, CN, OR²⁶, NR²⁷R²⁸, and CONH₂, wherein R²⁶ is selected from the group consisting of H, CN, a halogen, methyl optionally substituted with a F, OCH₃ optionally substituted with a F, OCH₂CH₃ optionally substituted with a F, OH, and R²⁹—CONH— wherein R²⁹ is selected from C₁₋₃ alkyl and cyclopropyl, R²⁷ is selected from the group consisting of H, CN, a halogen, methyl optionally substituted with a F, OCH₃ optionally substituted with a F, OCH₂CH₃ optionally substituted with a F, OH, and R³⁰—CONH— wherein R^{3c} is selected from C₁₋₃ alkyl and cyclopropyl, and R²⁸ is selected from the group consisting of H, CN, a halogen, methyl optionally substituted with a F, OCH₃ optionally substituted with a F, OCH₂CH₃ optionally substituted with a F, OH, and R³¹—CONH— wherein R³¹ is selected from C₁₋₃ alkyl and cyclopropyl, e) cyclic OC₃₋₆ alkyl optionally substituted with one or more halogen, CN, OR³², NR³³R³⁴, and CONH₂, wherein R³² is selected from the group consisting of H, CN, a halogen, methyl optionally substituted with a F, OCH₃ optionally substituted with a F, OCH₂CH₃ optionally substituted with a F, OH, and R³⁵—CONH— wherein R³⁵ is selected from C₁₋₃ alkyl and cyclopropyl, R³³ is selected from the group consisting of H, CN, a halogen, methyl optionally substituted with a F, OCH₃ optionally substituted with a F, OCH₂CH₃ optionally substituted with a F, OH, and R³⁶—CONH— wherein R³⁶ is selected from C₁₋₃ alkyl and cyclopropyl, and R³⁴ is selected from the group consisting of H, CN, a halogen, methyl optionally substituted with a F, OCH₃ optionally substituted with a F, OCH₂CH₃ optionally substituted with a F, OH, and R³⁷—CONH— wherein R³⁷ is selected from C₁₋₃ alkyl and cyclopropyl, and f) a fluorine (F); or a pharmaceutically acceptable salt or solvate thereof.

In an embodiment C¹ is a phenyl, optionally substituted with a group selected from a halogen; CN; —COOH; —CONR²R³, wherein R² and R³ are independently selected from H, C₁₋₃ alkyl, cyclopropyl, and iso-propyl, or R² and R³ together with the nitrogen may form a heterocycloalkyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR⁴R⁵, wherein R⁴ and R⁵ are independently selected from H, C₁₋₃ alkyl and isopropyl; OH; and R⁶—CONH— wherein R⁶ is selected from C₁₋₃ alkyl and cyclopropyl; or b) a heterocycle, such as heteroaryl or heterocycloalkyl, optionally substituted with a group selected from a halogen; a spiro heterocycle; CN; —COOH; —CONR⁷R⁸, wherein R⁷ and R⁸ are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR⁹R¹⁰, wherein R⁹ and R¹⁰ are independently selected from H, C₁₋₃ alkyl, cyclopropyl and isopropyl, C(═O)—R¹¹, wherein R¹¹ is selected from H and C₁₋₃ alkyl; OH; and R¹²—CONH— wherein R¹² is selected from C₁₋₃ alkyl and cyclopropyl.

In a further embodiment C¹ is a phenyl, optionally substituted with a group selected from a halogen; CN; —COOH; —CONR²R³, wherein R² and R³ are independently selected from H, C₁₋₃ alkyl, cyclopropyl, and iso-propyl, or R² and R³ together with the nitrogen may form a heterocycloalkyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR⁴R⁵, wherein R⁴ and R⁵ are independently selected from H, C₁₋₃ alkyl and isopropyl; OH; and R⁶—CONH— wherein R⁶ is selected from C₁₋₃ alkyl and cyclopropyl; provided that both ortho carbon positions of the phenyl ring in relation to the covalent bond to the pyrazol ring have hydrogens attached to the carbon atoms, or b) a heterocycle, such as heteroaryl or heterocycloalkyl, optionally substituted with a group selected from a halogen; a spiro heterocycle; CN; —COOH; —CONR⁷R⁸, wherein R⁷ and R⁸ are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR⁹R¹⁰, wherein R⁹ and R¹⁰ are independently selected from H, C₁₋₃ alkyl, cyclopropyl and isopropyl, C(═O)—R¹¹, wherein R¹¹ is selected from H and C₁₋₃ alkyl; OH; and R¹²—CONH— wherein R¹² is selected from C₁₋₃ alkyl and cyclopropyl; provided that ortho carbon positions of the heterocycle ring in relation to the covalent bond to the pyrazol ring have hydrogens attached to the carbon atoms.

In a further embodiment C¹ is a phenyl substituted with a halogen, such one to three from the group consisting of F, Cl, Br and I. Preferably, provided that both ortho carbon positions of the phenyl ring in relation to the covalent bond to the pyrazol ring have hydrogens attached to the carbon atoms.

In another embodiment C¹ is a heteroaryl, optionally substituted with a group selected from a halogen; a spiro heterocycle; CN; —COOH; —CONR⁷R⁸, wherein R⁷ and R⁸ are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR⁹R¹⁰, wherein R⁹ and R¹⁰ are independently selected from H, C₁₋₃ alkyl, cyclopropyl and isopropyl, C(═O)—R¹¹, wherein R¹¹ is selected from H and C₁₋₃ alkyl; OH; and R¹²—CONH— wherein R¹² is selected from C₁₋₃ alkyl and cyclopropyl. Preferably, provided that ortho carbon positions of the heteroaryl ring in relation to the covalent bond to the pyrazol ring have hydrogens attached to the carbon atoms.

In a further embodiment C₁ is a pyridinyl substituted with a halogen, such one to three form the group consisting of F, Cl, Br and I. Preferably, provided that ortho carbon positions of the pyridinyl ring in relation to the covalent bond to the pyrazol ring have hydrogens attached to the carbon atoms.

In a still further embodiment C² is selected from H and C₁₋₄ alkyl, such as H and methyl.

In a further embodiment B1 is selected from the group consisting of pyrrolyl, furanyl, thienyl, pyrazolyl, isoxazolyl, isothiazolyl, imidazolyl, oxazolyl, thiazolyl, triazolyl, oxadiazolyl, thiadiazolyl, phenyl, or indolyl; optionally substituted with a group selected from a) C₁₋₆ alkyl optionally substituted with one or more of C₁₋₆ alkyl, halogen, hydroxy, C₁₋₆ alkoxy, carboxy, alkoxycarbonyl, H₂NCO, b) R¹³—C₁₋₆ alkyl, c) C₃₋₆ cycloalkyl optionally substituted with one or more of C₁₋₆ alkyl, halogen, or hydroxy, d) C₁₋₆ alkoxy, e) C₁₋₆ alkylthio, f) C₁₋₆ alkylsulfonyl, g) carbonyl substituted with any one of hydroxy, C₁₋₆ alkoxy, C₁₋₆ alkylNH, ((R¹⁴)(R¹⁵)N)C₁₋₆ alkylNH, or (pyridinyl)C₁₋₆ alkylNH, h) (R¹⁶)(R¹⁷)N, j) R¹³, and k) C₁₋₆ alkenyl, wherein R¹³, R¹⁴, R¹⁵, R¹⁶ and R¹⁷ are as defined above under the definitions for the compound of formula (I).

In a still further embodiment B1 is triazolyl substituted with a group selected from a) C₁₋₆ alkyl optionally substituted with one or more of halogen, and j) R¹³; wherein R¹³ is selected from the group consisting of phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolo[4,5-b]pyridinyl, thiazolyl, triazolyl, oxadiazolyl, or thiadiazolyl; optionally substituted with one or more substituents selected from the group consisting of cyano, nitro, OH, C₂-alkynyl, halogen, C₁₋₆ alkyl, halo-C₁₋₆ alkyl, C₃₋₆ cycloalkyl, C₁₋₆ alkoxy, halo-C₁₋₆ alkoxy, C₁₋₆ alkylthio, carboxy, C₁₋₆ alkoxycarbonyl, and CONH₂.

In a further embodiment B1 is triazolyl substituted with a group selected from a) C₁₋₆ alkyl optionally substituted with one or more of halogen, and j) R¹³; wherein R¹³ is selected from the group consisting of phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of cyano, nitro, OH, C₂-alkynyl, halogen, C₁₋₆ alkyl, halo-C₁₋₆ alkyl, C₃₋₆ cycloalkyl, C₁₋₆ alkoxy, halo-C₁₋₆ alkoxy, C₁₋₆ alkylthio, carboxy, C₁₋₆ alkoxycarbonyl, and CONH₂.

In a further embodiment R¹ is selected from H, OH, OC₁₋₄ alkyl, such as O-methyl, O-ethyl, or O-isopropyl, or OC₁₋₄ alkyl substituted with at least one from the group consisting of phenyl and phenyl substituted with one or more groups selected from OH and halogen. Typically, R¹ is selected from OH and OC₁₋₃ alkyl.

In a further aspect the present invention concerns a β-D-galactopyranose compound of formula (I) selected from any one of the group consisting of:
3-{5-{3-[4-(4-Chloro-3,5-difluorophenyl)-1H-1,2-pyrazol-1-yl]-3-deoxy-3-D-galactopyranosyl}-3-methyl-1H-1,2,4-triazol-1-yl}-5-chloro-2-cyclopropylpyridine,
6-{3-{3-Deoxy-3-[4-(3,4,5-trifluorophenyl)-1H-1,2-pyrazol-1-yl]-β-D-galactopyranosyl}-4H-1,2,4-triazol-4-yl}-2-methylbenzothiazole,
5-Chloro-1-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-(trifluoromethyl)benzene,
1-{5-{3-Deoxy-3-[4-(3,4,5-trifluorophenyl)-1H-1,2-pyrazol-1-yl]-β-D-galactopyranosyl}-3-methyl-1H-1,2,4-triazol-1-yl}-2-(trifluoromethyl)-2-(3,4,5-trifluorophenyl)benzene,
1-{5-{3-[4-(4-Chloro-3,5-difluorophenyl)-1H-1,2-pyrazol-1-yl]-3-deoxy-β-D-galactopyranosyl}-3-methyl-1H-1,2,4-triazol-1-yl}-5-chloro-2-(trifluoromethyl)benzene,
5-Chloro-1-{5-{3-deoxy-3-[4-(6-fluoropyridin-2-yl)-1H-1,2-pyrazol-1-yl]-β-D-galactopyranosyl}-3-methyl-1H-1,2,4-triazol-1-yl}-2-(trifluoromethyl)benzene,
5-Chloro-1-{5-{3-deoxy-3-[4-(4-fluoropyridin-2-yl)-1H-1,2-pyrazol-1-yl]-β-D-galactopyranosyl}-3-methyl-1H-1,2,4-triazol-1-yl}-2-(trifluoromethyl)benzene,
6-{5-{3-Deoxy-3-[4-(3,4,5-trifluorophenyl)-1H-1,2-pyrazol-1-yl]-β-D-galactopyranosyl}-3-methyl-1H-1,2,4-triazol-1-yl}-2-methylbenzothiazole,
6-{5-{3-[4-(4-Chloro-3,5-difluorophenyl)-1H-1,2-pyrazol-1-yl]-3-deoxy-β-D-galactopyranosyl}-3-methyl-1H-1,2,4-triazol-1-yl}-2-methylbenzothiazole,
1-{3-{3-[4-(4-Chloro-3,5-difluorophenyl)-1H-1,2-pyrazol-1-yl]-3-deoxy-β-D-galactopyranosyl}-5-methyl-4H-1,2,4-triazol-4-yl}-5-chloro-2-(trifluoromethyl)benzene,
1-{3-{3-[4-(4-Chloro-3,5-difluorophenyl)-1H-1,2-pyrazol-1-yl]-3-deoxy-β-D-galactopyranosyl}-5-methyl-4H-1,2,4-triazol-4-yl}-5-chloro-2-(trifluoromethyl)benzene, and
1-{5-{3-[4-(4-Chloro-3,5-difluorophenyl)-3-methyl-1H-1,2-pyrazol-1-yl]-3-deoxy-β-D-galactopyranosyl}-3-methyl-1H-1,2,4-triazol-1-yl}-5-chloro-2-(trifluoromethyl)benzene; or a pharmaceutically acceptable salt or solvat thereof.

In a still further aspect the present invention concerns a β-D-galactopyranose compound of formula (I) selected from any one of the group consisting of:
5-Chloro-3-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-cyclopropylpyridine,
1-{5-{3-[4-(5-Chloro-4-fluoropyridin-2-yl)-1H-1,2-pyrazol-1-yl]-3-deoxy-β-D-galactopyranosyl}-3-methyl-1H-1,2,4-triazol-1-yl}-5-chloro-2-(trifluoromethyl)benzene,
5-Chloro-1-{5-{3-deoxy-3-[4-(4-fluoro-5-methylpyridin-2-yl)-1H-1,2-pyrazol-1-yl]-β-D-galactopyranosyl}-3-methyl-1H-1,2,4-triazol-1-yl}-2-(trifluoromethyl)benzene,
1-{5-{3-[4-(5-Chloro-4-methylpyridin-2-yl)-1H-1,2-pyrazol-1-yl]-3-deoxy-β-D-galactopyranosyl}-3-methyl-1H-1,2,4-triazol-1-yl}-5-chloro-2-(trifluoromethyl)benzene,
5-Chloro-1-{5-{3-deoxy-3-[4-(4-ethynyl-3,5-difluorophenyl)-1H-1,2-pyrazol-1-yl]-β-D-galactopyranosyl}-3-methyl-1H-1,2,4-triazol-1-yl}-2-(trifluoromethyl)benzene,
5-Chloro-1-{5-{3-deoxy-3-{4-[3,5-difluoro-4-(2-propyn-1-yl)phenyl]-1H-1,2-pyrazol-1-yl}-β-D-galactopyranosyl}-3-methyl-1H-1,2,4-triazol-1-yl}-2-(trifluoromethyl)benzene,
5-Bromo-3-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}pyridine,
5-Chloro-3-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-(trifluoromethyl)pyridine,
5-Chloro-2-cyano-3-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}pyridine,
5-Bromo-2-cyano-3-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}pyridine,
3-Chloro-2-cyano-5-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}pyridine,
5-Bromo-1-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-(trifluoromethyl)benzene,
5-Bromo-3-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-(trifluoromethyl)pyridine, and
5-Bromo-2-cyano-1-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}benzene; or a pharmaceutically acceptable salt or solvat thereof.

In a still further aspect the present invention concerns a β-D-galactopyranose compound of formula (I) selected from any one of the group consisting of:
5-Chloro-2-cyano-1-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}benzene,
3-{5-{3-[4-(4-Chloro-3,5-difluorophenyl)-1H-1,2-pyrazol-1-yl]-3-deoxy-β-D-galactopyranosyl}-3-methyl-1H-1,2,4-triazol-1-yl}-5-chloro-2-(trifluoromethyl)pyridine,
1-{5-{3-[4-(4-Chloro-3,5-difluorophenyl)-3-ethoxycarbonyl-1H-1,2-pyrazol-1-yl]-3-deoxy-β-D-galactopyranosyl}-3-methyl-1H-1,2,4-triazol-1-yl}-5-chloro-2-(trifluoromethyl)benzene,
1-{5-{3-[3-Carboxy-4-(4-chloro-3,5-difluorophenyl)-1H-1,2-pyrazol-1-yl]-3-deoxy-β-D-galactopyranosyl}-3-methyl-1H-1,2,4-triazol-1-yl}-5-chloro-2-(trifluoromethyl)benzene,
1-{5-{3-[4-(4-Chloro-3,5-difluorophenyl)-3-cyano-1H-1,2-pyrazol-1-yl]-3-deoxy-β-D-galactopyranosyl}-3-methyl-1H-1,2,4-triazol-1-yl}-5-chloro-2-(trifluoromethyl)benzene,
1-{5-{3-[3-Aminocarbonyl-4-(4-chloro-3,5-difluorophenyl)-1H-1,2-pyrazol-1-yl]-3-deoxy-β-D-galactopyranosyl}-3-methyl-1H-1,2,4-triazol-1-yl}-5-chloro-2-(trifluoromethyl)benzene,
5-Chloro-2-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-thiazole,
6-{5-{3-Deoxy-3-[4-(3,4,5-trifluorophenyl)-1H-1,2-pyrazol-1-yl]-β-D-galactopyranosyl}-3-methyl-1H-1,2,4-triazol-1-yl}benzothiazole,
6-{5-{3-Deoxy-3-[4-(3,4,5-trifluorophenyl)-1H-1,2-pyrazol-1-yl]-β-D-galactopyranosyl}-3-methyl-1H-1,2,4-triazol-1-yl}-2-ethylbenzothiazole,
6-{5-{3-Deoxy-3-[4-(3,4,5-trifluorophenyl)-1H-1,2-pyrazol-1-yl]-β-D-galactopyranosyl}-3-methyl-1H-1,2,4-triazol-1-yl}-2-(trifluoromethyl)benzothiazole,
6-{5-{3-Deoxy-3-[4-(3,4,5-trifluorophenyl)-1H-1,2-pyrazol-1-yl]-β-D-galactopyranosyl}-3-methyl-1H-1,2,4-triazol-1-yl}-5-fluoro-2-methylbenzothiazole,
6-{5-{3-Deoxy-3-[4-(3,4,5-trifluorophenyl)-1H-1,2-pyrazol-1-yl]-β-D-galactopyranosyl}-3-methyl-1H-1,2,4-triazol-1-yl}-7-fluoro-2-methylbenzothiazole,
6-{5-{3-Deoxy-3-[4-(3,4,5-trifluorophenyl)-1H-1,2-pyrazol-1-yl]-β-D-galactopyranosyl}-3-methyl-1H-1,2,4-triazol-1-yl}-4-fluoro-2-methylbenzothiazole,
7-Chloro-6-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-methylbenzothiazole,
4-Chloro-6-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-methylbenzothiazole,
4-Cyano-6-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-methylbenzothiazole,
5-Bromo-6-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-methylbenzothiazole,
5-Cyano-6-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-methylbenzothiazole,
6-{5-{3-Deoxy-3-[4-(3,4,5-trifluorophenyl)-1H-1,2-pyrazol-1-yl]-β-D-galactopyranosyl}-3-methyl-1H-1,2,4-triazol-1-yl}-2-methylthiazolo[4,5-b]pyridine,
5-Cyano-1-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-(trifluoromethyl)benzene,
5-Chloro-1-{5-{3-deoxy-3-[4-(3,4,5-trifluorophenyl)-3-phenyl-1H-1,2-pyrazol-1-yl]-β-D-galactopyranosyl}-3-methyl-1H-1,2,4-triazol-1-yl}-2-(trifluoromethyl)benzene,
5-Chloro-1-{5-{3-deoxy-3-[4-(3,4,5-trifluorophenyl)-3-(2-fluorophenyl)-1H-1,2-pyrazol-1-yl]-β-D-galactopyranosyl}-3-methyl-1H-1,2,4-triazol-1-yl}-2-(trifluoromethyl)benzene,
5-Chloro-1-{5-{3-deoxy-3-[4-(3,4,5-trifluorophenyl)-3-methyl-1H-1,2-pyrazol-1-yl]-β-D-galactopyranosyl}-3-methyl-1H-1,2,4-triazol-1-yl}-2-(trifluoromethyl)benzene,
1-{5-{3-[3-Benzyl-4-(3,4,5-trifluorophenyl)-1H-1,2-pyrazol-1-yl]-3-deoxy-β-D-galactopyranosyl}-3-methyl-1H-1,2,4-triazol-1-yl}-5-chloro-2-(trifluoromethyl)benzene,
5-Chloro-1-{5-{3-[3-cyano-4-(3,4,5-trifluorophenyl)-1H-1,2-pyrazol-1-yl]-3-deoxy-β-D-galactopyranosyl}-3-methyl-1H-1,2,4-triazol-1-yl}-2-(trifluoromethyl)benzene,
5-Chloro-1-{5-{3-deoxy-3-[4-(3,4,5-trifluorophenyl)-3-(2-methoxyphenyl)-1H-1,2-pyrazol-1-yl]-β-D-galactopyranosyl}-3-methyl-1H-1,2,4-triazol-1-yl}-2-(trifluoromethyl)benzene,
5-Chloro-1-{5-{3-deoxy-3-[4-(3,4,5-trifluorophenyl)-3-(2-methylphenyl)-1H-1,2-pyrazol-1-yl]-β-D-galactopyranosyl}-3-methyl-1H-1,2,4-triazol-1-yl}-2-(trifluoromethyl)benzene,
5-Chloro-1-{5-{3-deoxy-3-[4-(3,4,5-trifluorophenyl)-3-(3-fluorophenyl)-1H-1,2-pyrazol-1-yl]-β-D-galactopyranosyl}-3-methyl-1H-1,2,4-triazol-1-yl}-2-(trifluoromethyl)benzene,
5-Chloro-1-{5-{3-deoxy-3-[4-(3,4,5-trifluorophenyl)-3-(4-fluorophenyl)-1H-1,2-pyrazol-1-yl]-β-D-galactopyranosyl}-3-methyl-1H-1,2,4-triazol-1-yl}-2-(trifluoromethyl)benzene,
5-Chloro-1-{5-{3-deoxy-3-{4-[4-(trifluoromethyl)phenyl]-1H-1,2-pyrazol-1-yl}-β-D-galactopyranosyl}-3-methyl-1H-1,2,4-triazol-1-yl}-2-(trifluoromethyl)benzene,
5-Chloro-1-{5-{3-deoxy-3-[4-(3-fluoro-4-methylphenyl)-1H-1,2-pyrazol-1-yl]-β-D-galactopyranosyl}-3-methyl-1H-1,2,4-triazol-1-yl}-2-(trifluoromethyl)benzene,
1-{5-{3-[4-(3-Chlorophenyl)-1H-1,2-pyrazol-1-yl]-3-deoxy-β-D-galactopyranosyl}-3-methyl-1H-1,2,4-triazol-1-yl}-5-chloro-2-(trifluoromethyl)benzene, and
1-{5-{3-[4-(4-Chlorophenyl)-1H-1,2-pyrazol-1-yl]-3-deoxy-β-D-galactopyranosyl}-3-methyl-1H-1,2,4-triazol-1-yl}-5-chloro-2-(trifluoromethyl)benzene; or a pharmaceutically acceptable salt or solvate thereof.

In a further aspect the present invention relates to a compound of formula (I) for use as a medicine.

In a still further aspect, the present invention relates to a pharmaceutical composition comprising the compound of any one of the previous claims and optionally a pharmaceutically acceptable additive, such as a carrier and/or excipient.

In a further aspect the present invention relates to a compound of formula (I) of the present invention for use in a method for treating a disorder relating to the binding of a galectin-3 to a ligand in a mammal, such as a human. In a further embodiment the disease or disorder is selected from the group consisting of inflammation, such as acute post myocardial infarctions (MI), acute coronary syndrome, acute stent occlusion, acute myocardial reperfusion injury, acute pneumonitidies, acute lung injury (ALI), acute kidney injury (AKI), acute hepatitis, acute on chronic liver failure, acute alcohol hepatitis, acute pancreatitis, acute uveitis, acute pancreatitis related liponecrosis, acute retinitis, acute nephritis, acute myocarditis, chronic autoimmune diseases in all organs, (e.g. lung, liver, kidney, heart, skin, muscle, gut), chronic bacterial infections, chronic viral related inflammation; fibrosis, such as pulmonary fibrosis, liver fibrosis, kidney fibrosis, ophthalmological fibrosis and fibrosis of the skin and heart, acute post-surgical ocular fibrosis, acute transplantation rejection of the kidney, heart, lung, liver, and pancreas, acute post explosion/improvised explosive devices, acute post toxic dust (such as dust from terror attack known as 9/11), acute chemical exposure, chronic lung fibrosis, interstitial lung fibrosis (IPF), Interstitial Lung Disease (ILD), Childhood ILD (ChILD); chronic liver fibrosis, chronic alcohol fibrosis, chronic viral fibrosis, chronic diabetic fibrosis, diabetic nephropathy, chronic glomerulonephritis, renal artery stenosis, endometriosis; scarring; keloid formation; aberrant scar formation; surgical adhesions; scleroderma; systemic sclerosis; septic shock; cancers, such as carcinomas, sarcomas, leukemias and lymphomas, such as T-cell lymphomas; metastasising cancers; autoimmune diseases, such as psoriasis, rheumatoid arthritis, Crohn's disease, ulcerative colitis, intestinal fibrosis, ankylosing spondylitis, systemic lupus erythematosus; metabolic disorders; coagulopathies, such as thrombosis proneness idiopathic (thrombophilia), autoimmune based thrombophilia, microthrombosis at multiorgan failure, COVID-19 related coagulopathy, thrombophilia in cancer disease; cardiovascular disorders, such as cardiac fibrosis, cardiac failure, left and right atrial fibrillation, atheromatosis, arterial inflammation, arterial calcification, aortic stenosis; heart disease; heart failure; aortic stenosis, atherosclerosis, pathological angiogenesis, such as ocular angiogenesis or a disease or condition associated with ocular angiogenesis, e.g. neovascularization related to cancer; and eye diseases, such as age-related macular degeneration and corneal neovascularization; atherosclerosis; endocrine disorders, such as Addison, autoimmune hypophysitis; metabolic diseases such as diabetes; type 2 diabetes; insulin resistance; obesity; Diastolic HF; atrophic diseases in the brain, such as Alzheimer's and Parkinson's, atrophic diseases in the cerebellum, such as cerebellar atrophy, atrophic spinal diseases such as ALS; disorders related to transplantation in organs, such as anti-rejection prophylaxis, anti-acute rejection, anti-chronic rejection; acute burn; acute inflammatory reaction; chronic acute skin graft rejection; chronic scarring; asthma and other interstitial lung diseases, including Hermansky-Pudlak syndrome, pulmonary arterial hypertension, Rheumatoid disease associated interstitial lung disease RA-ILD, Systemic Sclerosis SSc-ILD, lung disease with fibrosis such as COPD (Chronic Obstructive Pulmonary Disease) and asthma; Otosclerosis, mesothelioma; post-surgery disorders, such as anti-keloid, anti-stricture, anti-adhesion, anti-thrombosis, fibrosis/scar reduction following cosmetic procedures; toxin exposure disorders, such as toxic hepatitis, cholera toxin related, mushroom toxin based acute renal failure, pertussis toxin, Aeromonas hydrophila enterotoxin, cadmium induced cardiac toxicity, helicobacter O-antigen related toxicity, LPS based toxicity, Streptozotocin toxicity, asbestos exposure, Nephrogenic Systemic Fibrosis (Post Contrast Agents); Tissue injury, such as Spinal cord injury, Peripheral nerve repair; congenital hepatic fibrosis; hereditary fibrosing poikiloderma with tendon contractures, myopathy, and pulmonary fibrosis; liver disorders, such as non-alcoholic steatohepatitis (NASH) or non-alcoholic fatty liver disease, liver cirrhosis of various origins, such as alcoholic and non-alcoholic, autoimmune cirrhosis such as primary biliary cirrhosis and sclerosing cholangitis, virally induced cirrhosis, cirrhosis induced by genetic disease; Liver cancer, cholangiocarcinoma, biliary tract cancer; neurodegenerative disorders such as Parkinsons disease, Alzheimers disease, cognitive impairment, cerebrovascular diseases such as stroke, traumatic brain injury, Huntington's disease, amyotrophic lateral sclerosis (ALS), multiple sclerosis (MS), peripheral nephropathy.

In a still further aspect the present invention relates to a method for treatment of a disease or disorder relating to the binding of a galectin-3 to a ligand in a mammal, such as a human, wherein a therapeutically effective amount of at least one compound of formula (I) of the present invention is administered to a mammal in need of said treatment.

In a further embodiment the disease or disorder is selected from the group consisting of inflammation, such as acute post myocardial infarctions (MI), acute coronary syndrome, acute stent occlusion, acute myocardial reperfusion injury, acute pneumonitidies, acute lung injury (ALI), acute kidney injury (AKI), acute hepatitis, acute on chronic liver failure, acute alcohol hepatitis, acute pancreatitis, acute uveitis, acute pancreatitis related liponecrosis, acute retinitis, acute nephritis, acute myocarditis, chronic autoimmune diseases in all organs, (e.g. lung, liver, kidney, heart, skin, muscle, gut), chronic bacterial infections, chronic viral related inflammation; fibrosis, such as pulmonary fibrosis, liver fibrosis, kidney fibrosis, ophthalmological fibrosis and fibrosis of the skin and heart, acute post-surgical ocular fibrosis, acute transplantation rejection of the kidney, heart, lung, liver, and pancreas, acute post explosion/improvised explosive devices, acute post toxic dust (such as dust from terror attack known as 9/11), acute chemical exposure, chronic lung fibrosis, interstitial lung fibrosis (IPF), Interstitial Lung Disease (ILD), Childhood ILD (ChILD); chronic liver fibrosis, chronic alcohol fibrosis, chronic viral fibrosis, chronic diabetic fibrosis, diabetic nephropathy, chronic glomerulonephritis, renal artery stenosis, endometriosis; scarring; keloid formation; aberrant scar formation; surgical adhesions; scleroderma; systemic sclerosis; septic shock; cancers, such as carcinomas, sarcomas, leukemias and lymphomas, such as T-cell lymphomas; metastasising cancers; autoimmune diseases, such as psoriasis, rheumatoid arthritis, Crohn's disease, ulcerative colitis, intestinal fibrosis, ankylosing spondylitis, systemic lupus erythematosus; metabolic disorders; coagulopathies, such as thrombosis proneness idiopathic (thrombophilia), autoimmune based thrombophilia, microthrombosis at multiorgan failure, COVID-19 related coagulopathy, thrombophilia in cancer disease; cardiovascular disorders, such as cardiac fibrosis, cardiac failure, left and right atrial fibrillation, atheromatosis, arterial inflammation, arterial calcification, aortic stenosis; heart disease; heart failure; aortic stenosis, atherosclerosis, pathological angiogenesis, such as ocular angiogenesis or a disease or condition associated with ocular angiogenesis, e.g. neovascularization related to cancer; and eye diseases, such as age-related macular degeneration and corneal neovascularization; atherosclerosis; endocrine disorders, such as Addison, autoimmune hypophysitis; metabolic diseases such as diabetes; type 2 diabetes; insulin resistance; obesity; Diastolic HF; atrophic diseases in the brain, such as Alzheimer's and Parkinson's, atrophic diseases in the cerebellum, such as cerebellar atrophy, atrophic spinal diseases such as ALS; disorders related to transplantation in organs, such as anti-rejection prophylaxis, anti-acute rejection, anti-chronic rejection; acute burn; acute inflammatory reaction; chronic acute skin graft rejection; chronic scarring; asthma and other interstitial lung diseases, including Hermansky-Pudlak syndrome, pulmonary arterial hypertension, Rheumatoid disease associated interstitial lung disease RA-ILD, Systemic Sclerosis SSc-ILD, lung disease with fibrosis such as COPD (Chronic Obstructive Pulmonary Disease) and asthma; Otosclerosis, mesothelioma; post-surgery disorders, such as anti-keloid, anti-stricture, anti-adhesion, anti-thrombosis, fibrosis/scar reduction following cosmetic procedures; toxin exposure disorders, such as toxic hepatitis, cholera toxin related, mushroom toxin based acute renal failure, pertussis toxin, Aeromonas hydrophila enterotoxin, cadmium induced cardiac toxicity, helicobacter O-antigen related toxicity, LPS based toxicity, Streptozotocin toxicity, asbestos exposure, Nephrogenic Systemic Fibrosis (Post Contrast Agents); Tissue injury, such as Spinal cord injury, Peripheral nerve repair; congenital hepatic fibrosis; hereditary fibrosing poikiloderma with tendon contractures, myopathy, and pulmonary fibrosis; liver disorders, such as non-alcoholic steatohepatitis (NASH) or non-alcoholic fatty liver disease, liver cirrhosis of various origins, such as alcoholic and non-alcoholic, autoimmune cirrhosis such as primary biliary cirrhosis and sclerosing cholangitis, virally induced cirrhosis, cirrhosis induced by genetic disease; Liver cancer, cholangiocarcinoma, biliary tract cancer; neurodegenerative disorders such as Parkinsons disease, Alzheimers disease, cognitive impairment, cerebrovascular diseases such as stroke, traumatic brain injury, Huntington's disease, amyotrophic lateral sclerosis (ALS), multiple sclerosis (MS), peripheral nephropathy.

Another aspect of the present invention concerns combination therapy involving administering a compound of formula (I) of the present invention together with a therapeutically active compound different from the compound of formula (I) (interchangeable with “a different therapeutically active compound”). In one embodiment the present invention relates to a combination of a compound of formula (I) and a different therapeutically active compound for use in treatment of a disorder relating to the binding of a galectin-3 to a ligand in a mammal. Such disorders are disclosed below.

In an embodiment of the present invention, a therapeutically effective amount of at least one compound of formula (I) of the present invention is administered to a mammal in need thereof in combination with a different therapeutically active compound. In a further embodiment, said combination of a compound of formula (I) together with a different therapeutically active compound is administered to a mammal suffering from a disorder selected from the group consisting of inflammation; fibrosis, such as pulmonary fibrosis, liver fibrosis, kidney fibrosis, ophthalmological fibrosis and fibrosis of the skin and heart; scarring; keloid formation; aberrant scar formation; surgical adhesions; septic shock; cancer, such as carcinomas, sarcomas, leukemias and lymphomas, such as T-cell lymphomas; metastasising cancers; autoimmune diseases, such as psoriasis, rheumatoid arthritis, Crohn's disease, ulcerative colitis, ankylosing spondylitis, systemic lupus erythematosus; metabolic disorders; heart disease; heart failure; pathological angiogenesis, such as ocular angiogenesis or a disease or condition associated with ocular angiogenesis, e.g. neovascularization related to cancer; and eye diseases, such as age-related macular degeneration and corneal neovascularization; atherosclerosis; metabolic diseases such as diabetes; type 2 diabetes; insulin resistens; obesity; Diastolic HF; asthma and other interstitial lung diseases, including Hermansky-Pudlak syndrome, mesothelioma; liver disorders, such as non-alcoholic steatohepatitis or non-alcoholic fatty liver disease.

A non-limiting group of cancers given as examples of cancers, including both solid and liquid cancers, that may be treated, managed and/or prevented by administration of a compound of formula (I) in combination with a different therapeutically active compound is selected from: colon carcinoma, breast cancer, head and neck cancer, testis cancer, urothelial cancer, pancreatic cancer, ovarian cancer, prostate cancer, fibrosarcoma, myxosarcoma, liposarcoma, chondrosarcoma, osteogenic sarcoma, chordoma, angiosarcoma, endotheliosarcoma, lymphangeosarcoma, lymphangeoendothelia sarcoma, synovioma, mesothelioma, Ewing's sarcoma, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystandeocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioblastomas, neuronomas, craniopharingiomas, schwannomas, glioma, astrocytoma, medulloblastoma, craniopharyngioma, ependymoma, pinealoma, hemangioblastoma, acoustic neuroama, oligodendroglioma, meningioma, melanoma, neuroblastoma, retinoblastoma, leukemias and lymphomas, acute lymphocytic leukemia and acute myelocytic polycythemia vera, multiple myeloma, Waldenstrom's macroglobulinemia, and heavy chain disease, acute nonlymphocytic leukemias, chronic lymphocytic leukemia, chronic myelogenous leukemia, Hodgkin's Disease, non-Hodgkin's lymphomas, rectum cancer, urinary cancers, uterine cancers, oral cancers, skin cancers, stomach cancer, brain tumors, liver cancer, laryngeal cancer, esophageal cancer, mammary tumors, childhood-null acute lymphoid leukemia (ALL), thymic ALL, B-cell ALL, acute myeloid leukemia, myelomonocytoid leukemia, acute megakaryocytoid leukemia, Burkitt's lymphoma, acute myeloid leukemia, chronic myeloid leukemia, and T cell leukemia, small and large non-small cell lung carcinoma, acute granulocytic leukemia, germ cell tumors, endometrial cancer, gastric cancer, cancer of the head and neck, chronic lymphoid leukemia, hairy cell leukemia and thyroid cancer.

In some aspects of the present invention, the administration of at least one compound of formula (I) of the present invention and at least one additional therapeutic agent demonstrates therapeutic synergy. In some aspects of the methods of the present invention, a measurement of response to treatment observed after administering both at least one compound of formula (I) of the present invention and the additional therapeutic agent is improved over the same measurement of response to treatment observed after administering either the at least one compound of formula (I) of the present invention or the additional therapeutic agent alone.

A further aspect of the present invention concerns combination therapy involving administering a compound of formula (I) of the present invention together with an anti-fibrotic compound different from the compound of formula (I) to a mammal in need thereof. In a further embodiment, such anti-fibrotic compound may be selected from the following non-limiting group of anti-fibrotic compounds: pirfenidone, nintedanib, simtuzumab (GS-6624, AB0024), BG00011 (STX100), PRM-151, PRM-167, PEG-FGF21, BMS-986020, FG-3019, MN-001, IW001, SAR156597, GSK2126458, PAT-1251 and PBI-4050.

A further aspect of the present invention concerns combination therapy involving administering a compound of formula (I) of the present invention together with an anti-cardiovascular compound different from the compound of formula (I) to a mammal in need thereof.

A still further aspect of the present invention concerns combination therapy involving administering a compound of formula (I) in combination with a further conventional cancer treatment such as chemotherapy and/or radiotherapy, and/or treatment with immunostimulating substances, and/or gene therapy, and/or treatment with antibodies and/or treatment using dendritic cells, to a mammal in need thereof.

In an embodiment the compound of formula (I) is administered together with at least one additional therapeutic agent selected from an antineoplastic chemotherapy agent. In a further embodiment, the antineoplastic chemotherapeutic agent is selected from: all-trans retinoic acid, Actimide, Azacitidine, Azathioprine, Bleomycin, Carboplatin, Capecitabine, Cisplatin, Chlorambucil, Cyclophosphamide, Cytarabine, Daunorubicin, Docetaxel, Doxifluridine, Doxorubicin, Epirubicin, Etoposide, Fludarabine, Fluorouracil, Gemcitabine, Hydroxyurea, Idarubicin, Irinotecan, Lenalidomide, Leucovorin, Mechlorethamine, Melphalan, Mercaptopurine, Methotrexate, Mitoxantrone, Oxaliplatin, Paclitaxel, Pemetrexed, Revlimid, Temozolomide, Teniposide, Thioguanine, Valrubicin, Vinblastine, Vincristine, Vindesine and Vinorelbine. In one embodiment, a chemotherapeutic agent for use in the combination of the present agent may, itself, be a combination of different chemotherapeutic agents. Suitable combinations include FOLFOX and IFL. FOLFOX is a combination which includes 5-fluorouracil (5-FU), leucovorin, and oxaliplatin. IFL treatment includes irinotecan, 5-FU, and leucovorin.

In a further embodiment of the present invention, the further conventional cancer treatment includes radiation therapy. In some embodiments, radiation therapy includes localized radiation therapy delivered to the tumor. In some embodiments, radiation therapy includes total body irradiation.

In other embodiments of the present invention the further cancer treatment is selected from the group of immunostimulating substances e.g. cytokines and antibodies. Such cytokines may be selected from the group consisting of, but not limited to: GM-CSF, type I IFN, interleukin 21, interleukin 2, interleukin 12 and interleukin 15. The antibody is preferably an immunostimulating antibody such as anti-CD40 or anti-CTLA-4 antibodies. The immunostimulatory substance may also be a substance capable of depletion of immune inhibitory cells (e.g. regulatory T-cells) or factors, said substance may for example be E3 ubiquitin ligases. E3 ubiquitin ligases (the HECT, RING and U-box proteins) have emerged as key molecular regulators of immune cell function, and each may be involved in the regulation of immune responses during infection by targeting specific inhibitory molecules for proteolytic destruction. Several HECT and RING E3 proteins have now also been linked to the induction and maintenance of immune self-tolerance: c-Cbl, Cbl-b, GRAIL, Itch and Nedd4 each negatively regulate T cell growth factor production and proliferation.

In some embodiments of the present invention the compound of formula (I) is administered together with at least one additional therapeutic agent selected from a checkpoint inhibitor. In some embodiments of the invention, the checkpoint inhibitor is acting on one or more of the following, non-limiting group of targets: CEACAM1, galectin-9, TIM3, CD80, CTLA4, PD-1, PD-L1, HVEM, BTLA, CD160, VISTA, B7-H4, B7-2, CD155, CD226, TIGIT, CD96, LAG3, GITF, OX40, CD137, CD40, IDO, and TDO. These are known targets and some of these targets are described in Melero et al., Nature Reviews Cancer (2015). Examples of check point inhibitors administered together with the compound of formula (I) are Anti-PD-1: Nivolumab, Pembrolizumab, Cemiplimab. Anti-PD-L1: Atezolizumab, Avelumab, Durvalumab and one Anti-CTLA-4: Ipilimumab. Each one of these check point inhibitors can be made the subject of an embodiment in combination with any one of the compounds of formula (I).

In some embodiments of the present invention the compound of formula (I) is administered together with at least one additional therapeutic agent selected from an inhibitor of indoleamine-2,3-dioxygenase (IDO).

In some embodiments of the present invention the compound of formula (I) is administered together with at least one additional therapeutic agent selected from one or more inhibitors of the CTLA4 pathway. In some embodiments, the inhibitor of the CTLA4 pathway is selected from one or more antibodies against CTLA4.

In some embodiments of the present invention the compound of formula (I) is administered together with at least one additional therapeutic agent selected from one or more inhibitors of the PD-1/PD-L pathway. In some embodiments, the one or more inhibitors of the PD-1/PD-L pathway are selected from one or more antibodies or antibody fragments against PD-1, PD-L1, and/or PD-L2, or other ways by which an anti-PD1 antibodies can be induced such as mRNA based introduction of genetic material which sets forth in-body production of anti-PD1 or anti-PDL1 antibodies or fragments of such antibodies.

In a still further aspect the present invention relates to a process of preparing a compound of formula II or a pharmaceutically acceptable salt or solvate thereof comprising the step a1 where C¹, C² and B¹ are defined as above under formula I;

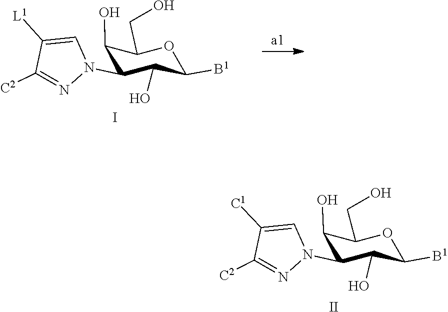

a1) Reacting a compound of the formula I wherein L¹ is a halide such as bromine or iodine with a compound of the formula C¹—X¹, wherein X¹ is defined as a boronic acid, borinatester, tinalkyl or zincalkyl suitable for cross-coupling reactions such as Suzuki, Stille or Negishi couplings in the presence of a catalyst such as Pd(PPh₃)₄ or Pd(dppf)Cl₂ in a suitable solvent such as 1,4-dioxane/water optionally in the presence of a base such as K₂CO₃, optionally at elevated temperatures to give a compound of formula II; alternatively reacting a compound of formula I wherein L¹ is defined as a boronic acid or ester, borinatester, tinalkyl or zincalkyl suitable for cross-coupling reactions such as Suzuki, Stille or Negishi couplings with a compound of formula C¹—X² wherein X² is defined as a halide such as bromine or iodine in the presence of a catalyst such as Pd(PPh₃)₄ or Pd(dppf)Cl₂ and a base such as K₂CO₃ in a suitable solvent such as 1,4-dioxane and water optionally at elevated temperatures to give a compound of formula II.
In a still further aspect the present invention relates to a process of preparing a compound of formula IV or a pharmaceutically acceptable salt or solvate thereof comprising the step a2 where C¹, C², B¹ and R¹ are defined as above under formula I;

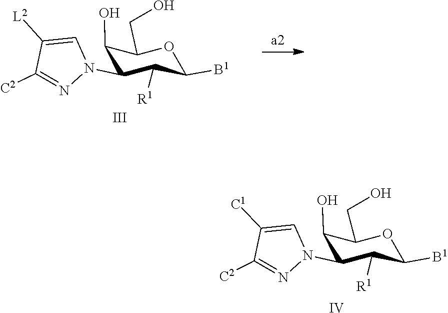

a2) Reacting a compound of the formula III wherein L² is a halide such as bromine or iodine with a compound of the formula C¹—X³, wherein X³ is defined as a boronic acid, borinatester, tinalkyl or zincalkyl suitable for cross-coupling reactions such as Suzuki, Stille or Negishi couplings in the presence of a catalyst such as Pd(PPh₃)₄ or Pd(dppf)Cl₂ in a suitable solvent such as 1,4-dioxane/water optionally in the presence of a base such as K₂CO₃, optionally at elevated temperatures to give a compound of formula IV; alternatively reacting a compound of formula III wherein L² is defined as a boronic acid or ester, borinatester, tinalkyl or zincalkyl suitable for cross-coupling reactions such as Suzuki, Stille or Negishi couplings with a compound of formula C¹—X⁴ wherein X⁴ is defined as a halide such as bromine or iodine in the presence of a catalyst such as Pd(PPh₃)₄ or Pd(dppf)Cl₂ and a base such as K₂CO₃ in a suitable solvent such as 1,4-dioxane and water optionally at elevated temperatures to give a compound of formula IV.
In a still further aspect the present invention relates to a process of preparing a compound of formula VI or a pharmaceutically acceptable salt or solvate thereof comprising the step a3 where C¹ and B¹ are defined as above under formula I;

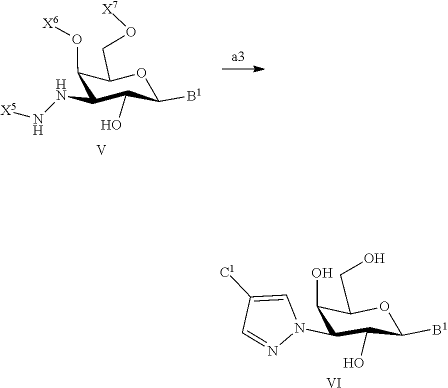

a3) Reacting a compound of the formula V wherein X⁵ is a protective group such as a boc-group and X⁶ and X⁷ together form a protective group such as benzylidene with a compound of the formula VII

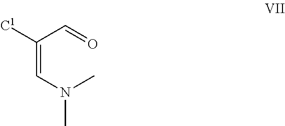

in the presence of an acid such as HCl in a suitable solvent such as EtOH, optionally at elevated temperatures to give a compound of formula VI.

In a still further aspect the present invention relates to a process of preparing a compound of formula IX or a pharmaceutically acceptable salt or solvate thereof comprising the step a4 where C¹, B¹ and R¹ are defined as above under formula I;

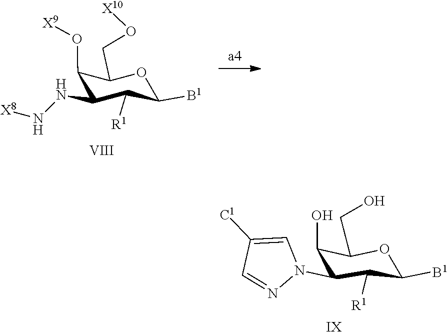

a4) Reacting a compound of the formula VIII wherein X⁸ is a protective group such as a boc-group and X⁹ and X¹⁰ together form a protective group such as benzylidene with a compound of the formula VII

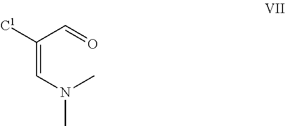

in the presence of an acid such as HCl in a suitable solvent such as ethanol, optionally at elevated temperatures to give a compound of formula IX.
In a still further aspect the present invention relates to a process of preparing a compound of formula XI or a pharmaceutically acceptable salt or solvate thereof comprising the step a5 where C¹, C² and B¹ are defined as above under formula I;

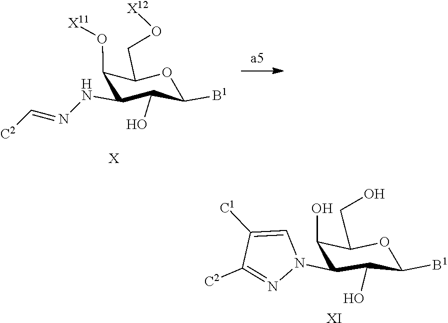

a5) Reacting a compound of the formula X wherein X¹¹ and X¹² together form a protective group such as benzylidene with a compound of the formula XII

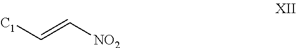

in the presence of a base such as potassium tert-butoxide in an inert solvent such as THF at −78° C., followed by treatment with an acid such as TFA to give a compound of formula XI.
In a still further aspect the present invention relates to a process of preparing a compound of formula XIV or a pharmaceutically acceptable salt or solvate thereof comprising the step a6 where R¹, C¹, C² and B¹ are defined as above under formula I;

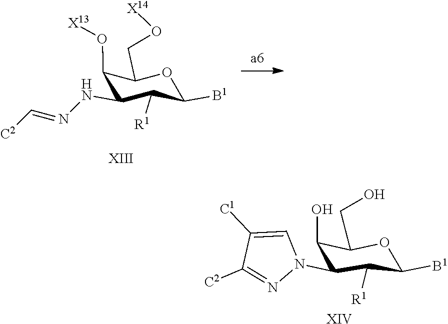

a6) Reacting a compound of the formula XIII wherein X¹³ and X¹⁴ together form a protective group such as benzylidene with a compound of the formula XII

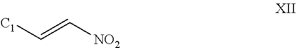

in the presence of a base such as potassium tert-butoxide in an inert solvent such as THF at −78° C., followed by treatment with an acid such as TFA to give a compound of formula XIV.
In a still further aspect the present invention relates to a process of preparing a compound of formula XVII or a pharmaceutically acceptable salt or solvate thereof comprising the step a7 where C¹ and C² are defined as above under formula I, X¹⁵ is selected from the substituents under formula I under B¹, Z¹ is an aryl or a heteroaryl selected under formula I under B¹, L⁴ is a cyano group;

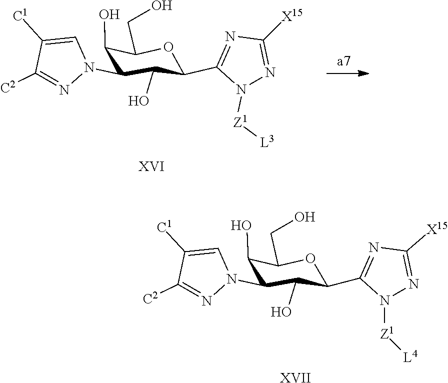

a7) Reacting a compound of the formula XVI wherein L³ is a halide such as a bromine with a cyanide reagent such as copper cyanide in an inert solvent such as DMSO at elevated temperatures to give a compound of formula XVII; alternatively reacting a compound of formula XVI wherein L³ is a halide such as bromine with a cyanide reagent such as zinc cyanide in the presence of zinc, 1,1′-bis(diphenylphosphino)ferrocene Pd₂(dba)₃ in an inert solvent such as DMF optionally at elevated temperatures to give a compound of formula XVII.
In a still further aspect the present invention relates to a process of preparing a compound of formula XIX or a pharmaceutically acceptable salt or solvate thereof comprising the step a8 where C¹ and B¹ are defined as above under formula I, X¹⁶ is selected from the substituents under formula I under C²;

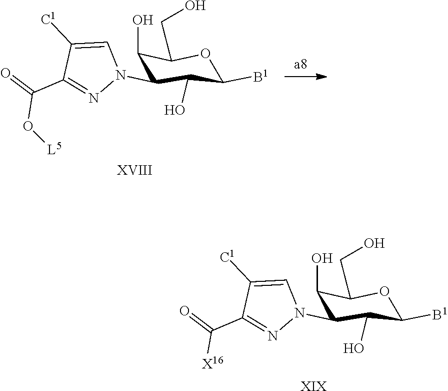

a8) Reacting a compound of the formula XVIII wherein L⁵ is an alkyl such as ethyl with a nucleophile such as hydroxide or ammonia in a suitable solvent such as methanol optionally at elevated temperatures to give a compound of formula XIX.
In a still further aspect the present invention relates to a process of preparing a compound of formula XXI or a pharmaceutically acceptable salt or solvate thereof comprising the step a9 where C¹ and B¹ are defined as above under formula I;

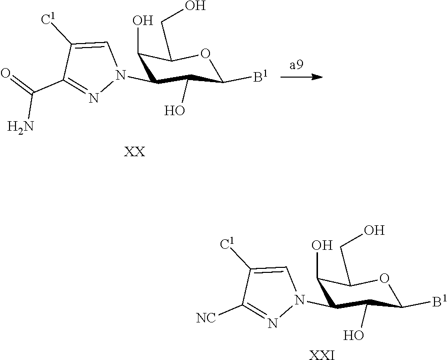

a9) Reacting a compound of the formula XX with trifluoroacetic anhydride and pyridine in an inert solvent such as THF optionally at elevated temperatures to give a compound of formula XXI.
In a still further aspect the present invention relates to a process of preparing a compound of formula XXIII or a pharmaceutically acceptable salt or solvate thereof comprising the step a10 where C¹ and B¹ are defined as above under formula I, Z² is an aryl or a heteroaryl selected under formula I under C¹, L⁷ is an alkyl group;

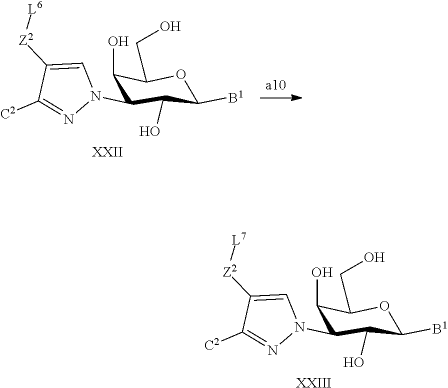

a10) Reacting a compound of the formula XXII wherein L⁶ is an alkenyl group with hydrogen in the presence of a suitable catalyst such as platinum(IV) oxide in an inert solvent such as THF to give a compound of formula XXIII.
In a still further aspect the present invention relates to a process of preparing a compound of formula XXIX or a pharmaceutically acceptable salt or solvate thereof comprising the steps a11-a15 wherein B¹, C² and R¹ are defined as above under formula I, X¹⁷ and X¹⁸ together form a protective group such as benzylidene, X¹⁹ is a protective group such as a boc-group, L⁸ is defined as a halogen such as iodine or bromine and L⁹ is defined as a boronic acid or a borinate.

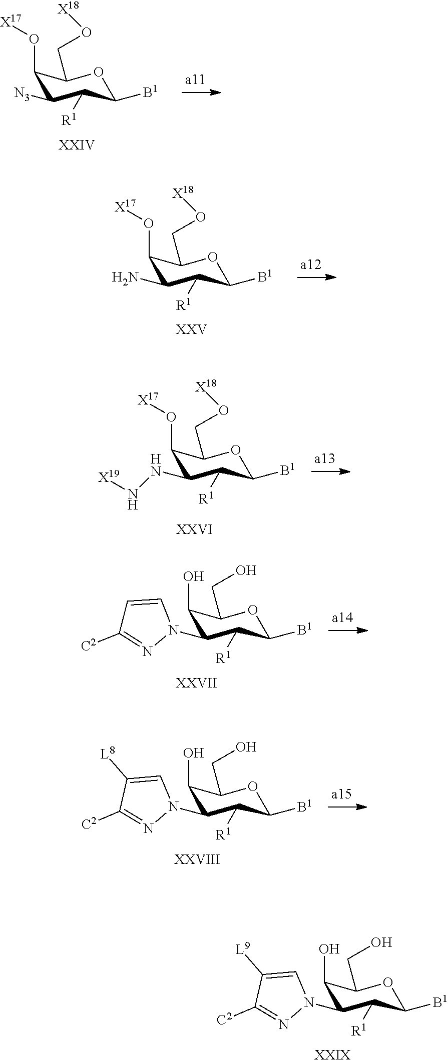

a11) Reacting a compound of formula XXIV with a reducing agent such as triphenylphosphine in an inert solvent such as THF and water optionally at elevated temperatures to give a compound of formula XXV.
a12) Reacting a compound of the formula XXV with N-tert-butyloxycarbonyl-3-(4-cyanophenyl)oxaziridine in an inert solvent such as DCM to give a compound of formula XXVI.
a13) Reacting a compound of the formula XXVI with a deprotecting agent such as TFA in an inert solvent such as DCM to give an intermediate which is further reacted with either 1,1,3,3-tetraethoxypropane or a compound of the formula

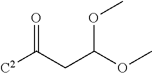

in the presence of an acid such as HCl in a suitable solvent such as ethanol to give a compound of formula XXVII.
a14) Reacting a compound of the formula XXVII with a halogenating agent such as N-bromosuccinimide or N-iodosuccinimide in an inert solvent such as THF to give a compound of formula XXVIII.
a15) Reacting compound of formula XXVIII with bis(pinacolato)diboron in the presence of a catalyst such as Pd(dppf)Cl₂ and a base such as potassium acetate in an inert solvent such as DMSO optionally at elevated temperatures to give a compound of formula XXIX.
In a still further aspect the present invention relates to a process of preparing a compound of formula XXXIV or a pharmaceutically acceptable salt or solvate thereof comprising the steps a16-a19 wherein R¹³ is defined as above under formula I and X²⁴ is selected from the substituents under formula I under B¹.

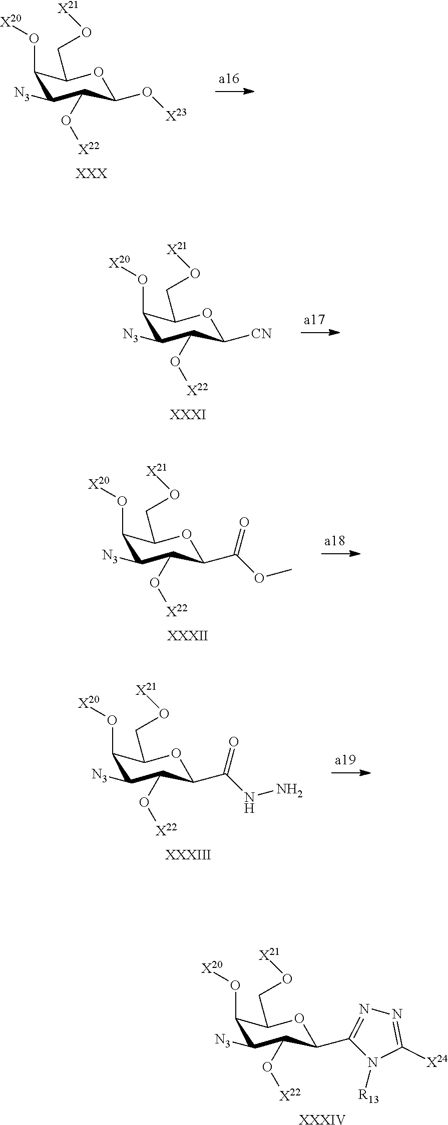

a16) Reacting a compound of the formula XXX wherein X²⁰—X²³ is a protective group such as acetate, with a cyanide reagent such as trimethylsilyl cyanide in the presence of a reagent such as boron trifluoride diethyl etherate in an inert solvent such as nitromethane at 0° C. to give a compound of the formula XXXI.
a17) Reacting a compound of the formula XXXI wherein X²⁰—X²² is a protective group such as acetate with acetyl chloride in methanol optionally at elevated temperatures giving a product which is further reacted with benzaldehyde dimethylacetal in the presence of D(+)-10-camphorsulfonic acid to give a compound of formula XXXII, wherein X²⁰ and X²¹ together form a protective group such as benzylidene and X²² is a hydrogen; Optionally the compound of formula XXXII, wherein X²⁰ and X²¹ together form a protective group such as benzylidene and X²² is a hydrogen, could be reacted with an alkyl halide such as iodomethane in the presence of a base such as cesium carbonate in an inert solvent such as DMF to give another compound of formula XXXII wherein X²⁰ and X²¹ together form a protective group such as benzylidene and X²² is an alkyl group such as methyl.
a18) Reacting a compound of formula XXXII defined as above with hydrazine at elevated temperatures in a solvent such as ethanol to give a compound of formula XXXIII wherein X²⁰ and X²¹ together form a protective group such as benzylidene and X²² is an alkyl group such as methyl or a hydrogen.
a19) Reacting a compound of formula XXXIII defined as above with N,N-dimethylformamide dimethyl acetal yielding a product which is further reacted with R¹³—NH₂ and acetic acid to give a compound of formula XXXIV wherein X²⁰ and X²¹ together form a protective group such as benzylidene and X²² is an alkyl group such as methyl or a hydrogen; Optionally reacting a compound of formula XXXIII defined as above with a compound of the formula XXXV

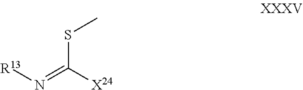

in the presence of p-toluenesulfonic acid in an inert solvent such as DMF to give a compound of formula XXXIV wherein X²⁰ and X²¹ together form a protective group such as benzylidene and X²² is an alkyl group such as methyl or a hydrogen.
In a still further aspect the present invention relates to a process of preparing a compound of formula XXXVIII or a pharmaceutically acceptable salt or solvate thereof comprising the steps a20-a21, X²⁵ and X²⁶ together form a protective group such as benzylidene and X²⁷ is an alkyl group such as methyl or a hydrogen, R¹³ is defined as above under formula I and X²⁸ is selected from the substituents under formula I under B¹;

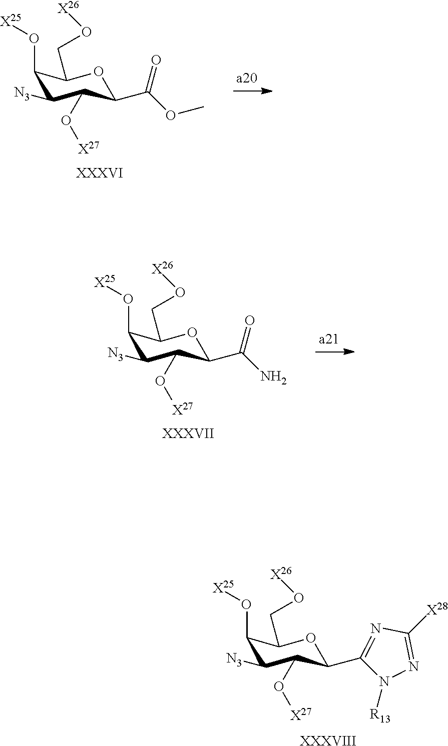

a20) Reacting a compound of formula XXXVI with NH₃ in a solvent such as methanol to give a compound of formula XXXVII.
a21) Reacting a compound of the formula XXXVII with a compound of the formula XXXIX

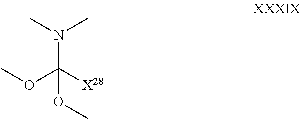

in a solvent such as 1,4-dioxane optionally at elevated temperatures resulting in a product, which is further reacted with acetic acid and R₁₃—NHNH₂ in a solvent such as acetonitrile at elevated temperatures to give a compound of formula XXXVIII.
In a still further aspect the present invention relates to a process of preparing a compound of formula XXXXI or a pharmaceutically acceptable salt or solvate thereof comprising the step a22 where B¹, C² and R¹ are defined as above under formula I, X²⁹ and X³⁰ are either hydrogens or together form a protective group such as benzylidene and X³¹ is a protective group such as a boc-group;

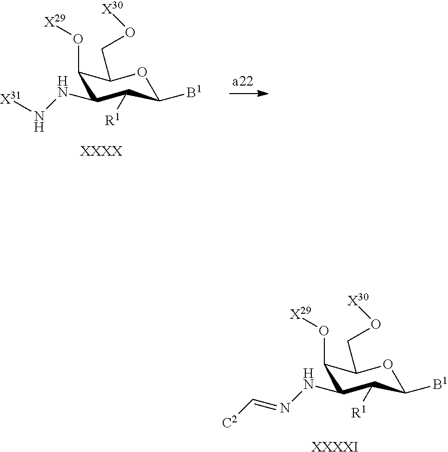

a22) Reacting a compound of formula XXXX with a compound of the formula

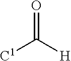

in the presence of an acid such as HCl in a suitable solvent such as ethanol to give a compound of formula XXXXI.
In a still further aspect the present invention relates to a process of preparing a compound of formula VII or a pharmaceutically acceptable salt or solvate thereof comprising the step a23 where C¹ is defined as above under formula I;

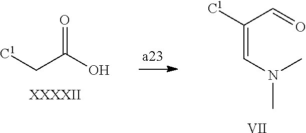

a23) Reacting a compound of the formula XXXXII with DMF and phosphoryl trichloride at elevated temperatures to give a product, which is further reacted with K₂CO₃ in toluene at elevated temperatures to give a compound of the formula VII.
In a still further aspect the present invention relates to a process of preparing a compound of formula XXXV or a pharmaceutically acceptable salt or solvate thereof comprising the steps a24-a26 where R¹³ is defined as above under formula I and X²⁴ is selected from the substituents under formula I under B¹;

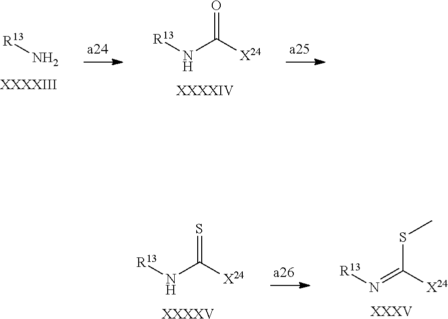

a24) Reacting a compound of formula XXXXIII with an activated ester such as an anhydride of the formula (X²⁴CO)₂O in the presence of pyridine in an inert solvent such as DCM to give a compound of formula XXXXIV.
a25) Reacting a compound of formula XXXXIV with Lawesson's reagent in a solvent such as toluene at elevated temperatures to give a compound of formula XXXXV.
a26) Reacting a compound of formula XXXXV with iodomethane in the presence of a base such as K₂CO₃ in a solvent such as acetonitrile to give a compound of formula XXXV.
In a still further aspect the present invention relates to a process of preparing a compound of formula XII or a pharmaceutically acceptable salt or solvate thereof comprising the step a27 where C¹ is defined as above under formula I;

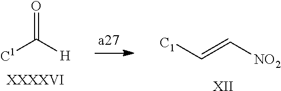

a27) Reacting a compound of the formula XXXXVI with ammonium acetate and nitromethane in acetic acid at elevated temperatures to give a compound of the formula XII.
In a still further aspect the present invention relates to a process of preparing a compound of formula XXXXVIII or a pharmaceutically acceptable salt or solvate thereof comprising the step a28 where R¹³ is defined as above under formula I;

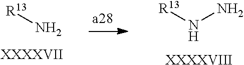

a28) Reacting a compound of the formula XXXXVII with sodium nitrite in the presence of an acid such as HCl in water to give a product that is reacted with tin(II)chloride to give a compound of formula XXXXVIII.
In a still further aspect the present invention relates to a process of preparing a compound of formula L or a pharmaceutically acceptable salt or solvate thereof comprising the step a29 where R¹³ is defined as above under formula I;

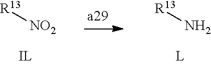

a29) Reacting a compound of the formula IL with ammonium chloride in the presence of iron in a solvent mixture, such as ethanol/water at elevated temperature to give a compound of the formula L.
In a still further aspect the present invention relates to a process of preparing a compound of formula LIII or a pharmaceutically acceptable salt or solvate thereof comprising the step a30;

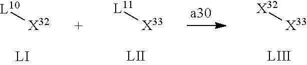

a30) Reacting a compound of the formula LI wherein X³² is defined as a heterocycle, aromatic ring or an c1-c6 alkyl or cycloalkyl and L¹⁰ is defined as a boron substitutent such as boronic acid, trifluoroborate or bronic ester with a compound of formula LII wherein X³³ is a heterocycle or aromatic ring and L¹¹ is a halide such as chlorine, bromine or iodine in the presence of a catalyst such as Pd(dppf)Cl₂ and a base such as K₂CO₃ in an inert solvent such as 1,4 dioxane to give a compound of the formula LIII.
In a still further aspect the present invention relates to a process of preparing a compound of formula LV or a pharmaceutically acceptable salt or solvate thereof comprising the step a31 where Z³ is an aryl or a heteroaryl selected under formula I under C₁;

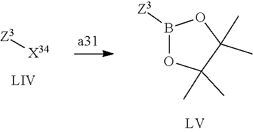

a31) Reacting a compound of the formula LIV wherein X³⁴ is a halide such as bromine or iodine with bis(pinacolato)diboron in the presence of a metalloorganic reagent such as Pd(dppf)Cl₂ and a base such as potassium acetate in a solvent such as DMSO to give a compound of formula LV.
In a still further aspect the present invention relates to a process of preparing a compound of formula LVII or a pharmaceutically acceptable salt or solvate thereof comprising the step a32 where R¹³ is defined as above under formula I;

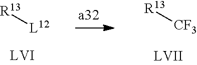

a32) Reacting a compound of formula LVI wherein L¹² is a leaving group such as a halide such as bromine with methyl 2,2-difluoro-2-fluorosulfonylacetate in the presence of a copper salt such as CuI in an inert solvent such as DMF optionally at elevated temperature to give a compound of formula LVII.

### DETAILED DESCRIPTION OF THE INVENTION

The present compounds of formula (I) differ from prior art compounds particularly in that the pyranose ring is β-D-galactopyranose. It is important to emphasize that alpha and beta anomers are very different isomers and it is by no means considered to be obvious to the skilled person to expect same or similar activity of both anomers. Consequently, alpha and beta anomers do not in general posses the same activity, and this is common knowledge to the skilled person. The compounds of the present invention are novel β-D-galactopyranose compounds that unexpectedly have shown very high affinity and specificity for galectin-3 and are considered novel potent drug candidates. In vitro PK properties as exemplified by compounds herein suggests high uptake over the intestine and low hepatic metabolism. This data suggests high bioavailability and long halflife in man after for example oral administration. Some of the novel β-D-galactopyranose compounds are atropoisomers and the compounds of formula (I) includes such atropoisomers.

In broad first aspect the present invention concerns a β-D-galactopyranose compound of formula (I)

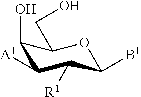

wherein
the pyranose ring is β-D-galactopyranose, and A¹, R¹ and B¹ are as defined above.

In an embodiment C¹ is selected from a) an aryl optionally substituted with a group selected from a C₁, F or I; CN; an ethynyl; a spiro heterocycle; —COOH; —CONR²R³, wherein R² and R³ are independently selected from H, C₁₋₃ alkyl, and cyclopropyl, or R² and R³ together with the nitrogen may form a C₃₋₆ heterocycloalkyl; C₁₋₃ alkyl optionally substituted with a F; C₁₋₃ alkyl, optionally substituted with an ethynyl; cyclopropyl optionally substituted with a F; OC₁₋₃ alkyl optionally substituted with a F; O-cyclopropyl, optionally substituted with a F; NR⁴R⁵, wherein R⁴ and R⁵ are independently selected from H, C₁₋₃ alkyl and cyclopropyl; C(═O)—R^{11a}, wherein R^{11 a} is selected from H and C₁₋₃ alkyl OH; and R⁶—CONH— wherein R⁶ is selected from C₁₋₃ alkyl and cyclopropyl.

In a further embodiment C₁ is selected from a) an aryl optionally substituted with a group selected from a C₁ or F; CN; —COOH; —CONR²R³, wherein R² and R³ are independently selected from H, C₁₋₃ alkyl, cyclopropyl, and iso-propyl, or R² and R³ together with the nitrogen may form a heterocycloalkyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR⁴R⁵, wherein R⁴ and R⁵ are independently selected from H, C₁₋₃ alkyl and isopropyl; OH; and R⁶—CONH— wherein R⁶ is selected from C₁₋₃ alkyl and cyclopropyl.

In a still further embodiment C¹ is a phenyl, optionally substituted with a group selected from a halogen; CN; —COOH; —CONR²R³, wherein R² and R³ are independently selected from H, C₁₋₃ alkyl, cyclopropyl, and iso-propyl, or R² and R³ together with the nitrogen may form a heterocycloalkyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR⁴R⁵, wherein R⁴ and R⁵ are independently selected from H, C₁₋₃ alkyl and isopropyl; OH; and R⁶—CONH— wherein R⁶ is selected from C₁₋₃ alkyl and cyclopropyl. Preferably, provided that both ortho carbon positions of the phenyl ring in relation to the covalent bond to the pyrazol ring have hydrogens attached to the carbon atoms.

In a further embodiment C¹ is a phenyl.

In a still further embodiment C¹ is a phenyl substituted with one to three selected from F and C₁. Preferably, provided that both ortho carbon positions of the phenyl ring in relation to the covalent bond to the pyrazol ring have hydrogens attached to the carbon atoms.

In a further embodiment C¹ is a phenyl substituted with three selected from F, Br and C₁, such as two F and one C₁, or two F and one Br or three F. Typically all three substituents are in meta and para positions. Preferably, provided that both ortho carbon positions of the phenyl ring in relation to the covalent bond to the pyrazol ring have hydrogens attached to the carbon atoms.

In a still further embodiment C¹ is a phenyl substituted with three selected from OC₁₋₃ alkyl optionally substituted with a F, C₁₋₃ alkyl optionally substituted with a F, C₁₋₃ alkyl and F. Typically, OCH₃, CF₃, isopropyl and F. In a further embodiment C¹ is a phenyl substituted with two F and one selected from OCH₃, CF₃ and isopropyl. Preferably, provided that both ortho carbon positions of the phenyl ring in relation to the covalent bond to the pyrazol ring have hydrogens attached to the carbon atoms.

In a still further embodiment C¹ is a phenyl substituted with two selected from F and C₁, such as one F and one C₁ or two F. Preferably, provided that both ortho carbon positions of the phenyl ring in relation to the covalent bond to the pyrazol ring have hydrogens attached to the carbon atoms.

In a further embodiment C¹ is a phenyl substituted with two selected from F and C₁₋₃ alkyl, such as one F and one methyl. Typically, the two substituents are in meta and para. Preferably, provided that both ortho carbon positions of the phenyl ring in relation to the covalent bond to the pyrazol ring have hydrogens attached to the carbon atoms.

In a still further embodiment C¹ is a phenyl substituted with one to three selected from F and ethynyl, such as two F and one ethynyl. Typically, all three substituents are in meta and para positions. Preferably, provided that both ortho carbon positions of the phenyl ring in relation to the covalent bond to the pyrazol ring have hydrogens attached to the carbon atoms.

In a still further embodiment C¹ is a phenyl substituted with one to three selected from F and C₂₋₄ alkenyl, such as two F and one propenyl. Typically, all three substituents are in meta and para positions. Preferably, provided that both ortho carbon positions of the phenyl ring in relation to the covalent bond to the pyrazol ring have hydrogens attached to the carbon atoms.

In a further embodiment C¹ is a phenyl substituted with three selected from F and C₁₋₃ alkyl, such as two F and one methyl. Typically, all three substituents are in meta and para position. Preferably, provided that both ortho carbon positions of the phenyl ring in relation to the covalent bond to the pyrazol ring have hydrogens attached to the carbon atoms.

In a still further embodiment C¹ is a phenyl substituted with one selected from C₁₋₃ alkyl, optionally substituted with a F and halogen, such as CF₃ and C₁. Typically, CF₃ in para position or C₁ in meta or para position. Preferably, provided that both ortho carbon positions of the phenyl ring in relation to the covalent bond to the pyrazol ring have hydrogens attached to the carbon atoms.
In another embodiment C¹ is a heterocycle, such as heteroaryl or heterocycloalkyl, optionally substituted with a group selected from a halogen; a spiro heterocycle; CN; —COOH; —CONR⁷R⁸, wherein R⁷ and R⁸ are independently selected from H, C₁₋₃ alkyl, and cyclopropyl, or R⁷ and R⁸ together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR⁹R¹⁰, wherein R⁹ and R¹⁰ are independently selected from H, C₁₋₃ alkyl, and cyclopropyl; C(═O)—R¹¹, wherein R¹¹ is selected from H and C₁₋₃ alkyl; OH; and R¹²—CONH— wherein R¹² is selected from C₁₋₃ alkyl and cyclopropyl. Preferably, provided that ortho carbon positions of the heterocycle ring in relation to the covalent bond to the pyrazol ring have hydrogens attached to the carbon atoms.
In a further embodiment C¹ is a heterocycle optionally substituted with a group selected from a halogen; a spiro heterocycle; CN; —COOH; —CONR⁷R⁸, wherein R⁷ and R⁸ are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR⁹R¹⁰, wherein R⁹ and R¹⁰ are independently selected from H, C₁₋₃ alkyl, cyclopropyl and isopropyl, C(═O)—R¹¹, wherein R¹¹ is selected from H and C₁₋₃ alkyl; OH; and R¹²—CONH— wherein R¹² is selected from C₁₋₃ alkyl and cyclopropyl. Preferably, provided that ortho carbon positions of the heterocycle ring in relation to the covalent bond to the pyrazol ring have hydrogens attached to the carbon atoms.

In a further embodiment C¹ is a heteroaryl optionally substituted with a group selected from a F or C₁; a spiro heterocycle; CN; —COOH; —CONR⁷R⁸, wherein R⁷ and
R⁸ are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR⁹R¹⁰, wherein R⁹ and R¹⁰ are independently selected from H, C₁₋₃ alkyl, cyclopropyl and isopropyl, C(═O)—R¹¹, wherein R¹¹ is selected from H and C₁₋₃ alkyl; OH; and R¹²—CONH— wherein R¹² is selected from C₁₋₃ alkyl and cyclopropyl. Preferably, provided that ortho carbon positions of the heteroaryl ring in relation to the covalent bond to the pyrazol ring have hydrogens attached to the carbon atoms.

In a still further embodiment C¹ is a pyridinyl.

In a further embodiment C¹ is a pyridinyl substituted with one to three selected from F and C₁, such as one C₁ and one F. Preferably, provided that ortho carbon positions of the pyridinyl ring in relation to the covalent bond to the pyrazol ring have hydrogens attached to the carbon atoms.

In a further embodiment C¹ is a pyridinyl substituted with one to three selected from F and C₁₋₃ alkyl, such as one methyl and one F. Preferably, provided that ortho carbon positions of the pyridinyl ring in relation to the covalent bond to the pyrazol ring have hydrogens attached to the carbon atoms.

In a further embodiment C¹ is a pyridinyl substituted with one to three selected from C₁ and C₁₋₃ alkyl, such as one methyl and one C₁. Preferably, provided that ortho carbon positions of the pyridinyl ring in relation to the covalent bond to the pyrazol ring have hydrogens attached to the carbon atoms.

In a still further embodiment C² is selected from the group consisting of
H; C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{4a}R^{5a}, wherein R^{4a} and R^{5a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{2a}R^{3a}, wherein R^{2a} and R^{3a} are independently selected from H, C₁₋₃ alkyl optionally substituted with a halogen, and cyclopropyl optionally substituted with a halogen; and S(O₂)NR^{6a}R^{7a} wherein R^{6a} and R^{7a} are independently selected from H, C₁₋₆ alkyl optionally substituted with a halogen, and cyclopropyl optionally substituted with a halogen.

In a further embodiment C² is selected from the group consisting of
phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{4b}R^{5b}, wherein R^{4b} and R^{5b} are independently selected from H, C₁₋₆ alkyl optionally substituted with a halogen, C₃_6 cycloalkyl optionally substituted with a halogen, C(O)C₁₋₆ alkyl optionally substituted with a halogen, and S(O₂)C₁₋₆ alkyl optionally substituted with a halogen; C(O)C₃_6 cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{2b}R^{3b}, wherein R^{2b} and R^{3b} are independently selected from H, C₁₋₃ alkyl optionally substituted with a halogen, and cyclopropyl optionally substituted with a halogen; and S(O₂)NR^{6b}R^{7b} wherein R^{6b} and R^{7b} are independently selected from H, C₁₋₆ alkyl optionally substituted with a halogen, and cyclopropyl optionally substituted with a halogen.

In a still further embodiment C² is selected from the group Y—Z wherein
Y is linked to the pyrazole of A¹ and is selected from the group consisting of S, Se, SO, SO₂, O, C═O, and CR^{2c}R^{3c} wherein R^{2c} and R^{3c} are independently selected from hydrogen, OH, or halogen; and
Z is selected from the group consisting of phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{4c}R^{5c}, wherein R^{4c} and R^{5c} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{2d}R^{3d}, wherein R^{2d} and R^{3d} are independently selected from H, C₁₋₃ alkyl optionally substituted with a halogen, and cyclopropyl optionally substituted with a halogen; and S(O₂)NR^{6c}R^{7c} wherein R^{6c} and R^{7c} are independently selected from H, C₁₋₆ alkyl optionally substituted with a halogen, and cyclopropyl optionally substituted with a halogen.

In a still further embodiment C² is H.

In another embodiment C² is C₁₋₆ alkyl optionally substituted with a halogen, such as methyl.

In a still further embodiment C² is CN.

In a further embodiment C² is phenyl optionally substituted with a group selected from halogen, OC₁₋₃ alkyl substituted with a halogen, and C₁₋₃ alkyl.

In a still further embodiment C² is phenyl.

In a further embodiment C² is phenyl substituted with one group selected from F, OCF₃ and CH₃.

In a still further embodiment C² is selected from the group Y—Z wherein
Y is linked to the pyrazole of A¹ and is CR^{2c}R^{3C} wherein R^{2c} and R^{3c} are both hydrogen, and Z is a phenyl.

In a further embodiment C² is selected from the group consisting of C(O)OC₁₋₃ alkyl, COOH and C(O)NH₂. Typically, C² is selected from the group consisting of C(O)OC₂ alkyl, COOH and C(O)NH₂.

In a further embodiment B1 is selected from the group consisting of pyrrolyl, furanyl, thienyl, pyrazolyl, isoxazolyl, isothiazolyl, imidazolyl, oxazolyl, thiazolyl, triazolyl, oxadiazolyl, thiadiazolyl, phenyl, or indolyl substituted with a group selected from a) C₁₋₆ alkyl optionally substituted with one or more of C₁₋₆ alkyl, halogen, hydroxy, C₁₋₆ alkoxy, carboxy, alkoxycarbonyl, H₂NCO, b) R¹³—C₁₋₆ alkyl, c) C₃₋₆ cycloalkyl optionally substituted with one or more of C₁₋₆ alkyl, halogen, or hydroxy, d) C₁₋₆ alkoxy, e) C₁₋₆ alkylthio, f) C₁₋₆ alkylsulfonyl, g) carbonyl substituted with any one of hydroxy, C₁₋₆ alkoxy, C₁₋₆ alkylNH, ((R¹⁴)(R¹⁵)N)C₁₋₆ alkylNH, or (pyridinyl)C₁₋₆ alkylNH, h) (R¹⁶)(R¹⁷)N, j) R¹³, and k) C₁₋₆ alkenyl, wherein R¹³, R¹⁴, R¹, R¹⁶ and R¹⁷ are as defined above under the definitions for the compound of formula (I).

In a still further embodiment B1 is triazolyl substituted with a group selected from a) C₁₋₆ alkyl optionally substituted with one or more of halogen, and j) R¹³; wherein R¹³ is selected from the group consisting of phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolo[4,5-b]pyridinyl, thiazolyl, triazolyl, oxadiazolyl, or thiadiazolyl; optionally substituted with one or more substituents selected from the group consisting of cyano, nitro, OH, C₂-alkynyl, halogen, C₁₋₆ alkyl, C₁₋₆ alkyl optionally substituted with a halogen, C₃₋₆ cycloalkyl, C₁₋₆ alkoxy, halo-C₁₋₆ alkoxy, C₁₋₆ alkylthio, carboxy, C₁₋₆ alkoxycarbonyl, and CONH₂.

In a further embodiment B1 is triazolyl substituted with a group selected from a) C₁₋₆ alkyl, and j) R¹³, wherein R¹³ is selected from the group consisting of phenyl, pyridinyl, thiazolo[4,5-b]pyridinyl, thiazolyl, biphenyl, and benzothiazolyl, optionally substituted with one or more substituents selected from the group consisting of cyano, halogen, C₁₋₆ alkyl, and C₁₋₆ alkyl optionally substituted with a halogen.

In a still further embodiment B1 is triazolyl substituted with one group selected from j) R¹³, wherein R¹³ is selected from the group consisting of phenyl, pyridinyl, thiazolyl, biphenyl, thiazolo[4,5-b]pyridinyl and benzothiazolyl, optionally substituted with one or more substituents selected from the group consisting of cyano, halogen, C₁₋₆ alkyl, and C₁₋₆ alkyl optionally substituted with a halogen.

In a further embodiment B¹ is triazolyl, such as 1,2,4-triazolyl, substituted with one group selected from C₁₋₆ alkyl, and one group selected from phenyl optionally substituted with one or more substituents selected from the group consisting of cyano, halogen, and C₁₋₆ alkyl optionally substituted with a halogen.

Typically, the phenyl is substituted with one, two or three substituents, such as two substituents, selected from the group consisting of cyano, C₁, Br and CF₃.

In a still further embodiment B¹ is triazolyl, such as 1,2,4-triazolyl, substituted with one group selected from C₁₋₆ alkyl, and one group selected from pyridinyl optionally substituted with one or more substituents selected from the group consisting of cyano, halogen, C₁₋₆ alkyl, and C₁₋₆ alkyl optionally substituted with a halogen. Typically, the pyridinyl is substituted with one, two or three substituents, such as one or two substituents, selected from the group consisting of cyano, C₁, Br and CF₃.

In a further embodiment B1 is triazolyl, such as 1,2,4-triazolyl, substituted with one group selected from C₁₋₆ alkyl, and one group selected from thiazolyl optionally substituted with one or more substituents selected from the group consisting of cyano, halogen, C₁₋₆ alkyl, and C₁₋₆ alkyl optionally substituted with a halogen. Typically, the thiazolyl is substituted with one, two or three substituents, such as one substituent, selected from the group consisting of halogen, e.g. one C₁.

In a still further embodiment B¹ is triazolyl, such as 1,2,4-triazolyl, substituted with one group selected from C₁₋₆ alkyl, and one group selected from biphenyl optionally substituted with one or more substituents selected from the group consisting of cyano, halogen, C₁₋₆ alkyl, and C₁₋₆ alkyl optionally substituted with a halogen. Typically, the biphenyl is substituted with one, two, three or four substituents selected from the group consisting of F and CF₃, such as one CF₃ on the phenyl attached to the triazolyl and three F atoms on the other phenyl.

In a further embodiment B1 is triazolyl, such as 1,2,4-triazolyl, substituted with one group selected from C₁₋₆ alkyl, and one group selected from benzothiazolyl optionally substituted with one or more substituents selected from the group consisting of cyano, halogen, C₁₋₆ alkyl, and C₁₋₆ alkyl optionally substituted with a halogen. Typically, the benzothiazolyl is unsubstituted or is substituted with one or two substituents selected from the group consisting of C₁₋₄ alkyl, CF₃, F, Cl, Br, and cyano.

In a further embodiment B¹ is triazolyl, such as 1,2,4-triazolyl, substituted with one group selected from C₁₋₆ alkyl, and one group selected from thiazolo[4,5-b]pyridinyl optionally substituted with one or more substituents selected from the group consisting of cyano, halogen, C₁₋₆ alkyl, and C₁₋₆ alkyl optionally substituted with a halogen. Typically, the thiazolo[4,5-b]pyridinyl, is substituted with one C₁₋₄ alkyl; such as one methyl.

When B¹ is triazolyl it is preferred that the pyranose ring is linked to a carbon atom of said triazolyl. Preferably the triazolyl is a 1,2,4-triazolyl.

In a further embodiment R¹ is OH.

In a still further embodiment R¹ is OC₁₋₄ alkyl.

In a further embodiment R¹ is selected from O-methyl, O-ethyl, or O-isopropyl. Typically, R¹ is selected from OH and methoxy.

In a further aspect the present invention concerns a β-D-galactopyranose compound of formula (I) selected from any one of the exemplified compounds of examples 1-59, or a pharmaceutically acceptable salt thereof.

The skilled person will understand that it may be necessary to adjust or change the order of steps in the processes a1-a32, and such change of order is encompassed by the aspects of the process as described above in the reaction schemes and accompanying description of the process steps.

Furthermore, the skilled person will understand that the processes described above and hereinafter the functional groups of intermediate compounds may need to be protected by protecting groups.

Functional groups that it is desirable to protect include hydroxy, amino and carboxylic acid. Suitable protecting groups for hydroxy include optionally substituted and/or unsaturated alkyl groups (e.g. methyl, allyl, benzyl or tert-butyl), trialkyl silyl or diarylalkylsilyl groups (e.g. t-butyldimethylsilyl, t-butyldipheylsilyl or trimethylsilyl), AcO(acetoxy), TBS(t-butyldimethylsilyl), TMS(trimethylsilyl), PMB (p-methoxybensyl), and tetrahydropyranyl. Suitable protecting groups for carboxylic acid include (C₁₋₆)-alkyl or benzyl esters. Suitable protecting groups for amino include t-butyloxycarbonyl, benzyloxycarbonyl, 2-(trimethylsilyl)-ethoxy-methyl or 2-trimethylsilylethoxycarbonyl (Teoc). Suitable protecting groups for S include S—C(═N)NH₂, TIPS.

The protection and deprotection of functional groups may take place before or after any reaction in the above-mentioned processes.

Furthermore the skilled person will appreciate, that, in order to obtain compounds of the invention in an alternative, and on some occasions more convenient manner, the individual process steps mentioned hereinbefore may be performed in different order, and/or the individual reactions may be performed at a different stage in the overall route (i.e. substituents may be added to and/or chemical transformations performed upon, different intermediates to those mentioned hereinbefore in conjunction with a particular reaction). This may negate, or render necessary, the need for protecting groups.

In a still further embodiment the compound (1) is on free form. “On free form” as used herein means a compound of formula (I), either an acid form or base form, or as a neutral compound, depending on the substitutents. The free form does not have any acid salt or base salt in addition. In one embodiment the free form is an anhydrate. In another embodiment the free form is a solvate, such as a hydrate.

In a further embodiment the compound of formula (I) is a crystalline form. The skilled person may carry out tests in order to find polymorphs, and such polymorphs are intended to be encompassed by the term “crystalline form” as used herein.

Whenever a “compound of formula (I)” is used herein it means the compound of formula (I) in any form incl the free form or as a salt thereof, such as a pharmaceutically acceptable salt thereof, unless otherwise indicated herein or clearly contradicted by context.

When the compounds and pharmaceutical compositions herein disclosed are used for the above treatment, a therapeutically effective amount of at least one compound is administered to a mammal in need of said treatment.

The term “C₁₋ₓ alkyl” as used herein means an alkyl group containing 1-x carbon atoms, e.g. C₁₋₅ or C₁₋₆, such as methyl, ethyl, propyl, butyl, pentyl or hexyl.

The term “branched C₃₋₆ alkyl” as used herein means a branched alkyl group containing 3-6 carbon atoms, such as isopropyl, isobutyl, tert-butyl, isopentyl, 3-methylbutyl, 2,2-dimethylpropyl, n-hexyl, 2-methylpentyl, 2,2-dimethylbutyl, 2,3-dimethylbutyl.

The term “C₃₋ₓ cycloalkyl” as used herein means a cyclic alkyl group containing 3-x carbon atoms, e.g. C₃_6 or C₃_7, such as cyclopropyl, cyclobutyl, cyclopentyl, cyclohexyl, and cycloheptyl.

The term “C₅₋₇ cycloalkyl” as used herein means a cyclic alkyl group containing 5-7 carbon atoms, such as cyclopentyl, cyclohexyl, or cycloheptyl.

The term “Oxo” as used herein means an oxygen atom with double bonds, also indicated as ═O.

The term “CN” as used herein means a nitril.

The term “halogen” as used herein means Cl, F, Br or I.

The term “halo-C₁₋₆ alkyl” as used herein means one or more halogens linked to a C₁₋₆ alkyl, such as CF₃, CH(C₁)CHF₂.

The term “C₁₋₆ alkoxy” as used herein means an oxygen linked to a C₁₋₆ alkyl, such as methoxy or ethoxy.

The term “C₁₋₆ alkylthio” as used herein means a sulphur linked to a C₁₋₆ alkyl, such as thiomethoxy or thioethoxy.

The term “halo-C₁₋₆ alkoxy” as used herein means one or more halogens linked to a C₁₋₆ alkoxy, such as CH(F₂)CH(Br)O—.

The term “C₁₋₆ alkoxycarbonyl” as used herein means a C₁₋₆ alkoxy linked to a carbonyl, such as methoxycarbonyl (CH₂OC(═O)).

The term “a five or six membered heteroaromatic ring” as used herein means one five membered heteroaromatic ring or one six membered heteroaromatic ring. The five membered heteroaromatic ring contains 5 ring atoms of which one to four are heteroatoms selected from N, O, and S. The six membered heteroaromatic ring contains 6 ring atoms of which one to five are heteroatoms selected from N, O and S. Examples include thiophene, furan, pyran, pyrrole, imidazole, pyrazole, isothiazole, isooxazole, pyridine, pyrazine, pyrimidine and pyridazine. When such heteroaromatic rings are substituents they are termed thiophenyl, furanyl, pyranyl, pyrrolyl, imidazolyl, pyrazolyl, isothiazolyl, isooxazolyl, pyridinyl, pyrazinyl, pyrimidinyl and pyridazinyl. Also included are oxazoyl, thiazoyl, thiadiazoly, oxadiazoyl, and pyridonyl.

The term “a heterocycle, such as heteroaryl or heterocycloalkyl” as used herein means a heterocycle consisting of one or more 3-7 membered ring systems containing one or more heteroatoms and wherein such ring systems may optionally be aromatic. The term “a heteroaryl” as used herein means a mono or bicyclic aromatic ringsystem containing one or more heteroatoms, such as 1-10, e.g. 1-6, selected from O, S, and N, including but not limited to oxazolyl, oxadiazolyl, thiophenyl, thiadiazolyl, thiazolyl, pyridyl, pyrimidinyl, pyridonyl, pyrimidonyl, quinolinyl, azaquionolyl, isoquinolinyl, azaisoquinolyl, quinazolinyl, azaquinazolinyl, bensozazoyl, azabensoxazoyl, bensothiazoyl, or azabensothiazoyl. The term “a heterocycloalkyl” as used herein means a mono or bicyclic 3-7 membered alifatic heterocycle containing one or more heteroatoms, such as 1-7, e.g. 1-5, selected from O, S, and N, including but not limited to piperidinyl, tetrahydropyranyl, tetrahydrothipyranyl, or piperidonyl.

The term “a spiro heterocycle” as used herein means a two-ring system connected by a common carbon atom, and containing from 5 to 12 ring members wherein from 2 to 11 are carbon atoms and at least one is a heteroatom, such as a hetero atom selected from one or more N, S, O; one example is N-(2-oxa)-6-azaspiro[3.3]heptanyl.

When referring to “both ortho carbon positions of the phenyl ring in relation to the covalent bond to the pyrazol ring have hydrogens attached to the carbon atoms.” as used herein the two arrows in below formula points to the two ortho positions:

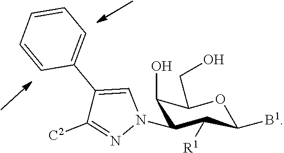

When referring to “ortho carbon positions of the pyridinyl ring in relation to the covalent bond to the pyrazol ring have hydrogens attached to the carbon atoms.” as used herein the one arrow in below formula points to the only ortho carbon atom position:

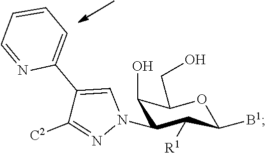

and
the two arrows in below formula points to the two ortho carbon atoms positions:

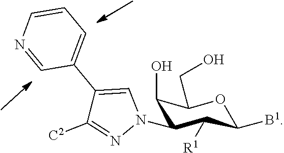

The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. The treatment may either be performed in an acute or in a chronic way. The patient to be treated is preferably a mammal; in particular, a human being, but it may also include animals, such as dogs, cats, cows, sheep and pigs.

The term “a therapeutically effective amount” of a compound of formula (I) of the present invention as used herein means an amount sufficient to cure, alleviate or partially arrest the clinical manifestations of a given disease and its complications. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

In a still further aspect, the present invention relates to a pharmaceutical composition comprising the compound of formula (I) and optionally a pharmaceutically acceptable additive, such as a carrier or an excipient.

As used herein “pharmaceutically acceptable additive” is intended without limitation to include carriers, excipients, diluents, adjuvant, colorings, aroma, preservatives etc. that the skilled person would consider using when formulating a compound of the present invention in order to make a pharmaceutical composition.

The adjuvants, diluents, excipients and/or carriers that may be used in the composition of the invention must be pharmaceutically acceptable in the sense of being compatible with the compound of formula (I) and the other ingredients of the pharmaceutical composition, and not deleterious to the recipient thereof. It is preferred that the compositions shall not contain any material that may cause an adverse reaction, such as an allergic reaction. The adjuvants, diluents, excipients and carriers that may be used in the pharmaceutical composition of the invention are well known to a person skilled within the art.

As mentioned above, the compositions and particularly pharmaceutical compositions as herein disclosed may, in addition to the compounds herein disclosed, further comprise at least one pharmaceutically acceptable adjuvant, diluent, excipient and/or carrier. In some embodiments, the pharmaceutical compositions comprise from 1 to 99% by weight of said at least one pharmaceutically acceptable adjuvant, diluent, excipient and/or carrier and from 1 to 99% by weight of a compound as herein disclosed. The combined amount of the active ingredient and of the pharmaceutically acceptable adjuvant, diluent, excipient and/or carrier may not constitute more than 100% by weight of the composition, particularly the pharmaceutical composition.

In some embodiments, only one compound as herein disclosed is used for the purposes discussed above.

In some embodiments, two or more of the compounds as herein disclosed are used in combination for the purposes discussed above.

The composition, particularly pharmaceutical composition comprising a compound set forth herein may be adapted for oral, intravenous, topical, intraperitoneal, nasal, buccal, sublingual, or subcutaneous administration, or for administration via the respiratory tract in the form of, for example, an aerosol or an air-suspended fine powder. Therefore, the pharmaceutical composition may be in the form of, for example, tablets, capsules, powders, nanoparticles, crystals, amorphous substances, solutions, transdermal patches or suppositories.

Further embodiments of the process are described in the experimental section herein, and each individual process as well as each starting material constitutes embodiments that may form part of embodiments.

The above embodiments should be seen as referring to any one of the aspects (such as ‘method for treatment’, ‘pharmaceutical composition’, ‘compound for use as a medicament’, or ‘compound for use in a method’) described herein as well as any one of the embodiments described herein unless it is specified that an embodiment relates to a certain aspect or aspects of the present invention.

All references, including publications, patent applications and patents, cited herein are hereby incorporated by reference to the same extent as if each reference was individually and specifically indicated to be incorporated by reference and was set forth in its entirety herein.

All headings and sub-headings are used herein for convenience only and should not be construed as limiting the invention in any way.

Any combination of the above-described elements in all possible variations thereof is encompassed by the invention unless otherwise indicated herein or otherwise clearly contradicted by context.

The terms “a” and “an” and “the” and similar referents as used in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also pro-vide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The citation and incorporation of patent documents herein is done for convenience only and does not reflect any view of the validity, patentability and/or enforceability of such patent documents.

The term “and/or” as used herein is intended to mean both alternatives as well as each of the alternatives individually. For instance, the expression “xxx and/or yyy” means “xxx and yyy”; “xxx”; or “yyy”, all three alternatives are subject to individual embodiments.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The present invention is further illustrated by the following examples that, however, are not to be construed as limiting the scope of protection. The features disclosed in the foregoing description and in the following examples may, both separately and in any combination thereof, be material for realizing the invention indiverse forms thereof.

### Experimental Procedures (Evaluation of Kd Values)

The affinity of Example 1-59 for galectins were determined by a fluorescence anisotropy assay where the compound was used as an inhibitor of the interaction between galectin and a fluorescein tagged saccharide probe as described Sörme, P., Kahl-Knutsson, B., Huflejt, M., Nilsson, U. J., and Leffler H. (2004) Fluorescence polarization as an analytical tool to evaluate galectin-ligand interactions. Anal. Biochem. 334: 36-47, (Sarme et al., 2004)

| | | | Gal-3 |
|---|---|---|---|
| | | | Kd |
| Example | Name | Structure | (μM) |
| 1 | 3-{5-{3-[4-(4-Chloro-3,5- difluorophenyl)-1H-1,2- pyrazol-1-yl]-3-deoxy-β- D-galactopyranosyl}-3- methyl-1H-1,2,4-triazol- 1-yl}-5-chloro-2-cyclo- propylpyridine | 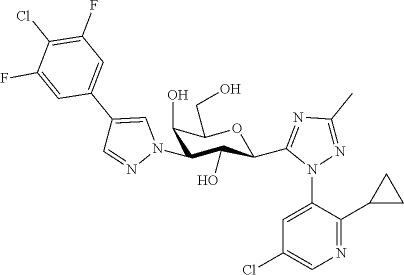 | 0.032 |
| 2 | 6-{3-{3-Deoxy-3-[4- (3,4,5-trifluorophenyl)- 1H-1,2-pyrazol-1-yl]-β- D-galactopyranosyl}-4H- 1,2,4-triazol-4-yl}-2- methylbenzothiazole | 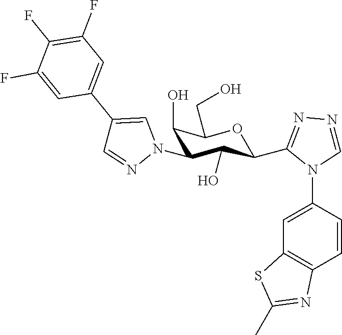 | 0.039 |
| 3 | 5-Chloro-1-{5-{3-deoxy- 3-[4-(3,4,5-trifluorophen- yl)-1H-1,2-pyrazol-1-yl]- β-D-galactopyranosyl}-3- methyl-1H-1,2,4-triazol- 1-yl}-2-(trifluoromethyl)- benzene | 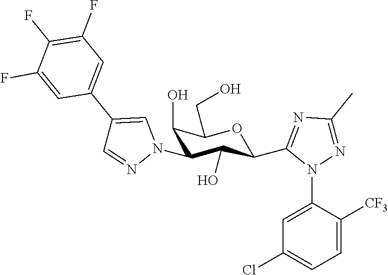 | 0.009 |
| 4 | 1-{5-{3-Deoxy-3-[4- (3,4,5-trifluorophenyl)- 1H-1,2-pyrazol-1-yl]-β- D-galactopyranosyl}-3- methyl-1H-1,2,4-triazol- 1-yl}-2-(trifluoromethyl)- 2-(3,4,5-trifluorophenyl)- benzene | 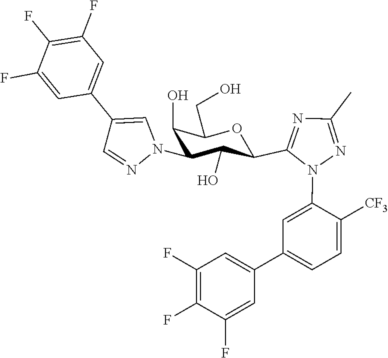 | 0.20 |
| 5 | 1-{5-{3-[4-(4-Chloro-3,5- difluorophenyl)-1H-1,2- pyrazol-1-yl]-3-deoxy-β- D-galactopyranosyl}-3- methyl-1H-1,2,4-triazol-1- yl}-5-chloro-2-(trifluoro- methyl)benzene | 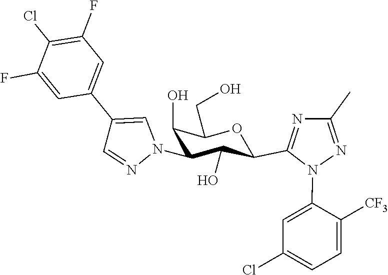 | 0.006 |
| 6 | 5-Chloro-1-{5-{3-deoxy- 3-[4-(6-fluoropyridin-2- yl)-1H-1,2-pyrazol-1-yl]- B-D-galactopyranosyl}-3- methyl-1H-1,2,4-triazol- 1-yl}-2-(trifluoromethyl)- benzene | 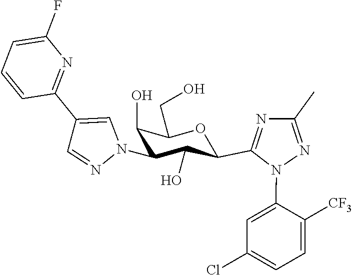 | 0.13 |
| 7 | 5-Chloro-1-{5-{3-deoxy- 3-[4-(4-fluoropyridin-2- yl)-1H-1,2-pyrazol-1-yl]- β-D-galactopyranosyl}-3- methyl-1H-1,2,4-triazol- 1-yl}-2-(trifluoromethyl)- benzene | 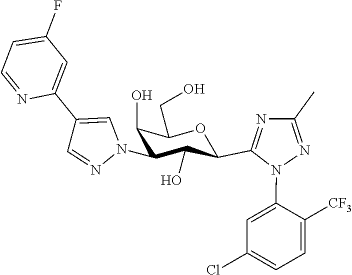 | 0.082 |
| 8 | 6-{5-{3-Deoxy-3-[4- (3,4,5-trifluorophenyl)- 1H-1,2-pyrazol-1-yl]-β- D-galactopyranosyl}-3- methyl-1H-1,2,4-triazol- 1-yl}-2-methylbenzo- thiazole | 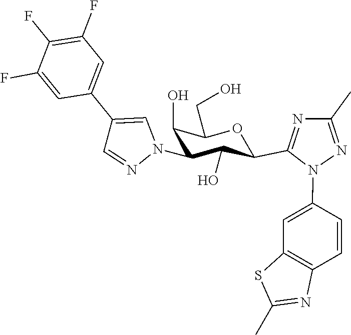 | 0.022 |
| 9 | 6-{5-{3-[4-(4-Chloro-3,5- difluorophenyl)-1H-1,2- pyrazol-1-yl]-3-deoxy-β- D-galactopyranosyl}-3- methyl-1H-1,2,4-triazol- 1-yl}-2-methylbenzo- thiazole | 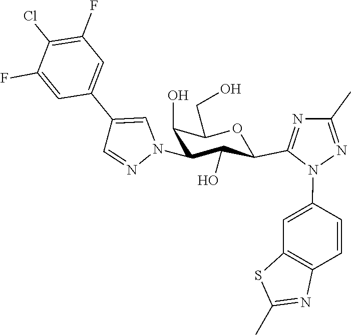 | 0.012 |
| 10 | 1-{3-{3-[4-(4-Chloro-3,5- difluorophenyl)-1H-1,2- pyrazol-1-yl]-3-deoxy-β- D-galactopyranosyl}-5- methyl-4H-1,2,4-triazol-4- yl}-5-chloro-2-(trifluoro- methyl)benzene | 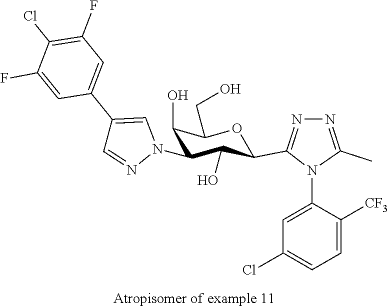 | 0.37 |
| 11 | 1-{3-{3-[4-(4-Chloro-3,5- difluorophenyl)-1H-1,2- pyrazol-1-yl]-3-deoxy-β- D-galactopyranosyl}-5- methyl-4H-1,2,4-triazol-4- yl}-5-chloro-2-(trifluoro- methyl)benzene | 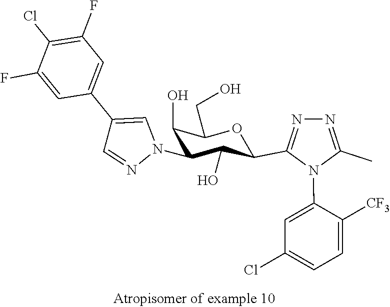 | 0.007 |
| 12 | 1-{5-{3-[4-(4-Chloro-3,5- difluorophenyl)-3-meth- yl-1H-1,2-pyrazol-1-yl]- 3-deoxy-β-D-galacto- pyranosyl}-3-methyl- 1H-1,2,4-triazol-1-yl}-5- chloro-2-(trifluorometh- yl)benzene | 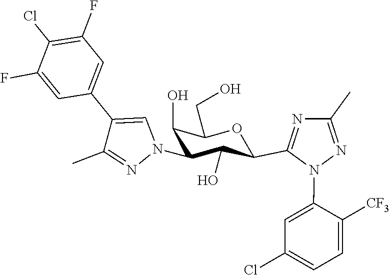 | 0.011 |
| 13 | 5-Chloro-3-{5-{3-deoxy- 3-[4-(3,4,5-trifluorophen- yl)-1H-1,2-pyrazol-1-yl]- β-D-galactopyranosyl}-3- methyl-1H-1,2,4-triazol- 1-yl}-2-cyclopropyl- pyridine | 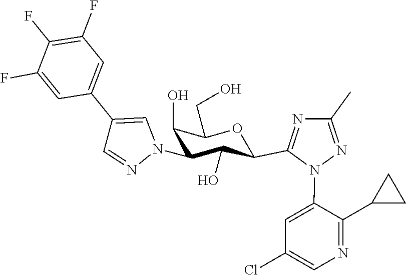 | 0.015 |
| 14 | 1-{5-{3-[4-(5-Chloro-4- fluoropyridin-2-yl)-1H- 1,2-pyrazol-1-yl]-3- deoxy-β-D-galactopyran- osyl}-3-methyl-1H-1,2,4- triazol-1-yl}-5-chloro-2- (trifluoromethyl)benzene | 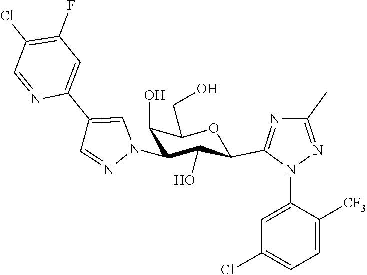 | 0.024 |
| 15 | 5-Chloro-1-{5-{3-deoxy- 3-[4-(4-fluoro-5-methyl- pyridin-2-yl)-1H-1,2- pyrazol-1-yl]-β-D-galacto- pyranosyl}-3-methyl-1H- 1,2,4-triazol-1-yl}-2- (trifluoromethyl)benzene | 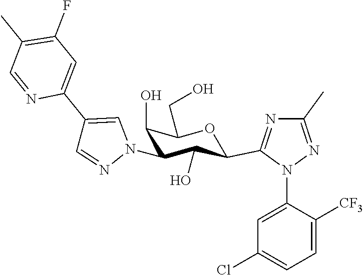 | 0.013 |
| 16 | 1-{5-{3-[4-(5-Chloro-4- methylpyridin-2-yl)-1H- 1,2-pyrazol-1-yl]-3- deoxy-β-D-galactopyran- osyl}-3-methyl-1H-1,2,4- triazol-1-yl}-5-chloro-2- (trifluoromethyl)benzene | 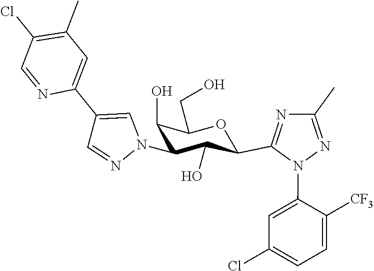 | 0.14 |
| 17 | 5-Chloro-1-{5-{3-deoxy- 3-[4-(4-ethynyl-3,5- difluorophenyl)-1H-1,2- pyrazol-1-yl]-β-D-galac- topyranosyl}-3-methyl- 1H-1,2,4-triazol-1-yl}-2- (trifluoromethyl)benzene | 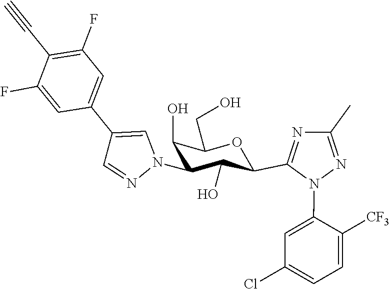 | 0.060 |
| 18 | 5-Chloro-1-{5-{3-deoxy- 3-{4-[3,5-difluoro-4-(2- propyn-1-yl)phenyl]-1H- 1,2-pyrazol-1-yl}-β-D- galactopyranosyl}-3- methyl-1H-1,2,4-triazol- 1-yl}-2-(trifluoromethyl)- benzene | 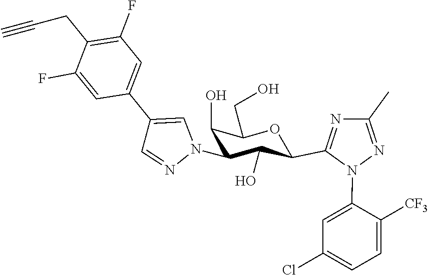 | 0.026 |
| 19 | 5-Bromo-3-{5-{3-deoxy- 3-[4-(3,4,5-trifluorophen- yl)-1H-1,2-pyrazol-1-yl]- β-D-galactopyranosyl}-3- methyl-1H-1,2,4-triazol- 1-yl}pyridine | 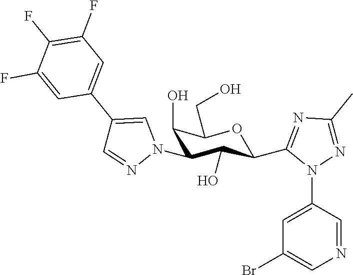 | 0.025 |
| 20 | 5-Chloro-3-{5-{3-deoxy- 3-[4-(3,4,5-trifluorophen- yl)-1H-1,2-pyrazol-1-yl]- β-D-galactopyranosyl}-3- methyl-1H-1,2,4-triazol- 1-yl}-2-(trifluoromethyl)- pyridine | 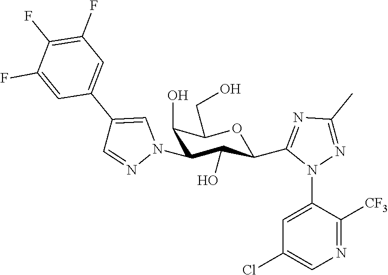 | 0.011 |
| 21 | 5-Chloro-2-cyano-3-{5- {3-deoxy-3-[4-(3,4,5- trifluorophenyl)-1H-1,2- pyrazol-1-yl]-β-D-galac- topyranosyl}-3-methyl- 1H-1,2,4-triazol-1-yl}- pyridine | 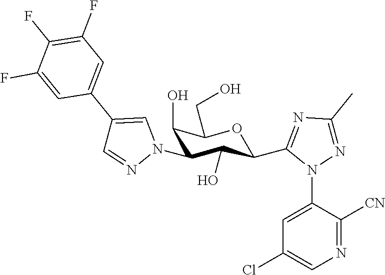 | 0.082 |
| 22 | 5-Bromo-2-cyano-3-{5- {3-deoxy-3-[4-(3,4,5- trifluorophenyl)-1H-1,2- pyrazol-1-yl]-β-D-galac- topyranosyl}-3-methyl- 1H-1,2,4-triazol-1-yl}- pyridine | 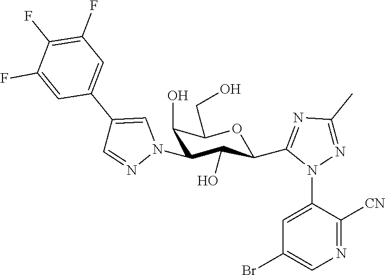 | 0.033 |
| 23 | 3-Chloro-2-cyano-5-{5- {3-deoxy-3-[4-(3,4,5- trifluorophenyl)-1H-1,2- pyrazol-1-yl]-β-D-galac- topyranosyl}-3-methyl- 1H-1,2,4-triazol-1-yl}- pyridine | 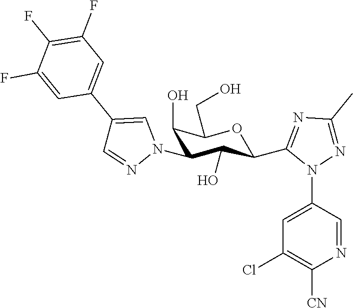 | 0.24 |
| 24 | 5-Bromo-1-{5-{3-deoxy- 3-[4-(3,4,5-trifluorophen- yl)-1H-1,2-pyrazol-1-yl]- β-D-galactopyranosyl}-3- methyl-1H-1,2,4-triazol- 1-yl}-2-(trifluoromethyl)- benzene | 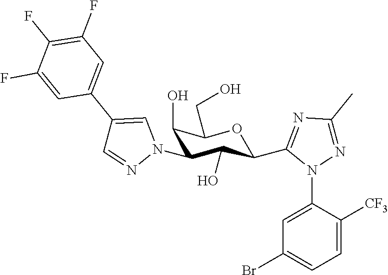 | 0.002 |
| 25 | 5-Bromo-3-{5-{3-deoxy- 3-[4-(3,4,5-trifluorophen- yl)-1H-1,2-pyrazol-1-yl]- β-D-galactopyranosyl}-3- methyl-1H-1,2,4-triazol- 1-yl}-2-(trifluoromethyl)- pyridine | 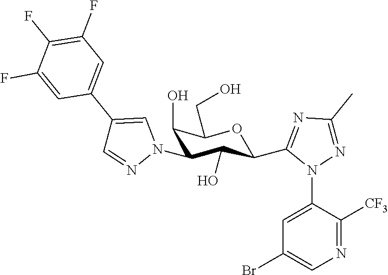 | 0.011 |
| 26 | 5-Bromo-2-cyano-1-{5- {3-deoxy-3-[4-(3,4,5- trifluorophenyl)-1H-1,2- pyrazol-1-yl]-β-D-galc- topyranosyl}-3-methyl- 1H-1,2,4-triazol-1-yl}- benzene | 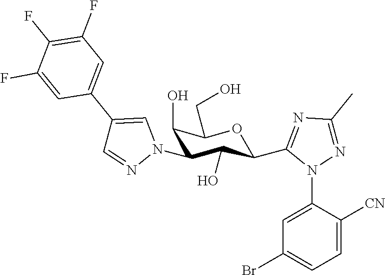 | 0.015 |
| 27 | 5-Chloro-2-cyano-1-{5- {3-deoxy-3-[4-(3,4,5- trifluorophenyl)-1H-1,2- pyrazol-1-yl]-β-D-galac- topyranosyl}-3-methyl- 1H-1,2,4-triazol-1-yl}- benzene | 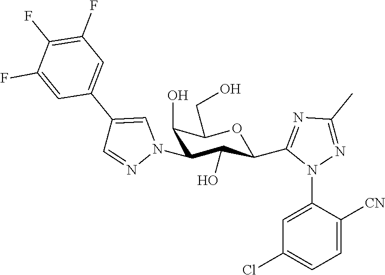 | 0.037 |
| 28 | 3-{5-{3-[4-(4-Chloro-3,5- difluorophenyl)-1H-1,2- pyrazol-1-yl]-3-deoxy-β- D-galactopyranosyl}-3- methyl-1H-1,2,4-triazol- 1-yl}-5-chloro-2- (trifluoromethyl)pyridine | 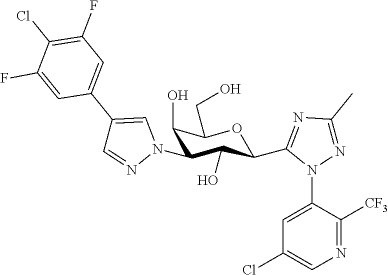 | 0.015 |
| 29 | 1-{5-{3-[4-(4-Chloro-3,5- difluorophenyl)-3-ethoxy- carbonyl-1H-1,2-pyrazol- 1-yl]-3-deoxy-β-D-galac- topyranosyl}-3-methyl- 1H-1,2,4-triazol-1-yl}-5- chloro-2-(trifluoromethyl)- benzene | 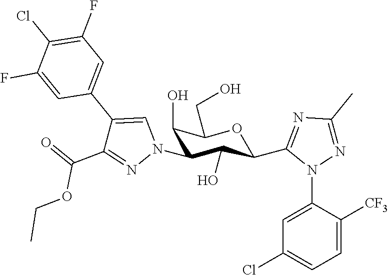 | 0.028 |
| 30 | 1-{5-{3-[3-Carboxy-4-(4- chloro-3,5-difluorophen- yl)-1H-1,2-pyrazol-1-yl]- 3-deoxy-β-D-galacto- pyranosyl}-3-methyl-1H- 1,2,4-triazol-1-yl}-5- chloro-2-(trifluoromethyl)- benzene | 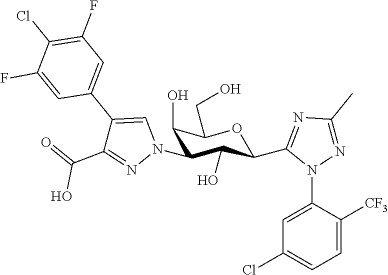 | 0.006 |
| 31 | 1-{5-{3-[4-(4-Chloro-3,5- difluorophenyl)-3-cyano- 1H-1,2-pyrazol-1-yl]-3- deoxy-β-D-galactopyran- osyl}-3-methyl-1H-1,2,4- triazol-1-yl}-5-chloro-2- (trifluoromethyl)benzene | 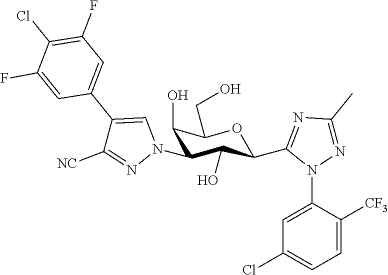 | 0.012 |
| 32 | 1-{5-{3-[3-Aminocar- bonyl-4-(4-chloro-3,5- difluorophenyl)-1H-1,2- pyrazol-1-yl]-3-deoxy-β- D-galactopyranosyl}-3- methyl-1H-1,2,4-triazol- 1-yl}-5-chloro-2-(trifluoro- methyl)benzene | 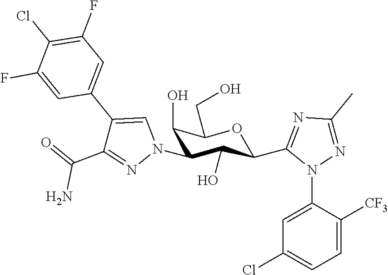 | 0.023 |
| 33 | 5-Chloro-2-{5-{3-deoxy- 3-[4-(3,4,5-trifluorophen- yl)-1H-1,2-pyrazol-1-yl]- β-D-galactopyranosyl}-3- methyl-1H-1,2,4-triazol- 1-yl}thiazole | 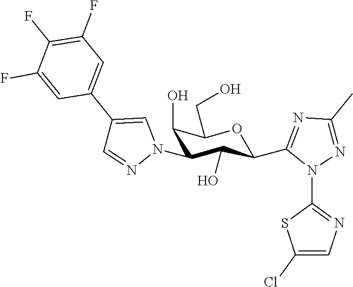 | 0.52 |
| 34 | 6-{5-{3-Deoxy-3-[4- (3,4,5-trifluorophenyl)- 1H-1,2-pyrazol-1-yl]-β- D-galactopyranosyl}-3- methyl-1H-1,2,4-triazol- 1-yl}benzothiazole | 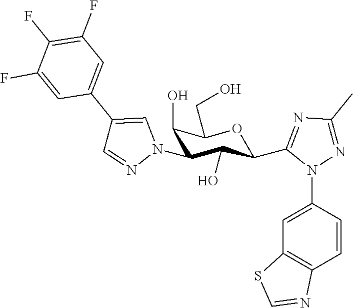 | 0.009 |
| 35 | 6-{5-{3-Deoxy-3-[4- (3,4,5-trifluorophenyl)- 1H-1,2-pyrazol-1-yl]-β- D-galactopyranosyl}-3- methyl-1H-1,2,4-triazol- 1-yl}-2-ethylbenzothiazole | 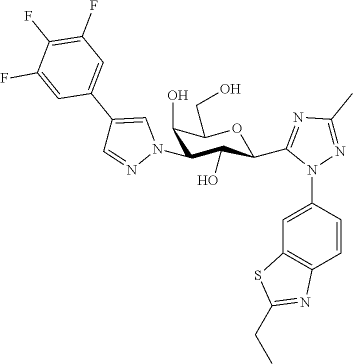 | 0.012 |
| 36 | 6-{5-{3-Deoxy-3-[4- (3,4,5-trifluorophenyl)- 1H-1,2-pyrazol-1-yl]-β- D-galactopyranosyl}-3- methyl-1HI-1,2,4-triazol- 1-yl}-2-(trifluoromethyl)- benzothiazole | 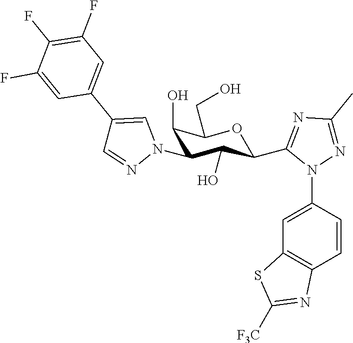 | 0.23 |
| 37 | 6-{5-{3-Deoxy-3-[4- (3,4,5-trifluorophenyl)- 1H-1,2-pyrazol-1-yl]-β- D-galactopyranosyl}-3- methyl-1H-1,2,4-triazol- 1-yl}-5-fluoro-2-methyl- benzothiazole | 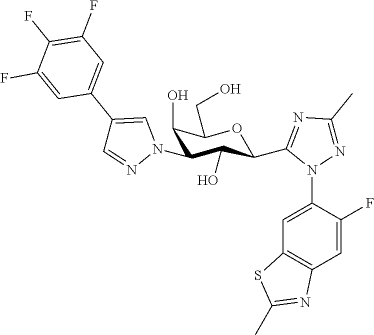 | 0.016 |
| 38 | 6-{5-{3-Deoxy-3-[4- (3,4,5-trifluorophenyl)- 1H-1,2-pyrazol-1-yl]-β- D-galactopyranosyl}-3- methyl-1H-1,2,4-triazol- 1-yl}-7-fluoro-2-methyl- benzothiazole | 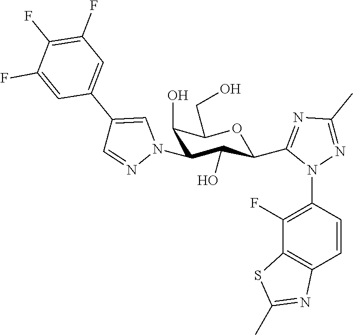 | 0.21 |
| 39 | 6-{5-{3-Deoxy-3-[4- (3,4,5-trifluorophenyl)- 1H-1,2-pyrazol-1-yl]-β- D-galactopyranosyl}-3- methyl-1H-1,2,4-triazol- 1-yl}-4-fluoro-2-methyl- benzothiazole | 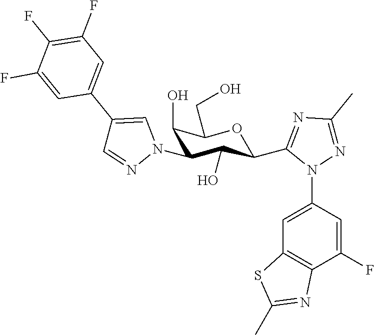 | 0.010 |
| 40 | 7-Chloro-6-{5-{3-deoxy- 3-[4-(3,4,5-trifluorophen- yl)-1H-1,2-pyrazol-1-yl]- β-D-galactopyranosyl}-3- methyl-1H-1,2,4-triazol- 1-yl}-2-methylbenzo- thiazole | 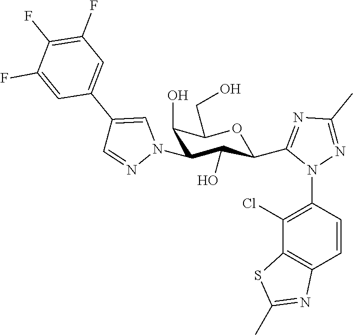 | 0.26 |
| 41 | 4-Chloro-6-{5-{3-deoxy- 3-[4-(3,4,5-trifluorophen- yl)-1H-1,2-pyrazol-1-yl]- β-D-galactopyranosyl}-3- methyl-1H-1,2,4-triazol- 1-yl}-2-methylbenzo- thiazole | 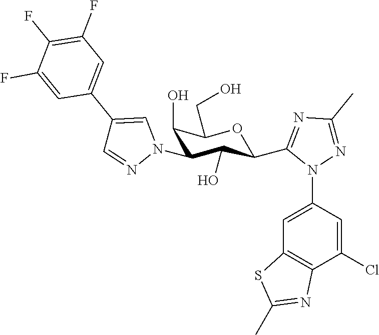 | 0.010 |
| 42 | 4-Cyano-6-{5-{3-deoxy- 3-[4-(3,4,5-trifluorophen- yl)-1H-1,2-pyrazol-1-yl]- β-D-galactopyranosyl}-3- methyl-1H-1,2,4-triazol- 1-yl}-2-methylbenzo- thiazole | 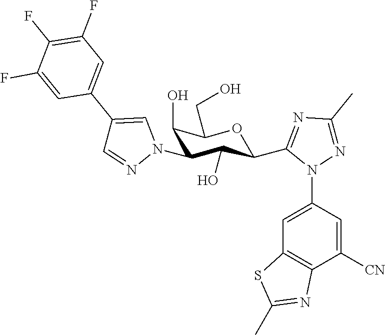 | 0.012 |
| 43 | 5-Bromo-6-{5-{3-deoxy- 3-[4-(3,4,5-trifluorophen- yl)-1H-1,2-pyrazol-1-yl]- β-D-galactopyranosyl}-3- methyl-1H-1,2,4-triazol- 1-yl}-2-methylbenzo- thiazole | 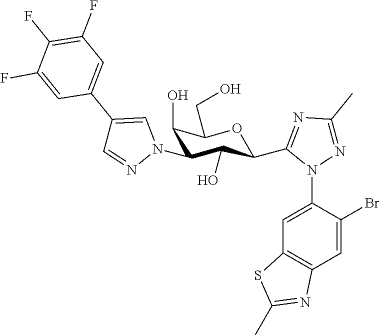 | 0.011 |
| 44 | 5-Cyano-6-{5-{3-deoxy- 3-[4-(3,4,5-trifluorophen- yl)-1H-1,2-pyrazol-1-yl]- β-D-galactopyranosyl}-3- methyl-1H-1,2,4-triazol- 1-yl}-2-methylbenzo- thiazole | 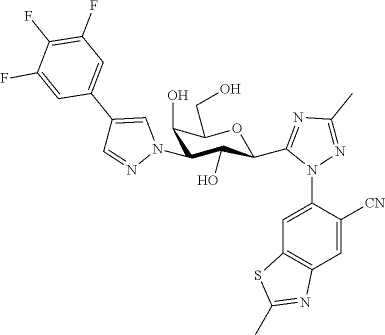 | 0.017 |
| 45 | 6-{5-{3-Deoxy-3-[4- (3,4,5-trifluorophenyl)- 1H-1,2-pyrazol-1-yl]-β- D-galactopyranosyl}-3- methyl-1H-1,2,4-triazol- 1-yl}-2-methylthiazolo- [4,5-b]pyridine | 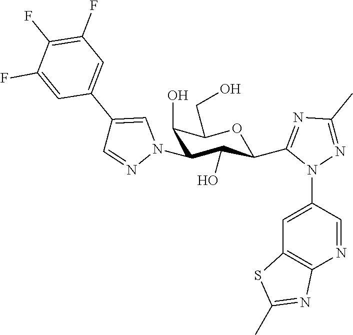 | 0.035 |
| 46 | 5-Cyano-1-{5-{3-deoxy- 3-[4-(3,4,5-trifluorophen- yl)-1H-1,2-pyrazol-1-yl]- β-D-galactopyranosyl}-3- methyl-1H-1,2,4-triazol- 1-yl}-2-(trifluorometh- yl)benzene | 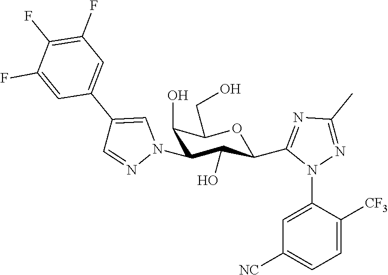 | 0.019 |
| 47 | 5-Chloro-1-{5-{3-deoxy- 3-[4-(3,4,5-trifluorophen- yl)-3-phenyl-1H-1,2- pyrazol-1-yl]-β-D-galac- topyranosyl}-3-methyl- 1H-1,2,4-triazol-1-yl}-2- (trifluoromethyl)benzene | 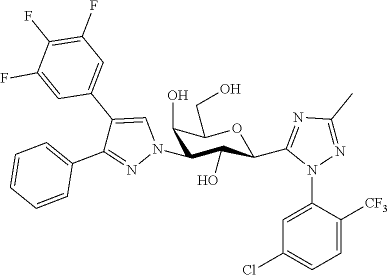 | 0.080 |
| 48 | 5-Chloro-1-{5-{3-deoxy- 3-[4-(3,4,5-trifluorophen- yl)-3-(2-fluorophenyl)-1H- 1,2-pyrazol-1-yl]-β-D- galactopyranosyl}-3- methyl-1H-1,2,4-triazol- 1-yl}-2-(trifluoromethyl)- benzene | 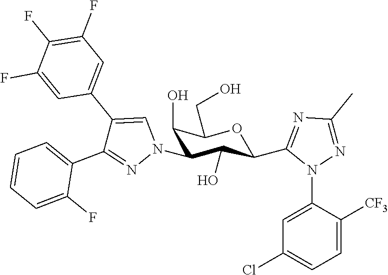 | 0.054 |
| 49 | 5-Chloro-1-{5-{3-deoxy- 3-[4-(3,4,5-trifluorophen- yl)-3-methyl-1H-1,2- pyrazol-1-yl]-β-D-galac- topyranosyl}-3-methyl- 1H-1,2,4-triazol-1-yl}-2- (trifluoromethyl)benzene | 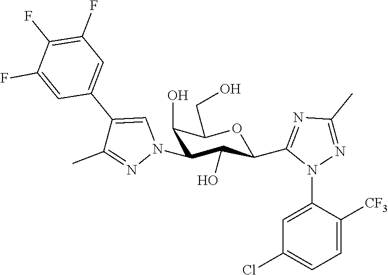 | 0.013 |
| 50 | 1-{5-{3-[3-Benzyl-4- (3,4,5-trifluorophenyl)- 1H-1,2-pyrazol-1-yl]-3- deoxy-β-D-galactopyran- osyl}-3-methyl-1H-1,2,4- triazol-1-yl}-5-chloro-2- (trifluoromethyl)benzene | 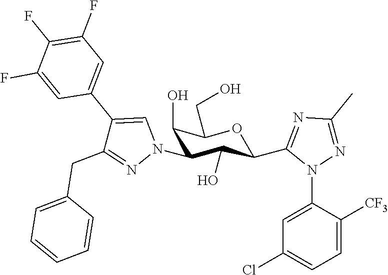 | 0.036 |
| 51 | 5-Chloro-1-{5-{3-[3- cyano-4-(3,4,5-trifluoro- phenyl)-1H-1,2-pyrazol- 1-yl]-3-deoxy-β-D- galactopyranosyl}-3- methyl-1H-1,2,4-triazol- 1-yl}-2-(trifluoromethyl)- benzene | 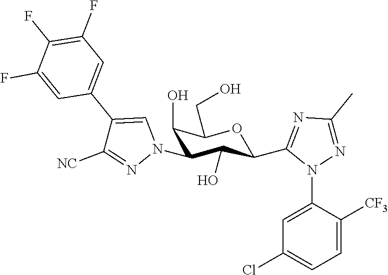 | 0.015 |
| 52 | 5-Chloro-1-{5-{3-deoxy- 3-[4-(3,4,5-trifluorophen- yl)-3-(2-methoxyphenyl)- 1H-1,2-pyrazol-1-yl]-β- D-galactopyranosyl}-3- methyl-1H-1,2,4-triazol- 1-yl}-2-(trifluoromethyl)- benzene | 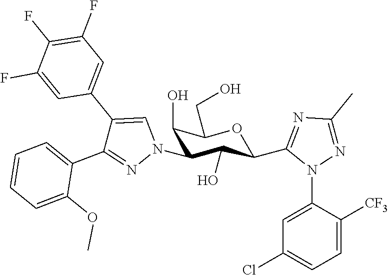 | 0.020 |
| 53 | 5-Chloro-1-{5-{3-deoxy- 3-[4-(3,4,5-trifluorophen- yl)-3-(2-methylphenyl)- 1H-1,2-pyrazol-1-yl]-β- D-galactopyranosyl}-3- methyl-1H-1,2,4-triazol- 1-yl}-2-(trifluoromethyl)- benzene | 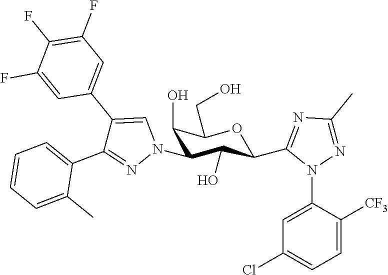 | 0.092 |
| 54 | 5-Chloro-1-{5-{3-deoxy- 3-[4-(3,4,5-trifluorophen- yl)-3-(3-fluorophenyl)- 1H-1,2-pyrazol-1-yl]-β- D-galactopyranosyl}-3- methyl-1H-1,2,4-triazol- 1-yl}-2-(trifluoromethyl)- benzene | 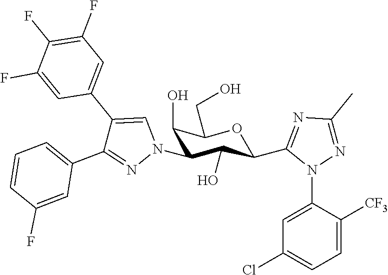 | 0.24 |
| 55 | 5-Chloro-1-{5-{3-deoxy- 3-[4-(3,4,5-trifluorophen- yl)-3-(4-fluorophenyl)- 1H-1,2-pyrazol-1-yl]-β- D-galactopyranosyl}-3- methyl-1H-1,2,4-triazol- 1-yl}-2-(trifluoromethyl)- benzene | 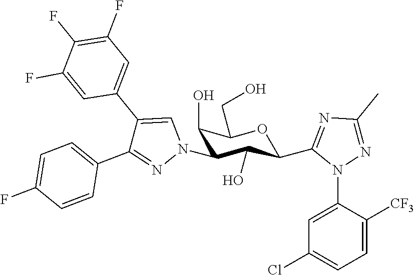 | 0.10 |
| 56 | 5-Chloro-1-{5-{3-deoxy- 3-{4-[4-(trifluoromethyl)- phenyl]-1H-1,2-pyrazol- 1-yl}-β-D-galactopyran- osyl}-3-methyl-1H-1,2,4- triazol-1-yl}-2-(trifluoro- methyl)benzene | 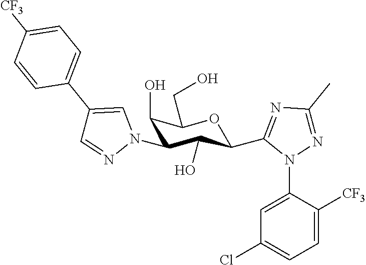 | 0.11 |
| 57 | 5-Chloro-1-{5-{3-deoxy- 3-[4-(3-fluoro-4-methyl- phenyl)-1H-1,2-pyrazol- 1-yl]-β-D-galactopyran- osyl}-3-methyl-1H-1,2,4- triazol-1-yl}-2-(trifluoro- methyl)benzene | 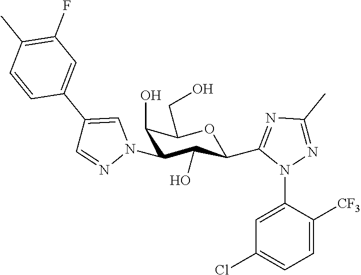 | 0.011 |
| 58 | 1-{5-{3-[4-(3-Chloro- phenyl)-1H-1,2-pyrazol- 1-yl]-3-deoxy-β-D- galactopyranosyl}-3- methyl-1H-1,2,4-triazol- 1-yl}-5-chloro-2- (trifluoromethyl)benzene | 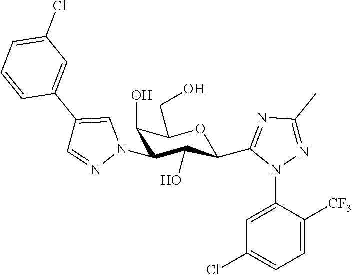 | 0.097 |
| 59 | 1-{5-{3-[4-(4-Chloro- phenyl)-1H-1,2-pyrazol- 1-yl]-3-deoxy-β-D- galactopyranosyl}-3- methyl-1H-1,2,4-triazol- 1-yl}-5-chloro-2- (trifluoromethyl)benzene | 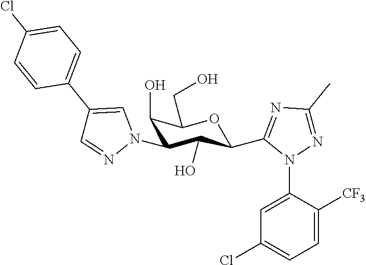 | 0.051 |

### In Vitro Adme Properties

In vitro PK properties as exemplified by examples 1, 5, 8, 9 and 13 suggests high uptake over the intestine and low hepatic metabolism. This data suggests high bioavailability and long halflife in man after for example oral administration.

| | CACO-2 A > B | Human hepatic Clint |
|---|---|---|
| Example | 10\hat{ }−6 cm/s | (ml/min/kg) |
| 1 | 18 | 1.3 |
| 5 | 18 | 0.4 |
| 8 | 17 | 0.3 |
| 9 | 20 | 0.6 |
| 13 | 10 | 0.2 |

### Synthesis of Examples and Intermediates

### General Experimental

Nuclear Magnetic Resonance (NMR) spectra were recorded on a 400 MHz Bruker AVANCE III 500 instrument or a Varian instrument at 400 MHz, at 25° C. Chemical shifts are reported in ppm (d) using the residual solvent as internal standard. Peak multiplicities are expressed as follow: s, singlet; d, doublet; dd, doublet of doublets; t, triplet; dt, doublet of triplet; q, quartet; m, multiplet; br s, broad singlet. LC-MS were acquired on an Agilent 1200 HPLC coupled with an Agilent MSD mass spectrometer operating in ES (+) ionization mode. Column: XBridge C18 (4.6×50 mm,3.5 μm) or SunFire C18 (4.6×50 mm, 3.5 μm). Solvent A water+0.1% TFA and solvent B Acetonitrile+0.1% TFA or solvent A water (10 mM Ammonium hydrogen carbonate) and solvent B Acetonitrile. Wavelength: 254 nM. Alternatively, LC-MS were acquired on an Agilent 1100 HPLC coupled with an Agilent MSD mass spectrometer operating in ES (+) ionization mode. Column: Waters symmetry 2.1×30 mm C18 or Chromolith RP-18 2×50 mm. Solvent A water+0.1% TFA and solvent B Acetonitrile+0.1% TFA. Wavelength 254 nm.

Preparative HPLC was performed on a Gilson 215. Flow: 25 mL/min Column: XBridge prep C18 10 μm OBD (19×250 mm) column. Wavelength: 254 nM. Solvent A water (10 mM Ammonium hydrogen carbonate) and solvent B Acetonitrile. Alternatively, preparative HPLC were acquired on a Gilson system. Flow: 15 ml/min Column: kromasil 100-5-C18 column. Wavelength: 220 nm. Solvent A water+0.1% TFA and solvent B Acetonitrile+0.1% TFA.

The following abbreviations are used
aq: aqueous
Calcd: Calculated
MeCN: Acetonitrile
CuI: Copper Iodide
DCM: Dichloromethane
DIPEA: Diisopropylethylamine
DMF: N,N-dimethylformamide
ESI-MS: Electrospray ionization mass spectrometry
EtOAc or EA: Ethylacetate
Et₃N: Triethylamine
h: hour(s)
HPLC: High performance liquid chromatography
K₂CO₃: Potassium carbonate
LC: Liquid Chromatography
MeCN: Acetonitrile
mL: milliliter
MeOH: Methanol
MeOD: Deuterated methanol
mm: millimeter
mM: millimolar
MS: Mass spectroscopy
nm: nanometer
NaOMe: Sodium methoxide
N₂: Nitrogen gas
NMR: Nuclear magnetic resonance
Pd₂(dba)₃: tris(dibenzylideneacetone)dipalladium(0)
Pd(dppf)Cl₂: [1,1′-bis(diphenylphosphino)ferrocene]dichloropalladium(II)
Pd(PPh₃)₄: tetrakis(triphenylphosphine)palladium(0)
PE: petroleum ether
pH: acidity
Prep: preparative
rt: room temperature
TBAF: tetrabutylammonium fluoride
TFA: trifluoroacetic acid
THF: Tetrahydrofuran
TMS: Trimethylsilyl
UV: Ultraviolet
Å: Angström

### Synthesis of Example 1-59 from their Respective Intermediates 1-59

When naming examples and intermediates with aryl groups connected directly to C₁ of the galactose unit the following methodology has been used. The highest priority has been given to the aryl furthest away from the galactose carbon1 (C₁), regardless of IUPAC rules.

### Example 1

### 3-{5-{3-[4-(4-Chloro-3,5-difluorophenyl)-1H-1,2-pyrazol-1-yl]-3-deoxy-β-D-galactopyranosyl}-3-methyl-1H-1,2,4-triazol-1-yl}-5-chloro-2-cyclopropylpyridine

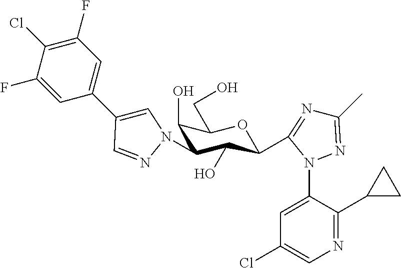

To a solution of 3-{5-[3-(4-bromo-1H-1,2-pyrazol-1-yl)-3-deoxy-β-D-galactopyranosyl]-3-methyl-1H-1,2,4-triazol-1-yl}-5-chloro-2-cyclopropylpyridine (55 mg, 0.11 mmol) and (4-chloro-3,5-difluorophenyl)boronic acid (40.2 mg, 0.21 mmol) in 1,4-dioxane/H₂O (18 mL, 2:1) Pd(PPh₃)₄ (12.1 mg, 0.011 mmol) and K₂CO₃ (43.4 mg, 0.31 mmol) were added. The mixture was purged three times with nitrogen, and stirred 3 h at 80° C. The mixture was filtered, and the filtrate was acidified to pH 3-4 using TFA. The filtrate was concentrated and purified by prep HPLC [MeCN/H₂O (10 mmol/L TFA), X-Select10 μm 19*250 mm, 20 m/min, UV 254] to afford the title compound (9.93 mg, 16%). ESI-MS m/z calcd for [C₂₆H₂₄Cl₂F₂N₆O₄] [M+H]⁺: 593.1; found: 593.0. ¹H NMR (400 MHz, Methanol-d₄) δ 8.48 (d, J=2.4 Hz, 1H), 8.11 (s, 1H), 7.83 (s, 1H), 7.80 (s, 1H), 7.28-7.24 (m, 2H), 4.61-4.56 (m, 1H), 4.32-4.26 (m, 2H), 3.98 (d, J=2.8 Hz, 1H), 3.65-3.52 (m, 3H), 2.36 (s, 3H), 1.60-1.53 (m, 1H), 1.03-0.82 (m, 4H).

### Example 2

### 6-{3-{3-Deoxy-3-[4-(3,4,5-trifluorophenyl)-1H-1,2-pyrazol-1-yl]-β-D-galactopyranosyl}-4H-1,2,4-triazol-4-yl}-2-methylbenzothiazole

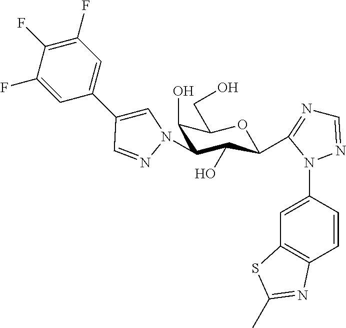

A nitrogen purged solution of 6-{3-[3-(4-bromo-1H-1,2-pyrazol-1-yl)-3-deoxy-β-D-galactopyranosyl]-4H-1,2,4-triazol-4-yl}-2-methylbenzothiazole (56 mg, 0.11 mmol), (3,4,5-trifluorophenyl)boronic acid (38.8 mg, 0.22 mmol), K₂CO₃ (45.7 mg, 0.33 mmol) and Pd(dppf)Cl₂ (16.1 mg, 0.022 mmol) in 1,4-dioxane (2 mL) and water (1 mL) was stirred 3 h at 80° C. The mixture was filtered, concentrated, and purified by prep HPLC (MeCN/H₂O (10 mmol/LTFA), X-Select10 um 19*250 mm, 20 m/min, UV 254) to the title compound (5.69 mg, 9%). ESI-MS m/z calcd for [C₂₅H₂₁F₃N₆O₄S][M+H]⁺: 559.1 found: 559.0. ¹H NMR (400 MHz, Methanol-d₄) δ 8.75 (s, 1H), 8.26 (d, J=2.0 Hz, 1H), 8.07 (s, 1H), 7.99 (d, J=8.4 Hz, 1H), 7.78 (s, 1H), 7.64 (dd, J=8.4, 2.4 Hz, 1H), 7.25 (dd, J=9.6, 6.4 Hz, 2H), 4.87-4.78 (m, 1H), 4.34 (d, J=9.6 Hz, 1H), 4.30 (dd, J=10.8, 2.8 Hz, 1H), 3.98 (d, J=2.8 Hz, 1H), 3.71-3.58 (m, 3H), 2.80 (s, 3H).

### Example 3

### 5-Chloro-1-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-(trifluoromethyl)benzene

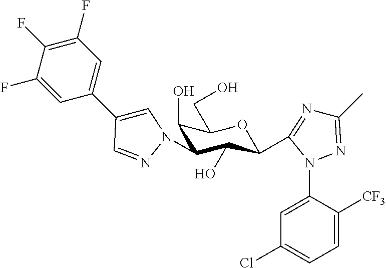

and

### Example 4

### 1-{5-{3-Deoxy-3-[4-(3,4,5-trifluorophenyl)-1H-1,2-pyrazol-1-yl]-β-D-galactopyranosyl}-3-methyl-1H-1,2,4-triazol-1-yl}-2-(trifluoromethyl)-2-(3,4,5-trifluorophenyl)benzene

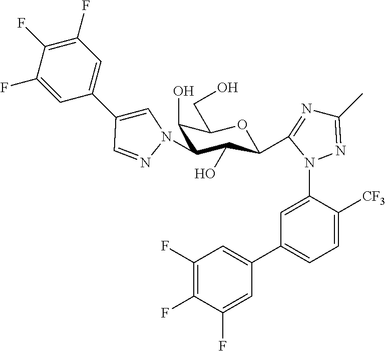

To a solution of 1-{5-[3-(4-bromo-1H-1,2-pyrazol-1-yl)-3-deoxy-β-D-galactopyranosyl]-3-methyl-1H-1,2,4-triazol-1-yl}-5-chloro-2-(trifluoromethyl)benzene (50 mg, 0.091 mmol) and (3,4,5-trifluorophenyl)boronic acid (31.8 mg, 0.18 mmol) in 1,4-dioxane/H₂O (3.0 mL, 2:1) Pd(PPh₃)₄ (10.5 mg, 0.0091 mmol) and K₂CO₃ (37.5 mg, 0.27 mmol) were added. The mixture was purged three times with nitrogen, and stirred 3 h at 80° C. The mixture was filtered, and the filtrate was acidified to pH 3-4 using TFA. The mixture was concentrated and purified by column chromatography (MeOH/DCM=0/1˜1/10, Silica-CS 12 g, 20 mL/min, silica gel, UV 254). The obtained material was further purified by prep HPLC [MeCN/H₂O (10 mmol/L TFA), X-Select 10 μm 19*250 mm, 20 mL/min, UV 254] to afford the two title compounds (example 3, 4.68 mg, 9%, and example 4, 4.88 mg, 8%).

### 5-Chloro-1-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-(trifluoromethyl)benzene

ESI-MS m/z calcd for [C₂₅H₂₀ClF₆N₅O₄] [M+H]⁺: 604.1; found: 603.8. ¹H NMR (400 MHz, Methanol-d₄) δ 8.09 (s, 1H), 7.86 (d, J=8.4 Hz, 1H), 7.80 (s, 1H), 7.76 (d, J=8.8 Hz, 1H), 7.67 (s, 1H), 7.34-7.22 (m, 2H), 4.71-4.58 (m, 1H), 4.28 (dd, J=10.8, 2.8 Hz, 1H), 4.18 (d, J=9.2 Hz, 1H), 3.99 (d, J=2.4 Hz, 1H), 3.61-3.51 (m, 3H), 2.34 (s, 3H).

### 1-{5-{3-Deoxy-3-[4-(3,4,5-trifluorophenyl)-1H-1,2-pyrazol-1-yl]-β-D-galactopyranosyl}-3-methyl-1H-1,2,4-triazol-1-yl}-2-(trifluoromethyl)-2-(3,4,5-trifluorophenyl)benzene

ESI-MS m/z calcd for [C₃₁H₂₂F₉N₅O₄] [M+H]⁺: 700.2; found: 699.7. ¹H NMR (400 MHz, Methanol-d₄) δ 8.08 (s, 1H), 8.01-7.96 (m, 1H), 7.94 (d, J=8.4 Hz, 1H), 7.82 (s, 1H), 7.79 (s, 1H), 7.55-7.47 (m, 2H), 7.29-7.22 (m, 2H), 4.72-4.60 (m, 1H), 4.27 (dd, J=10.8, 2.8 Hz, 1H), 4.24 (d, J=10.8 Hz, 1H), 3.95 (d, J=2.8 Hz, 1H), 3.63-3.44 (m, 3H), 2.36 (s, 3H).

### Example 5

### 1-{5-{3-[4-(4-Chloro-3,5-difluorophenyl)-1H-1,2-pyrazol-1-yl]-3-deoxy-β-D-galactopyranosyl}-3-methyl-1H-1,2,4-triazol-1-yl}-5-chloro-2-(trifluoromethyl)benzene

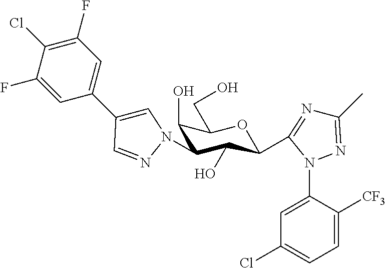

To a solution of 5-chloro-1-{5-[3-deoxy-3-(4-iodo-1H-1,2-pyrazol-1-yl)-β-D-galactopyranosyl]-3-methyl-1H-1,2,4-triazol-1-yl}-2-(trifluoromethyl)benzene (55 mg, 0.092 mmol) and (4-chloro-3,5-difluorophenyl)boronic acid (35.3 mg, 0.18 mmol) in 1,4-dioxane/H₂O (3.0 mL, 2:1) Pd(PPh₃)₄ (10.6 mg, 0.0092 mmol) and K₂CO₃ (38.0 mg, 0.28 mmol) were added. The mixture was purged three times with nitrogen, and stirred 2 h at 60° C. The mixture was filtered, and the filtrate was acidified to pH 3-4 using TFA. The mixture was concentrated and purified by column chromatography (MeOH/DCM=0˜1/10, Silica-CS 12 g, 20 mL/min, silica gel, UV 254). The obtained material was purified by prep HPLC [MeCN/H₂O (10 mmol/L TFA), X-Select 10 μm 19*250 mm, 20 mL/min, UV 254] to afford the title compound (7.12 mg, 13%). ESI-MS m/z calcd for [C₂₅H₂₀Cl₂F₅N₅O₄] [M+H]⁺: 620.1; found: 619.7. ¹H NMR (400 MHz, Methanol-d₄) δ 8.15 (s, 1H), 7.90-7.82 (m, 2H), 7.76 (dd, J=1.2, 8.4 Hz, 1H), 7.67 (s, 1H), 7.30 (d, J=4.8 Hz, 1H), 7.27 (d, J=4.4 Hz, 1H), 4.72-4.60 (m, 1H), 4.29 (dd, J=10.8, 2.8 Hz, 1H), 4.19 (d, J=9.2 Hz, 1H), 3.99 (d, J=2.8 Hz, 1H), 3.61-3.50 (m, 3H), 2.34 (s, 3H).

### Example 6

### 5-Chloro-1-{5-{3-deoxy-3-[4-(6-fluoropyridin-2-yl)-1H-1,2-pyrazol-1-yl]-β-D-galactopyranosyl}-3-methyl-1H-1,2,4-triazol-1-yl}-2-(trifluoromethyl)benzene

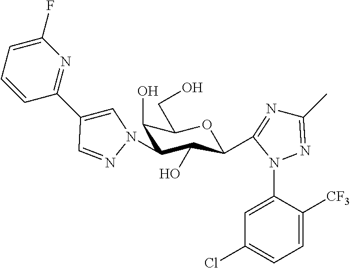

To a solution of 5-chloro-1-{5-[3-deoxy-3-(4-iodo-1H-1,2-pyrazol-1-yl)-β-D-galactopyranosyl]-3-methyl-1H-1,2,4-triazol-1-yl}-2-(trifluoromethyl)benzene (45 mg, 0.075 mmol) and (6-fluoropyrid-2-yl)boronic acid (31.7 mg, 0.23 mmol) in 1,4-dioxane/H₂O (3.0 mL, 2:1) Pd(dppf)Cl₂ (11 mg, 0.015 mmol) and K₂CO₃ (51.9 mg, 0.38 mmol) were added. The mixture was purged three times with nitrogen, and stirred 2 h at 65° C. The mixture was filtered, and the filtrate was acidified to pH 3-4 using TFA. The mixture was evaporated and purified by prep HPLC [MeCN/H₂O (10 mmol/L NH₄HCO₃), X-Select 10 μm 19*250 mm, 20 mL/min, UV 254] to afford the title compound (16.1 mg, 38%). ESI-MS m/z calcd for [C₂₄H₂₁ClF₄N₆O₄] [M+H]⁺: 569.1; found: 568.8. ¹H NMR (400 MHz, Methanol-d₄) δ 8.27 (s, 1H), 7.96 (s, 1H), 7.86 (d, J=8.4 Hz, 1H), 7.83-7.73 (m, 2H), 7.67 (s, 1H), 7.45 (dd, J=7.6, 2.0 Hz, 1H), 6.73 (dd, J=8.0, 2.4 Hz, 1H), 4.74-4.59 (m, 1H), 4.31 (dd, J=10.8, 2.8 Hz, 1H), 4.20 (d, J=9.2 Hz, 1H), 4.00 (d, J=2.8 Hz, 1H), 3.62-3.49 (m, 3H), 2.34 (s, 3H).

### Example 7

### 5-Chloro-1-{5-{3-deoxy-3-[4-(4-fluoropyridin-2-yl)-1H-1,2-pyrazol-1-yl]-β-D-galactopyranosyl}-3-methyl-1H-1,2,4-triazol-1-yl}-2-(trifluoromethyl)benzene

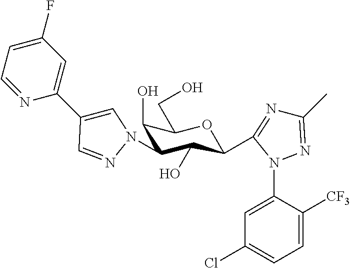

To a solution of 1-{5-[3-(4-borono-1H-1,2-pyrazol-1-yl)-3-deoxy-β-D-galactopyranosyl]-3-methyl-1H-1,2,4-triazol-1-yl}-5-chloro-2-(trifluoromethyl)benzene (55 mg, 0.11 mmol) and 2-bromo-4-fluoropyridine (93.5 mg, 0.53 mmol) in 1,4-dioxane/H₂O (3.0 mL, 2:1) Pd(dppf)Cl₂ (15.5 mg, 0.021 mmol) and K₂CO₃ (73.4 mg, 0.53 mmol) were added. The mixture was purged three times with nitrogen, and stirred 2 h at 80° C. The mixture was filtered, and the filtrate was acidified to pH 3-4 using TFA. The mixture was evaporated and purified by prep HPLC [MeCN/H₂O (10 mmol/L NH₄HCO₃), X-Select 10 μm 19*250 mm, 20 m/min, UV 254] to afford the title compound (19 mg, 32%). ESI-MS m/z calcd for [C₂₄H₂₁ClF₄N₆O₄] [M+H]⁺: 569.1; found: 568.8. ¹H NMR (400 MHz, Methanol-d₄) δ 8.39 (dd, J=8.8, 6.0 Hz, 1H), 8.30 (s, 1H), 7.99 (s, 1H), 7.86 (d, J=8.4 Hz, 1H), 7.76 (dd, J=1.2, 8.4 Hz, 1H), 7.67 (s, 1H), 7.41 (dd, J=10.4, 2.4 Hz, 1H), 6.96-6.92 (m, 1H), 4.73-4.61 (m, 1H), 4.32 (dd, J=10.8, 2.8 Hz, 1H), 4.20 (d, J=9.2 Hz, 1H), 4.01 (d, J=2.8 Hz, 1H), 3.64-3.50 (m, 3H), 2.34 (s, 3H).

### Example 8

### 6-{5-{3-Deoxy-3-[4-(3,4,5-trifluorophenyl)-1H-1,2-pyrazol-1-yl]-β-D-galactopyranosyl}-3-methyl-1H-1,2,4-triazol-1-yl}-2-methylbenzothiazole

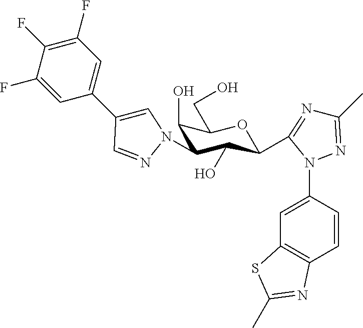

A solution of 6-{5-[3-(4-bromo-1H-1,2-pyrazol-1-yl)-3-deoxy-β-D-galactopyranosyl]-3-methyl-1H-1,2,4-triazol-1-yl}-2-methylbenzothiazole (65 mg, 0.13 mmol), K₂CO₃ (86.1 mg, 0.62 mmol), (3,4,5-trifluorophenyl)boronic acid (110 mg, 0.62 mmol), and Pd(PPh₃)₄ (28.8 mg, 0.025 mmol) in 1,4-dioxane (4 mL) and water (2 mL) was stirred 12 h at 80° C. The mixture was filtered, concentrated and purified by prep HPLC (MeCN/H₂O (10 mmol/L TFA), X-Select 10 μm 19*250 mm, 20 mL/min, UV 254) to give the title compound (12 mg, 17%). ESI-MS m/z calcd for [C₂₆H₂₃F₃N₆O₄S][M+H]⁺: 573.1; found: 572.8. ¹H NMR (400 MHz, Methanol-d₄) δ 8.36 (d, J=2.0 Hz, 1H), 8.19 (s, 1H), 8.06 (d, J=8.8 Hz, 1H), 7.89 (s, 1H), 7.79 (dd, J=8.8, 2.0 Hz, 1H), 7.35 (dd, J=9.2, 6.4 Hz, 2H), 4.91 (d, J=10.8 Hz, 1H), 4.48 (d, J=9.6 Hz, 1H), 4.42 (dd, J=10.8, 2.8 Hz, 1H), 4.09 (d, J=3.2 Hz, 1H), 3.84-3.78 (m, 2H), 3.74-3.67 (m, 1H), 2.89 (s, 3H), 2.47 (s, 3H).

### Example 9

### 6-{5-{3-[4-(4-Chloro-3,5-difluorophenyl)-1H-1,2-pyrazol-1-yl]-3-deoxy-β-D-galactopyranosyl}-3-methyl-1H-1,2,4-triazol-1-yl}-2-methylbenzothiazole

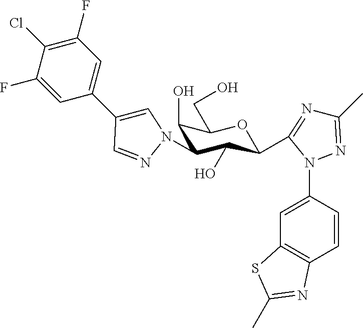

A solution of 6-{5-[3-(4-bromo-1H-1,2-pyrazol-1-yl)-3-deoxy-β-D-galactopyranosyl]-3-methyl-1H-1,2,4-triazol-1-yl}-2-methylbenzothiazole (110 mg, 0.21 mmol), K₂CO₃ (146 mg, 1.05 mmol), (4-chloro-3,5-difluorophenyl)boronic acid (203 mg, 1.05 mmol), and Pd(dppf)Cl₂ (30.9 mg, 0.042 mmol) in 1,4-dioxane (4 mL) and water (2 mL) was stirred 12 h at 80° C. The mixture was filtered, concentrated and purified by prep HPLC (MeCN/H₂O (10 mmol/L TFA), X-Select 10 μm 19*250 mm, 20 mL/min, UV 254) to give the title compound (13 mg, 11%). ESI-MS m/z calcd for [C₂₆H₂₃ClF₂N₆O₄S][M+H]⁺: 589.1; found: 588.8. ¹H NMR (400 MHz, Methanol-d₄) δ 8.27 (d, J=2.0 Hz, 1H), 8.15 (s, 1H), 7.96 (d, J=8.8 Hz, 1H), 7.84 (s, 1H), 7.69 (dd, J=8.4, 2.0 Hz, 1H), 7.28 (dd, J=12.8, 4.4 Hz, 2H), 4.82 (d, J=10.4 Hz, 1H), 4.39 (d, J=9.6 Hz, 1H), 4.33 (dd, J=10.8, 2.8 Hz, 1H), 4.00 (d, J=2.8 Hz, 1H), 3.75-3.68 (m, 2H), 3.64-3.60 (m, 1H), 2.79 (s, 3H), 2.37 (s, 3H).

### Example 10 (Atropisomer of Example 11)

### 1-{3-{3-[4-(4-Chloro-3,5-difluorophenyl)-1H-1,2-pyrazol-1-yl]-3-deoxy-β-D-galactopyranosyl}-5-methyl-4H-1,2,4-triazol-4-yl}-5-chloro-2-(trifluoromethyl)benzene

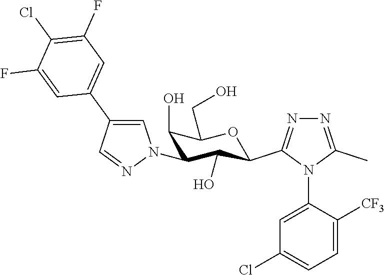

A solution of 5-chloro-1-{3-[3-deoxy-3-(4-iodo-1H-1,2-pyrazol-1-yl)-β-D-galactopyranosyl]-5-methyl-4H-1,2,4-triazol-4-yl}-2-(trifluoromethyl)benzene (atropisomer 1) (119 mg, 0.16 mmol), K₂CO₃ (65.7 mg, 0.48 mmol), (4-chloro-3,5-difluorophenyl)boronic acid (61.1 mg, 0.32 mmol), and Pd(PPh₃)₄ (18.3 mg, 0.016 mmol) in DMF (2 mL) and water (1 mL) was stirred 3 h at 60° C. The mixture was filtered, concentrated and purified by prep HPLC (MeCN/H₂O (10 mmol/L HCOOH), X-Select 10 μm 19*250 mm, 20 mL/min, UV 254) to give the title compound (4.4 mg, 4%). ESI-MS m/z calcd for [C₂₅H₂₀Cl₂F₅N₅O₄] [M+H]⁺: 620.1 found: 619.8. ¹H NMR (400 MHz, Methanol-d₄) δ 8.14 (s, 1H), 7.93 (d, J=8.8 Hz, 1H), 7.83 (s, 1H), 7.81 (d, J=9.2 Hz, 1H), 7.54 (d, J=2.0 Hz, 1H), 7.29 (s, 1H), 7.27 (s, 1H), 4.57 (t, J=10.0 Hz, 1H), 4.25 (dd, J=10.8, 2.8 Hz, 1H), 4.08 (d, J=9.2 Hz, 1H), 3.98 (d, J=2.4 Hz, 1H), 3.57-3.51 (m, 3H), 2.15 (s, 3H).

### Example 11 (Atropisomer of Example 10)

### 1-{3-{3-[4-(4-Chloro-3,5-difluorophenyl)-1H-1,2-pyrazol-1-yl]-3-deoxy-β-D-galactopyranosyl}-5-methyl-4H-1,2,4-triazol-4-yl}-5-chloro-2-(trifluoromethyl)benzene

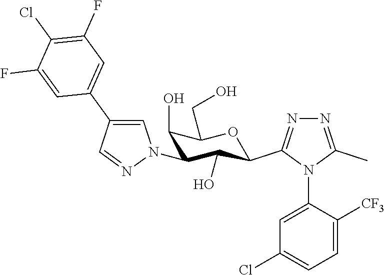

A solution of 5-chloro-1-{3-[3-deoxy-3-(4-iodo-1H-1,2-pyrazol-1-yl)-β-D-galactopyranosyl]-5-methyl-4H-1,2,4-triazol-4-yl}-2-(trifluoromethyl)benzene (atropisomer 2) (63 mg, 0.11 mmol), K₂CO₃ (43.5 mg, 0.32 mmol), (4-chloro-3,5-difluorophenyl)boronic acid (40.4 mg, 0.21 mmol), and Pd(dppf)Cl₂ (7.7 mg, 0.011 mmol) in DMF (2 mL) and water (1 mL) was stirred 3 h at 60° C. The mixture was filtered, concentrated and purified by prep HPLC (MeCN/H₂O (10 mmol/L HCOOH), X-Select 10 μm 19*250 mm, 20 mL/min, UV 254) to give the title compound (6.3 mg, 10%). ESI-MS m/z calcd for [C₂₅H₂₀Cl₂F₅N₅O₄] [M+H]⁺: 620.1 found: 619.8. ¹H NMR (400 MHz, Methanol-d₄) δ 8.17 (s, 1H), 7.91 (d, J=8.8 Hz, 1H), 7.85 (d, J=0.4 Hz, 1H), 7.81 (d, J=8.4 Hz, 1H), 7.75 (d, J=1.6 Hz, 1H), 7.31 (s, 1H), 7.29 (s, 1H), 4.89 (t, J=10.0 Hz, 1H), 4.28 (dd, J=10.8, 2.8 Hz, 1H), 4.09 (d, J=9.2 Hz, 1H), 3.95 (d, J=2.0 Hz, 1H), 3.46-3.44 (m, 2H), 3.40-3.38 (m, 1H), 2.17 (s, 3H).

### Example 12

### 1-{5-{3-[4-(4-Chloro-3,5-difluorophenyl)-3-methyl-1H-1,2-pyrazol-1-yl]-3-deoxy-β-D-galactopyranosyl}-3-methyl-1H-1,2,4-triazol-1-yl}-5-chloro-2-(trifluoromethyl)benzene

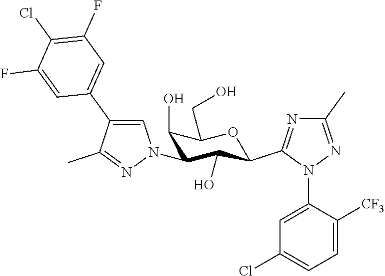

To a solution of 5-chloro-1-{5-[3-deoxy-3-(4-iodo-3-methyl-1H-1,2-pyrazol-1-yl)-β-D-galactopyranosyl]-3-methyl-1H-1,2,4-triazol-1-yl}-2-(trifluoromethyl)benzene (55 mg, 0.090 mmol) and (4-chloro-3,5-difluorophenyl)boronic acid (51.7 mg, 0.27 mmol) in 1,4-dioxane/H₂O (3.0 mL, 2:1) Pd(dppf)Cl₂ (13.1 mg, 0.018 mmol) and K₂CO₃ (61.9 mg, 0.45 mmol) were added. The mixture was purged three times with nitrogen, and stirred 2 h at 65° C. The mixture was filtered, and the filtrate was acidified to pH 3-4 using TFA. The mixture was concentrated and purified by prep HPLC [MeCN/H₂O (10 mmol/L NH₄HCO₃), X-Select 10 μm 19*250 mm, 20 mL/min, UV 254] to afford the title compound (31.6 mg, 56%). ESI-MS m/z calcd for [C₂₆H₂₂Cl₂F₅N₅O₄] [M+H]⁺: 634.1; found: 633.8. ¹H NMR (400 MHz, Methanol-d₄) δ 7.93 (s, 1H), 7.85 (d, J=8.4 Hz, 1H), 7.75 (d, J=8.8 Hz, 1H), 7.67 (s, 1H), 7.13 (d, J=8.4 Hz, 2H), 4.73-4.57 (m, 1H), 4.24-4.13 (m, 2H), 3.97 (d, J=2.4 Hz, 1H), 3.63-3.48 (m, 3H), 2.34 (s, 3H), 2.29 (s, 3H).

### Example 13

### 5-Chloro-3-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-cyclopropylpyridine

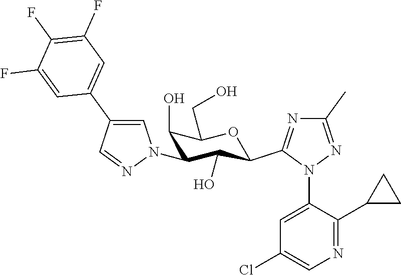

To a solution of 5-chloro-3-{5-[3-deoxy-3-(4-iodo-1H-1,2-pyrazol-1-yl)-β-D-galactopyranosyl]-3-methyl-1H-1,2,4-triazol-1-yl}-2-cyclopropylpyridine (50 mg, 0.087 mmol) and (3,4,5-trifluorophenyl)boronic acid (15.4 mg, 0.087 mmol) in 1,4-dioxane/H₂O (6 mL, 2:1) Pd(dppf)Cl₂ (12.8 mg, 0.018 mmol) and K₂CO₃ (24.1 mg, 0.17 mmol) were added. The mixture was purged three times with nitrogen, and stirred 3 h at 40° C. The mixture was filtered, and the filtrate was acidified to pH 3-4 using TFA. The filtrate was concentrated and purified by prep HPLC [MeCN/H₂O (10 mmol/L TFA), X-Select10 μm 19*250 mm, 20 mL/min, UV 254] to afford the title compound (9.93 mg, 16%). ESI-MS m/z calcd for [C₂₆H₂₄ClF₃N₆O₄] [M+H]⁺: 577.1; found: 576.9. ¹H NMR (400 MHz, DMSO-d₆) δ 8.68 (d, J=2.0 Hz, 1H), 8.34 (s, 1H), 8.00 (s, 1H), 7.92 (s, 1H), 7.64-7.60 (m, 2H), 5.25 (d, J=6.8 Hz, 1H), 5.07 (d, J=5.6 Hz, 1H), 4.67-4.63 (m, 2H), 4.42 (d, J=8.0 Hz, 1H), 4.27 (d, J=8.8 Hz, 1H), 3.90 (s, 1H), 3.63-3.60 (m, 1H), 3.45-3.39 (m, 2H), 2.39 (s, 3H), 1.65-1.64 (m, 1H), 1.04-0.91 (m, 4H).

### Example 14

### 1-{5-{3-[4-(5-Chloro-4-fluoropyridin-2-yl)-1H-1,2-pyrazol-1-yl]-3-deoxy-β-D-galactopyranosyl}-3-methyl-1H-1,2,4-triazol-1-yl}-5-chloro-2-(trifluoromethyl)benzene

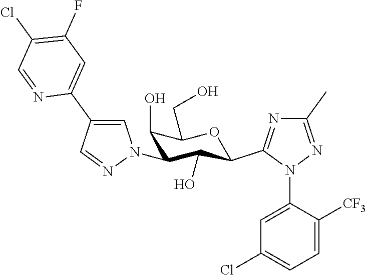

To a solution of 1-{5-[3-(4-borono-1H-1,2-pyrazol-1-yl)-3-deoxy-β-D-galactopyranosyl]-3-methyl-1H-1,2,4-triazol-1-yl}-5-chloro-2-(trifluoromethyl)benzene (30 mg, 0.058 mmol) and 2-bromo-5-chloro-4-fluoropyridine (61 mg, 0.29 mmol) in 1,4-dioxane/H₂O (2.5 mL, 4:1) Pd(dppf)Cl₂ (8.5 mg, 0.012 mmol) and K₂CO₃ (40 mg, 0.29 mmol) were added. The mixture was purged three times with nitrogen, and stirred 2 h at 80° C. The mixture was filtered, and the filtrate was acidified to pH 3-4 using TFA. The mixture was evaporated and purified by prep HPLC [MeCN/H₂O (10 mmol/L NH₄HCO₃), X-Select 10 μm 19*250 mm, 20 m/min, UV 254] to afford the title compound (6.5 mg, 19%). ESI-MS m/z calcd for [C₂₄H₂₀Cl₂F₄N₆O₄] [M+H]⁺: 603.1; found: 602.8. ¹H NMR (400 MHz, Methanol-d₄) δ 8.44 (d, J=9.6 Hz, 1H), 8.30 (s, 1H), 7.98 (s, 1H), 7.86 (d, J=8.4 Hz, 1H), 7.76 (dd, J=8.4, 1.2 Hz, 1H), 7.67 (s, 1H), 7.54 (d, J=10.4 Hz, 1H), 4.75-4.60 (m, 1H), 4.32 (dd, J=10.8, 2.8 Hz, 1H), 4.20 (d, J=9.6 Hz, 1H), 4.00 (d, J=2.8 Hz, 1H), 3.62-3.51 (m, 3H), 2.34 (s, 3H).

### Example 15

### 5-Chloro-1-{5-{3-deoxy-3-[4-(4-fluoro-5-methylpyridin-2-yl)-1H-1,2-pyrazol-1-yl]-β-D-galactopyranosyl}-3-methyl-1H-1,2,4-triazol-1-yl}-2-(trifluoromethyl)benzene

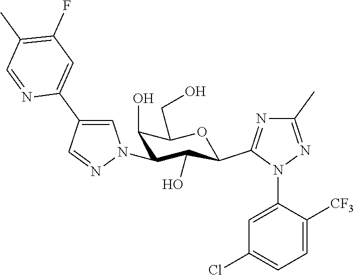

To a solution of 1-{5-[3-(4-borono-1H-1,2-pyrazol-1-yl)-3-deoxy-β-D-galactopyranosyl]-3-methyl-1H-1,2,4-triazol-1-yl}-5-chloro-2-(trifluoromethyl)benzene (30 mg, 0.058 mmol) and 2-bromo-4-fluoro-5-methylpyridine (55.1 mg, 0.29 mmol) in 1,4-dioxane/H₂O (2.5 mL, 4:1) Pd(dppf)Cl₂ (8.5 mg, 0.012 mmol) and K₂CO₃ (40 mg, 0.29 mmol) were added. The mixture was purged three times with nitrogen, and stirred 2 h at 80° C. The mixture was filtered, and the filtrate was acidified to pH 3-4 using TFA. The mixture was evaporated and purified by prep HPLC [MeCN/H₂O (10 mmol/L NH₄HCO₃), X-Select 10 μm 19*250 mm, 20 m/min, UV 254] to afford the title compound (7.3 mg, 22%). ESI-MS m/z calcd for [C₂₅H₂₃ClF₄N₆O₄] [M+H]⁺: 583.1; found: 582.8. ¹H NMR (400 MHz, Methanol-d₄) δ 8.31-8.20 (m, 2H), 7.95 (s, 1H), 7.86 (d, J=8.4 Hz, 1H), 7.76 (dd, J=8.4, 1.2 Hz, 1H), 7.68 (s, 1H), 7.34 (d, J=10.8 Hz, 1H), 4.71-4.61 (m, 1H), 4.31 (dd, J=10.8, 2.8 Hz, 1H), 4.20 (d, J=9.2 Hz, 1H), 4.00 (d, J=2.4 Hz, 1H), 3.61-3.52 (m, 3H), 2.34 (s, 3H), 2.18 (s, 3H).

### Example 16

### 1-{5-{3-[4-(5-Chloro-4-methylpyridin-2-yl)-1H-1,2-pyrazol-1-yl]-3-deoxy-β-D-galactopyranosyl}-3-methyl-1H-1,2,4-triazol-1-yl}-5-chloro-2-(trifluoromethyl)benzene

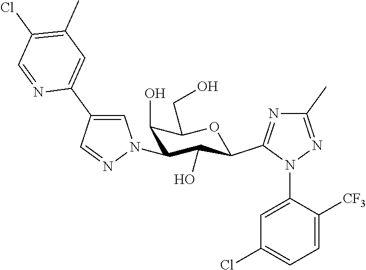

To a solution of 1-{5-[3-(4-borono-1H-1,2-pyrazol-1-yl)-3-deoxy-3-D-galactopyranosyl]-3-methyl-1H-1,2,4-triazol-1-yl}-5-chloro-2-(trifluoromethyl)benzene (30 mg, 0.058 mmol) and 2-bromo-5-chloro-4-methylpyridine (60 mg, 0.29 mmol) in 1,4-dioxane/H₂O (2.5 mL, 4:1) Pd(dppf)Cl₂ (8.5 mg, 0.012 mmol) and K₂CO₃ (40 mg, 0.29 mmol) were added. The mixture was purged three times with nitrogen, and stirred 2 h at 80° C. The mixture was filtered, and the filtrate was acidified to pH 3-4 using TFA. The mixture was evaporated and purified by prep HPLC [MeCN/H₂O (10 mmol/L NH₄HCO₃), X-Select 10 μm 19*250 mm, 20 m/min, UV 254] to afford the title compound (18.4 mg, 53%). ESI-MS m/z calcd for [C₂₅H₂₃Cl₂F₃N₆O₄] [M+H]⁺: 599.1; found: 598.8. ¹H NMR (400 MHz, Methanol-d₄) δ 8.37 (s, 1H), 8.34 (s, 1H), 8.04 (s, 1H), 7.94 (d, J=8.8 Hz, 1H), 7.85 (dd, J=8.8, 1.2 Hz, 1H), 7.76 (s, 1H), 7.64 (s, 1H), 4.83-4.68 (m, 1H), 4.40 (dd, J=10.4, 2.8 Hz, 1H), 4.29 (d, J=9.2 Hz, 1H), 4.09 (d, J=2.4 Hz, 1H), 3.73-3.59 (m, 3H), 2.43 (s, 3H), 2.42 (s, 3H).

### Example 17

### 5-Chloro-1-{5-{3-deoxy-3-[4-(4-ethynyl-3,5-difluorophenyl)-1H-1,2-pyrazol-1-yl]-β-D-galactopyranosyl}-3-methyl-1H-1,2,4-triazol-1-yl}-2-(trifluoromethyl)benzene

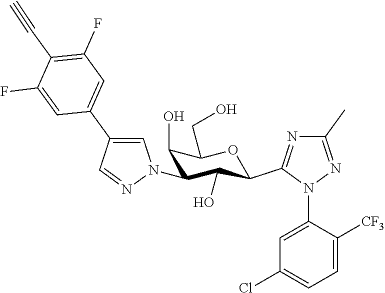

To a solution of 1-{5-[3-(4-borono-1H-1,2-pyrazol-1-yl)-3-deoxy-β-D-galactopyranosyl]-3-methyl-1H-1,2,4-triazol-1-yl}-5-chloro-2-(trifluoromethyl)benzene (30 mg, 0.058 mmol) and 2-(4-bromo-2,6-difluorophenyl)ethynyl(trimethyl)silane (279 mg, 30% purity, 0.29 mmol) in 1,4-dioxane/H₂O (2.5 mL, 4:1) Pd(dppf)Cl₂ (8.5 mg, 0.012 mmol) and K₂CO₃ (40 mg, 0.29 mmol) were added. The mixture was purged three times with nitrogen, and stirred 2 h at 80° C. KF (6.7 mg, 0.12 mmol) was added and the mixture was stirred 3 h at rt. The mixture was filtered, and the filtrate was acidified to pH 3-4 using TFA. The mixture was evaporated and purified by prep HPLC [MeCN/H₂O (0.05% TFA), X-Select 10 μm 19*250 mm, 20 m/min, UV 254] to afford the title compound (5.3 mg, 15%).

ESI-MS m/z calcd for [C₂₇H₂₁ClF₅N₅O₄] [M+H]⁺: 610.1; found: 609.8. ¹H NMR (400 MHz, Methanol-d₄) δ 8.17 (s, 1H), 7.90-7.83 (m, 2H), 7.76 (dd, J=8.4, 1.2 Hz, 1H), 7.68 (s, 1H), 7.25-7.14 (m, 2H), 4.74-4.55 (m, 1H), 4.30 (dd, J=10.4, 2.8 Hz, 1H), 4.19 (d, J=9.2 Hz, 1H), 3.99 (d, J=2.8 Hz, 1H), 3.93 (s, 1H), 3.66-3.50 (m, 3H), 2.35 (s, 3H).

### Example 18

### 5-Chloro-1-{5-{3-deoxy-3-{4-[3,5-difluoro-4-(2-propyn-1-yl)phenyl]-1H-1,2-pyrazol-1-yl}-β-D-galactopyranosyl}-3-methyl-1H-1,2,4-triazol-1-yl}-2-(trifluoromethyl)benzene

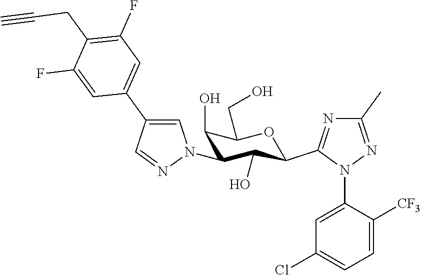

To a solution of 1-{5-{3-{4-{4-{3-[tert-butyl(dimethyl)silyl]prop-2-ynyl}-3,5-difluorophenyl}-1H-1,2-pyrazol-1-yl}-3-deoxy-β-D-galactopyranosyl}-3-methyl-1H-1,2,4-triazol-1-yl}-5-chloro-2-(trifluoromethyl)benzene (16 mg, 0.022 mmol) in MeCN (3 mL) TBAF (0.5 mL, 1 M in THF, 0.5 mmol) was added and the mixture was stirred 5 h at rt. EtOAc (10 mL) was added and the mixture was washed with 2N HCl (5×5 mL). The organic phase was dried, concentrated and purified by prep HPLC [MeCN/H₂O (10 mmol/L NH₄HCO₃), X-Select 10 μm 19*250 mm, 20 m/min, UV 254] to afford the title compound (5.1 mg, 37%). ESI-MS m/z calcd for [C₂₈H₂₃ClF₅N₅O₄] [M+H]⁺: 624.1; found: 623.8. ¹H NMR (400 MHz, Methanol-d₄) δ 8.11 (s, 1H), 7.86 (d, J=8.4 Hz, 1H), 7.82 (s, 1H), 7.76 (dd, J=8.8, 1.2 Hz, 1H), 7.68 (s, 1H), 7.17-7.09 (m, 2H), 4.74-4.62 (m, 1H), 4.28 (dd, J=10.4, 2.8 Hz, 1H), 4.19 (d, J=9.2 Hz, 1H), 3.99 (d, J=2.8 Hz, 1H), 3.64-3.52 (m, 3H), 3.45 (d, J=2.8 Hz, 2H), 2.34 (s, 3H), 2.22 (t, J=2.8 Hz, 1H).

### Example 19

### 5-Bromo-3-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}pyridine

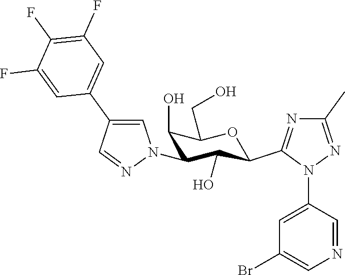

A nitrogen purged solution of 5-bromo-3-{5-[3-deoxy-3-(4-iodo-1H-1,2-pyrazol-1-yl)-β-D-galactopyranosyl]-3-methyl-1H-1,2,4-triazol-1-yl}pyridine (47 mg, 0.081 mmol), (3,4,5-trifluorophenyl)boronic acid (14.3 mg, 0.081 mmol), Na₂CO₃ (25.9 mg, 0.24 mmol) and Pd(dppf)Cl₂ (16.1 mg, 0.022 mmol) in THF (2 mL) and water (0.5 mL) was stirred 2 h at 30° C. The mixture was filtered, concentrated and the residue was purified by prep HPLC (MeCN/H₂O (10 mmol/L HCOOH), X-Select10 μm 19*250 mm, 20 m/min, UV 254) to give the title compound (5.3 mg, 11%). ESI-MS m/z calcd for [C₂₃H₂₀BrF₃N₆O₄] [M+H]⁺: 581.1; found: 580.8. ¹H NMR (400 MHz, DMSO-d₆) δ 8.90 (d, J=2.0 Hz, 1H), 8.88 (d, J=2.0 Hz, 1H), 8.41 (t, J=2.4 Hz, 1H), 8.38 (s, 1H), 8.02 (s, 1H), 7.64 (dd, J=9.2, 6.4 Hz, 2H), 5.31 (d, J=6.8 Hz, 1H), 5.15 (d, J=5.6 Hz, 1H), 4.83-4.78 (m, 2H), 4.51-4.48 (m, 2H), 3.96 (dd, J=5.6, 2.4 Hz, 1H), 3.82 (t, J=5.6 Hz, 1H), 3.53-3.47 (m, 2H), 2.39 (s, 3H).

### Example 20

### 5-Chloro-3-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-(trifluoromethyl)pyridine

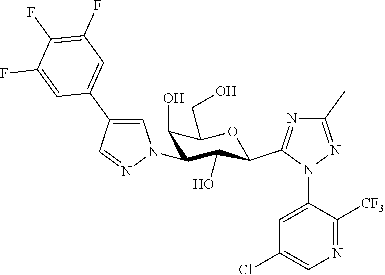

A nitrogen purged solution of 5-chloro-3-{5-[3-deoxy-3-(4-iodo-1H-1,2-pyrazol-1-yl)-β-D-galactopyranosyl]-3-methyl-1H-1,2,4-triazol-1-yl}-2-(trifluoromethyl)pyridine (45 mg, 0.075 mmol), (3,4,5-trifluorophenyl)boronic acid (39.5 mg, 0.23 mmol), K₂CO₃ (51.8 mg, 0.38 mmol) and Pd(dppf)Cl₂ (16.4 mg, 0.023 mmol) in 1,4-dioxane (2.0 mL) and water (1.0 mL) was stirred 1.5 h at 60° C. under nitrogen. The mixture was filtered, and the filtrate was acidified to pH 3-4 using TFA. The mixture was evaporated and purified by prep HPLC [MeCN/H₂O (10 mmol/L TFA), X-Select 10 μm 19*250 mm, 20 m/min, UV 254] to afford the title compound (11.8 mg, 26%). ESI-MS m/z calcd for [C₂₄H₁₉ClF₆N₆O₄] [M+H]⁺: 605.1; found: 604.8. ¹H NMR (400 MHz, Methanol-d₄) δ 8.93 (d, J=2.0 Hz, 1H), 8.32 (d, J=1.6 Hz, 1H), 8.16 (s, 1H), 7.88 (s, 1H), 7.45-7.31 (m, 2H), 4.69 (t, J=10.0 Hz, 1H), 4.41 (d, J=9.2 Hz, 1H), 4.38 (dd, J=10.8, 2.8 Hz, 1H), 4.06 (d, J=2.4 Hz, 1H), 3.68 (t, J=6.0 Hz, 1H), 3.63-3.51 (m, 2H), 2.43 (s, 3H).

### Example 21

### 5-Chloro-2-cyano-3-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}pyridine

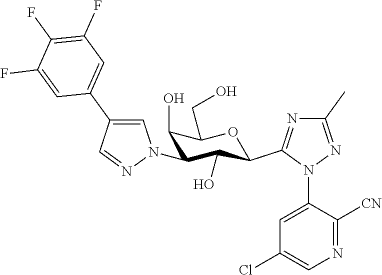

To a solution of 2-bromo-5-chloro-3-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}pyridine (80 mg, 0.13 mmol) in DMSO (2 mL) copper cyanide (46.5 mg, 0.52 mmol) was added and the mixture was stirred 3 h at 110° C. The mixture was poured into water (20 mL) and extracted with DCM (2×10 mL). The combined organic phases were dried over Na₂SO₄, concentrated, and purified by prep HPLC (MeCN/H₂O (10 mmol/L NH₄HCO₃), X-Select 10 μm 19*250 mm, 20 m/min, UV 254) to afford the title compound (17.3 mg, 24%). ESI-MS m/z calcd for [C₂₄H₁₉ClF₃N₇O₄] [M+H]⁺: 567.1; found: 561.9. ¹H NMR (400 MHz, Methanol-d₄) δ 8.90 (d, J=2.0 Hz, 1H), 8.50 (d, J=2.0 Hz, 1H), 8.18 (s, 1H), 7.90 (s, 1H), 7.36 (dd, J=9.2, 6.4 Hz, 2H), 4.72 (dd, J=10.4, 9.6 Hz, 1H), 4.58 (d, J=9.2 Hz, 1H), 4.43 (dd, J=10.4, 2.4 Hz, 1H), 4.08 (d, J=2.0 Hz, 1H), 3.78 (t, J=6.0 Hz, 1H), 3.69-3.57 (m, 2H), 2.48 (s, 3H).

### Example 22

### 5-Bromo-2-cyano-3-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}pyridine

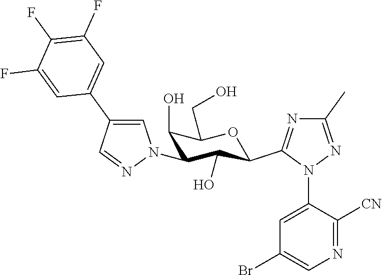

To a solution of 2,5-dibromo-3-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}pyridine (100 mg, 0.15 mmol) in DMSO (3 mL) copper cyanide (54.3 mg, 0.61 mmol) was added and the mixture was stirred 3 h at 100° C. The mixture was poured into water (20 mL) and extracted with DCM (2×10 mL). The combined organic phases were dried over Na₂SO₄, concentrated and purified by prep HPLC (MeCN/H₂O (10 mmol/L NH₄HCO₃), X-Select 10 μm 19*250 mm, 20 m/min, UV 254) to afford the title compound (30 mg, 33%). ESI-MS m/z calcd for [C₂₄H₁₉BrF₃N₇O₄] [M+H]⁺: 606.1; found: 605.8. ¹H NMR (400 MHz, DMSO-d₆) δ 9.12 (d, J=2.0 Hz, 1H), 8.67 (d, J=2.0 Hz, 1H), 8.35 (s, 1H), 8.01 (s, 1H), 7.62 (dd, J=9.6, 6.8, 2H), 5.38 (d, J=6.8 Hz, 1H), 5.10 (d, J=5.6 Hz, 1H), 4.70-4.64 (m, 2H), 4.55 (d, J=9.2 Hz, 1H), 4.42 (dd, J=10.4, 2.8 Hz, 1H), 3.93 (dd, J=5.6, 2.4 Hz, 1H), 3.70 (t, J=6.4 Hz, 1H), 3.43 (t, J=5.6 Hz, 2H), 2.42 (s, 3H).

### Example 23

### 3-Chloro-2-cyano-5-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}pyridine

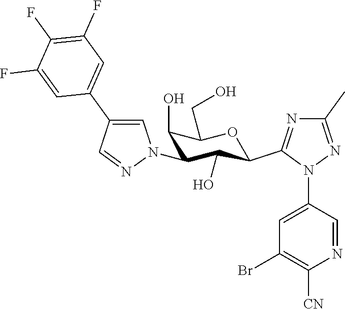

To a solution of 2-bromo-3-chloro-5-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}pyridine (140 mg, 0.23 mmol) in DMSO (3 mL) copper cyanide (102 mg, 1.14 mmol) was added and the mixture was stirred 6 h at 120° C. The mixture was poured into water (20 mL) and extracted with DCM (2×10 mL). The combined organic phases were dried over Na₂SO₄, concentrated, and purified by prep HPLC (MeCN/H₂O (10 mmol/L NH₄HCO₃), X-Select 10 μm 19*250 mm, 20 m/min, UV 254) to afford the title compound (10 mg, 8%). ESI-MS m/z calcd for [C₂₄H₁₉ClF₃N₇O₄] [M+H]⁺: 562.1; found: 561.8. ¹H NMR (400 MHz, DMSO-d₆) δ 9.05 (d, J=2.0 Hz, 1H), 8.65 (d, J=2.0 Hz, 1H), 8.39 (s, 1H), 8.02 (s, 1H), 7.64 (dd, J=9.6, 6.8 Hz, 2H), 5.39 (s, 1H), 5.20 (d, J=4.0 Hz, 1H), 4.85-4.80 (m, 2H), 4.62 (d, J=9.2 Hz, 1H), 4.47 (dd, J=10.8, 2.8 Hz, 1H), 3.97 (s, 1H), 3.86 (dd, J=6.8, 4.8 Hz, 1H), 3.54-3.48 (m, 2H), 2.42 (s, 3H).

### Example 24

### 5-Bromo-1-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-(trifluoromethyl)benzene

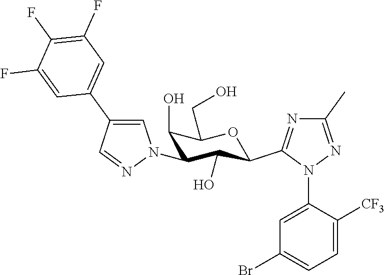

To a solution of 1-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-5-bromo-2-(trifluoromethyl)benzene (80 mg, 0.13 mmol) in EtOH (5 mL) 3-(dimethylamino)-2-(3,4,5-trifluorophenyl)prop-2-enal (30.1 mg, 0.13 mmol) and concentrated HCl (0.25 mL) were added and the mixture was stirred 2 h at 80° C. The mixture was concentrated and purified by prep HPLC [MeCN/H₂O (10 mmol/L NaHCO₃), X-Select 10 μm 19*250 mm, 20 mL/min, UV 254] to afford the title compound (29 mg, 37%). ESI-MS m/z calcd for [C₂₅H₂₀BrF₆N₅O₄] [M+H]⁺: 648.1; found: 647.8. ¹H NMR (400 MHz, Methanol-d₄) δ 8.19 (s, 1H), 8.01 (dd, J=8.4, 0.8 Hz, 1H), 7.95-7.82 (m, 3H), 7.47-7.29 (m, 2H), 4.82-4.70 (m, 1H), 4.38 (dd, J=10.4, 2.4 Hz, 1H), 4.28 (d, J=9.2 Hz, 1H), 4.09 (d, J=2.4 Hz, 1H), 3.70-3.62 (m, 3H), 2.44 (s, 3H).

### Example 25

### 5-Bromo-3-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-(trifluoromethyl)pyridine

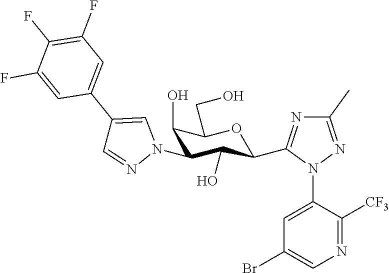

To a solution of 1-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-5-bromo-2-(trifluoromethyl)pyridine (50 mg, 0.075 mmol) in EtOH (5 mL) 3-(dimethylamino)-2-(3,4,5-trifluorophenyl)prop-2-enal (18.8 mg, 0.082 mmol) and concentrated HCl (0.25 mL) were added and the mixture was stirred 2 h at 80° C. The mixture was concentrated and purified by prep HPLC [MeCN/H₂O (10 mmol/L NaHCO₃), X-Select 10 μm 19*250 mm, 20 mL/min, UV 254] to afford the title compound (17 mg, 34%). ESI-MS m/z calcd for [C₂₄H₁₉BrF₆N₆O₄] [M+H]⁺: 649.1; found: 648.7. ¹H NMR (400 MHz, Methanol-d₄) δ 9.03 (d, J=2.0 Hz, 1H), 8.45 (d, J=1.2 Hz, 1H), 8.16 (s, 1H), 7.89 (s, 1H), 7.40-7.31 (m, 2H), 4.68 (t, J=10.0 Hz, 1H), 4.42 (d, J=9.2 Hz, 1H), 4.38 (dd, J=10.4, 2.8 Hz, 1H), 4.07 (d, J=2.4 Hz, 1H), 3.70-3.55 (m, 3H), 2.44 (s, 3H).

### Example 26

### 5-Bromo-2-cyano-1-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}benzene

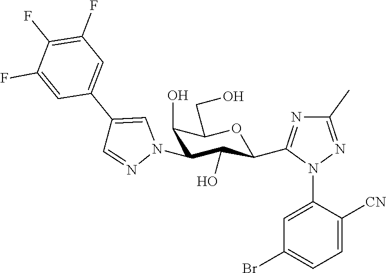

To a solution of 1-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-5-bromo-2-cyanobenzene (30 mg, 0.048 mmol) in EtOH (3.0 mL) 3-(dimethylamino)-2-(3,4,5-trifluorophenyl)prop-2-enal (13.1 mg, 0.057 mmol) and concentrated HCl (0.1 mL) were added and the mixture was stirred 2 h at 80° C. The mixture was concentrated and purified by prep HPLC [MeCN/H₂O (10 mmol/L NaHCO₃), X-Select 10 μm 19*250 mm, 20 mL/min, UV 254] to afford the title compound (18 mg, 62%). ESI-MS m/z calcd for [C₂₅H₂₀BrF₃N₆O₄] [M+H]⁺: 605.1; found: 604.8. ¹H NMR (400 MHz, DMSO-d₆) δ 8.35 (s, 1H), 8.09-8.01 (m, 4H), 7.65-7.61 (m, 2H), 5.35 (d, J=6.8 Hz, 1H), 5.09 (d, J=6.0 Hz, 1H), 4.77-4.70 (m, 1H), 4.67 (t, J=6.0 Hz, 1H), 4.47-4.41 (m, 2H), 3.94 (d, J=5.2, 2.4 Hz, 1H), 3.69 (t, J=6.0 Hz, 1H), 3.45 (t, J=6.0 Hz, 2H), 2.40 (s, 3H).

### Example 27

### 5-Chloro-2-cyano-1-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}benzene

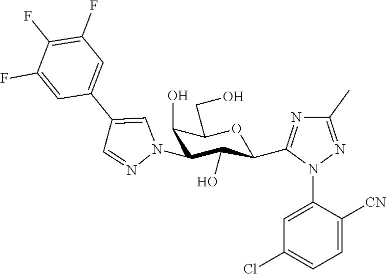

A nitrogen purged solution of 2-bromo-5-chloro-1-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}benzene (53 mg, 0.086 mmol) in DMF (1.5 mL) Zn (56.4 mg, 0.86 mmol), Zn(CN)₂ (101 mg, 0.86 mmol), 1,1′-bis(diphenylphosphino)ferrocene (9.73 mg, 0.017 mmol) and Pd₂(dba)₃ (15.8 mg, 0.017 mmol) were added and the mixture was stirred 15 h at 115° C. under nitrogen atmosphere. The mixture was filtered and purified by prep HPLC [MeCN/H₂O (10 mmol/L NH₄HCO₃), X-Select 10 μm 19*250 mm, 20 m/min, UV 254] to afford the title compound (4.3 mg, 9%). ESI-MS m/z calcd for [C₂₅H₂₀ClF₃N₆O₄] [M+H]⁺: 561.1; found: 561.2. ¹H NMR (400 MHz, Methanol-d₄) δ 8.18 (s, 1H), 7.99-7.97 (m, 2H), 7.89 (s, 1H), 7.80 (dd, J=8.4, 2.0 Hz, 1H), 7.35 (dd, J=9.2, 6.4 Hz, 2H), 4.80-4.75 (m, 1H) 4.48 (d, J=9.2 Hz, 1H), 4.42 (dd, J=10.8, 2.8 Hz, 1H), 4.10 (d, J=2.0 Hz, 1H), 3.79-3.66 (m, 3H), 2.47 (s, 3H).

### Example 28

### 3-{5-{3-[4-(4-Chloro-3,5-difluorophenyl)-1H-1,2-pyrazol-1-yl]-3-deoxy-β-D-galactopyranosyl}-3-methyl-1H-1,2,4-triazol-1-yl}-5-chloro-2-(trifluoromethyl)pyridine

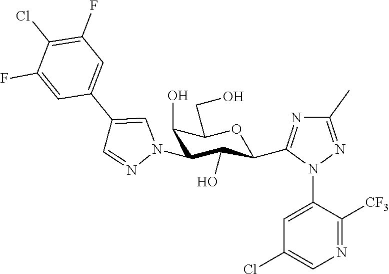

A nitrogen purged solution of 5-chloro-3-{5-[3-deoxy-3-(4-iodo-1H-1,2-pyrazol-1-yl)-β-D-galactopyranosyl]-3-methyl-1H-1,2,4-triazol-1-yl}-2-(trifluoromethyl)pyridine (45 mg, 0.075 mmol), (4-chloro-3,5-trifluorophenyl)boronic acid (51.8 mg, 0.38 mmol), K₂CO₃ (51.8 mg, 0.38 mmol) and Pd(dppf)Cl₂ (16.4 mg, 0.023 mmol) in 1,4-dioxane (2.0 mL) and water (1.0 mL) was stirred 1 h at 60° C. under nitrogen atmosphere. The mixture was filtered, and the filtrate was acidified to pH 3-4 using TFA. The mixture was evaporated and purified by prep HPLC [MeCN/H₂O (10 mmol/L NH₄CO₃), X-Select 10 μm 19*250 mm, 20 mL/min, UV 254] to afford the title compound (9.4 mg, 20%). ESI-MS m/z calcd for [C₂₄H₁₉Cl₂F₅N₆O₄] [M+H]⁺: 621.1; found: 620.8. ¹H NMR (400 MHz, Methanol-d₄) δ 8.94 (d, J=2.0 Hz, 1H), 8.33 (d, J=1.6 Hz, 1H), 8.22 (s, 1H), 7.94 (s, 1H), 7.42-7.33 (m, 2H), 4.70 (t, J=10.0 Hz, 1H), 4.45-4.36 (m, 2H), 4.07 (d, J=2.4 Hz, 1H), 3.68 (t, J=6.0 Hz, 1H), 3.64-3.55 (m, 2H), 2.44 (s, 3H).

### Example 29

### 1-{5-{3-[4-(4-Chloro-3,5-difluorophenyl)-3-ethoxycarbonyl-1H-1,2-pyrazol-1-yl]-3-deoxy-β-D-galactopyranosyl}-3-methyl-1H-1,2,4-triazol-1-yl}-5-chloro-2-(trifluoromethyl)benzene

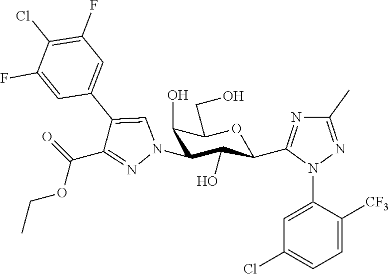

A nitrogen purged solution of 5-chloro-1-{5-[3-deoxy-3-β-ethoxycarbonyl-4-iodo-1H-1,2-pyrazol-1-yl)-β-D-galactopyranosyl]-3-methyl-1H-1,2,4-triazol-1-yl}-2-(trifluoromethyl)benzene (54 mg, 0.080 mmol), (4-chloro-3,5-difluorophenyl)boronic acid (30.9 mg, 0.16 mmol), K₂CO₃ (33.5 mg, 0.24 mmol) and Pd(dppf)Cl₂ (5.88 mg, 0.0080 mmol) in 1,4-dioxane (2 mL) and water (0.5 mL) was stirred 2 h at 40° C. The mixture was filtered, concentrated, and purified by prep HPLC (MeCN/H₂O (10 mmol/L NH₄CO₃), X-Select10 μm 19*250 mm, 20 m/min, UV 254) to give the title compound (31 mg, 56%). ESI-MS m/z calcd for [C₂₈H₂₄Cl₂F₅N₅O₆] [M+H]⁺: 692.1; found: 691.8. ¹H NMR (400 MHz, Methanol-d₄) δ 8.09 (s, 1H), 7.95 (d, J=8.4 Hz, 1H), 7.85 (d, J=8.8 Hz, 1H), 7.77 (s, 1H), 7.36-7.33 (m, 2H), 4.82-4.76 (m, 1H), 4.48 (dd, J=10.8, 2.8 Hz, 1H), 4.34-4.29 (m, 3H), 4.11 (d, J=2.8 Hz, 1H), 3.67-3.63 (m, 3H), 2.44 (s, 3H), 1.29 (t, J=7.2 Hz, 3H).

### Example 30

### 1-{5-{3-[3-Carboxy-4-(4-chloro-3,5-difluorophenyl)-1H-1,2-pyrazol-1-yl]-3-deoxy-β-D-galactopyranosyl}-3-methyl-1H-1,2,4-triazol-1-yl}-5-chloro-2-(trifluoromethyl)benzene

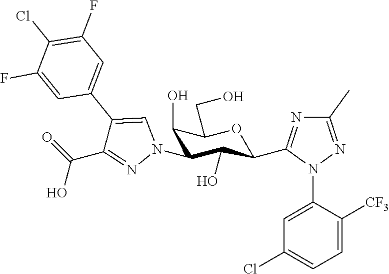

To a solution of 1-{5-{3-[4-(4-chloro-3,5-difluorophenyl)-3-ethoxycarbonyl-1H-1,2-pyrazol-1-yl]-3-deoxy-β-D-galactopyranosyl}-3-methyl-1H-1,2,4-triazol-1-yl}-5-chloro-2-(trifluoromethyl)benzene (10 mg, 0.014 mmol) in MeOH (2 mL) lithium hydroxide monohydrate (6.1 mg, 0.14 mmol) and few drops of water were added. The mixture was stirred 4 h at 30° C. The mixture was concentrated and then acidified with HCl (2 M aq solution) to pH=5. The solution was concentrated and purified by prep HPLC [MeCN/H₂O (10 mmol/L NH₄HCO₃), X-Select 10 μm 19*250 mm, 20 mL/min, UV 254] to give the title compound (3.9 mg, 41%). ESI-MS m/z calcd for [C₂₆H₂₀Cl₂F₅N₅O₆] [M+H]⁺: 664.1; found: 663.7. ¹H NMR (400 MHz, Methanol-d₄) δ 8.05 (s, 1H), 7.95 (d, J=8.4 Hz, 1H), 7.85 (d, J=8.8 Hz, 1H), 7.78 (s, 1H), 7.41 (d, J=8.8 Hz, 2H), 4.80-4.75 (m, 1H), 4.45-4.41 (m, 1H), 4.29 (d, J=9.2 Hz, 1H), 4.12 (s, 1H), 3.65-3.63 (m, 3H), 2.43 (s, 3H).

### Example 31

### 1-{5-{3-[4-(4-Chloro-3,5-difluorophenyl)-3-cyano-1H-1,2-pyrazol-1-yl]-3-deoxy-β-D-galactopyranosyl}-3-methyl-1H-1,2,4-triazol-1-yl}-5-chloro-2-(trifluoromethyl)benzene

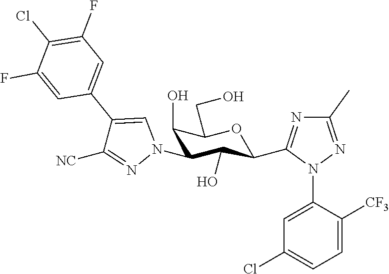

A solution of 1-{5-{3-[4-(4-chloro-3,5-difluorophenyl)-3-ethoxycarbonyl-1H-1,2-pyrazol-1-yl]-3-deoxy-β-D-galactopyranosyl}-3-methyl-1H-1,2,4-triazol-1-yl}-5-chloro-2-(trifluoromethyl)benzene (21 mg, 0.03 mmol) in ammonia (3.0 mL, 7 M in MeOH) was stirred 48 h at 60° C. in a sealed tube. The mixture was concentrated, and the crude product was dissolved in THF (2.0 mL). Trifluoroacetic anhydride (42.2 μL) and pyridine (24.5 μL) were added, and the mixture was stirred 2 h at rt. The mixture was concentrated, water (1.0 mL) was added, and the mixture was extracted with EtOAc (3×1.0 mL). The combined organic phases were dried, evaporated and the residue was dissolved in MeOH (2.0 mL). Water (0.5 mL) and Et₃N (0.5 mL) were added, and the mixture was stirred 1 h at rt. The mixture was concentrated and purified by prep HPLC [MeOH/H₂O (10 mmol/L, 0.05% TFA), X-Select 10 μm 19*250 mm, 20 m/min, UV 254] to give the title compound (3.4 mg, 17%). ESI-MS m/z calcd for [C₂₆H₁₉Cl₂F₅N₆O₄] [M+H]⁺: 645.1; found: 644.7. ¹H NMR (400 MHz, Methanol-d₄) δ 8.40 (s, 1H), 7.96 (d, J=8.8 Hz, 1H), 7.86 (dd, J=8.4, 1.2 Hz, 1H), 7.76 (s, 1H), 7.52-7.48 (m, 2H), 4.92-4.89 (m, 1H), 4.54 (dd, J=10.8, 3.2 Hz, 1H), 4.30 (d, J=9.6 Hz, 1H), 4.09 (d, J=2.8 Hz, 1H), 3.67-3.63 (m, 3H), 2.45 (s, 3H).

### Example 32

### 1-{5-{3-[3-Aminocarbonyl-4-(4-chloro-3,5-difluorophenyl)-1H-1,2-pyrazol-1-yl]-3-deoxy-β-D-galactopyranosyl}-3-methyl-1H-1,2,4-triazol-1-yl}-5-chloro-2-(trifluoromethyl)benzene

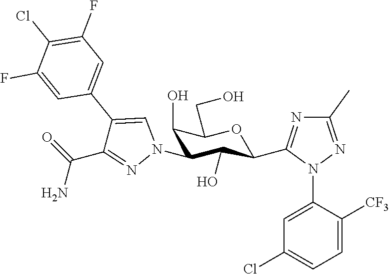

A solution of 1-{5-{3-[4-(4-chloro-3,5-difluorophenyl)-3-ethoxycarbonyl-1H-1,2-pyrazol-1-yl]-3-deoxy-β-D-galactopyranosyl}-3-methyl-1H-1,2,4-triazol-1-yl}-5-chloro-2-(trifluoromethyl)benzene (19 mg, 0.027 mmol) in ammonia (3.0 mL, 7 M in MeOH) was stirred 48 h at 60° C. in a sealed tube. The mixture was concentrated and purified by prep HPLC (MeCN/H₂O (10 mmol/L NH₄HCO₃), X-Select 10 μm 19*250 mm, 20 m/min, UV 254) to afford the title compound (13.9 mg, 76%). ESI-MS m/z calcd for [C₂₆H₂₁Cl₂F₅N₆O₅] [M+H]⁺: 663.1.2; found: 662.7. ¹H NMR (400 MHz, Methanol-d₄) δ 8.08 (s, 1H), 7.96 (d, J=8.8 Hz, 1H), 7.85 (d, J=8.4 Hz, 1H), 7.78 (s, 1H), 7.47-7.42 (m, 2H), 4.85-4.84 (m, 1H), 4.46 (dd, J=10.8, 2.8 Hz, 1H), 4.28 (d, J=9.6 Hz, 1H), 4.13 (d, J=2.4 Hz, 1H), 3.68-3.66 (m, 3H), 2.44 (s, 3H).

### Example 33

### 5-Chloro-2-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-thiazole

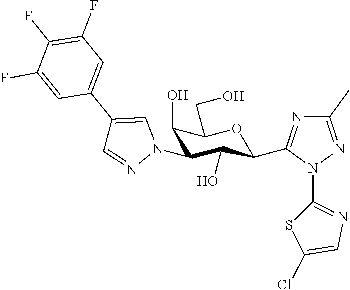

To a solution of 2-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-5-chlorothiazole (59 mg, 0.10 mmol) in EtOH (5 mL) 3-(dimethylamino)-2-(3,4,5-trifluorophenyl)prop-2-enal (35.9 mg, 0.16 mmol) and concentrated HCl (0.2 mL) were added and the mixture was stirred 2 h at 80° C. The mixture was concentrated and purified by prep HPLC (MeCN/H₂O (10 mmol/L NH₄HCO₃), X-Select 10 μm 19*250 mm, 20 m/min, UV 254) to afford the title compound (19.8 mg, 35%). ESI-MS m/z calcd for [C₂₁H₁₈ClF₃N₆O₄S] [M+H]⁺: 543.1; found: 543.1. ¹H NMR (400 MHz, Methanol-d₄) δ 8.22 (s, 1H), 7.92 (s, 1H), 7.62 (s, 1H), 7.39-7.34 (m, 2H), 5.60 (d, J=9.6 Hz, 1H), 4.92-4.88 (m, 1H), 4.53 (dd, J=10.8, 2.4 Hz, 1H), 4.18 (d, J=2.4 Hz, 1H), 3.91 (t, J=5.6 Hz, 1H), 3.72-3.69 (m, 2H), 2.43 (s, 3H).

### Example 34

### 6-{5-{3-Deoxy-3-[4-(3,4,5-trifluorophenyl)-1H-1,2-pyrazol-1-yl]-β-D-galactopyranosyl}-3-methyl-1H-1,2,4-triazol-1-yl}benzothiazole

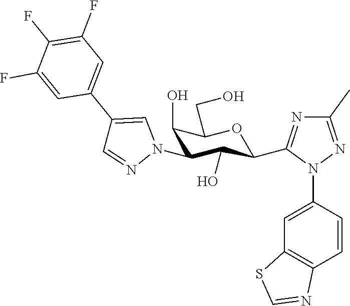

To a solution of 6-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}benzothiazole (140 mg, 0.24 mmol) in EtOH (5 mL) 3-(dimethylamino)-2-(3,4,5-trifluorophenyl)prop-2-enal (66.3 mg, 0.29 mmol) and concentrated HCl (0.2 mL) were added and the mixture was stirred 2 h at 80° C. The pH was adjusted to 8 by addition of aq NaHCO₃. The mixture was concentrated and purified by column chromatography (MeOH/DCM=0/1˜1/10, Silica-CS 12 g, 20 mL/min, silica gel, UV 254) to afford the title compound (50 mg, 37%). ESI-MS m/z calcd for [C₂₅H₂₁F₃N₆O₄S] [M+H]⁺: 559.1; found: 558.9. ¹H NMR (400 MHz, DMSO-d₆) δ 9.54 (s, 1H), 8.60 (d, J=2.0 Hz, 1H), 8.40 (s, 1H), 8.25 (d, J=8.8 Hz, 1H), 8.01 (s, 1H), 7.83 (dd, J=8.8, 2.4 Hz, 1H), 7.65 (dd, J=9.6, 6.8 Hz, 2H), 5.26 (d, J=7.6 Hz, 1H), 5.13 (d, J=6.0 Hz, 1H), 4.93-4.86 (m, 2H), 4.45 (dd, J=10.8, 2.8 Hz, 1H), 4.41 (d, J=9.6 Hz, 1H), 3.95 (dd, J=6.0, 3.2 Hz, 1H), 3.77 (dd, J=7.6, 4.8 Hz, 1H), 3.63-3.57 (m, 1H), 3.53-3.49 (m, 1H), 2.41 (s, 3H).

### Example 35

### 6-{5-{3-Deoxy-3-[4-(3,4,5-trifluorophenyl)-1H-1,2-pyrazol-1-yl]-β-D-galactopyranosyl}-3-methyl-1H-1,2,4-triazol-1-yl}-2-ethylbenzothiazole

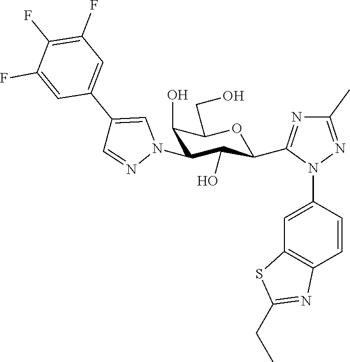

To a solution of 6-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-2-ethylbenzothiazole (100 mg, 0.16 mmol) in EtOH (5 mL) 3-(dimethylamino)-2-(3,4,5-trifluorophenyl)prop-2-enal (45.2 mg, 0.20 mmol) and concentrated HCl (0.2 mL) were added and the mixture was stirred 2 h at 80° C. The mixture was concentrated and purified by prep HPLC (MeCN/H₂O (10 mmol/L NH₄HCO₃), X-Select 10 μm 19*250 mm, 20 mL/min, UV 254) to afford the title compound (28 mg, 29%). ESI-MS m/z calcd for [C₂₇H₂₅F₃N₆O₄S] [M+H]⁺: 587.2; found: 586.8. ¹H NMR (400 MHz, DMSO-d₆) δ 8.47 (d, J=2.0 Hz, 1H), 8.40 (s, 1H), 8.08 (d, J=8.8 Hz, 1H), 8.01 (s, 1H), 7.75 (dd, J=8.8, 2.4 Hz, 1H), 7.68-7.63 (m, 2H), 5.26 (d, J=7.2 Hz, 1H), 5.13 (d, J=5.6 Hz, 1H), 4.92-4.85 (m, 2H), 4.45 (dd, J=10.4, 2.8 Hz, 1H), 4.38 (d, J=9.2 Hz, 1H), 3.94 (dd, J=5.6, 2.8 Hz, 1H), 3.75 (dd, J=7.2, 4.4 Hz, 1H), 3.62-3.56 (m, 1H), 3.52-3.47 (m, 1H), 3.18 (q, J=7.6 Hz, 2H), 2.44 (s, 3H), 1.41 (t, J=7.6 Hz, 3H).

### Example 36

### 6-{5-{3-Deoxy-3-[4-(3,4,5-trifluorophenyl)-1H-1,2-pyrazol-1-yl]-β-D-galactopyranosyl}-3-methyl-1H-1,2,4-triazol-1-yl}-2-(trifluoromethyl)benzothiazole

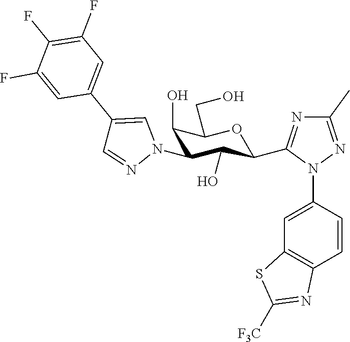

To a solution of 6-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-2-(trifluoromethyl)benzothiazole (65 mg, 0.10 mmol) in EtOH (5 mL) 3-(dimethylamino)-2-(3,4,5-trifluorophenyl)prop-2-enal (25.3 mg, 0.11 mmol) and concentrated HCl (0.2 mL) were added and the mixture was stirred 2 h at 80° C. The pH was adjusted to 8 by addition of aq NaHCO₃. The mixture was concentrated and purified by prep HPLC (MeCN/H₂O (10 mmol/L NH₄HCO₃), X-Select 10 μm 19*250 mm, 20 mL/min, UV 254) to afford the title compound (6.1 mg, 10%). ESI-MS m/z calcd for [C₂₆H₂₀F₆N₆O₄S] [M+H]⁺: 627.1; found: 628.8. ¹H NMR (400 MHz, DMSO-d₆) δ 8.83 (d, J=2.0 Hz, 1H), 8.44 (d, J=8.8 Hz, 1H), 8.40 (s, 1H), 8.03-8.01 (m, 2H), 7.67-7.63 (m, 2H), 5.30 (d, J=6.8 Hz, 1H), 5.16 (d, J=5.6 Hz, 1H), 4.99 (t, J=5.2 Hz, 1H), 4.93-4.87 (m, 1H), 4.47-4.41 (m, 2H), 3.96 (t, J=2.8 Hz, 1H), 3.82-3.79 (m, 1H), 3.64-3.58 (m, 1H), 3.55-3.49 (m, 1H), 2.42 (s, 3H).

### Example 37

### 6-{5-{3-Deoxy-3-[4-(3,4,5-trifluorophenyl)-1H-1,2-pyrazol-1-yl]-β-D-galactopyranosyl}-3-methyl-1H-1,2,4-triazol-1-yl}-5-fluoro-2-methylbenzothiazole

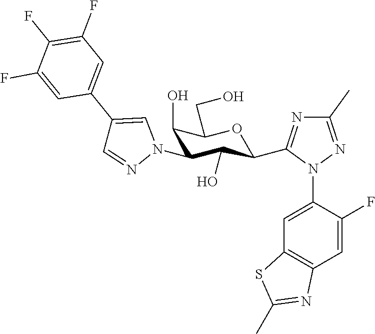

To a solution of 6-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-5-fluoro-2-methylbenzothiazole (60 mg, 0.098 mmol) in EtOH (5 mL) 3-(dimethylamino)-2-(3,4,5-trifluorophenyl)prop-2-enal (24.7 mg, 0.11 mmol) and concentrated HCl (0.2 mL) were added and the mixture was stirred 2 h at 80° C. The mixture was concentrated and purified by prep HPLC (MeCN/H₂O (10 mmol/L NH₄HCO₃), X-Select 10 μm 19*250 mm, 20 mL/min, UV 254) to afford the title compound (19.5 mg, 34%). ESI-MS m/z calcd for [C₂₆H₂₂F₄N₆O₄S] [M+H]⁺: 591.1; found: 590.8. ¹H NMR (400 MHz, Methanol-d₄) δ 8.30 (d, J=7.2 Hz, 1H), 8.17 (s, 1H), 7.90-7.87 (m, 2H), 7.38-7.33 (m, 2H), 4.77 (t, J=10.0 Hz, 1H), 4.41-4.35 (m, 2H), 4.05 (d, J=2.4 Hz, 1H), 3.73-3.62 (m, 3H), 2.89 (s, 3H), 2.47 (s, 3H).

### Example 38

### 6-{5-{3-Deoxy-3-[4-(3,4,5-trifluorophenyl)-1H-1,2-pyrazol-1-yl]-β-D-galactopyranosyl}-3-methyl-1H-1,2,4-triazol-1-yl}-7-fluoro-2-methylbenzothiazole

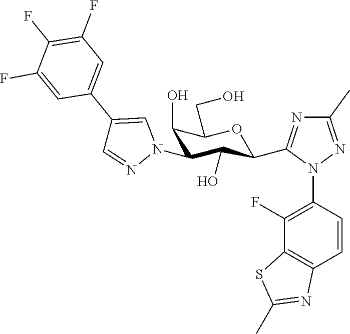

To a solution of 6-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-7-fluoro-2-methylbenzothiazole (70 mg, 0.11 mmol) in EtOH (5.0 mL) 3-(dimethylamino)-2-(3,4,5-trifluorophenyl)prop-2-enal (18.8 mg, 0.082 mmol) and concentrated HCl (0.25 mL) were added and the mixture was stirred 2 h at 80° C. The mixture was concentrated and purified by prep HPLC (MeCN/H₂O (10 mmol/L NH₄HCO₃), X-Select 10 μm 19*250 mm, 20 mL/min, UV 254) to afford the title compound (14.5 mg, 23%). ESI-MS m/z calcd for [C₂₆H₂₂F₄N₆O₄S] [M+H]⁺: 591.1; found: 590.8. ¹H NMR (400 MHz, DMSO-d₆) δ 8.34 (s, 1H), 7.99 (s, 1H), 7.94 (d, J=8.4 Hz, 1H), 7.71 (t, J=7.6 Hz, 1H), 7.62 (dd, J=9.6, 6.8 Hz, 2H), 5.28 (d, J=7.2 Hz, 1H), 5.06 (d, J=5.6 Hz, 1H), 4.75-4.65 (m, 2H), 4.38 (dd, J=11.2, 2.8 Hz, 1H), 4.31 (d, J=9.2 Hz, 1H), 3.90 (d, J=2.4 Hz, 1H), 3.59 (t, J=6.0 Hz, 1H), 3.43 (t, J=5.6 Hz, 2H), 2.91 (s, 3H), 2.39 (s, 3H).

### Example 39

### 6-{5-{3-Deoxy-3-[4-(3,4,5-trifluorophenyl)-1H-1,2-pyrazol-1-yl]-β-D-galactopyranosyl}-3-methyl-1H-1,2,4-triazol-1-yl}-4-fluoro-2-methylbenzothiazole

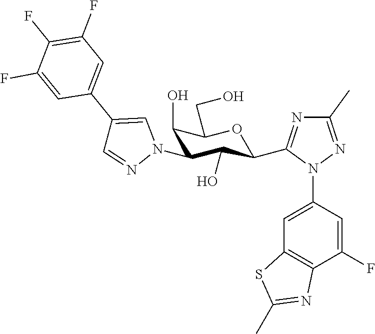

To a solution of 6-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-4-fluoro-2-methylbenzothiazole (70 mg, 0.11 mmol) in EtOH (5 mL) 3-(dimethylamino)-2-(3,4,5-trifluorophenyl)prop-2-enal (28.8 mg, 0.13 mmol) and concentrated HCl (0.25 mL) were added and the mixture was stirred 2 h at 80° C. The mixture was concentrated and purified by prep HPLC (MeCN/H₂O (10 mmol/L NH₄HCO₃), X-Select 10 μm 19*250 mm, 20 m/min, UV 254) to afford the title compound (23.5 mg, 35%). ESI-MS m/z calcd for [C₂₆H₂₂F₄N₆O₄S] [M+H]⁺: 591.1; found: 590.8. ¹H NMR (400 MHz, DMSO-d₆) δ 8.39 (s, 1H), 8.34 (d, J=1.6 Hz, 1H), 8.01 (s, 1H), 7.74-7.59 (m, 3H), 5.31 (d, J=7.2 Hz, 1H), 5.16 (d, J=6.0 Hz, 1H), 4.93 (t, J=5.6 Hz, 1H), 4.91-4.82 (m, 1H), 4.53-4.42 (m, 2H), 3.95 (dd, J=6.0, 2.8 Hz, 1H), 3.79 (dd, J=7.2, 4.0 Hz, 1H), 3.66-3.55 (m, 1H), 3.55-3.46 (m, 1H), 2.89 (s, 3H), 2.40 (s, 3H).

### Example 40

### 7-Chloro-6-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-methylbenzothiazole

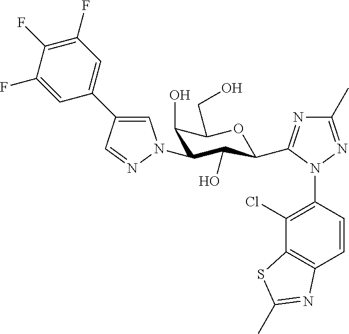

To a solution of 6-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-7-chloro-2-methylbenzothiazole (110 mg, 0.18 mmol) in EtOH (5 mL) 3-(dimethylamino)-2-(3,4,5-trifluorophenyl)prop-2-enal (44.1 mg, 0.19 mmol) and concentrated HCl (0.25 mL) were added and the mixture was stirred 2 h at 80° C. The mixture was concentrated and purified by prep HPLC (MeCN/H₂O (10 mmol/L NH₄HCO₃), X-Select 10 μm 19*250 mm, 20 mL/min, UV 254) to afford the title compound (41.6 mg, 39%). ESI-MS m/z calcd for [C₂₆H₂₂ClF₃N₆O₄S] [M+H]⁺: 607.1; found: 606.8. ¹H NMR (400 MHz, DMSO-d₆) δ 8.34 (s, 1H), 8.05 (d, J=8.8 Hz, 1H), 7.99 (s, 1H), 7.68 (d, J=8.8 Hz, 1H), 7.62 (dd, J=9.2, 6.8 Hz, 2H), 5.31 (d, J=6.8 Hz, 1H), 5.08 (d, J=5.6 Hz, 1H), 4.79-4.61 (m, 2H), 4.37 (dd, J=10.8, 2.4 Hz, 1H), 4.20 (d, J=9.2 Hz, 1H), 3.89 (dd, J=5.6, 2.8 Hz, 1H), 3.54 (t, J=6.4 Hz, 1H), 3.47-3.41 (m, 2H), 2.91 (s, 3H), 2.40 (s, 3H).

### Example 41

### 4-Chloro-6-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-methylbenzothiazole

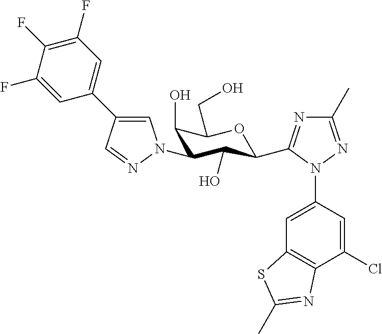

To a solution of 6-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-4-chloro-2-methylbenzothiazole (51 mg, 0.081 mmol) in EtOH (5 mL) 3-(dimethylamino)-2-(3,4,5-trifluorophenyl)prop-2-enal (20.4 mg, 0.089 mmol) and concentrated HCl (0.2 mL) were added and the mixture was stirred 2 h at 80° C. The mixture was concentrated and purified by prep HPLC (MeCN/H₂O (10 mmol/L NH₄HCO₃), X-Select 10 μm 19*250 mm, 20 mL/min, UV 254) to afford the title compound (12.4 mg, 25%). ESI-MS m/z calcd for [C₂₆H₂₂ClF₃N₆O₄S] [M+H]⁺: 607.1; found: 606.8. ¹H NMR (400 MHz, Methanol-d₄) δ 8.36 (d, J=2.0 Hz, 1H), 8.18 (s, 1H), 7.89 (s, 1H), 7.88 (d, J=2.0 Hz, 1H), 7.35 (dd, J=9.2, 6.8 Hz, 2H), 4.92-4.89 (m, 1H), 4.51 (d, J=9.6 Hz, 1H), 4.43 (dd, J=10.8, 2.8 Hz, 1H), 4.10 (d, J=2.8 Hz, 1H), 3.83-3.78 (m, 2H), 3.74-3.70 (m, 1H), 2.92 (s, 3H), 2.46 (s, 3H).

### Example 42

### 4-Cyano-6-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-methylbenzothiazole

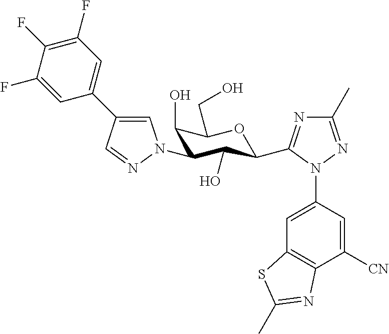

To a solution of 4-bromo-6-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-methylbenzothiazole (45 mg, 0.069 mmol) in DMF (2.5 mL) Pd₂(dba)₃ (31.6 mg, 0.035 mmol), 1,1′-bis(diphenylphosphino)ferrocene (39.0 mg, 0.069 mmol), zinc cyanide (32.5 mg, 0.28 mmol) and Zn (45.2 mg, 0.069 mmol) were added and the mixture was stirred 2 h at 100° C. The mixture was filtered and purified by prep HPLC (MeCN/H₂O (10 mmol/L NH₄HCO₃), X-Select 10 μm 19*250 mm, 20 m/min, UV 254) to afford the title compound (11.8 mg, 29%). ESI-MS m/z calcd for [C₂₇H₂₂F₃N₇O₄S] [M+H]⁺: 598.1; found: 597.8. ¹H NMR (400 MHz, DMSO-d₆) δ 8.82 (d, J=2.4 Hz, 1H), 8.38 (s, 1H), 8.28 (d, J=2.0 Hz, 1H), 8.00 (s, 1H), 7.67-7.61 (m, 2H), 5.32 (d, J=7.2 Hz, 1H), 5.13 (d, J=5.6 Hz, 1H), 4.90 (t, J=5.6 Hz, 1H), 4.86-4.83 (m, 1H), 4.49-4.44 (m, 2H), 3.95-3.94 (m, 1H), 3.82-3.77 (m, 1H), 3.59-3.48 (m, 2H), 2.95 (s, 3H), 2.40 (s, 3H).

### Example 43

### 5-Bromo-6-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-methylbenzothiazole

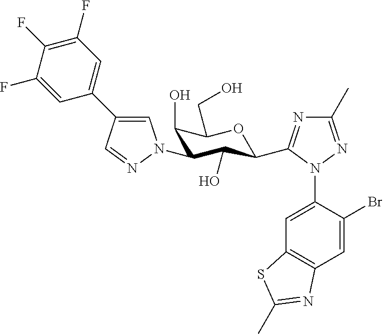

To a solution of 6-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-5-bromo-2-methylbenzothiazole (115 mg, 0.17 mmol) in EtOH (5 mL) 3-(dimethylamino)-2-(3,4,5-trifluorophenyl)prop-2-enal (47.0 mg, 0.21 mmol) and concentrated HCl (0.2 mL) were added and the mixture was stirred 2 h at 80° C. The pH was adjusted to 8 by addition of aq NaHCO₃. The mixture was concentrated and purified by column chromatography (DCM/MeOH=1/0˜10/1, Silica-CS 20 g, 30 mL/min, silica gel, UV 254) to afford the product (70 mg, 63%). Some of the product (15 mg) was further purified by prep HPLC (MeCN/H₂O (10 mmol/L NH₄HCO₃), X-Select 10 μm 19*250 mm, 20 mL/min, UV 254) to afford the title compound (7.0 mg). ESI-MS m/z calcd for [C₂₆H₂₂BrF₃N₆O₄S] [M+H]⁺: 651.1; found: 650.8. ¹H NMR (400 MHz, DMSO-d₆) δ 8.43-8.10 (m, 3H), 7.99 (s, 1H), 7.66-7.60 (m, 2H), 5.25 (d, J=7.2 Hz, 1H), 5.02 (d, J=5.2 Hz, 1H), 4.70-4.60 (m, 2H), 4.36 (dd, J=10.8, 2.8 Hz, 1H), 4.13-4.10 (m, 1H), 3.88 (s, 1H), 3.47-3.37 (m, 3H), 2.88 (s, 3H), 2.38 (s, 3H).

### Example 44

### 5-Cyano-6-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-methylbenzothiazole

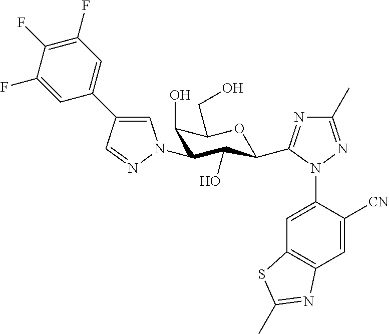

To a solution of 5-bromo-6-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-methylbenzothiazole (50 mg, 0.077 mmol) in DMSO (3 mL) copper cyanide (20.6 mg, 0.23 mmol) was added and the mixture was stirred 6 h at 120° C. The mixture was poured into water (20 mL) and extracted with DCM (2×10 mL). The combined organic phases were dried over Na₂SO₄, concentrated, and purified by prep HPLC (MeCN/H₂O (10 mmol/L NH₄HCO₃), X-Select 10 μm 19*250 mm, 20 m/min, UV 254) to afford the title compound (7.0 mg, 15%). ESI-MS m/z calcd for [C₂₇H₂₂F₃N₇O₄S] [M+H]⁺: 598.1; found: 597.9. ¹H NMR (400 MHz, DMSO-d₆) δ 8.71 (s, 1H), 8.61 (s, 1H), 8.36 (s, 1H), 7.99 (s, 1H), 7.65-7.61 (m, 2H), 5.33 (d, J=7.2 Hz, 1H), 5.07 (d, J=5.6 Hz, 1H), 4.82-4.76 (m, 2H), 4.39 (dd, J=11.2, 3.2 Hz, 1H), 4.34 (d, J=9.2 Hz, 1H), 3.89 (dd, J=6.0, 3.2 Hz, 1H), 3.61 (t, J=6.4 Hz, 1H), 3.53-3.48 (m, 1H), 3.47-3.37 (m, 1H), 2.91 (s, 3H), 2.41 (s, 3H).

### Example 45

### 6-{5-{3-Deoxy-3-[4-(3,4,5-trifluorophenyl)-1H-1,2-pyrazol-1-yl]-β-D-galactopyranosyl}-3-methyl-1H-1,2,4-triazol-1-yl}-2-methylthiazolo[4,5-b]pyridine

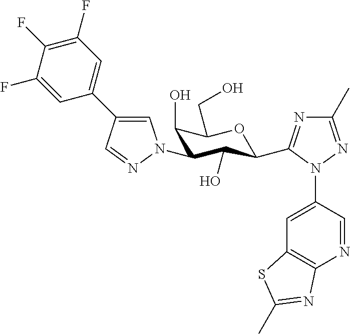

To a solution of 6-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-2-methylthiazolo[4,5-b]pyridine (109 mg, 0.18 mmol) in EtOH (5 mL) 3-(dimethylamino)-2-(3,4,5-trifluorophenyl)prop-2-enal (46.1 mg, 0.20 mmol) and concentrated HCl (0.2 mL) were added and the mixture was stirred 2 h at 80° C. The mixture was concentrated and purified by prep HPLC [MeCN/H₂O (10 mmol/L NH₄HCO₃), X-Select 10 μm 19*250 mm, 20 m/min, UV 254] to afford the title compound (19.1 mg, 18%). ESI-MS m/z calcd for [C₂₅H₂₂F₃N₇O₄S] [M+H]⁺: 574.1; found: 573.9. ¹H NMR (400 MHz, Methanol-d₄) δ 8.90 (d, J=2.4 Hz, 1H), 8.89 (d, J=2.4 Hz, 1H), 8.18 (s, 1H), 7.89 (s, 1H), 7.39-7.32 (m, 2H), 4.86-4.83 (m, 1H), 4.54 (d, J=9.6 Hz, 1H), 4.44 (dd, J=10.4, 2.8 Hz, 1H), 4.10 (d, J=2.4 Hz, 1H), 3.85-3.79 (m, 2H), 3.72-3.69 (m, 1H), 2.96 (s, 3H), 2.48 (s, 3H).

### Example 46

### 5-Cyano-1-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-(trifluoromethyl)benzene

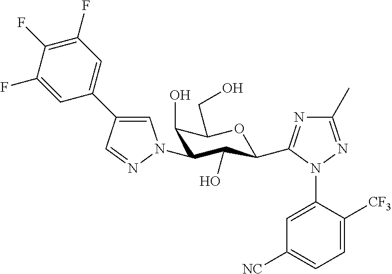

To a solution of 5-bromo-1-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-(trifluoromethyl)benzene (30 mg, 0.046 mmol) in DMF (4.0 mL) Zn (3.0 mg, 0.046 mmol), Zn(CN)₂ (16.3 mg, 0.14 mmol), 1,1′-bis(diphenylphosphino)ferrocene (2.6 mg, 0.0046 mmol) and Pd₂(dba)₃ (4.2 mg, 0.0046 mmol) were added. The mixture was purged three times with nitrogen, and stirred 2.5 h at 100° C. The mixture was filtered, and the filtrate was purified by prep HPLC [MeCN/H₂O (10 mmol/L NH₄HCO₃), X-Select 10 μm 19*250 mm, 20 mL/min, UV 254] to afford the title compound (12.8 mg, 47%). ESI-MS m/z calcd for [C₂₆H₂₀F₆N₆O₄] [M+H]⁺: 595.1; found: 594.8. ¹H NMR (400 MHz, Methanol-d₄) δ 8.25-8.07 (m, 4H), 7.89 (s, 1H), 7.42-7.30 (m, 2H), 4.82-4.69 (m, 1H), 4.37 (dd, J=10.8, 2.8 Hz, 1H), 4.32 (d, J=9.2 Hz, 1H), 4.06 (d, J=2.8 Hz, 1H), 3.69-3.57 (m, 3H), 2.44 (s, 3H).

### Example 47

### 5-Chloro-1-{5-{3-deoxy-3-[4-(3,4,5-trifluorophenyl)-3-phenyl-1H-1,2-pyrazol-1-yl]-β-D-galactopyranosyl}-3-methyl-1H-1,2,4-triazol-1-yl}-2-(trifluoromethyl)benzene

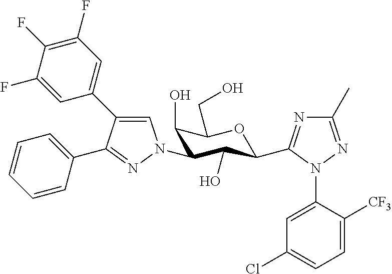

To a solution of 5-chloro-1-{5-{3-deoxy-3-[2-(phenylmethylene)hydrazinyl]-β-D-galactopyranosyl}-3-methyl-1H-1,2,4-triazol-1-yl}-2-(trifluoromethyl)benzene (200 mg, 0.32 mmol) in anhydrous THF (2.5 mL) at −78° C. a potassium tert-butoxide solution (0.16 mL, 1 M in THF, 0.16 mmol) was added dropwise under N₂ atmosphere. After stirring 10 min at −78° C., (E)-1,2,3-trifluoro-5-(2-nitrovinyl)benzene (31.9 mg, 0.16 mmol) in anhydrous THF (0.5 mL) was added dropwise. After 15 minutes, TFA (0.040 mL, 0.52 mmol) was added. The mixture was stirred 2 h at −78° C., it was then warmed to rt overnight. The mixture was concentrated and purified by prep HPLC [MeCN/H₂O (10 mmol/L TFA), X-Select 10 μm 19*250 mm, 20 mL/min, UV 254] to afford the title compound (2.1 mg, 3%). ESI-MS m/z calcd for [C₃₁H₂₄ClF₆N₅O₄][M+H]⁺: 680.1; found: 679.7. ¹H NMR (400 MHz, Methanol-d₄) δ 8.04 (s, 1H), 7.96 (d, J=8.4 Hz, 1H), 7.86 (dd, J=8.4, 1.2 Hz, 1H), 7.78 (s, 1H), 7.47-7.34 (m, 5H), 7.05-6.93 (m, 2H), 4.84-4.72 (m, 1H), 4.42 (dd, J=10.8, 2.8 Hz, 1H), 4.31 (d, J=9.2 Hz, 1H), 4.14 (d, J=2.8 Hz, 1H), 3.72-3.61 (m, 3H), 2.44 (s, 3H).

### Example 48

### 5-Chloro-1-{5-{3-deoxy-3-[4-(3,4,5-trifluorophenyl)-3-(2-fluorophenyl)-1H-1,2-pyrazol-1-yl]-β-D-galactopyranosyl}-3-methyl-1H-1,2,4-triazol-1-yl}-2-(trifluoromethyl)benzene

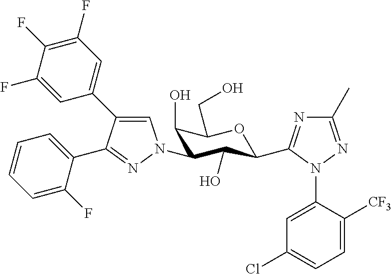

To a solution of 5-chloro-1-{5-{3-deoxy-3-{2-[(2-fluorophenyl)methylene]hydrazinyl}-β-D-galactopyranosyl}-3-methyl-1H-1,2,4-triazol-1-yl}-2-(trifluoromethyl)benzene (50 mg, 0.092 mmol) in anhydrous THF (2.5 mL) at −78° C. a potassium tert-butoxide solution (0.11 mL, 1 M in THF, 0.11 mmol) was added dropwise under N₂ atmosphere. After stirring 10 min at −78° C., (E)-1,2,3-trifluoro-5-(2-nitrovinyl)benzene (22.4 mg, 0.11 mmol) in anhydrous THF (0.5 mL) was added dropwise. After 15 minutes, TFA (0.015 mL, 0.18 mmol) was added. The mixture was stirred 2 h at −78° C., it was then warmed to rt overnight. The mixture was concentrated and purified by prep HPLC [MeCN/H₂O (10 mmol/L TFA), X-Select 10 μm 19*250 mm, 20 m/min, UV 254] to afford the title compound (16.4 mg, 26%). ESI-MS m/z calcd for [C₃₁H₂₃ClF₇N₅O₄] [M+H]⁺: 698.1; found: 697.8. ¹H NMR (400 MHz, Methanol-d₄) δ 8.14 (s, 1H), 7.95 (d, J=8.4 Hz, 1H), 7.85 (dd, J=8.4, 1.2 Hz, 1H), 7.78 (s, 1H), 7.55-7.42 (m, 2H), 7.28 (td, J=7.6, 1.2 Hz, 1H), 7.18-7.08 (m, 1H), 6.98-6.86 (m, 2H), 4.85-4.75 (m, 1H), 4.44 (dd, J=10.4, 2.8 Hz, 1H), 4.31 (d, J=9.6 Hz, 1H), 4.15 (d, J=2.8 Hz, 1H), 3.72-3.63 (m, 3H), 2.44 (s, 3H).

### Example 49

### 5-Chloro-1-{5-{3-deoxy-3-[4-(3,4,5-trifluorophenyl)-3-methyl-1H-1,2-pyrazol-1-yl]-β-D-galactopyranosyl}-3-methyl-1H-1,2,4-triazol-1-yl}-2-(trifluoromethyl)benzene

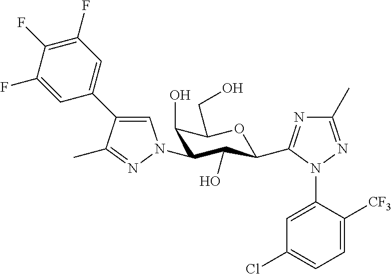

To a solution of 5-chloro-1-{5-[3-deoxy-3-(4-iodo-3-methyl-1H-1,2-pyrazol-1-yl)-3-D-galactopyranosyl]-3-methyl-1H-1,2,4-triazol-1-yl}-2-(trifluoromethyl)benzene (30 mg, 0.049 mmol) and (3,4,5-trifluorophenyl)boronic acid (25.8 mg, 0.15 mmol) in 1,4-dioxane/H₂O (3.0 mL, 2:1) Pd(dppf)Cl₂ (7.2 mg, 0.0098 mmol) and K₂CO₃ (33.8 mg, 0.24 mmol) were added. The mixture was purged three times with nitrogen, and stirred 2 h at 65° C. The mixture was filtered, and the filtrate was acidified to pH 3-4 using TFA. The mixture was concentrated and purified by prep HPLC [MeCN/H₂O (10 mmol/L NH₄HCO₃), X-Select 10 μm 19*250 mm, 20 mL/min, UV 254] to afford the title compound (6.5 mg, 22%). ESI-MS m/z calcd for [C₂₆H₂₂ClF₆N₅O₄] [M+H]⁺: 618.1; found: 617.9. ¹H NMR (400 MHz, Methanol-d₄) δ 8.00-7.93 (m, 2H), 7.85 (dd, J=8.8, 1.2 Hz, 1H), 7.77 (s, 1H), 7.25-7.16 (m, 2H), 4.78-4.66 (m, 1H), 4.32-4.23 (m, 2H), 4.06 (d, J=2.8 Hz, 1H), 3.68-3.61 (m, 3H), 2.43 (s, 3H), 2.37 (s, 3H).

### Example 50

### 1-{5-{3-[3-Benzyl-4-(3,4,5-trifluorophenyl)-1H-1,2-pyrazol-1-yl]-3-deoxy-β-D-galactopyranosyl}-3-methyl-1H-1,2,4-triazol-1-yl}-5-chloro-2-(trifluoromethyl)benzene

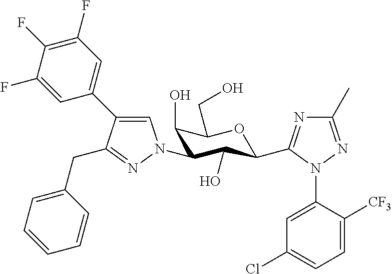

To a solution of 1-{5-[3-(3-benzyl-4-iodo-1H-1,2-pyrazol-1-yl)-3-deoxy-β-D-galactopyranosyl]-3-methyl-1H-1,2,4-triazol-1-yl}-5-chloro-2-(trifluoromethyl)benzene (28 mg, 0.041 mmol) in 1,4-dioxane/H₂O (3.0 mL, 2:1) (3,4,5-trifluorophenyl)boronic acid (21.4 mg, 0.12 mmol), Pd(dppf)Cl₂ (8.9 mg, 0.012 mmol) and K₂CO₃ (28 mg, 0.20 mmol) were added. The mixture was purged three times with nitrogen, and stirred 1.5 h at 60° C. The mixture was filtered, and the filtrate was acidified to pH 3-4 using TFA. The mixture was concentrated and purified by prep HPLC [MeCN/H₂O (10 mmol/L TFA), X-Select 10 μm 19*250 mm, 20 mL/min, UV 254] to afford the title compound (8.5 mg, 30%). ESI-MS m/z calcd for [C₃₂H₂₆ClF₆N₅O₄] [M+H]⁺: 694.2; found: 693.8. ¹H NMR (400 MHz, Methanol-d₄) δ 8.00 (s, 1H), 7.95 (d, J=8.8 Hz, 1H), 7.85 (dd, J=8.8, 0.8 Hz, 1H), 7.77 (s, 1H), 7.26-7.19 (m, 2H), 7.19-7.11 (m, 3H), 7.06-6.97 (m, 2H), 4.82-4.69 (m, 1H), 4.37 (dd, J=10.8, 2.8 Hz, 1H), 4.29 (d, J=9.2 Hz, 1H), 4.14-4.09 (m, 3H), 3.69-3.62 (m, 3H), 2.44 (s, 3H).

### Example 51

### 5-Chloro-1-{5-{3-[3-cyano-4-(3,4,5-trifluorophenyl)-1H-1,2-pyrazol-1-yl]-3-deoxy-β-D-galactopyranosyl}-3-methyl-1H-1,2,4-triazol-1-yl}-2-(trifluoromethyl)benzene

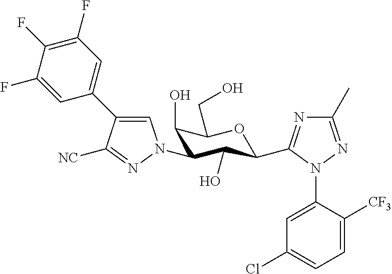

A solution of 5-chloro-1-{5-{3-deoxy-3-[4-(3,4,5-trifluorophenyl)-3-ethoxycarbonyl-1H-1,2-pyrazol-1-yl]-β-D-galactopyranosyl}-3-methyl-1H-1,2,4-triazol-1-yl}-2-(trifluoromethyl)benzene (42 mg, 0.062 mmol) in ammonia (3.0 mL, 7 M in MeOH) was stirred 48 h at 60° C. in a sealed tube. The mixture was concentrated, and the crude product was dissolved in THF (2.0 mL). Trifluoroacetic anhydride (86.4 μL) and pyridine (50.3 μL) were added, and the mixture was stirred 2 h at rt. The mixture was concentrated, water (1.0 mL) was added, and the mixture was extracted with EtOAc (3×1.0 mL). The combined organic phases were dried, evaporated and the residue was dissolved in MeOH (2.0 mL). Water (0.5 mL) and Et₃N (0.5 mL) were added, and the mixture was stirred 1 h at rt. The mixture was concentrated and purified by prep HPLC [MeOH/H₂O (10 mmol/L, 0.05% TFA), X-Select 10 μm 19*250 mm, 20 mL/min, UV 254] to give the title compound (6.0 mg, 15%). ESI-MS m/z calcd for [C₂₆H₁₉ClF₆N₆O₄] [M+H]⁺: 629.1; found: 629.2. ¹H NMR (400 MHz, Methanol-d₄) δ 8.34 (s, 1H), 7.95 (d, J=8.8 Hz, 1H), 7.85 (dd, J=8.8, 1.2 Hz, 1H), 7.76 (br s, 1H), 7.48 (dd, J=9.2, 6.8 Hz, 2H), 4.83-4.79 (m, 1H), 4.53 (dd, J=10.8, 2.8 Hz, 1H), 4.30 (d, J=9.2 Hz, 1H), 4.09 (d, J=2.4 Hz, 1H), 3.67-3.64 (m, 3H), 2.44 (s, 3H).

### Example 52

### 5-Chloro-1-{5-{3-deoxy-3-[4-(3,4,5-trifluorophenyl)-3-(2-methoxyphenyl)-1H-1,2-pyrazol-1-yl]-β-D-galactopyranosyl}-3-methyl-1H-1,2,4-triazol-1-yl}-2-(trifluoromethyl)benzene

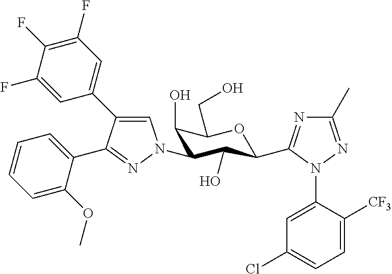

To a solution of 5-chloro-1-{5-{3-deoxy-3-{2-[(2-methoxyphenyl)methylene]hydrazinyl}-β-D-galactopyranosyl}-3-methyl-1H-1,2,4-triazol-1-yl}-2-(trifluoromethyl)benzene (30 mg, 0.054 mmol) in anhydrous THF (2.5 mL) at −78° C. a potassium tert-butoxide solution (0.054 mL, 1 M in THF, 0.054 mmol) was added dropwise under N₂ atmosphere. After stirring 10 min at −78° C., (E)-1,2,3-trifluoro-5-(2-nitrovinyl)benzene (13.2 mg, 0.065 mmol) in anhydrous THF (0.5 mL) was added dropwise. After 15 minutes, TFA (8.3 μL, 0.11 mmol) was added. The mixture was stirred 2 h at −78° C., it was then warmed to rt overnight. The mixture was concentrated and purified by prep HPLC [MeCN/H₂O (10 mmol/L TFA), X-Select 10 μm 19*250 mm, 20 m/min, UV 254] to afford the title compound (4.1 mg, 11%). ESI-MS m/z calcd for [C₃₂H₂₆ClF₆N₅O₅] [M+H]⁺: 710.2; found: 709.8. ¹H NMR (400 MHz, Methanol-d₄) δ 8.11 (s, 1H), 7.95 (d, J=8.4 Hz, 1H), 7.85 (dd, J=8.4, 1.6 Hz, 1H), 7.78 (s, 1H), 7.48-7.41 (m, 1H), 7.38 (dd, J=7.6, 1.6 Hz, 1H), 7.10-6.99 (m, 2H), 6.94-6.86 (m, 2H), 4.83-4.78 (m, 1H), 4.40 (dd, J=10.8, 2.8 Hz, 1H), 4.30 (d, J=9.6 Hz, 1H), 4.13 (d, J=2.4 Hz, 1H), 3.71-3.63 (m, 3H), 3.49 (s, 3H), 2.44 (s, 3H).

### Example 53

### 5-Chloro-1-{5-{3-deoxy-3-[4-(3,4,5-trifluorophenyl)-3-(2-methylphenyl)-1H-1,2-pyrazol-1-yl]-β-D-galactopyranosyl}-3-methyl-1H-1,2,4-triazol-1-yl}-2-(trifluoromethyl)benzene

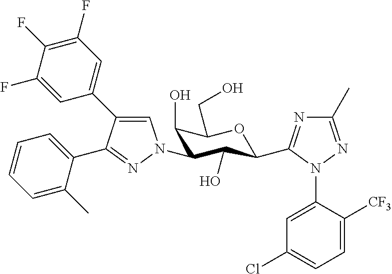

To a solution of 5-chloro-1-{5-{3-deoxy-3-[4-iodo-3-(2-methylphenyl)-1H-1,2-pyrazol-1-yl]-β-D-galactopyranosyl}-3-methyl-1H-1,2,4-triazol-1-yl}-2-(trifluoromethyl)benzene (35 mg, 0.051 mmol) in 1,4-dioxane/H₂O (3.0 mL, 2:1) (3,4,5-trifluorophenyl)boronic acid (26.8 mg, 0.15 mmol), Pd(dppf)Cl₂ (11.1 mg, 0.015 mmol) and K₂CO₃ (35.1 mg, 0.25 mmol) were added. The mixture was purged three times with nitrogen, and stirred 1 h at 60° C. The mixture was filtered, and the filtrate was acidified to pH 3-4 using TFA. The mixture was evaporated and purified by prep HPLC [MeCN/H₂O (10 mmol/L NH₄HCO₃), X-Select 10 μm 19*250 mm, 20 mL/min, UV 254] to afford the title compound (8.84 mg, 25%). ESI-MS m/z calcd for [C₃₂H₂₆ClF₆N₆O₄] [M+H]⁺: 694.2; found: 694.2. ¹H NMR (400 MHz, Methanol-d₄) δ 8.20 (s, 1H), 7.95 (d, J=8.4 Hz, 1H), 7.85 (d, J=8.4 Hz, 1H), 7.78 (s, 1H), 7.41-7.33 (m, 1H), 7.30 (d, J=7.2 Hz, 1H), 7.28-7.23 (m, 2H), 6.91-6.81 (m, 2H), 4.82-4.77 (m, 1H), 4.41 (dd, J=10.8, 2.8 Hz, 1H), 4.30 (d, J=9.2 Hz, 1H), 4.14 (d, J=2.8 Hz, 1H), 3.72-3.63 (m, 3H), 2.45 (s, 3H), 2.06 (s, 3H).

### Example 54

### 5-Chloro-1-{5-{3-deoxy-3-[4-(3,4,5-trifluorophenyl)-3-(3-fluorophenyl)-1H-1,2-pyrazol-1-yl]-β-D-galactopyranosyl}-3-methyl-1H-1,2,4-triazol-1-yl}-2-(trifluoromethyl)benzene

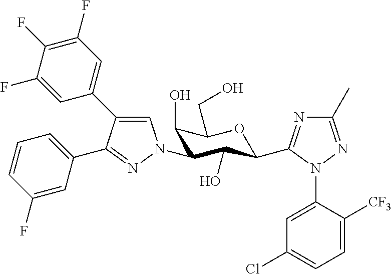

To a solution of 5-chloro-1-{5-{3-deoxy-3-[3-(3-fluorophenyl)-4-iodo-1H-1,2-pyrazol-1-yl]-β-D-galactopyranosyl}-3-methyl-1H-1,2,4-triazol-1-yl}-2-(trifluoromethyl)benzene (26 mg, 0.038 mmol) in 1,4-dioxane/H₂O (5 mL, 4:1) (3,4,5-trifluorophenyl)boronic acid (19.8 mg, 0.11 mmol), Pd(dppf)Cl₂ (8.2 mg, 0.011 mmol) and K₂CO₃ (25.9 mg, 0.19 mmol) were added. The mixture was purged three times with nitrogen, and stirred 1 h at 60° C. The mixture was filtered, evaporated, and purified by prep HPLC [MeCN/H₂O (10 mmol/L NH₄HCO₃), X-Select 10 μm 19*250 mm, 20 m/min, UV 254] to afford the title compound (4.67 mg, 18%). ESI-MS m/z calcd for [C₃₁H₂₃ClF₇N₅O₄] [M+H]⁺: 698.1; found: 698.2. ¹H NMR (400 MHz, Methanol-d₄) δ 8.03 (s, 1H), 7.95 (d, J=8.4 Hz, 1H), 7.85 (d, J=8.8 Hz, 1H), 7.78-7.76 (m, 1H), 7.40-7.34 (m, 1H), 7.22-7.17 (m, 2H), 7.13-7.07 (m, 1H), 7.03-6.98 (m, 2H), 4.81-4.79 (m, 1H), 4.43 (dd, J=10.8, 2.8 Hz, 1H), 4.31 (d, J=9.2 Hz, 1H), 4.15 (d, J=2.8 Hz, 1H), 3.70-3.65 (m, 3H), 2.44 (s, 3H).

### Example 55

### 5-Chloro-1-{5-{3-deoxy-3-[4-(3,4,5-trifluorophenyl)-3-(4-fluorophenyl)-1H-1,2-pyrazol-1-yl]-β-D-galactopyranosyl}-3-methyl-1H-1,2,4-triazol-1-yl}-2-(trifluoromethyl)benzene

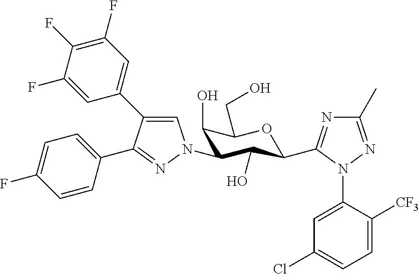

To a solution of 5-chloro-1-{5-{3-deoxy-3-[3-(4-fluorophenyl)-4-iodo-1H-1,2-pyrazol-1-yl]-β-D-galactopyranosyl}-3-methyl-1H-1,2,4-triazol-1-yl}-2-(trifluoromethyl)benzene (32 mg, 0.046 mmol) in 1,4-dioxane/H₂O (3 mL, 2:1) (3,4,5-trifluorophenyl)boronic acid (24.3 mg, 0.14 mmol), Pd(dppf)Cl₂ (10.1 mg, 0.014 mmol) and K₂CO₃ (31.9 mg, 0.23 mmol) were added. The mixture was purged three times with nitrogen, and stirred 1 h at 60° C. The mixture was filtered, and the filtrate was acidified to pH 3-4 using TFA. The mixture was evaporated and purified by prep HPLC [MeCN/H₂O (10 mmol/L NH₄HCO₃), X-Select 10 μm 19*250 mm, 20 mL/min, UV 254] to afford the title compound (9.52 mg, 30%). ESI-MS m/z calcd for [C₃₁H₂₃ClF₇N₅O₄] [M+H]⁺: 698.1; found: 698.2. ¹H NMR (400 MHz, Methanol-d₄) δ 8.03 (s, 1H), 7.96 (d, J=8.4 Hz, 1H), 7.86 (dd, J=8.4, 1.2 Hz, 1H), 7.78 (s, 1H), 7.48-7.41 (m, 2H), 7.16-7.08 (m, 2H), 7.02-6.94 (m, 2H), 4.83-4.77 (m, 1H), 4.41 (dd, J=10.4, 2.8 Hz, 1H), 4.30 (d, J=9.2 Hz, 1H), 4.15 (d, J=2.8 Hz, 1H), 3.70-3.64 (m, 3H), 2.44 (s, 3H).

### Example 56

### 5-Chloro-1-{5-{3-deoxy-3-{4-[4-(trifluoromethyl)phenyl]-1H-1,2-pyrazol-1-yl}-β-D-galactopyranosyl}-3-methyl-1H-1,2,4-triazol-1-yl}-2-(trifluoromethyl)benzene

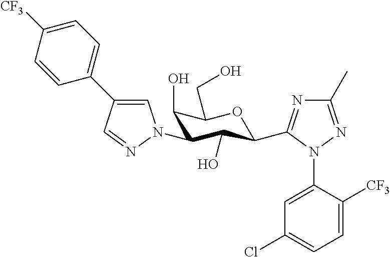

To a solution of 1-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-5-chloro-2-(trifluoromethyl)benzene (50 mg, 0.080 mmol) in EtOH (5 mL) 3-(dimethylamino)-2-[4-(trifluoromethyl)phenyl]prop-2-enal (21.4 mg, 0.088 mmol) and concentrated HCl (0.25 mL) were added and the mixture was stirred 2 h at 80° C. The mixture was concentrated and purified by prep HPLC [MeCN/H₂O (10 mmol/L NaHCO₃), X-Select 10 μm 19*250 mm, 20 mL/min, UV 254] to afford the title compound (11.7 mg, 24%). ESI-MS m/z calcd for [C₂₆H₂₂ClF₆N₅O₄] [M+H]⁺: 618.1; found: 618.2. ¹H NMR (400 MHz, Methanol-d₄) δ 8.26 (s, 1H), 7.97-7.94 (m, 2H), 7.85 (d, J=8.4 Hz, 1H), 7.77-7.75 (m, 3H), 7.64 (d, J=8.4 Hz, 2H), 4.82-4.73 (m, 1H), 4.40 (dd, J=10.8, 2.8 Hz, 1H), 4.29 (d, J=9.2 Hz, 1H), 4.11 (d, J=2.8 Hz, 1H), 3.71-3.62 (m, 3H), 2.44 (s, 3H).

### Example 57

### 5-Chloro-1-{5-{3-deoxy-3-[4-(3-fluoro-4-methylphenyl)-1H-1,2-pyrazol-1-yl]-β-D-galactopyranosyl}-3-methyl-1H-1,2,4-triazol-1-yl}-2-(trifluoromethyl)benzene

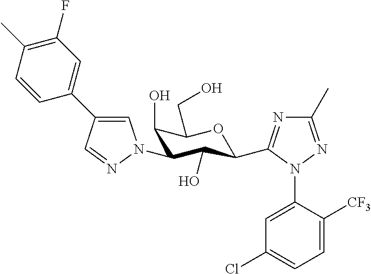

To a solution of 1-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-5-chloro-2-(trifluoromethyl)benzene (50 mg, 0.080 mmol) in EtOH (5 mL) 3-(dimethylamino)-2-(3-fluoro-4-methylphenyl)prop-2-enal (33.1 mg, 0.16 mmol) and concentrated HCl (0.2 mL) were added and the mixture was stirred 2 h at 80° C. The mixture was concentrated and purified by prep HPLC [MeCN/H₂O (10 mmol/L NaHCO₃), X-Select 10 μm 19*250 mm, 20 mL/min, UV 254] to afford the title compound (16.7 mg, 36%). ESI-MS m/z calcd for [C₂₆H₂₄ClF₄N₅O₄] [M+H]⁺: 582.2; found: 582.2. ¹H NMR (400 MHz, Methanol-d₄) δ 8.12 (s, 1H), 7.95 (d, J=8.8 Hz, 1H), 7.88-7.84 (m, 2H), 7.77 (br s, 1H), 7.28-7.18 (m, 3H), 4.82-4.73 (m, 1H), 4.36 (dd, J=10.8, 2.8 Hz, 1H), 4.28 (d, J=9.2 Hz, 1H), 4.09 (d, J=2.4 Hz, 1H), 3.69-3.62 (m, 3H), 2.44 (s, 3H), 2.24 (s, 3H).

### Example 58

### 1-{5-{3-[4-(3-Chlorophenyl)-1H-1,2-pyrazol-1-yl]-3-deoxy-β-D-galactopyranosyl}-3-methyl-1H-1,2,4-triazol-1-yl}-5-chloro-2-(trifluoromethyl)benzene

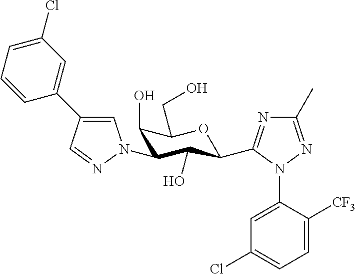

To a solution of 1-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-5-chloro-2-(trifluoromethyl)benzene (50 mg, 0.080 mmol) in EtOH (5.0 mL) 3-(dimethylamino)-2-(3-chlorophenyl)prop-2-enal (18.4 mg, 0.088 mmol) and concentrated HCl (0.25 mL) were added and the mixture was stirred 2 h at 80° C. The mixture was concentrated and purified by prep HPLC [MeCN/H₂O (10 mmol/L NH₄HCO₃), X-Select 10 μm 19*250 mm, 20 m/min, UV 254] to afford the title compound (12.4 mg, 27%). ESI-MS m/z calcd for [C₂₅H₂₂Cl₂F₃N₅O₄] [M+H]⁺: 584.1; found: 584.2. ¹H NMR (400 MHz, Methanol-d₄) δ 8.17 (s, 1H), 7.95 (d, J=8.8 Hz, 1H), 7.89 (s, 1H), 7.85 (d, J=8.4 Hz, 1H), 7.77 (s, 1H), 7.59 (s, 1H), 7.50 (d, J=8.0 Hz, 1H), 7.33 (t, J=8.0 Hz, 1H), 7.20 (d, J=8.0 Hz, 1H), 4.79-4.74 (m, 1H), 4.37 (dd, J=10.8, 2.8 Hz, 1H), 4.28 (d, J=9.6 Hz, 1H), 4.09 (d, J=2.8 Hz, 1H), 3.68-3.63 (m, 3H), 2.44 (s, 3H).

### Example 59

### 1-{5-{3-[4-(4-Chlorophenyl)-1H-1,2-pyrazol-1-yl]-3-deoxy-β-D-galactopyranosyl}-3-methyl-1H-1,2,4-triazol-1-yl}-5-chloro-2-(trifluoromethyl)benzene

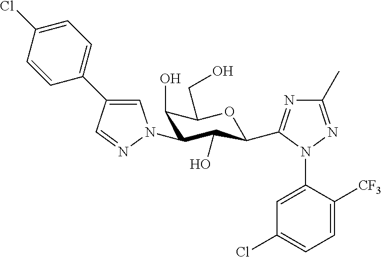

To a solution of 1-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-5-chloro-2-(trifluoromethyl)benzene (50 mg, 0.080 mmol) in EtOH (5 mL) 3-(dimethylamino)-2-(4-chlorophenyl)prop-2-enal (25.1 mg, 0.12 mmol) and concentrated HCl (0.2 mL) were added and the mixture was stirred 2 h at 80° C. The mixture was concentrated and purified by prep HPLC [MeCN/H₂O (10 mmol/L NH₄HCO₃), X-Select 10 μm 19*250 mm, 20 m/min, UV 254] to afford the title compound (19.2 mg, 41%). ESI-MS m/z calcd for [C₂₅H₂₂Cl₂F₃N₅O₄] [M+H]⁺: 584.1; found: 584.2. ¹H NMR (400 MHz, Methanol-d₄) δ 8.14 (s, 1H), 7.95 (d, J=8.4 Hz, 1H), 7.87-7.83 (m, 2H), 7.77 (br s, 1H), 7.55 (d, J=8.4 Hz, 2H), 7.35 (d, J=8.4 Hz, 2H), 4.81-4.73 (m, 1H), 4.37 (dd, J=10.8, 2.8 Hz, 1H), 4.28 (d, J=9.2 Hz, 1H), 4.09 (d, J=2.8 Hz, 1H), 3.68-3.64 (m, 3H), 2.44 (s, 3H).

### Intermediate 1

### 5-Chloro-2-cyclopropyl-3-nitropyridine

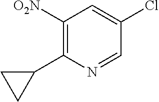 [SMILES: O=[N+]([O-])c1cc(Cl)cnc1C1CC1]

To a solution of 2,5-dichloro-3-nitropyridine (6.0 g, 31.1 mmol) in 1,4-dioxane (120 mL) and H₂O (24 mL) potassium cyclopropyltrifluoroborate (7.36 g, 46.6 mmol), K₂CO₃ (12.9 g, 93.3 mmol) and Pd(dppf)Cl₂ (1.14 g, 1.55 mmol) were added and the mixture was stirred 16 h at 100° C. under nitrogen atmosphere. The mixture was partitioned between water (100 mL) and DCM (100 mL) and the aqueous phase was extracted with DCM (2×100 mL). The combined organic phases were washed with water (100 mL) and brine (50 mL), dried over Na₂SO₄, evaporated, and purified by column chromatography (PE/EtOAc=10/1-2/1, Silica-CS 80 g, 40 m/min, silica gel, UV 254) to afford the product (3.3 g, 53%). ¹H NMR (400 MHz, CDCl₃) δ 8.50 (d, J=2.0 Hz, 1H), 8.06 (d, J=2.4 Hz, 1H), 2.67-2.60 (m, 1H), 1.19-1.06 (m, 4H).

### 5-Chloro-2-cyclopropylpyridin-3-amine

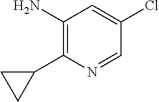 [SMILES: Nc1cc(Cl)cnc1C1CC1]

To a solution of 5-chloro-2-cyclopropyl-3-nitropyridine (3.3 g, 16.6 mmol) in EtOH (100 mL) iron (4.64 g, 8.31 mmol) and NH₄Cl (4.44 g, 8.31 mmol) were added and the mixture was stirred 16 h at rt under hydrogen atmosphere. The mixture was filtered and evaporated to afford the product (2.00 g, 71%). ESI-MS m/z calcd for [C₈H₉ClN₂][M+H]⁺: 169.0; found: 169.0. ¹H NMR (400 MHz, DMSO-d₆) δ 7.57 (d, J=2.0 Hz, 1H), 6.91 (d, J=2.4 Hz, 1H), 2.06-1.99 (m, 1H), 0.85-0.77 (m, 4H).

### (5-Chloro-2-cyclopropyl-3-pyridyl)hydrazine

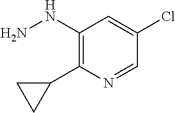 [SMILES: NNc1cc(Cl)cnc1C1CC1]

To a solution of 5-chloro-2-cyclopropylpyridin-3-amine (450 mg, 2.67 mmol) in water (7 mL) H₂SO₄ (7 mL, 98% w/w) was added. A solution of NaNO₂ (552 mg, 8.01 mmol) in water (2 mL) was added dropwise at −15° C. and the resulting mixture was stirred 2 h under nitrogen atmosphere at −15° C. Then tin(II) chloride dihydrate (3.61 g, 1.60 mmol) was added by small portion at −15° C. and the mixture was stirred 2 h under nitrogen atmosphere at −15° C. NaOH (50 mL, 5 M) was added at −15° C. followed by water (50 mL) and DCM (50 mL). The phases were separated, and the aqueous phase was extracted with DCM (2×50 mL). The combined organic phases were washed with brine (50 mL), dried over Na₂SO₄ and evaporated to afford the product (230 mg, 47%). ESI-MS m/z calcd for [C₈H₁₀ClN₃] [M+H]⁺: 184.1; found: 184.3. ¹H NMR (400 MHz, DMSO-d₆) δ 7.61 (d, J=2.4 Hz, 1H), 7.29 (d, J=2.0 Hz, 1H), 7.19 (s, 1H), 4.27 (s, 2H), 2.05-1.98 (m, 1H), 0.85-0.81 (m, 4H).

### 2,4,6-Tri-O-acetyl-3-azido-3-deoxy-β-D-galactopyranosyl cyanide

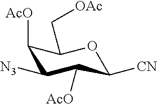

To a solution of 1,2,4,6-tetra-O-acetyl-3-azido-3-deoxy-β-D-galactopyranoside (10.0 g, 26.8 mmol) and trimethylsilyl cyanide (6.70 mL, 53.6 mmol) in nitromethane (100 mL) under argon at 0° C. boron trifluoride diethyl etherate (3.31 mL, 26.8 mmol) was added and the mixture was stirred 5 h at rt. The mixture was concentrated and purified by column chromatography (PE/EtOAc=5/1-2/1, Silica-CS 80 g, 30 m/min, silica gel, UV 254) to give the product (7.40 g, 81%). ESI-MS m/z calcd for [C₁₃H₁₆N₄O₇][M+NH₄]⁺: 358.1; found: 358.1. ¹H NMR (400 MHz, CDCl₃) δ 5.52-5.41 (m, 2H), 4.25 (d, J=10.0 Hz, 1H), 4.16-4.08 (m, 1H), 4.07-4.00 (m, 1H), 3.92-3.83 (m, 1H), 3.59 (dd, J=10.0, 3.2 Hz, 1H), 2.19 (s, 6H), 2.06 (s, 3H).

### Methyl 2,6-anhydro-4-azido-5,7-O-benzylidene-4-deoxy-D-glycero-L-manno-heptonate

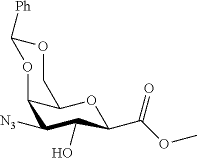

To a solution of 2,4,6-tri-O-acetyl-3-azido-3-deoxy-β-D-galactopyranosyl cyanide (13.8 g, 40.6 mmol) in MeOH (200 mL) at 0° C. acetyl chloride (15.9 g, 203 mmol) was added and the mixture was stirred 24 h at 65° C. The mixture was concentrated, and the residue was washed by DCM (100 mL). The obtained material was dissolved in DMF (30 mL). Benzaldehyde dimethylacetal (18.5 g, 121 mmol) followed by D(+)-10-camphorsulfonic acid (1.88 g, 8.09 mmol) were added and the mixture was stirred 2 h at 50° C. under reduced pressure. The mixture was partitioned between EtOAc and brine. The organic phase was concentrated and purified by column chromatography (PE/EtOAc=5/1˜1/1, Silica-CS 80 g, 50 mL/min, silica gel, UV 254) to give the product (7.1 g, 52%). ESI-MS m/z calcd for [C₁₅H₁₇N₃O₆] [M+NH₄]⁺: 353.1; found: 353.1. ¹H NMR (400 MHz, CDCl₃) δ 7.55-7.45 (m, 2H), 7.42-7.30 (m, 3H), 5.58 (s, 1H), 4.45-4.34 (m, 2H), 4.26 (d, J=2.8 Hz, 1H), 4.06 (dd, J=12.8, 2.0 Hz, 1H), 3.89 (d, J=9.6 Hz, 1H), 3.84 (s, 3H), 3.52 (d, J=1.2 Hz, 1H), 3.50-3.46 (m, 1H), 3.41 (dd, J=10.4, 3.6 Hz, 1H).

### 2,6-Anhydro-4-azido-5,7-O-benzylidene-4-deoxy-D-glycero-L-manno-heptonamide

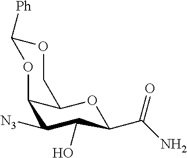

To a solution of methyl 2,6-anhydro-4-azido-5,7-O-benzylidene-4-deoxy-D-glycero-L-manno-heptonate (4.8 g, 14.3 mmol) in MeOH (50 mL) NH₃ (30 mL, 7 M in MeOH) was added and the mixture was stirred 3 h at 50° C. The mixture was evaporated to afford the product (4.0 g, 87%). ESI-MS m/z calcd for [C₁₄H₁₆N₄O₅] [M+NH₄]⁺: 338.1; found: 338.2. ¹H NMR (400 MHz, Methanol-d₄) δ 7.53-7.50 (m, 2H), 7.40-7.34 (m, 3H), 5.68 (s, 1H), 4.38 (d, J=2.4 Hz, 1H), 4.28-4.25 (m, 2H), 4.08-4.03 (m, 1H), 3.79 (d, J=7.2 Hz, 1H), 3.65 (d, J=1.2 Hz, 1H), 3.45 (dd, J=10.4, 3.6 Hz, 1H), 1.93 (s, 2H).

### 3-[5-(3-Azido-4,6-O-benzylidene-3-deoxy-β-D-galactopyranosyl)-3-methyl-1H-1,2,4-triazol-1-yl]-5-chloro-2-cyclopropylpyridine

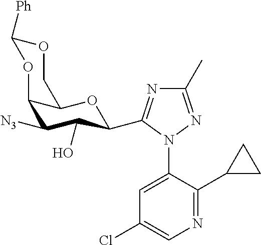

To a solution of 2,6-anhydro-4-azido-5,7-O-benzylidene-4-deoxy-D-glycero-L-manno-heptonamide (410 mg, 1.28 mmol) in 1,4-dioxane (15 mL) N,N-dimethylacetamide dimethyl acetal (188 mg, 1.41 mmol) was added and the mixture was stirred 3 h at 50° C. The mixture was evaporated and dissolved in MeCN (10 mL) and acetic acid (10 mL). (5-Chloro-2-cyclopropyl-3-pyridyl)hydrazine (282 mg, 1.54 mmol) was added and the mixture was stirred 2 h at 100° C. The mixture was evaporated and partitioned between water (20 mL) and DCM (20 mL). The aqueous phase was extracted with DCM (2×20 mL). The combined organic phases were washed with water (20 mL) and brine (20 mL), dried over Na₂SO₄, evaporated, and purified by column chromatography (PE/EtOAc=3/1˜1/2, Silica-CS 12 g, 40 mL/min, silica gel, UV 254) to afford the product (290 mg, 44%). ESI-MS m/z calcd for [C₂₄H₂₄C₁N₇O₄] [M+H]⁺: 510.2; found: 510.2. ¹H NMR (400 MHz, Chloroform-d) δ 8.44 (d, J=2.0 Hz, 1H), 7.66 (d, J=2.0 Hz, 1H), 7.47-7.45 (m, 2H), 7.36-7.34 (m, 3H), 5.53 (s, 1H), 4.86 (t, J=9.6 Hz, 1H), 4.35 (d, J=9.2 Hz, 1H), 4.24 (d, J=2.8 Hz, 1H), 3.95-3.80 (m, 2H), 3.42 (dd, J=10.4, 3.2 Hz, 1H), 3.30 (d, J=1.2 Hz, 1H), 2.05 (s, 3H), 1.60-1.54 (m, 1H), 1.09-0.93 (m, 4H).

### 3-[5-(3-Amino-4,6-O-benzylidene-3-deoxy-β-D-galactopyranosyl)-3-methyl-1H-1,2,4-triazol-1-yl]-5-chloro-2-cyclopropylpyridine

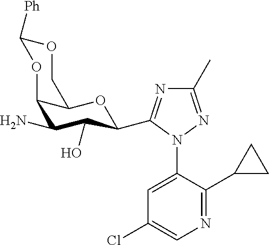

To a solution of 3-[5-(3-azido-4,6-O-benzylidene-3-deoxy-β-D-galactopyranosyl)-3-methyl-1H-1,2,4-triazol-1-yl]-5-chloro-2-cyclopropylpyridine (290 mg, 0.57 mmol) in THF (15 mL) and water (1.5 mL) triphenylphosphine (746 mg, 2.84 mmol) was added and the mixture was stirred 6 h at 50° C. The mixture was concentrated and purified by column chromatography (DCM/MeOH=1/0˜10/1, Silica-CS 12 g, 40 mL/min, silica gel, UV 254) to afford the product (275 mg, 82%). ESI-MS m/z calcd for [C₂₄H₂₆C₁N₅O₄] [M+H]⁺: 484.2; found: 484.3. ¹H NMR (400 MHz, Chloroform-d) δ 8.38 (d, J=2.0 Hz, 1H), 7.61 (d, J=2.0 Hz, 1H), 7.38-7.35 (m, 2H), 7.33-7.27 (m, 3H), 5.42 (s, 1H), 4.16 (d, J=9.2 Hz, 1H), 4.11-4.03 (m, 3H), 3.87 (dd, J=12.4, 1.6 Hz, 1H), 3.78 (dd, J=12.4, 1.2 Hz, 1H), 3.24 (s, 1H), 2.80 (dd, J=9.2, 2.4 Hz, 1H), 2.38 (s, 3H), 1.55-1.49 (m, 1H), 1.13-1.08 (m, 1H), 1.01-0.97 (m, 1H), 0.91-0.87 (m, 2H).

### 3-{5-{4,6-O-Benzylidene-3-[(2-tert-butoxycarbonyl)hydrazinyl]-3-deoxy-β-D-galactopyranosyl}-3-methyl-1H-1,2,4-triazol-1-yl}-5-chloro-2-cyclopropylpyridine

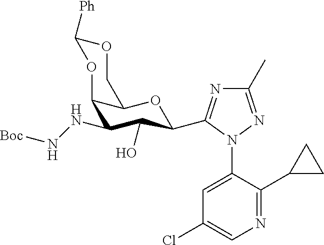

To a solution of 3-[5-(3-amino-4,6-O-benzylidene-3-deoxy-β-D-galactopyranosyl)-3-methyl-1H-1,2,4-triazol-1-yl]-5-chloro-2-cyclopropylpyridine (300 mg, 0.62 mmol) in DCM (5.0 mL) N-tert-butyloxycarbonyl-3-(4-cyanophenyl)oxaziridine (153 mg, 0.62 mmol) was added and the mixture was stirred overnight at rt. The mixture was concentrated and purified by column chromatography (PE/EtOAc=4/1˜0/1, Silica-CS 12 g, 20 mL/min, silica gel, UV 254) to afford the product (180 mg, 49%). ESI-MS m/z calcd for [C₂₉H₃₅ClN₆O₆] [M+H]⁺: 599.2; found: 598.8. ¹H NMR (400 MHz, Chloroform-d) δ 8.35 (d, J=2.0 Hz, 1H), 7.60 (d, J=2.0 Hz, 1H), 7.38-7.35 (m, 2H), 7.31-7.27 (m, 3H), 6.42 (s, 1H), 5.42 (s, 1H), 4.78-4.21 (m, 5H), 3.90-3.84 (m, 2H), 3.24 (s, 1H), 2.97 (dd, J=9.6, 3.2 Hz, 1H), 2.38 (s, 3H), 1.59-1.51 (m, 1H), 1.38 (s, 9H), 1.09-1.05 (m, 1H), 1.02-0.97 (m, 1H), 0.90-0.85 (m, 2H).

### 5-Chloro-3-{5-[3-deoxy-3-(1H-1,2-pyrazol-1-yl)-β-D-galactopyranosyl]-3-methyl-1H-1,2,4-triazol-1-yl}-2-cyclopropylpyridine

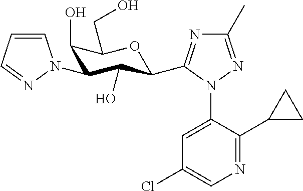

To a solution of 3-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-5-chloro-2-cyclopropylpyridine (180 mg, 0.30 mmol) in DCM (10 mL) TFA (0.50 mL) was added at 0° C. and the mixture was stirred overnight at rt. The mixture was concentrated, and the residue was dissolved in ethanol (10 mL). To the solution 1,1,3,3-tetraethoxypropane (331 mg, 1.50 mmol) was added, followed by concentrated HCl (0.25 mL). The mixture was stirred 3 h at rt, before the pH was adjusted to 4-5 using saturated aq NaHCO₃. The mixture was concentrated and purified by reversed-phase chromatography (MeCN/H₂O=1/20˜1/2, C-18 column, 20 m/min, UV 214) to give the product (65 mg, 48%). ESI-MS m/z calcd for [C₂₀H₂₃C₁N₆O₄] [M+H]⁺: 447.1; found: 447.2. ¹H NMR (400 MHz, Methanol-d₄) δ 8.57 (d, J=2.0 Hz, 1H), 7.89 (s, 1H), 7.76 (d, J=2.4 Hz, 1H), 7.51 (d, J=2.0 Hz, 1H), 6.33 (t, J=2.4 Hz, 1H), 4.64-4.62 (m, 1H), 4.38-4.34 (m, 2H), 4.03 (d, J=2.8 Hz, 1H), 3.72-3.60 (m, 3H), 2.45 (s, 3H), 1.68-1.62 (m, 1H), 1.13-0.91 (m, 4H).

### 3-{5-[3-(4-Bromo-1H-1,2-pyrazol-1-yl)-3-deoxy-β-D-galactopyranosyl]-3-methyl-1H-1,2,4-triazol-1-yl}-5-chloro-2-cyclopropylpyridine

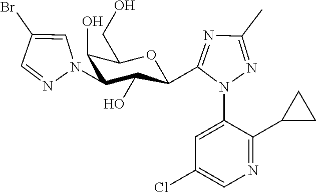

To a solution of 5-chloro-3-{5-[3-deoxy-3-(1H-1,2-pyrazol-1-yl)-3-D-galactopyranosyl]-3-methyl-1H-1,2,4-triazol-1-yl}-2-cyclopropylpyridine (65 mg, 0.15 mmol) in DCM (5 mL) N-bromosuccinimide (129 mg, 0.73 mmol) was added and the mixture was stirred overnight at rt. The mixture was concentrated and purified by reversed-phase chromatography (MeCN/H₂O=1/20˜1/2, C-18 column, 20 mL/min, UV 214) to give the product (60 mg, 79%). ESI-MS m/z calcd for [C₂₀H₂₂BrClN₆O₄][M+H]⁺: 525.1; found: 524.9. ¹H NMR (400 MHz, Methanol-d₄) δ 8.57 (d, J=2.0 Hz, 1H), 7.88 (s, 1H), 7.83 (s, 1H), 7.50 (s, 1H), 4.61-4.59 (m, 1H), 4.37-4.34 (m, 2H), 4.01 (d, J=2.4 Hz, 1H), 3.71-3.59 (m, 3H), 2.45 (s, 3H), 1.67-1.61 (m, 1H), 1.12-0.90 (m, 4H).

### Intermediate 2

### 2,6-Anhydro-4-azido-5,7-O-benzylidene-4-deoxy-D-glycero-L-manno-heptonohydrazide

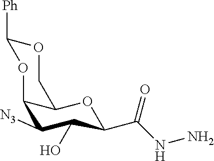

To a solution of methyl 2,6-anhydro-4-azido-5,7-O-benzylidene-4-deoxy-D-glycero-L-manno-heptonate (800 mg, 2.39 mmol) in EtOH (20 mL) hydrazine hydrate (0.514 mL, 10.6 mmol) was added and the mixture was stirred 16 h at 80° C. The mixture was concentrated and purified by prep HPLC (MeCN/H₂O (10 mmol/L NH₄HCO₃), X-Select10 μm 19*250 mm, 20 m/min, UV 254) to afford the product (705 mg, 88%). ESI-MS m/z calcd for [C₁₄H₁₇N₅O₅] [M+H]⁺: 336.1; found: 336.3. ¹H NMR (400 MHz, DMSO-d₆) δ 9.33 (s, 1H), 7.43-7.34 (m, 5H), 5.66-5.61 (m, 2H), 4.31-4.28 (m, 3H), 4.03-3.98 (m, 3H), 3.58 (d, J=9.2 Hz, 1H), 3.51 (s, 1H), 3.47-3.44 (m, 1H).

### 6-[3-(3-Azido-4,6-O-benzylidene-3-deoxy-β-D-galactopyranosyl)-4H-1,2,4-triazol-4-yl]-2-methylbenzothiazole

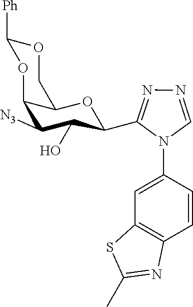

To a solution of 2,6-anhydro-4-azido-5,7-O-benzylidene-4-deoxy-D-glycero-L-manno-heptonohydrazide (705 mg, 2.10 mmol) in MeCN (20 mL) N,N-dimethylformamide dimethyl acetal (251 mg, 2.10 mol) was added and the mixture was stirred 30 min at 50° C. A solution of 6-amino-2-methylbenzothiazole (345 mg, 2.10 mol) in MeCN (10 mL) was added followed by acetic acid (2 mL) and the mixture was stirred 3 h 120° C. The mixture was concentrated and purified by column chromatography (EtOAc/MeOH=20:1, Silica-CS 40 g, 30 m/min, silica gel, UV 254) to give the product (760 mg, 74%). ESI-MS m/z calcd for [C₂₃H₂₁N₇O₄S] [M+H]⁺: 492.1; found: 492.1. ¹H NMR (400 MHz, CDCl₃) δ 8.28 (s, 1H), 7.97-7.93 (m, 2H), 7.50 (dd, J=8.4, 2.0 Hz, 1H), 7.40-7.30 (m, 5H), 5.50 (s, 1H), 4.78 (t, J=10.0 Hz, 1H), 4.44 (d, J=9.2 Hz, 1H), 4.22 (d, J=3.2 Hz, 1H), 4.12-3.95 (m, 2H), 3.48-3.44 (m, 1H), 3.31 (d, J=0.8 Hz, 1H), 2.83 (s, 3H).

### 6-[3-(3-Amino-4,6-O-benzylidene-3-deoxy-β-D-galactopyranosyl)-4H-1,2,4-triazol-4-yl]-2-methylbenzothiazole

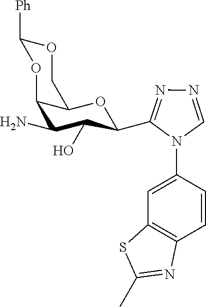

To a solution of 6-[3-(3-azido-4,6-O-benzylidene-3-deoxy-β-D-galactopyranosyl)-4H-1,2,4-triazol-4-yl]-2-methylbenzothiazole (150 mg, 0.31 mmol) in MeOH (5 mL) palladium on carbon (5% Pd, 30 mg) was added and the mixture was stirred 16 h under a hydrogen atmosphere at rt. The mixture was filtered, concentrated, and purified by prep HPLC (MeCN/H₂O, 10 mmol/L NH₄HCO₃), X-Select10 um 19*250 mm, 20 m/min, UV 254) to give the product (110 mg, 77%). ESI-MS m/z calcd for [C₂₃H₂₃N₅O₄S] [M+H]⁺: 466.1; found: 466.2. ¹H NMR (400 MHz, DMSO-d₆) 8.84 (s, 1H), 8.42 (d, J=2.4 Hz, 1H), 7.91 (d, J=8.8 Hz, 1H), 7.67 (dd, J=8.8, 2.4 Hz, 1H), 7.39-7.37 (m, 5H), 5.58 (s, 1H), 5.19 (d, J=5.2 Hz, 1H), 4.34 (d, J=9.6 Hz, 1H), 4.10-4.06 (m, 3H), 3.82-3.80 (m, 1H), 3.60 (s, 1H), 3.31 (s, 1H), 3.17 (d, J=5.2 Hz, 1H), 2.82 (s, 3H), 2.71 (dd, J=9.6, 3.2 Hz, 1H).

### 6-{3-{4,6-O-Benzylidene-3-[(2-tert-butoxycarbonyl)hydrazinyl]-3-deoxy-β-D-galactopyranosyl}-4H-1,2,4-triazol-4-yl}-2-methylbenzothiazole

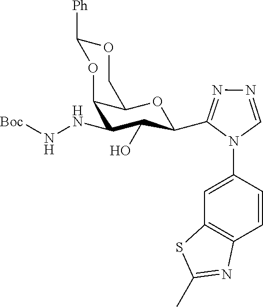

To a solution of 6-[3-(3-amino-4,6-O-benzylidene-3-deoxy-β-D-galactopyranosyl)-4H-1,2,4-triazol-4-yl]-2-methylbenzothiazole (630 mg, 1.35 mmol) in DCM (10 mL) N-tert-butyloxycarbonyl-3-(4-cyanophenyl)oxaziridine (467 mg, 1.89 mmol) was added and the mixture was stirred overnight at rt. The mixture was concentrated and purified by column chromatography (EtOAc/MeOH=10/1˜1/1, Silica-CS 12 g, 20 m/min, silica gel, UV 254) to afford the product (580 mg, 74%). ESI-MS m/z calcd for [C₂₈H₃₂N₆O₆S] [M+H]⁺: 581.2 found: 581.2. ¹H NMR (400 MHz, Chloroform-d) δ 8.18 (s, 1H), 7.91 (d, J=2.0 Hz, 1H), 7.86 (d, J=8.8 Hz, 1H), 7.46 (dd, J=8.8, 2.0 Hz, 1H), 7.26-7.17 (m, 5H), 6.77 (s, 1H), 5.41 (s, 1H), 4.41 (d, J=9.6 Hz, 1H), 4.30 (t, J=9.6 Hz, 1H), 4.25 (d, J=2.8 Hz, 1H), 4.02 (d, J=11.6 Hz, 1H), 3.90 (d, J=11.2 Hz, 1H), 3.30 (s, 1H), 2.95 (dd, J=9.2, 2.8 Hz, 1H), 2.72 (s, 3H), 1.32 (s, 9H).

### 6-{3-[3-Deoxy-3-(1H-1,2-pyrazol-1-yl)-β-D-galactopyranosyl]-4H-1,2,4-triazol-4-yl}-2-methylbenzothiazole

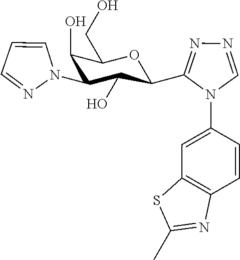

A solution of 6-{3-{4,6-O-benzylidene-3-[(2-tert-butoxycarbonyl)hydrazinyl]-3-deoxy-β-D-galactopyranosyl}-4H-1,2,4-triazol-4-yl}-2-methylbenzothiazole (150 mg, 0.26 mmol) in DCM/TFA (5.0 mL, 10:1) was stirred 16 h at rt. The mixture was concentrated and the residue was dissolved together with malondialdehyde bis(diethyl acetal) (171 mg, 0.78 mmol) in EtOH (5.0 mL). Concentrated HCl (0.2 mL) was added at 0° C. and the mixture was stirred 2 h at 0° C. The pH of the mixture was adjusted to 5 by addition of aq NaHCO₃. The mixture was concentrated and purified by prep HPLC [MeCN/H₂O (10 mmol/L/0.05% TFA), X-Select10 μm 19*250 mm, 20 mL/min, UV 254] to give the product (67 mg, 61%). ESI-MS m/z calcd for [C₁₉H₂₀N₆O₄S] [M+H]⁺: 429.1 found: 429.0. ¹H NMR (400 MHz, Methanol-d₄) δ 8.83 (s, 1H), 8.35 (d, J=2.0 Hz, 1H), 8.08 (d, J=8.4 Hz, 1H), 7.75 (d, J=2.4 Hz, 1H), 7.73 (dd, J=8.4, 2.8 Hz, 1H), 7.50 (d, J=2.0 Hz, 1H), 6.32 (t, J=2.0 Hz, 1H), 4.91-4.87 (m, 1H), 4.45 (d, J=9.6 Hz, 1H), 4.37 (dd, J=10.8, 2.8 Hz, 1H), 4.04 (d, J=2.4 Hz, 1H), 3.81-3.66 (m, 3H), 2.89 (s, 3H).

### 6-{3-[3-(4-Bromo-1H-1,2-pyrazol-1-yl)-3-deoxy-β-D-galactopyranosyl]-4H-1,2,4-triazol-4-yl}-2-methylbenzothiazole

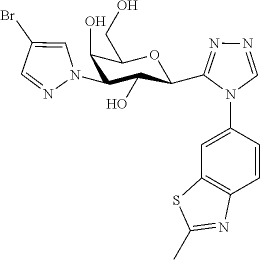

To a solution of 6-{3-[3-deoxy-3-(1H-1,2-pyrazol-1-yl)-β-D-galactopyranosyl]-4H-1,2,4-triazol-4-yl}-2-methylbenzothiazole (67 mg, 0.16 mmol) in THF (5 mL) N-bromosuccinimide (33.4 mg, 0.19 mmol) was added at 0° C. and the mixture was stirred 2 h at 0° C. The mixture was concentrated and purified by prep HPLC [MeOH/H₂O (10 mmol/L/0.05% TFA), X-Select10 μm 19*250 mm, 20 m/min, UV 254] to afford the product (56 mg, 71%). ESI-MS m/z calcd for [C₁₉H₁₉BrN₆O₄S] [M+H]⁺: 507.0 found: 507.0. ¹H NMR (400 MHz, Methanol-d₄) δ 8.87 (s, 1H), 8.34 (s, 1H), 8.07 (d, J=8.4 Hz, 1H), 7.84 (s, 1H), 7.72 (d, J=8.4 Hz, 1H), 7.50 (s, 1H), 4.91-4.87 (m, 1H), 4.50 (d, J=5.2 Hz, 1H), 4.38 (d, J=8.4 Hz, 1H), 4.04 (d, J=2.4 Hz, 1H), 3.78-3.61 (m, 3H), 2.89 (s, 3H).

### Intermediate 3

### 1-[5-(3-Azido-4,6-O-benzylidene-3-deoxy-β-D-galactopyranosyl)-3-methyl-1H-1,2,4-triazol-1-yl]-5-chloro-2-(trifluoromethyl)benzene

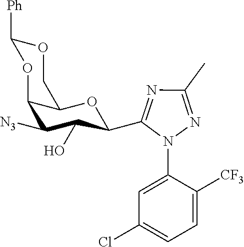

To a solution of 2,6-anhydro-4-azido-5,7-O-benzylidene-4-deoxy-D-glycero-L-manno-heptonamide (400 mg, 1.25 mmol) in 1,4-dioxane (10 mL) N,N-dimethylacetamide dimethyl acetal (175 mg, 1.31 mmol) was added and the mixture was stirred 3 h at 50° C. The mixture was evaporated, and the residue was dissolved in MeCN (10 mL) and acetic acid (4.0 mL). To the solution [5-chloro-2-(trifluoromethyl)phenyl]hydrazine (333 mg, 1.58 mmol) was added and the mixture was stirred 2 h at 100° C. The mixture was concentrated and partitioned between water (20 mL) and DCM (20 mL). The aqueous phase was extracted with DCM (2×20 mL). The combined organic phases were washed with water (20 mL) and brine (20 mL), dried over Na₂SO₄, evaporated, and purified by column chromatography (PE/EtOAc=3/1˜1/2, Silica-CS 12 g, 20 m/min, silica gel, UV 254) to afford the product (400 mg, 30%, purity 50%). ESI-MS m/z calcd for [C₂₃H₂₀ClF₃N₆O₄] [M+H]⁺: 537.1; found: 536.8.

### 1-[5-(3-Amino-4,6-O-benzylidene-3-deoxy-β-D-galactopyranosyl)-3-methyl-1H-1,2,4-triazol-1-yl]-5-chloro-2-(trifluoromethyl)benzene

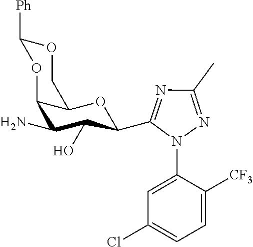

To a solution of 1-[5-(3-azido-4,6-O-benzylidene-3-deoxy-β-D-galactopyranosyl)-3-methyl-1H-1,2,4-triazol-1-yl]-5-chloro-2-(trifluoromethyl)benzene (1.50 g, 2.79 mmol) in THF (50 mL) and water (10 mL) triphenylphosphine (3.66 g, 14.0 mmol) was added and the mixture was stirred 6 h at 50° C. The mixture was concentrated and purified by column chromatography (MeOH/DCM=0˜1/10, Silica-CS 12 g, 40 mL/min, silica gel, UV 254) to afford the product (1.40 g, 98%). ESI-MS m/z calcd for [C₂₃H₂₂ClF₃N₄O₄] [M+H]⁺: 511.1; found: 510.9. ¹H NMR (400 MHz, Chloroform-d) δ 7.63 (d, J=8.4 Hz, 1H), 7.53 (s, 1H), 7.51 (d, J=8.4 Hz, 1H), 7.40-7.32 (m, 2H), 7.32-7.24 (m, 3H), 5.38 (s, 1H), 4.15 (d, J=9.2 Hz, 1H), 4.08-4.00 (m, 1H), 3.95 (t, J=9.2 Hz, 1H), 3.82 (dd, J=12.4, 1.6 Hz, 1H), 3.58 (d, J=12.0 Hz, 1H), 3.20 (d, J=0.8 Hz, 1H), 2.76 (dd, J=9.6, 3.2 Hz, 1H), 2.36 (s, 3H).

### 1-{5-{4,6-O-Benzylidene-3-[(2-tert-butoxycarbonyl)hydrazinyl]-3-deoxy-β-D-galactopyranosyl}-3-methyl-1H-1,2,4-triazol-1-yl}-5-chloro-2-(trifluoromethyl)benzene

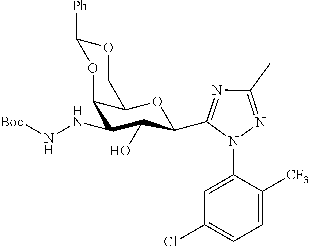

To a solution of 1-[5-(3-amino-4,6-O-benzylidene-3-deoxy-β-D-galactopyranosyl)-3-methyl-1H-1,2,4-triazol-1-yl]-5-chloro-2-(trifluoromethyl)benzene (1.40 g, 2.74 mmol) in DCM (30 mL) N-tert-butyloxycarbonyl-3-(4-cyanophenyl)oxaziridine (742 mg, 3.01 mmol) was added and the mixture was stirred overnight at rt. The mixture was concentrated and purified by column chromatography (PE/EtOAc=4/1˜0/1, Silica-CS 20 g, 30 mL/min, silica gel, UV 254) to afford the product (1.20 g, 70%). ESI-MS m/z calcd for [C₂₈H₃₁ClF₃N₅O₆] [M+H]⁺: 626.2; found: 625.8. ¹H NMR (400 MHz, Chloroform-d) δ 7.61 (d, J=8.4 Hz, 1H), 7.51 (s, 1H), 7.47 (d, J=8.4 Hz, 1H), 7.39-7.31 (m, 2H), 7.32-7.24 (m, 3H), 6.44 (br s, 1H), 5.37 (s, 1H), 4.58-4.36 (m, 2H), 4.30-4.17 (m, 3H), 3.82 (dd, J=12.4, 1.6 Hz, 1H), 3.63 (dd, J=12.4, 1.2 Hz, 1H), 3.19 (d, J=0.8 Hz, 1H), 3.03-2.94 (m, 1H), 2.36 (s, 3H), 1.38 (s, 9H).

### 5-Chloro-1-{5-[3-deoxy-3-(1H-1,2-pyrazol-1-yl)-β-D-galactopyranosyl]-3-methyl-1H-1,2,4-triazol-1-yl}-2-(trifluoromethyl)benzene

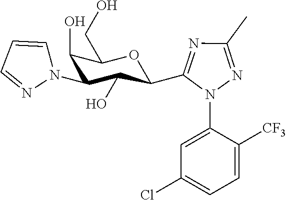

To a solution of 1-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-5-chloro-2-(trifluoromethyl)benzene (250 mg, 0.40 mmol) in DCM (10 mL) TFA (0.5 mL) was added at 0° C. and the mixture was stirred overnight at rt. The mixture was concentrated, and the residue was dissolved in ethanol (10 mL). To this solution 1,1,3,3-tetraethoxypropane (440 mg, 2.00 mmol) was added followed by concentrated HCl (0.25 mL). The mixture was stirred 3 h at rt. The pH was adjusted to 4-5 using saturated aq NaHCO₃. The mixture was concentrated and purified by reversed-phase chromatography (MeCN/H₂O=1/20˜1/2, C-18 column, 20 mL/min, UV 214) to give the product (75 mg, 40%). ESI-MS m/z calcd for [C₁₉H₁₉ClF₃N₅O₄] [M+H]⁺: 474.1; found: 474.0. ¹H NMR (400 MHz, Methanol-d₄) δ 7.85 (d, J=8.8 Hz, 1H), 7.75 (d, J=8.8 Hz, 1H), 7.69 (d, J=2.4 Hz, 1H), 7.67 (s, 1H), 7.43 (d, J=2.0 Hz, 1H), 6.24 (t, J=2.0 Hz, 1H), 4.68-4.52 (m, 1H), 4.26 (dd, J=10.8, 2.8 Hz, 1H), 4.16 (d, J=9.6 Hz, 1H), 3.95 (d, J=2.8 Hz, 1H), 3.59-3.48 (m, 3H), 2.33 (s, 3H).

### 1-{5-[3-(4-Bromo-1H-1,2-pyrazol-1-yl)-3-deoxy-β-D-galactopyranosyl]-3-methyl-1H-1,2,4-triazol-1-yl}-5-chloro-2-(trifluoromethyl)benzene

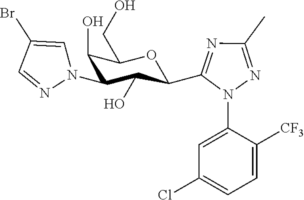

To a solution of 5-chloro-1-{5-[3-deoxy-3-(1H-1,2-pyrazol-1-yl)-β-D-galactopyranosyl]-3-methyl-1H-1,2,4-triazol-1-yl}-2-(trifluoromethyl)benzene (55 mg, 0.12 mmol) in THF (5.0 mL) N-bromosuccinimide (103 mg, 0.58 mmol) was added and the mixture was stirred overnight at rt. The mixture was concentrated and purified by reversed-phase chromatography (MeCN/H₂O=1/20˜1/2, C-18 column, 20 mL/min, UV 214) to give the product (55 mg, 86%). ESI-MS m/z calcd for [Cl₉H₁₈BrClF₃N₅O₄][M+H]⁺: 552.0; found: 551.7. ¹H NMR (400 MHz, Methanol-d₄) δ 7.85 (d, J=8.8 Hz, 1H), 7.79-7.72 (m, 2H), 7.66 (s, 1H), 7.40 (s, 1H), 4.70-4.48 (m, 1H), 4.25 (dd, J=10.8, 2.8 Hz, 1H), 4.16 (d, J=9.2 Hz, 1H), 3.93 (d, J=2.8 Hz, 1H), 3.57-3.48 (m, 3H), 2.33 (s, 3H).

### Intermediate 5

### 5-Chloro-1-{5-[3-deoxy-3-(4-iodo-1H-1,2-pyrazol-1-yl)-β-D-galactopyranosyl]-3-methyl-1H-1,2,4-triazol-1-yl}-2-(trifluoromethyl)benzene

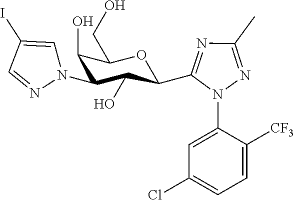

To a solution of 1-{5-[3-deoxy-3-(1H-1,2-pyrazol-1-yl)-β-D-galactopyranosyl]-3-methyl-1H-1,2,4-triazol-1-yl}-5-chloro-2-(trifluoromethyl)benzene (50 mg, 0.11 mmol) in THF (5.0 mL) N-iodosuccinimide (119 mg, 0.53 mmol) was added and the mixture was stirred overnight at rt. The mixture was concentrated and purified by reversed-phase chromatography (MeCN/H₂O=1/20˜1/2, C-18 column, 20 m/min, UV 214) to give the product (50 mg, 87%). ESI-MS m/z calcd for [C₁₉H₁₈ClF₃IN₅O₄][M+H]⁺: 600.0; found: 599.7. ¹H NMR (400 MHz, Methanol-d₄) δ 7.85 (d, J=8.8 Hz, 1H), 7.79-7.72 (m, 2H), 7.66 (s, 1H), 7.44 (s, 1H), 4.69-4.47 (m, 1H), 4.28 (dd, J=10.8, 2.8 Hz, 1H), 4.16 (d, J=9.2 Hz, 1H), 3.93 (d, J=2.8 Hz, 1H), 3.62-3.44 (m, 3H), 2.33 (s, 3H).

### Intermediate 7

### 1-{5-[3-(4-Borono-1H-1,2-pyrazol-1-yl)-3-deoxy-β-D-galactopyranosyl]-3-methyl-1H-1,2,4-triazol-1-yl}-5-chloro-2-(trifluoromethyl)benzene

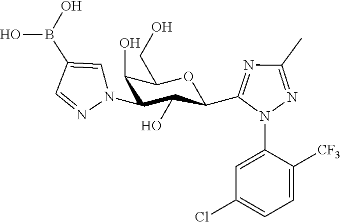

To a solution of 5-chloro-1-{5-[3-deoxy-3-(4-iodo-1H-1,2-pyrazol-1-yl)-β-D-galactopyranosyl]-3-methyl-1H-1,2,4-triazol-1-yl}-2-(trifluoromethyl)benzene (95 mg, 0.16 mmol) and bis(pinacolato)diboron (121 mg, 0.48 mmol) in DMSO (4.0 mL) Pd(dppf)Cl₂ (23.2 mg, 0.032 mmol) and potassium acetate (77.7 mg, 0.79 mmol) were added. The mixture was purged three times with nitrogen, and stirred 2 h at 60° C. The mixture was filtered and purified by reversed-phase chromatography (MeCN/H₂O (0.05% TFA)=1/20˜1/2, C-18 column, 20 m/min, UV 214) to afford the product (60 mg, 61%). ESI-MS m/z calcd for [C₁₉H₂₀BClF₃N₅O₆] [M+H]⁺: 518.1; found: 518.3. ¹H NMR (400 MHz, Methanol-d₄) δ 7.97 (s, 1H), 7.85 (d, J=8.4 Hz, 1H), 7.75 (dd, J=1.2, 8.8 Hz, 1H), 7.72-7.63 (m, 2H), 4.71-4.54 (m, 1H), 4.29 (dd, J=10.8, 2.8 Hz, 1H), 4.17 (d, J=9.2 Hz, 1H), 3.96 (d, J=2.8 Hz, 1H), 3.62-3.48 (m, 3H), 2.33 (s, 3H).

### Intermediate 8

### (2-Methyl-1,3-benzothiazol-6-yl)hydrazine

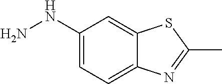

To a solution of 2-methyl-1,3-benzothiazol-6-amine (3.0 g, 18.3 mmol) in water (50 mL) hydrochloric acid (50 mL, 33% w/w) and acetic acid (15 mL) were added. A solution of NaNO₂ (1.51 g, 21.9 mmol) in water (50 mL) was added dropwise at −10° C. and the resulting mixture was stirred 2 h at −10° C. Tin(II) chloride dihydrate (8.24 g, 36.5 mmol) was added in small portions at −10° C. The resulting mixture was stirred 2 h at −10° C. NaHCO₃ (300 mL, 5% w/w) was added at 0° C. Water (200 mL) and DCM (100 mL) were added, and the phases were separated. The aqueous phase was extracted with DCM (2×100 mL). The combined organic phases were washed with water (50 mL) and brine (50 mL), dried over Na₂SO₄ and evaporated to afford the product (2.30 g, 70%). ESI-MS m/z calcd for [C₈H₉N₃S] [M+H]⁺: 180.1; found: 180.1. ¹H NMR (400 MHz, DMSO-d₆) δ 7.66 (d, J=8.8 Hz, 1H), 7.35 (d, J=2.0 Hz, 1H), 7.03 (s, 1H), 6.93 (dd, J=8.8, 2.0 Hz, 1H), 4.22 (s, 2H), 2.75 (s, 3H).

### 6-[5-(3-Azido-4,6-O-benzylidene-3-deoxy-β-D-galactopyranosyl)-3-methyl-1H-1,2,4-triazol-1-yl]-2-methylbenzothiazole

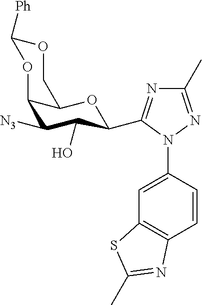

To a solution of 2,6-anhydro-4-azido-5,7-O-benzylidene-4-deoxy-D-glycero-L-manno-heptonamide (3.20 g, 9.99 mmol) in 1,4-dioxane (50 mL) N,N-dimethylacetamide dimethyl acetal (1.40 g, 10.5 mmol) was added and the mixture was stirred 3 h at 50° C. The mixture was evaporated and dissolved in MeCN (15 mL) and acetic acid (15 mL). (2-Methyl-1,3-benzothiazol-6-yl)hydrazine (1.79 g, 9.99 mmol) was added and the mixture was stirred 3 h at 80° C. The mixture was evaporated and partitioned between water (100 mL) and DCM (100 mL) and the aqueous phase was extracted with DCM (2×50 mL). The combined organic phases were washed with water (50 mL) and brine (3×50 mL), dried over Na₂SO₄, evaporated, and purified by column chromatography (PE/EtOAc=10/1˜0/1, Silica-CS 40 g, 40 m/min, silica gel, UV 254) to afford the product (1.50 g, 30%). ESI-MS m/z calcd for [C₂₄H₂₃N₇O₄S] [M+H]⁺: 506.2; found: 506.3. ¹H NMR (400 MHz, Methanol-d₄) δ 8.04 (d, J=2.0 Hz, 1H), 7.98 (d, J=8.8 Hz, 1H), 7.61 (dd, J=5.2, 2.0 Hz, 1H), 7.50-7.48 (m, 2H), 7.37-7.33 (m, 3H), 5.57 (s, 1H), 4.84 (t, J=9.6 Hz, 1H), 4.40 (d, J=9.2 Hz, 1H), 4.29 (d, J=2.8 Hz, 1H), 4.05-3.97 (m, 2H), 3.45-3.42 (m, 2H), 2.87 (s, 3H), 2.46 (s, 3H).

### 6-[5-(3-Amino-4,6-O-benzylidene-3-deoxy-β-D-galactopyranosyl)-3-methyl-1H-1,2,4-triazol-1-yl]-2-methylbenzothiazole

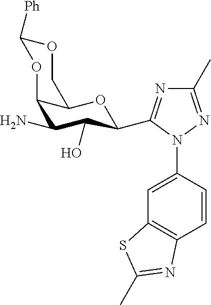

To a solution of 6-[5-(3-azido-4,6-O-benzylidene-3-deoxy-β-D-galactopyranosyl)-3-methyl-1H-1,2,4-triazol-1-yl]-2-methylbenzothiazole (1.00 g, 1.98 mmol) in MeOH (20 mL) palladium on carbon (50% Pd, 105 mg, 0.99 mmol) was added and the mixture was stirred overnight at rt under a hydrogen atmosphere. The mixture was concentrated and purified by column chromatography (PE/EtOAc=4/1˜0/1, Silica-CS 20 g, 20 mL/min, silica gel, UV 254) to give the product (900 mg, 95%). ESI-MS m/z calcd for [C₂₄H₂₅N₅O₄S] [M+H]⁺: 480.2; found: 480.1. ¹H NMR (400 MHz, DMSO-d₆) δ 8.27 (d, J=2.0 Hz, 1H), 7.97 (d, J=8.8 Hz, 1H), 7.68 (dd, J=8.8, 2.0 Hz, 1H), 7.47-7.44 (m, 2H), 7.43-7.38 (m, 3H), 5.61 (s, 1H), 5.18 (d, J=5.6 Hz, 1H), 4.29 (d, J=9.6 Hz, 1H), 4.12-4.04 (m, 3H), 3.90-3.84 (m, 1H), 3.64 (s, 1H), 2.83 (s, 3H), 2.68 (dd, J=9.6, 3.2 Hz, 1H), 2.35 (s, 3H), 1.77 (br s, 2H).

### 6-{5-{4,6-O-Benzylidene-3-[(2-tert-butoxycarbonyl)hydrazinyl]-3-deoxy-β-D-galactopyranosyl}-3-methyl-1H-1,2,4-triazol-1-yl}-2-methylbenzothiazole

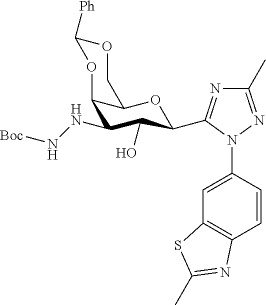

To a solution of 6-[5-(3-amino-4,6-O-benzylidene-3-deoxy-β-D-galactopyranosyl)-3-methyl-1H-1,2,4-triazol-1-yl]-2-methylbenzothiazole (900 mg, 1.88 mmol) in DCM (20 mL) N-tert-butyloxycarbonyl-3-(4-cyanophenyl)oxaziridine (462 mg, 1.88 mmol) was added and the mixture was stirred overnight at rt. The mixture was concentrated and purified by column chromatography (PE/EtOAc=4/1˜0/1, Silica-CS 20 g, 20 mL/min, silica gel, UV 254) to afford the product (650 mg, 58%). ESI-MS m/z calcd for [C₂₉H₃₄N₆O₆S] [M+H]⁺: 595.2; found: 595.2. ¹H NMR (400 MHz, DMSO-d₆) δ 8.35 (br s, 1H), 8.26 (d, J=2.4 Hz, 1H), 7.96 (d, J=8.8 Hz, 1H), 7.67 (dd, J=8.8, 2.4 Hz, 1H), 7.52-7.37 (m, 2H), 7.40-7.37 (m, 3H), 5.57 (s, 1H), 5.21 (d, J=5.2 Hz, 1H), 4.57 (br s, 1H), 4.39 (d, J=9.6 Hz, 1H), 4.21 (d, J=2.8 Hz, 1H), 4.12-4.00 (m, 3H), 3.65 (s, 1H), 2.90 (d, J=10.0 Hz, 1H), 2.83 (s, 3H), 2.35 (s, 3H), 1.37 (s, 9H).

### 6-{5-[3-Deoxy-3-(1H-1,2-pyrazol-1-yl)-β-D-galactopyranosyl]-3-methyl-1H-1,2,4-triazol-1-yl}-2-methylbenzothiazole

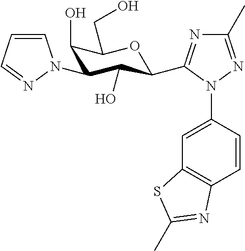

A solution of 6-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-2-methylbenzothiazole (220 mg, 0.37 mmol) in DCM/TFA (5.0 mL, 10:1) was stirred 16 h at rt. The mixture was concentrated and dissolved together with 1,1,3,3-tetraethoxypropane (245 mg, 1.11 mmol) in EtOH (5.0 mL). The mixture was cooled to 0° C., concentrated HCl (0.5 mL) was added and the mixture was stirred 2 h at 0° C. The pH was adjusted to 5 by addition of saturated aq NaHCO₃. The mixture was concentrated and purified by prep HPLC [MeCN/H₂O (10 mmol/L, 0.05% TFA), X-Select 10 μm 19*250 mm, 20 mL/min, UV 254] to give the product (75 mg, 46%). ESI-MS m/z calcd for [C₂₀H₂₂N₆O₄S] [M+H]⁺: 443.1; found: 443.0. ¹H NMR (400 MHz, Chloroform-d) δ 8.11 (d, J=2.0 Hz, 1H), 7.99 (d, J=8.4 Hz, 1H), 7.61 (dd, J=8.4, 2.0 Hz, 1H), 7.59 (d, J=2.0 Hz, 1H), 7.52 (d, J=1.6 Hz, 1H), 6.26 (t, J=2.0 Hz, 1H), 4.85 (t, J=10.0 Hz, 1H), 4.46 (d, J=9.2 Hz, 1H), 4.35 (s, 1H), 4.14 (dd, J=10.4, 2.4 Hz, 1H), 3.84 (dd, J=11.6, 6.4 Hz, 1H), 3.69 (dd, J=12.0, 3.6 Hz, 1H), 3.63 (dd, J=6.4, 4.4 Hz, 1H), 2.82 (s, 3H), 2.39 (s, 3H).

### 6-{5-[3-(4-Bromo-1H-1,2-pyrazol-1-yl)-3-deoxy-β-D-galactopyranosyl]-3-methyl-1H-1,2,4-triazol-1-yl}-2-methylbenzothiazole

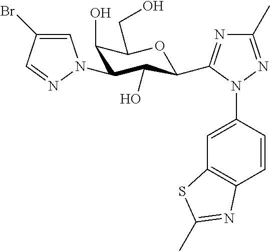

To a solution of 6-{5-[3-deoxy-3-(1H-1,2-pyrazol-1-yl)-β-D-galactopyranosyl]-3-methyl-1H-1,2,4-triazol-1-yl}-2-methylbenzothiazole (75 mg, 0.17 mmol) in THF (5 mL) N-bromosuccinimide (45.3 mg, 0.25 mmol) was added at 0° C. and the mixture was stirred 2 h at 0° C. The mixture was concentrated and purified by prep HPLC [MeOH/H₂O (10 mmol/L, 0.05% TFA), X-Select 10 μm 19*250 mm, 20 mL/min, UV 254] to give the product (65 mg, 74%). ESI-MS m/z calcd for [C₂₀H₂₁BrN₆O₄S][M+H]⁺: 521.1; found: 521.2. ¹H NMR (400 MHz, Chloroform-d) δ 8.09 (d, J=2.0 Hz, 1H), 7.97 (d, J=8.8 Hz, 1H), 7.64 (s, 1H), 7.59 (dd, J=8.8, 2.0 Hz, 1H), 7.47 (s, 1H), 4.84 (t, J=10.0 Hz, 1H), 4.42 (d, J=9.2 Hz, 1H), 4.34 (s, 1H), 4.10 (dd, J=10.4, 2.4 Hz, 1H), 3.81 (dd, J=11.6, 6.8 Hz, 1H), 3.68-3.60 (m, 2H), 2.82 (s, 3H), 2.37 (s, 3H).

### Intermediate 10

### N-[5-Chloro-2-(trifluoromethyl)phenyl]acetamide

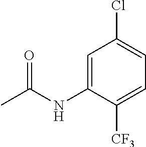

To a solution of 5-chloro-2-(trifluoromethyl)aniline (5.00 g, 25.6 mmol) and pyridine (3.03 g, 38.3 mmol) in DCM (50 mL) acetic anhydride (2.90 mL, 30.7 mmol) was added and the mixture was stirred overnight at rt. The mixture was poured into water (100 mL) and extracted with DCM (2×100 mL). The combined organic phases were washed with water (50 mL) and brine (50 mL), dried over Na₂SO₄, concentrated and purified by column chromatography (PE/EtOAc=10/1˜1/1, Silica-CS 40 g, 50 mL/min, silica gel, UV 254) to afford the product (2.50 g, 41%). ESI-MS m/z calcd for [C₉H₇ClF₃NO] [M+H]⁺: 238.0; found: 238.2. ¹H NMR (400 MHz, Chloroform-d) δ 8.33 (s, 1H), 7.52 (d, J=8.4 Hz, 1H), 7.40 (s, 1H), 7.19 (d, J=8.4 Hz, 1H), 2.22 (s, 3H).

### N-[5-Chloro-2-(trifluoromethyl)phenyl]thioacetamide

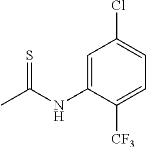

To a solution of N-[5-chloro-2-(trifluoromethyl)phenyl]acetamide (2.50 g, 10.5 mmol) in toluene (20 mL) Lawesson's reagent (2.34 g, 5.79 mmol) was added and the mixture was stirred 2 h at 110° C. The mixture was evaporated and purified by column chromatography (PE/EtOAc=10/1˜4/1, Silica-CS 40 g, 50 m/min, silica gel, UV 254) to afford the product (2.1 g, 79%). ESI-MS m/z calcd for [C₉H₇ClF₃NS] [M+H]⁺: 254.0; found: 254.0. ¹H NMR (400 MHz, Chloroform-d) δ 8.52 (s, 1H), 8.09 (s, 1H), 7.64 (d, J=8.4 Hz, 1H), 7.39 (d, J=8.4 Hz, 1H), 2.76 (s, 3H).

### Methyl N-[5-chloro-2-(trifluoromethyl)phenyl]ethanimidothioate

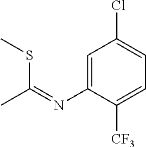 [SMILES: CS/C(C)=N\c1cc(Cl)ccc1C(F)(F)F]

To a solution of N-[5-chloro-2-(trifluoromethyl)phenyl]thioacetamide (2.10 g, 8.28 mmol) in MeCN (40 mL) K₂CO₃ (1.70 g, 12.3 mmol) and iodomethane (1.03 mL, 16.6 mmol) were added and the mixture was stirred 2 h at rt. The mixture was concentrated, dissolved in EtOAc and washed with water and brine. The organic phase was dried over Na₂SO₄, concentrated, and purified by column chromatography (PE/EtOAc=100/1˜50/1, Silica-CS 40 g, 50 m/min, silica gel, UV 254) to afford the product (1.80 g, 81%). ESI-MS m/z calcd for [C₁₀H₉ClF₃NS] [M+H]⁺: 268.0; found: 268.1. ¹H NMR (400 MHz, Chloroform-d) δ 7.52 (d, J=8.4 Hz, 1H), 7.09 (d, J=8.4, 1H), 6.75 (s, 1H), 2.40 (s, 3H), 2.06-1.91 (m, 3H).

### 1-[3-(3-Azido-4,6-O-benzylidene-3-deoxy-β-D-galactopyranosyl)-5-methyl-4H-1,2,4-triazol-4-yl]-5-chloro-2-(trifluoromethyl)benzene (atropisomers 1 and 2)

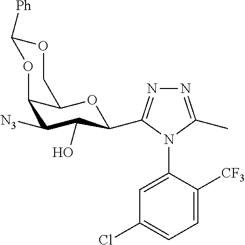

To a solution of 2,6-anhydro-4-azido-5,7-O-benzylidene-4-deoxy-D-glycero-L-manno-heptonohydrazide (1.00 g, 2.98 mmol) in DMF (20 mL) p-toluenesulfonic acid monohydrate (56.7 mg, 0.30 mmol) and methyl-N-[5-chloro-2-(trifluoromethyl)phenyl]ethanimidothioate (958 mg, 3.58 mmol) were added and the mixture was stirred 2 h at 160° C. The mixture was concentrated and purified by reverse-phase chromatography (MeCN/water 0.01% TFA=0-45%, C₁₈ 80 g, 50 m/min, UV 254). The obtained product was further purified by preparative-SFC to afford the two atropisomers (atropisomer 1, 340 mg, 21%, and atropisomer 2, 180 mg, 11%).

### Atropisomer 1

ESI-MS m/z calcd for [C₂₃H₂₀ClF₃N₆O₄] [M+H]⁺: 537.1; found: 536.8. ¹H NMR (400 MHz, Methanol-d₄) 7.81 (d, J=8.8 Hz, 1H), 7.63 (d, J=8.8 Hz, 1H), 7.61 (s, 1H), 7.48-7.45 (m, 2H), 7.36-7.34 (m, 3H), 5.56 (s, 1H), 4.31-4.29 (m, 2H), 4.22 (d, J=9.2 Hz, 1H), 4.02 (s, 2H), 3.45 (s, 1H), 3.36-3.32 (m, 1H), 2.20 (s, 3H).

Atropisomer 2 ESI-MS m/z calcd for [C₂₃H₂₀ClF₃N₆O₄] [M+H]⁺: 537.1; found: 536.8. ¹H NMR (400 MHz, Methanol-d₄) 7.72 (d, J=1.2 Hz, 1H), 7.67 (d, J=8.8 Hz, 1H), 7.39-7.37 (m, 5H), 7.12 (d, J=8.4 Hz, 1H), 5.49 (s, 1H), 4.44 (d, J=10 Hz, 1H), 4.30 (t, J=9.6 Hz, 1H), 4.24 (d, J=2.8 Hz, 1H), 4.00-3.92 (m, 2H), 3.43 (s, 1H), 3.38 (dd, J=10.0, 3.2 Hz, 1H), 2.20 (s, 3H).

### 1-[3-(3-Amino-4,6-O-benzylidene-3-deoxy-β-D-galactopyranosyl)-5-methyl-4H-1,2,4-triazol-4-yl]-5-chloro-2-(trifluoromethyl)benzene (atropisomer 1)

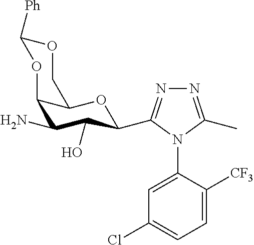

To a solution of 1-[3-(3-azido-4,6-O-benzylidene-3-deoxy-β-D-galactopyranosyl)-5-methyl-4H-1,2,4-triazol-4-yl]-5-chloro-2-(trifluoromethyl)benzene (atropisomer 1) (240 mg, 0.45 mmol) in THF (5.0 mL) and water (0.5 mL) triphenylphosphine (586 mg, 2.24 mmol) was added and the mixture was stirred 6 h at 50° C. The mixture was concentrated and purified by column chromatography (MeOH/DCM=0˜1/10, Silica-CS 12 g, 40 m/min, silica gel, UV 254) to afford the product (210 mg, 92%). ESI-MS m/z calcd for [C₂₃H₂₂ClF₃N₄O₄] [M+H]⁺: 511.1; found: 510.8. ¹H NMR (400 MHz, Chloroform-d) δ 7.57 (d, J=8.8 Hz, 1H), 7.45 (s, 1H), 7.42 (d, J=8.4 Hz, 1H), 7.30-7.28 (m, 2H), 7.23-7.21 (m, 3H), 5.32 (s, 1H), 4.12 (d, J=9.6 Hz, 1H), 4.00 (d, J=2.8 Hz, 1H), 3.88 (t, J=9.6 Hz, 1H), 3.78 (d, J=11.2 Hz, 1H), 3.65 (d, J=12.4 Hz, 1H), 3.35 (br s, 2H), 3.28 (d, J=2.0 Hz, 1H), 3.17 (s, 1H), 2.70 (dd, J=9.6, 2.8 Hz, 1H), 2.09 (s, 3H).

### 1-{3-{4,6-O-Benzylidene-3-[(2-tert-butoxycarbonyl)hydrazinyl]-3-deoxy-β-D-galactopyranosyl}-5-methyl-4H-1,2,4-triazol-4-yl}-5-chloro-2-(trifluoromethyl)benzene (atropisomer 1)

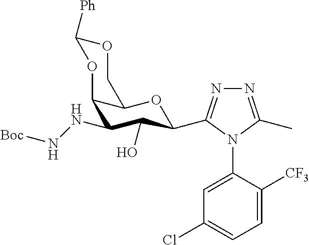

To a solution of 1-[3-(3-amino-4,6-O-benzylidene-3-deoxy-β-D-galactopyranosyl)-5-methyl-4H-1,2,4-triazol-4-yl]-5-chloro-2-(trifluoromethyl)benzene (atropisomer 1) (210 mg, 0.41 mmol) in DCM (5.0 mL) N-tert-butyloxycarbonyl-3-(4-cyanophenyl)oxaziridine (111 mg, 0.45 mmol) was added and the mixture was stirred overnight at rt. The mixture was concentrated and purified by column chromatography (PE/EtOAc=4/1˜0/1, Silica-CS 20 g, 20 mL/min, silica gel, UV 254) to afford the product (190 mg, 74%). ESI-MS m/z calcd for [C₂₈H₃₁ClF₃N₅O₆] [M+H]⁺: 626.2; found: 626.0. ¹H NMR (400 MHz, Chloroform-d) δ 7.65 (d, J=8.4 Hz, 1H), 7.52-7.49 (m, 1H), 7.45-7.33 (m, 7H), 5.39 (s, 1H), 4.44 (d, J=7.6 Hz, 1H), 4.32-4.28 (m, 2H), 3.83 (d, J=11.6 Hz, 1H), 3.53 (d, J=11.6 Hz, 1H), 3.21-3.11 (m, 2H), 2.24 (s, 3H), 1.44 (s, 9H).

### 5-Chloro-1-{3-[3-deoxy-3-(1H-1,2-pyrazol-1-yl)-β-D-galactopyranosyl]-5-methyl-4H-1,2,4-triazol-4-yl}-2-(trifluoromethyl)benzene (atropisomer 1)

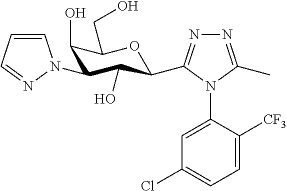

A solution of 1-{3-{4,6-O-benzylidene-3-[(2-tert-butoxycarbonyl)hydrazinyl]-3-deoxy-β-D-galactopyranosyl}-5-methyl-4H-1,2,4-triazol-4-yl}-5-chloro-2-(trifluoromethyl)benzene (atropisomer 1) (190 mg, 0.30 mmol) in DCM/TFA (5.0 mL, 10:1) was stirred 16 h at rt. The mixture was concentrated and dissolved together with malondialdehyde bis(diethyl acetal) (201 mg, 0.91 mmol) in EtOH (5.0 mL). The mixture was cooled to 0° C. Concentrated HCl (0.2 mL) was added, and the mixture was stirred 2 h at 0° C. The pH was adjusted to 5 by addition of saturated aq NaHCO₃. The mixture was concentrated and purified by prep HPLC [MeCN/H₂O (10 mmol/L, 0.05% TFA), X-Select 10 μm 19*250 mm, 20 m/min, UV 254] to give the product (83 mg, 58%). ESI-MS m/z calcd for [C₁₉H₁₉ClF₃N₅O₄] [M+H]⁺: 474.1; found: 474.0. ¹H NMR (400 MHz, Chloroform-d) 6 Methanol-d₄) δ 8.02 (d, J=8.4 Hz, 1H), 7.91 (dd, J=8.4, 1.6 Hz, 1H), 7.77 (d, J=2.4 Hz, 1H), 7.64 (d, J=1.6 Hz, 1H), 7.51 (d, J=1.6 Hz, 1H), 6.33 (t, J=2.0 Hz, 1H), 4.61 (t, J=10.0 Hz, 1H), 4.33 (dd, J=10.4, 2.4 Hz, 1H), 4.18 (d, J=9.6 Hz, 1H), 4.05 (d, J=2.4 Hz, 1H), 3.67-3.61 (m, 3H), 2.25 (s, 3H).

### 5-Chloro-1-{3-[3-deoxy-3-(4-iodo-1H-1,2-pyrazol-1-yl)-β-D-galactopyranosyl]-5-methyl-4H-1,2,4-triazol-4-yl}-2-(trifluoromethyl)benzene (atropisomer 1)

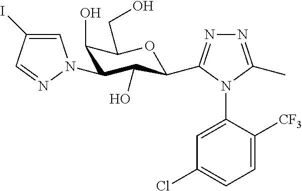

To a cooled (0° C.) solution of 5-chloro-1-{3-[3-deoxy-3-(1H-1,2-pyrazol-1-yl)-β-D-galactopyranosyl]-5-methyl-4H-1,2,4-triazol-4-yl}-2-(trifluoromethyl)benzene (atropisomer 1) (197 mg, 0.42 mmol) in THF (5 mL) N-iodosuccinimide (140 mg, 0.62 mmol) was added and the mixture was stirred 2 h at 0° C. The mixture was concentrated and purified by prep HPLC [MeOH/H₂O (10 mmol/L, 0.05% TFA), X-Select 10 μm 19*250 mm, 20 mL/min, UV 254] to give the product (119 mg, 48%). ESI-MS m/z calcd for [C₁₉H₁₈ClF₃IN₅O₄] [M+H]⁺: 600.0 found: 599.6. ¹H NMR (400 MHz, Methanol-d₄) δ 8.01 (d, J=8.8 Hz, 1H), 7.93 (d, J=8.4 Hz, 1H), 7.83 (d, J=1.6 Hz, 1H), 7.77 (s, 1H), 7.50 (s, 1H), 4.51-4.44 (m, 1H), 4.37 (dd, J=9.6, 2.4 Hz, 1H), 3.98 (d, J=2.0 Hz, 1H), 3.87-3.86 (m, 1H), 3.60 (t, J=6.4 Hz, 1H), 3.55-3.45 (m, 2H), 2.45 (s, 3H).

### Intermediate 11

### 1-[3-(3-Amino-4,6-O-benzylidene-3-deoxy-β-D-galactopyranosyl)-5-methyl-4H-1,2,4-triazol-4-yl]-5-chloro-2-(trifluoromethyl)benzene (atropisomer 2)

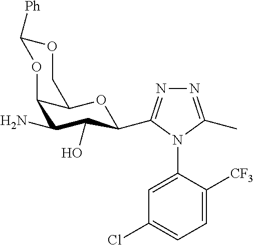

To a solution of 1-[3-(3-azido-4,6-O-benzylidene-3-deoxy-β-D-galactopyranosyl)-5-methyl-4H-1,2,4-triazol-4-yl]-5-chloro-2-(trifluoromethyl)benzene (atropisomer 2) (382 mg, 0.71 mmol) in THF (5.0 mL) and water (1 mL) triphenylphosphine (933 mg, 3.56 mmol) was added and the mixture was stirred 6 h at 50° C. The mixture was concentrated and purified by column chromatography (MeOH/DCM=0˜1/10, Silica-CS 12 g, 40 m/min, silica gel, UV 254) to afford the product (260 mg, 72%). ESI-MS m/z calcd for [C₂₃H₂₂ClF₃N₄O₄] [M+H]⁺: 511.1; found: 511.0. ¹H NMR (400 MHz, Methanol-d₄) δ 7.83 (d, J=8.8 Hz, 1H), 7.75 (d, J=1.6 Hz, 1H), 7.48 (dd, J=8.4, 1.2 Hz, 1H), 7.41-7.39 (m, 5H), 5.52 (s, 1H), 4.34 (d, J=9.6 Hz, 1H), 4.18 (d, J=3.2 Hz, 1H), 4.05 (t, J=9.6 Hz, 1H), 4.01 (d, J=10.0 Hz, 1H), 3.92 (d, J=11.6 Hz, 1H), 3.37 (s, 1H), 2.95 (dd, J=10.0, 3.2 Hz, 1H), 2.21 (s, 3H).

### 1-{3-{4,6-O-Benzylidene-3-[(2-tert-butoxycarbonyl)hydrazinyl]-3-deoxy-β-D-galactopyranosyl}-5-methyl-4H-1,2,4-triazol-4-yl}-5-chloro-2-(trifluoromethyl)benzene (atropisomer 2)

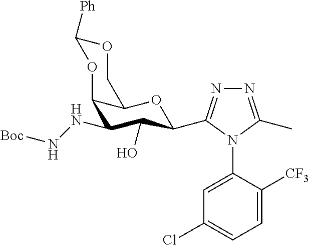

To a solution of 1-[3-(3-amino-4,6-O-benzylidene-3-deoxy-β-D-galactopyranosyl)-5-methyl-4H-1,2,4-triazol-4-yl]-5-chloro-2-(trifluoromethyl)benzene (atropisomer 2) (260 mg, 0.51 mmol) in DCM (6.0 mL) N-tert-butyloxycarbonyl-3-(4-cyanophenyl)oxaziridine (138 mg, 0.56 mmol) was added and the mixture was stirred overnight at rt. The mixture was concentrated and purified by column chromatography (PE/EtOAc=4/1˜0/1, Silica-CS 12 g, 20 mL/min, silica gel, UV 254) to afford the product (212 mg, 67%). ESI-MS m/z calcd for [C₂₈H₃₁ClF₃N₅O₆] [M+H]⁺: 626.2; found: 625.8. ¹H NMR (400 MHz, Chloroform-d) δ 7.68 (d, J=8.4 Hz, 1H), 7.51 (s, 1H), 7.49 (d, J=8.8 Hz, 1H), 7.36-7.34 (m, 2H), 7.30-7.26 (m, 3H), 6.64 (s, 1H), 5.38 (s, 1H), 4.46-4.41 (m, 2H), 4.23 (d, J=1.6 Hz, 1H), 4.16 (d, J=9.2 Hz, 1H), 3.81 (dd, J=12.4, 1.2 Hz, 1H), 3.68 (d, J=12.4 Hz, 1H), 3.08 (s, 1H), 3.01 (dd, J=10.0, 3.2 Hz, 1H), 2.15 (s, 3H), 1.36 (s, 9H).

### 5-Chloro-1-{3-[3-deoxy-3-(1H-1,2-pyrazol-1-yl)-β-D-galactopyranosyl]-5-methyl-4H-1,2,4-triazol-4-yl}-2-(trifluoromethyl)benzene_(atropisomer 2)

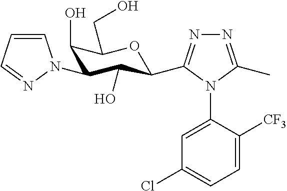

A solution of 1-{3-{4,6-O-benzylidene-3-[(2-tert-butoxycarbonyl)hydrazinyl]-3-deoxy-β-D-galactopyranosyl}-5-methyl-4H-1,2,4-triazol-4-yl}-5-chloro-2-(trifluoromethyl)benzene (atropisomer 2) (212 mg, 0.34 mmol) in DCM/TFA (5.0 mL, 10:1) was stirred 16 h at rt. The mixture was concentrated and dissolved together with malondialdehyde bis(diethyl acetal) (224 mg, 1.02 mmol) in EtOH (6.0 mL). The mixture was cooled to 0° C., concentrated HCl (0.5 mL) was added and the mixture was stirred 2 h at 0° C. The pH was adjusted to 5 by addition of saturated aq NaHCO₃. The mixture was concentrated and purified by prep HPLC [MeCN/H₂O (10 mmol/L, 0.05% TFA), X-Select 10 μm 19*250 mm, 20 m/min, UV 254] to give the product (83 mg, 52%). ESI-MS m/z calcd for [C₁₉H₁₉ClF₃N₅O₄] [M+H]⁺: 474.1; found: 474.0. ¹H NMR (400 MHz, Methanol-d₄) δ 7.91 (d, J=8.8 Hz, 1H), 7.82 (dd, J=8.4, 1.2 Hz, 1H), 7.76 (d, J=1.6 Hz, 1H), 7.73 (d, J=2.0 Hz, 1H), 7.46 (d, J=1.6 Hz, 1H), 6.26 (t, J=2.0 Hz, 1H), 4.84-4.82 (m, 1H), 4.27 (dd, J=10.8, 2.8 Hz, 1H), 4.10 (d, J=9.2 Hz, 1H), 3.93 (d, J=2.4 Hz, 1H), 3.44-3.38 (m, 3H), 2.19 (s, 3H).

### 5-Chloro-1-{3-[3-deoxy-3-(4-iodo-1H-1,2-pyrazol-1-yl)-β-D-galactopyranosyl]-5-methyl-4H-1,2,4-triazol-4-yl}-2-(trifluoromethyl)benzene (atropisomer 2)

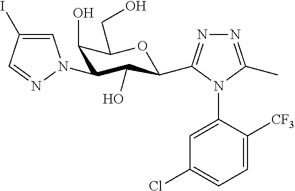

To a cooled (0° C.) solution of 5-chloro-1-{3-[3-deoxy-3-(1H-1,2-pyrazol-1-yl)-β-D-galactopyranosyl]-5-methyl-4H-1,2,4-triazol-4-yl}-2-(trifluoromethyl)benzene (atropisomer 2) (78 mg, 0.17 mmol) in THF (3 mL) N-iodosuccinimide (55.6 mg, 0.25 mmol) was added and the mixture was stirred 2 h at 0° C. The mixture was concentrated and purified by prep HPLC [MeOH/H₂O (10 mmol/L, 0.05% TFA), X-Select 10 μm 19*250 mm, 20 m/min, UV 254] to give the product (63 mg, 64%). ESI-MS m/z calcd for [C₁₉H₁₈ClF₃IN₅O₄] [M+H]⁺: 600.0 found: 599.7. ¹H NMR (400 MHz, Methanol-d₄) δ 7.99 (d, J=8.4 Hz, 1H), 7.91-7.83 (m, 2H), 7.85 (d, J=1.6 Hz, 1H), 7.52 (s, 1H), 4.87 (t, J=10.0 Hz, 1H), 4.37 (dd, J=10.8, 2.8 Hz, 1H), 4.21 (d, J=9.6 Hz, 1H), 3.99 (d, J=2.4 Hz, 1H), 3.51-3.46 (m, 3H), 2.29 (s, 3H).

### Intermediate 12

### 5-Chloro-1-{5-[3-deoxy-3-(3-methyl-1H-1,2-pyrazol-1-yl)-β-D-galactopyranosyl]-3-methyl-1H-1,2,4-triazol-1-yl}-2-(trifluoromethyl)benzene

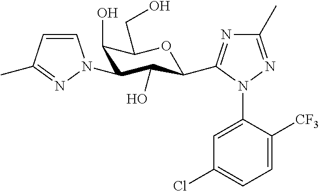

To a solution of 1-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-5-chloro-2-(trifluoromethyl)benzene (300 mg, 0.48 mmol) in DCM (10 mL) TFA (1.0 mL) was added and the mixture was stirred overnight at rt. The mixture was concentrated, and the residue was dissolved in 1,4-dioxane (5.0 mL). Acetylacetaldehyde dimethyl acetal (317 mg, 2.40 mmol) was added and the mixture was stirred 90 min at 90° C. The mixture was cooled to rt, and ethanol (5.0 mL) was added followed by concentrated HCl (1.0 mL). The mixture was stirred overnight at rt. The mixture was concentrated and purified by reversed-phase chromatography (MeCN/H₂O (0.5% TFA)=1/20˜1/2, C-18 column, 20 mL/min, UV 214). The obtained material was further purified by prep HPLC [MeCN/H₂O (10 mmol/L TFA), X-Select 10 μm 19*250 mm, 20 mL/min, UV 254] to give the product (60 mg, 26%). ESI-MS m/z calcd for [C₂₀H₂₁ClF₃N₅O₄][M+H]⁺: 488.1; found: 487.9. ¹H NMR (400 MHz, Methanol-d₄) δ 7.84 (d, J=8.4 Hz, 1H), 7.74 (dd, J=8.8, 1.2 Hz, 1H), 7.66 (s, 1H), 7.54 (d, J=2.4 Hz, 1H), 5.99 (d, J=2.0 Hz, 1H), 4.67-4.43 (m, 1H), 4.17-4.09 (m, 2H), 3.92 (d, J=2.8 Hz, 1H), 3.59-3.48 (m, 3H), 2.32 (s, 3H), 2.13 (s, 3H).

### 5-Chloro-1-{5-[3-deoxy-3-(4-iodo-3-methyl-1H-1,2-pyrazol-1-yl)-β-D-galactopyranosyl]-3-methyl-1H-1,2,4-triazol-1-yl}-2-(trifluoromethyl)benzene

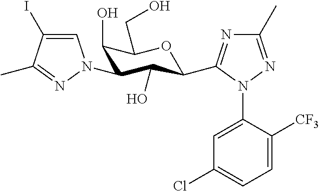

To a solution of 5-chloro-1-{5-[3-deoxy-3-(3-methyl-1H-1,2-pyrazol-1-yl)-β-D-galactopyranosyl]-3-methyl-1H-1,2,4-triazol-1-yl}-2-(trifluoromethyl)benzene (55 mg, 0.11 mmol) in THF (5 mL) N-iodosuccinimide (127 mg, 0.56 mmol) was added and the mixture was stirred overnight at rt. The mixture was concentrated and purified by reversed-phase chromatography (MeCN/H₂O=1/20˜1/2, C-18 column, 20 mL/min, UV 214) to give the product (60 mg, 87%). ESI-MS m/z calcd for [C₂₀H₂₀ClF₃IN₅O₄][M+H]⁺: 614.0; found: 613.8. ¹H NMR (400 MHz, Methanol-d₄) δ 7.84 (d, J=8.4 Hz, 1H), 7.75 (dd, J=8.4, 1.2 Hz, 1H), 7.71-7.62 (m, 2H), 4.65-4.45 (m, 1H), 4.21-4.09 (m, 2H), 3.91 (d, J=2.8 Hz, 1H), 3.59-3.46 (m, 3H), 2.33 (s, 3H), 2.10 (s, 3H).

### Intermediate 13

### 5-Chloro-3-{5-[3-deoxy-3-(4-iodo-1H-1,2-pyrazol-1-yl)-β-D-galactopyranosyl]-3-methyl-1H-1,2,4-triazol-1-yl}-2-cyclopropylpyridine

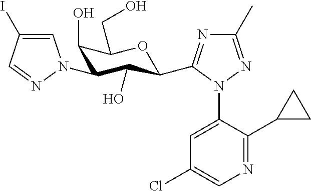

To a solution of 5-chloro-3-{5-[3-deoxy-3-(1H-1,2-pyrazol-1-yl)-β-D-galactopyranosyl]-3-methyl-1H-1,2,4-triazol-1-yl}-2-cyclopropylpyridine (100 mg, 0.22 mmol) in MeCN (5 mL) N-iodosuccinimide (503 mg, 2.24 mmol) was added and the mixture was stirred overnight at rt. The mixture was concentrated and purified by reversed-phase chromatography (MeCN/H₂O=1/20˜1/2, C-18 column, 20 mL/min, UV 214) to give the product (95 mg, 74%). ESI-MS m/z calcd for [C₂₀H₂₂ClIN₆O₄][M+H]⁺: 573.0; found: 572.8. ¹H NMR (400 MHz, Methanol-d₄) δ 8.48 (d, J=2.0 Hz, 1H), 7.79 (s, 1H), 7.74 (s, 1H), 7.43 (s, 1H), 4.52-4.49 (m, 1H), 4.29 (dd, J=10.8, 2.8 Hz, 1H), 4.25 (d, J=9.2 Hz, 1H), 3.91 (d, J=2.4 Hz, 1H), 3.61-3.49 (m, 3H), 2.35 (s, 3H), 1.58-1.52 (m, 1H), 1.03-0.80 (m, 4H).

### Intermediate 17

### 2-(4-Bromo-2,6-difluorophenyl)ethynyl(trimethyl)silane

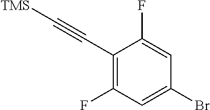 [SMILES: C[Si](C)(C)C#Cc1c(F)cc(Br)cc1F]

To a solution of 5-bromo-1,3-difluoro-2-iodobenzene (1.00 g, 3.14 mmol) and ethynyl(trimethyl)silane (462 mg, 4.70 mmol) in THF (16 mL) and DIPEA (4.0 mL) CuI (59.7 mg, 0.31 mmol) and Pd(PPh₃)₂Cl₂ (220 mg, 0.31 mmol) were added and the mixture was stirred overnight at rt. The mixture was concentrated and purified by column chromatography (PE) to give the product (1.1 g, 30% purity, 36%). ESI-MS m/z calcd for [C₁₁H₁₁BrF₂Si] [M]: 288.0; found: 288.0.

### Intermediate 18 (3-(4-Bromo-2,6-difluorophenyl)prop-1-ynyl)(tert-butyl)dimethylsilane

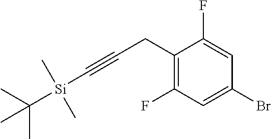 [SMILES: CC(C)(C)[Si](C)(C)C#CCc1c(F)cc(Br)cc1F]

To a solution of 5-bromo-2-(bromomethyl)-1,3-difluorobenzene (300 mg, 1.05 mmol) and (tert-butyl)(ethynyl)dimethylsilane (221 mg, 1.57 mmol) in MeCN (10 mL) K₂CO₃ (160 mg, 1.15 mmol), CuI (200 mg, 1.05 mmol) and tetrabutylammonium iodide (388 mg, 1.05 mmol) were added and the mixture was stirred overnight at 75° C. The mixture was quenched with concentrated aq NH₄Cl and extracted with EtOAc (2×20 mL). The combined organic phases were washed with brine, dried over Na₂SO₄, concentrated and purified by reversed-phase chromatography (H₂O/MeCN=20/1˜0/1, C-18 column, 20 mL/min, UV 214) to give the product (150 mg, 41%). ESI-MS m/z calcd for [C₁₅H₁₉BrF₂Si] [M-tert-butyl]: 287.0; found: 287.0. ¹H NMR (400 MHz, Chloroform-d) δ 7.12-7.03 (m, 2H), 3.56 (s, 2H), 0.89 (s, 9H), 0.06 (s, 6H).

### 1-{5-{3-{4-{4-{3-[tert-Butyl(dimethyl)silyl]prop-2-ynyl}-3,5-difluorophenyl}-1H-1,2-pyrazol-1-yl}-3-deoxy-β-D-galactopyranosyl}-3-methyl-1H-1,2,4-triazol-1-yl}-5-chloro-2-(trifluoromethyl)benzene

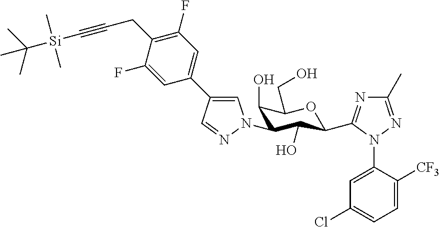

To a solution of 1-{5-[3-(4-borono-1H-1,2-pyrazol-1-yl)-3-deoxy-β-D-galactopyranosyl]-3-methyl-1H-1,2,4-triazol-1-yl}-5-chloro-2-(trifluoromethyl)benzene (30 mg, 0.058 mmol) and (3-(4-bromo-2,6-difluorophenyl)prop-1-ynyl)(tert-butyl)dimethylsilane (60 mg, 0.17 mmol) in 1,4-dioxane/H₂O (2.5 mL, 4:1) Pd(dppf)Cl₂ (8.5 mg, 0.012 mmol) and K₂CO₃ (40 mg, 0.29 mmol) were added. The mixture was purged three times with nitrogen, and stirred 2 h at 80° C. The mixture was filtered, evaporated, and purified by prep HPLC [MeCN/H₂O (10 mmol/L NH₄HCO₃), X-Select 10 μm 19*250 mm, 20 m/min, UV 254] to afford the product (15 mg, 35%). ESI-MS m/z calcd for [C₃₄H₃₇ClF₅N₅O₄Si] [M+H]⁺: 738.2; found: 737.8. ¹H NMR (400 MHz, Chloroform-d) δ 7.89 (s, 1H), 7.78 (s, 1H), 7.75 (d, J=8.4 Hz, 1H), 7.64 (d, J=8.8 Hz, 1H), 7.59 (s, 1H), 7.05-6.94 (m, 2H), 5.53 (s, 1H), 4.67 (t, J=9.2 Hz, 1H), 4.46 (s, 1H), 4.44-4.37 (m, 2H), 4.17 (dd, J=10.4, 2.4 Hz, 1H), 3.70-3.45 (m, 5H), 2.39 (s, 3H), 0.89 (s, 9H), 0.06-0.05 (m, 6H).

### Intermediate 19

### (5-Bromo-3-pyridyl)hydrazine

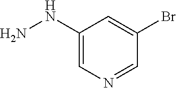

To a cooled (−5° C.) solution of 5-bromopyridin-3-amine (1.00 g, 5.78 mmol) in water (15 mL) HCl (15 mL, 33% wt) was added. A solution of NaNO₂ (479 mg, 6.94 mmol) in water (3 mL) was added dropwise and the mixture was stirred 1 h at −5° C. Tin(II) chloride dihydrate (3.26 g, 14.4 mmol) dissolved in concentrated HCl (5 mL) was added dropwise at −5° C. The mixture was stirred 2 h at −5° C. Then NaOH (100 mL, 10% wt) solution was added dropwise at 0° C. to adjust pH to 9. Water (50 mL) and DCM (50 mL) were added. The aqueous phase was extracted with DCM (2×50 mL). The combined organic phases were washed with water (50 mL) and brine (2×50 mL), dried over anhydrous Na₂SO₄, evaporated and purified by column chromatography (PE/EtOAc=1/0˜3/1, silica-CS 40 g 40 m/min, silica gel, UV 254 nm) to afford the product (220 mg, 20%). ESI-MS m/z calcd for [C₅H₆BrN₃] [M+H]⁺: 188.0; found: 188.0. ¹H NMR (400 MHz, DMSO-d₆) δ 8.01 (d, J=2.4 Hz, 1H), 7.81 (d, J=2.0 Hz, 1H), 7.32-7.31 (m, 2H), 4.28 (s, 2H).

### 2,6-Anhydro-4-azido-5,7-O-benzylidene-4-deoxy-D-glycero-L-manno-N-[1-(dimethylamino)ethylidene]heptonamide

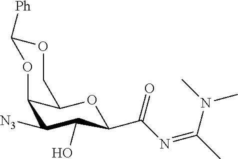

To a solution of 2,6-anhydro-4-azido-5,7-O-benzylidene-4-deoxy-D-glycero-L-manno-heptonamide (2.30 g, 7.18 mmol) in 1,4-dioxane (30 mL) N,N-dimethylacetamide dimethyl acetal (1.44 g, 10.8 mmol) was added and the mixture was stirred 2 h at 60° C. The mixture was evaporated to give the product (3.50 g, 79% purity, 99%), which was used without purification in subsequent steps. ESI-MS m/z calcd for [C₁₈H₂₃N₅O₅][M+H]⁺: 390.2; found: 390.2. ¹H NMR (400 MHz, Chloroform-d) δ 7.54-7.51 (m, 2H), 7.38-7.33 (m, 3H), 5.58 (s, 1H), 4.47 (dd, J=12.4, 1.6 Hz, 1H), 4.26-4.22 (m, 2H), 4.05 (dd, J=12.4, 1.6 Hz, 1H), 3.83 (d, J=9.6 Hz, 1H), 3.51 (d, J=1.2 Hz, 1H), 3.40 (dd, J=10.0, 3.2 Hz, 1H), 3.14 (s, 3H), 3.12 (s, 3H), 2.39 (s, 3H).

### 3-[5-(3-Azido-4,6-O-benzylidene-3-deoxy-β-D-galactopyranosyl)-3-methyl-1H-1,2,4-triazol-1-yl]-5-bromopyridine

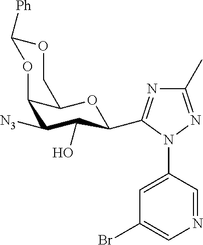

To a solution of 2,6-anhydro-4-azido-5,7-O-benzylidene-4-deoxy-D-glycero-L-manno-N-[1-(dimethylamino)ethylidene]heptonamide (500 mg, 1.28 mmol) in MeCN (5 mL) and acetic acid (1 mL) (5-bromo-3-pyridyl)hydrazine (290 mg, 1.54 mmol) was added and the mixture was stirred 3 h at 100° C. The mixture was concentrated and partitioned between water (20 mL) and DCM (20 mL). The aqueous phase was extracted with DCM (2×20 mL). The combined organic phases were washed with water (20 mL) and brine (20 mL), dried over Na₂SO₄, evaporated and purified by prep HPLC (MeCN/H₂O (10 mmol/L NH₄HCO₃), X-Select 10 um 19*250 mm, 20 m/min, UV 254) to give the product (306 mg, 46%). ESI-MS m/z calcd for [C₂₁H₂₀BrN₇O₄] [M+H]⁺: 514.1; found: 513.8. ¹H NMR (400 MHz, Chloroform-d) δ 8.71 (d, J=2.0 Hz, 1H), 8.61 (d, J=2.0 Hz, 1H), 8.08 (t, J=2.0 Hz, 1H), 7.41-7.39 (m, 2H), 7.28-7.24 (m, 3H), 5.44 (s, 1H), 4.68 (t, J=9.6 Hz, 1H), 4.28 (d, J=9.2 Hz, 1H), 4.11 (d, J=3.2 Hz, 1H), 4.07-4.03 (m, 1H), 3.93 (dd, J=12.4, 1.6 Hz, 1H), 3.40 (s, 1H), 3.35 (dd, J=10, 3.2 Hz, 1H), 2.33 (s, 3H).

### 3-[5-(3-Amino-4,6-O-benzylidene-3-deoxy-β-D-galactopyranosyl)-3-methyl-1H-1,2,4-triazol-1-yl]-5-bromopyridine

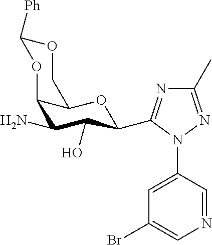

To a solution of 3-[5-(3-azido-4,6-O-benzylidene-3-deoxy-β-D-galactopyranosyl)-3-methyl-1H-1,2,4-triazol-1-yl]-5-bromopyridine (306 mg, 0.60 mmol) in THF (5 mL) and water (1 mL) triphenylphosphine (780 mg, 2.97 mmol) was added and the mixture was stirred 16 h at 70° C. The mixture was concentrated and purified by column chromatography (DCM/MeOH=1/0˜10/1, Silica-CS 12 g, 20 mL/min, silica gel, UV 254) to afford the product (240 mg, 83%). ESI-MS m/z calcd for [C₂₁H₂₂BrN₅O₄][M+H]⁺: 488.1; found: 488.2. ¹H NMR (400 MHz, Chloroform-d) δ 8.80 (d, J=2.0 Hz, 1H), 8.71 (d, J=1.6 Hz, 1H), 8.17 (t, J=2.0 Hz, 1H), 7.48-7.46 (m, 2H), 7.37-7.26 (m, 3H), 5.56 (s, 1H), 4.39-4.29 (m, 3H), 4.14-4.04 (m, 1H), 3.57 (s, 1H), 3.01 (d, J=6.4 Hz, 1H), 2.65 (brs, 3H), 2.39 (s, 3H).

### 3-{5-{4,6-O-Benzylidene-3-[(2-tert-butoxycarbonyl)hydrazinyl]-3-deoxy-β-D-galactopyranosyl}-3-methyl-1H-1,2,4-triazol-1-yl}-5-bromopyridine

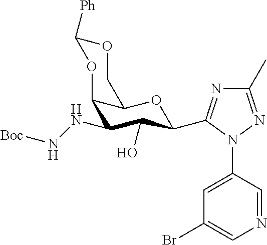

To a solution of 3-[5-(3-amino-4,6-O-benzylidene-3-deoxy-β-D-galactopyranosyl)-3-methyl-1H-1,2,4-triazol-1-yl]-5-bromopyridine (240 mg, 0.49 mmol) in DCM (5.0 mL) N-tert-butyloxycarbonyl-3-(4-cyanophenyl)oxaziridine (133 mg, 0.54 mmol) was added and the mixture was stirred overnight at rt. The mixture was concentrated and purified by column chromatography (PE/EtOAc=4/1˜0/1, Silica-CS 12 g, 20 mL/min, silica gel, UV 254) to afford the product (195 mg, 66%). ESI-MS m/z calcd for [C₂₆H₃₁BrN₆O₆] [M+H]⁺: 603.2; found: 602.8.

### 5-Bromo-3-{5-[3-deoxy-3-(1H-1,2-pyrazol-1-yl)-β-D-galactopyranosyl]-3-methyl-1H-1,2,4-triazol-1-yl}pyridine

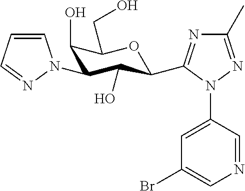

A solution of 3-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-5-bromopyridine (291 mg, 0.48 mmol) in DCM/TFA (5.0 mL, 10:1) was stirred 16 h at rt. The mixture was concentrated, and the residue was dissolved in ethanol (5.0 mL) and cooled to 0° C. To the solution 1,1,3,3-tetraethoxypropane (201 mg, 0.91 mmol) was added, followed by concentrated HCl (0.2 mL). The mixture was stirred 2 h at 0° C., before the pH was adjusted to 4-5 using saturated aq NaHCO₃. The mixture was concentrated and purified by prep HPLC [MeCN/H₂O (10 mmol/L, 0.05% TFA), X-Select 10 μm 19*250 mm, 20 mL/min, UV 254] to give the product (83 mg, 38%). ESI-MS m/z calcd for [C₁₇H₁₉BrN₆O₄] [M+H]⁺: 451.1 found: 450.8. ¹H NMR (400 MHz, Methanol-d₄) δ 8.79 (s, 1H), 8.73 (s, 1H), 8.35 (t, J=2.0 Hz, 1H), 7.69 (d, J=2.4 Hz, 1H), 7.43 (d, J=1.6 Hz, 1H), 6.24 (t, J=2.4 Hz, 1H), 4.66 (dd, J=10.4, 9.6 Hz, 1H), 4.43 (d, J=9.2 Hz, 1H), 4.35 (dd, J=10.4, 2.4 Hz, 1H), 3.99 (d, J=2.0 Hz, 1H), 3.75 (dd, J=7.2, 4.8 Hz, 1H), 3.69 (dd, J=11.6, 7.2 Hz, 1H), 3.60 (dd, J=11.6, 4.4 Hz, 1H), 2.33 (s, 3H).

### 5-Bromo-3-{5-[3-deoxy-3-(4-iodo-1H-1,2-pyrazol-1-yl)-β-D-galactopyranosyl]-3-methyl-1H-1,2,4-triazol-1-yl}pyridine

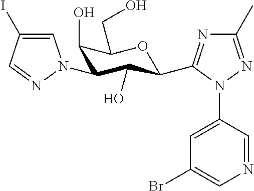

To a cooled (0° C.) solution of 5-bromo-3-{5-[3-deoxy-3-(1H-1,2-pyrazol-1-yl)-3-D-galactopyranosyl]-3-methyl-1H-1,2,4-triazol-1-yl}pyridine (83 mg, 0.18 mmol) in THF (5 mL) N-iodosuccinimde (197 mg, 0.88 mmol) was added and the mixture was stirred overnight at rt. The mixture was concentrated and purified by prep HPLC [MeOH/H₂O (10 mmol/L, 0.05% TFA), X-Select 10 μm 19*250 mm, 20 mL/min, UV 254] to give the product (47 mg, 47%). ESI-MS m/z calcd for [C₁₇H₁₈BrIN₆O₄][M+H]⁺: 577.0; found: 576.5. ¹H NMR (400 MHz, Methanol-d₄) δ 8.78 (d, J=1.6 Hz, 1H), 8.73 (d, J=1.6 Hz, 1H), 8.34 (t, J=2.0 Hz, 1H), 7.77 (s, 1H), 7.45 (s, 1H), 4.64 (dd, J=10.4, 9.6 Hz, 1H), 4.42 (d, J=9.6 Hz, 1H), 4.37 (dd, J=10.4, 2.4 Hz, 1H), 3.96 (d, J=2.4 Hz, 1H), 3.73 (dd, J=6.8, 4.8 Hz, 1H), 3.67 (dd, J=11.2, 7.2 Hz, 1H), 3.58 (dd, J=11.2, 4.4 Hz, 1H), 2.35 (s, 3H).

### Intermediate 20

### 5-Chloro-2-(trifluoromethyl)-3-nitropyridine

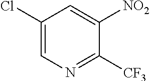 [SMILES: O=[N+]([O-])c1cc(Cl)cnc1C(F)(F)F]

A mixture of 2-bromo-5-chloro-3-nitropyridine (9.80 g, 41.3 mmol), methyl 2,2-difluoro-2-fluorosulfonylacetate (11.9 g, 61.9 mmol) and CuI (9.43 g, 49.5 mmol) in DMF (100 mL) was stirred 2 h at 100° C. After cooling to rt, the mixture was poured into water (300 mL) and then extracted with EtOAc (3×100 mL). The combined organic phases were washed with water (100 mL) and brine (100 mL), dried over Na₂SO₄, concentrated and purified by column chromatography (EtOAc/PE=0/1˜1/4, Silica-CS 80 g, 40 mL/min, silica gel, UV 254) to give the product (8.8 g, 94%). ESI-MS m/z calcd for [C₆H₂ClF₃N₂O₂] [M]: 226.0; found: 226.0. ¹H NMR (400 MHz, Chloroform-d) δ 8.79 (d, J=2.0 Hz, 1H), 8.16 (d, J=1.6 Hz, 1H).

### 3-Amino-5-chloro-2-(trifluoromethyl)pyridine

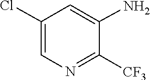 [SMILES: Nc1cc(Cl)cnc1C(F)(F)F]

A solution of 5-chloro-2-(trifluoromethyl)-3-nitropyridine (8.8 g, 38.8 mmol), ammonium hydrochloride (12.5 g, 233 mmol) and Fe (10.8 g, 194 mmol) in EtOH (100 mL) and H₂O (10 mL) was stirred 6 h at 85° C. The mixture was filtered, concentrated, and purified by column chromatography (EtOAc/PE=0/1˜1/4, Silica-CS 80 g, 40 m/min, silica gel, UV 254) to give the product (7.1 g, 93%). ESI-MS m/z calcd for [C₆H₄ClF₃N₂] [M+H]⁺: 197.0; found: 197.2. ¹H NMR (400 MHz, Chloroform-d) δ 7.92 (d, J=1.6 Hz, 1H), 7.04 (d, J=2.0 Hz, 1H), 4.25 (br s, 2H).

### [5-Chloro-2-(trifluoromethyl)-3-pyridyl]hydrazine

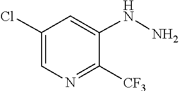 [SMILES: NNc1cc(Cl)cnc1C(F)(F)F]

To a cooled (−5° C.) solution of 3-amino-5-chloro-2-(trifluoromethyl)pyridine (2.50 g, 12.7 mmol) in acetic acid (8.0 mL) concentrated HCl (16.0 mL) was added. A solution of NaNO₂ (1.14 g, 16.5 mmol) in water (4.0 mL) was added dropwise over 5 min. The mixture was stirred 45 min at −5° C. Then a cooled (0° C.) solution of tin(II) chloride dihydrate (2.87 g, 12.7 mmol) in concentrated HCl (8.0 mL) was added dropwise over 10 min. The mixture was stirred 2 h at −5° C. Then aqueous NaOH (5 M) was added dropwise at −5° C. to adjust pH to 8. The mixture was extracted with EtOAc (2×100 mL). The combined organic phases were washed with water (100 mL) and brine (100 mL), dried over Na₂SO₄, evaporated, and purified by column chromatography (PE/EtOAc=1/0˜4/1, silica-CS 20 g 20 mL/min, silica gel, UV 254 nm) to afford the product (1.8 g, 67%). ESI-MS m/z calcd for [C₆H₅ClF₃N₃] [M+H]⁺: 212.0; found: 212.2. ¹H NMR (400 MHz, Chloroform-d) δ 7.88 (d, J=2.0 Hz, 1H), 7.81 (d, J=2.0 Hz, 1H), 5.94 (s, 1H), 3.65 (s, 2H).

### 3-[5-(3-Azido-4,6-O-benzylidene-3-deoxy-β-D-galactopyranosyl)-3-methyl-1H-1,2,4-triazol-1-yl]-5-chloro-2-(trifluoromethyl)pyridine

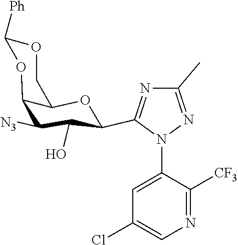

To a solution of 2,6-anhydro-4-azido-5,7-O-benzylidene-4-deoxy-D-glycero-L-manno-heptonamide (1.0 g, 3.12 mmol) in 1,4-dioxane (15.0 mL) N,N-dimethylacetamide dimethyl acetal (457 mg, 3.43 mmol) was added and the mixture was stirred 3 h at 50° C. The mixture was evaporated, and the residue was dissolved in acetic acid (10 mL). [5-Chloro-2-(trifluoromethyl)-3-pyridyl]hydrazine (793 mg, 3.75 mmol) was added and the mixture was stirred 2 h at 70° C. The mixture was concentrated and partitioned between water (20 mL) and DCM (20 mL). The aqueous phase was extracted with DCM (2×20 mL). The combined organic phases were washed with water (20 mL) and brine (20 mL), dried over Na₂SO₄, evaporated, and purified by column chromatography (EtOAc/PE=1/5˜2/1, Silica-CS 20 g, 30 mL/min, silica gel, UV 254). The obtained material was further purified by reversed-phase chromatography (MeCN/H₂O=1/20˜1/1, C-18 column, 20 m/min, UV 254) to afford the product (600 mg, 36%). ESI-MS m/z calcd for [C₂₂H₁₉ClF₃N₇O₄] [M+H]⁺: 538.1; found: 537.8. ¹H NMR (400 MHz, Chloroform-d) δ 8.55 (s, 1H), 7.81 (d, J=1.6 Hz, 1H), 7.38-7.26 (m, 5H), 5.39 (s, 1H), 4.49 (d, J=8.8 Hz, 1H), 4.25 (t, J=9.6 Hz, 1H), 4.14 (d, J=3.2 Hz, 1H), 3.81 (dd, J=12.8, 1.6 Hz, 1H), 3.51 (d, J=12.8 Hz, 1H), 3.36 (dd, J=10.4, 3.2 Hz, 1H), 3.29 (s, 1H), 2.39 (s, 3H).

### 3-[5-(3-Amino-4,6-O-benzylidene-3-deoxy-β-D-galactopyranosyl)-3-methyl-1H-1,2,4-triazol-1-yl]-5-chloro-2-(trifluoromethyl)pyridine

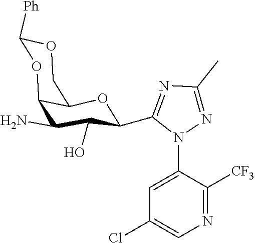

To a solution of 3-[5-(3-azido-4,6-O-benzylidene-3-deoxy-β-D-galactopyranosyl)-3-methyl-1H-1,2,4-triazol-1-yl]-5-chloro-2-(trifluoromethyl)pyridine (600 mg, 1.12 mmol) in THF (30 mL) and water (3.0 mL) triphenylphosphine (1.46 g, 5.58 mmol) was added and the mixture was stirred 6 h at 50° C. The mixture was concentrated and purified by column chromatography (DCM/MeOH=1/0˜10/1, Silica-CS 12 g, 40 m/min, silica gel, UV 254). The obtained material was further purified by reversed-phase chromatography (MeCN/H₂O=1/20˜1/1, C-18 column, 20 mL/min, UV 254) to afford the product (500 mg, purity 70%, 61%). ESI-MS m/z calcd for [C₂₂H₂₁ClF₃N₅O₄] [M+H]⁺: 512.1; found: 511.9. ¹H NMR (400 MHz, Chloroform-d) δ 8.63 (d, J=2.0 Hz, 1H), 7.85 (d, J=2.0 Hz, 1H), 7.35-7.25 (m, 5H), 5.36 (s, 1H), 4.29 (d, J=9.2 Hz, 1H), 4.04 (d, J=2.4 Hz, 1H), 3.86-3.75 (m, 3H), 3.44 (dd, J=12.8, 1.2 Hz, 1H), 3.26 (s, 1H), 2.78 (dd, J=9.6, 2.8 Hz, 1H), 2.37 (s, 3H).

### 3-{5-{4,6-O-Benzylidene-3-[(2-tert-butoxycarbonyl)hydrazinyl]-3-deoxy-β-D-galactopyranosyl}-3-methyl-1H-1,2,4-triazol-1-yl}-5-chloro-2-(trifluoromethyl)pyridine

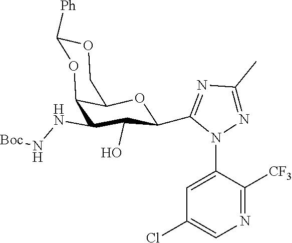

To a solution of 3-[5-(3-amino-4,6-O-benzylidene-3-deoxy-β-D-galactopyranosyl)-3-methyl-1H-1,2,4-triazol-1-yl]-5-chloro-2-(trifluoromethyl)pyridine (500 mg, purity 70%, 0.68 mmol) in DCM (10.0 mL) N-tert-butyloxycarbonyl-3-(4-cyanophenyl)oxaziridine (185 mg, 0.68 mmol) was added and the mixture was stirred overnight at rt. The mixture was concentrated and purified by column chromatography (PE/EtOAc=4/1˜0/1, Silica-CS 20 g, 30 mL/min, silica gel, UV 254) to afford the product (210 mg, 49%). ESI-MS m/z calcd for [C₂₇H₃₀ClF₃N₆O₆] [M+H]⁺: 627.2; found: 627.0. ¹H NMR (400 MHz, Chloroform-d) δ 8.61 (d, J=1.2 Hz, 1H), 7.89 (d, J=2.0 Hz, 1H), 7.41-7.30 (m, 5H), 6.44 (br s, 1H), 5.41 (s, 1H), 4.70 (br s, 1H), 4.51 (s, 1H), 4.43 (d, J=9.2 Hz, 1H), 4.28 (d, J=2.4 Hz, 1H), 4.14-4.08 (m, 1H), 3.87 (dd, J=12.4, 1.6 Hz, 1H), 3.59 (d, J=12.4 Hz, 1H), 3.31 (s, 1H), 3.05 (d, J=10.0 Hz, 1H), 2.42 (s, 3H), 1.44 (s, 9H).

### 5-Chloro-3-{5-[3-deoxy-3-(1H-1,2-pyrazol-1-yl)-β-D-galactopyranosyl]-3-methyl-1H-1,2,4-triazol-1-yl}-2-(trifluoromethyl)pyridine

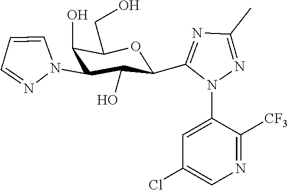

To a cooled (0° C.) solution of 3-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-5-chloro-2-(trifluoromethyl)pyridine (210 mg, 0.34 mmol) in DCM (10.0 mL) TFA (1.0 mL) was added and the mixture was stirred overnight at rt. The mixture was concentrated, and the residue was dissolved in ethanol (10.0 mL). 1,1,3,3-Tetraethoxypropane (369 mg, 1.67 mmol) was added, followed by concentrated HCl (0.25 mL). The mixture was stirred 1.5 h at rt, before the pH was adjusted to 7-8 using saturated aq NaHCO₃. The mixture was concentrated and purified by reversed-phase chromatography (MeCN/H₂O=1/20˜1/2, C-18 column, 20 m/min, UV 214) to give the product (90 mg, 57%). ESI-MS m/z calcd for [C₁₈H₁₈ClF₃N₆O₄] [M+H]⁺: 475.1; found: 474.9. ¹H NMR (400 MHz, Methanol-d₄) δ 8.92 (d, J=2.0 Hz, 1H), 8.31 (d, J=1.2 Hz, 1H), 7.76 (d, J=2.4 Hz, 1H), 7.51 (d, J=1.6 Hz, 1H), 6.32 (t, J=2.4 Hz, 1H), 4.62 (t, J=10.0 Hz, 1H), 4.39 (d, J=9.6 Hz, 1H), 4.35 (dd, J=10.4, 2.4 Hz, 1H), 4.02 (d, J=2.4 Hz, 1H), 3.66 (t, J=6.0 Hz, 1H), 3.62-3.53 (m, 2H), 2.42 (s, 3H).

### 5-Chloro-3-{5-[3-deoxy-3-(4-iodo-1H-1,2-pyrazol-1-yl)-β-D-galactopyranosyl]-3-methyl-1H-1,2,4-triazol-1-yl}-2-(trifluoromethyl)pyridine

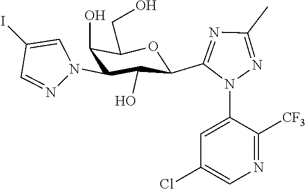

To a solution of 5-chloro-3-{5-[3-deoxy-3-(1H-1,2-pyrazol-1-yl)-3-D-galactopyranosyl]-3-methyl-1H-1,2,4-triazol-1-yl}-2-(trifluoromethyl)pyridine (45 mg, 0.095 mmol) in THF (5.0 mL) N-iodosuccinimde (107 mg, 0.47 mmol) was added and the mixture was stirred overnight at rt. The mixture was concentrated and purified by reversed-phase chromatography (MeCN/H₂O=1/20˜1/2, C-18 column, 20 mL/min, UV 214) to give the product (45 mg, 79%). ESI-MS m/z calcd for [C₁₈H₁₇ClF₃IN₆O₄][M+H]⁺: 601.0; found: 600.7. ¹H NMR (400 MHz, Methanol-d₄) δ 8.92 (d, J=1.6 Hz, 1H), 8.31 (s, 1H), 7.84 (s, 1H), 7.53 (s, 1H), 4.60 (t, J=9.6 Hz, 1H), 4.44-4.32 (m, 2H), 3.99 (d, J=2.0 Hz, 1H), 3.64 (t, J=6.0 Hz, 1H), 3.60-3.50 (m, 2H), 2.42 (s, 3H).

### Intermediate 21 (2-Bromo-5-chloro-3-pyridyl)hydrazine

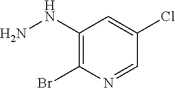

To a cooled (0° C.) solution of 2-bromo-5-chloropyridin-3-amine (2.00 g, 9.64 mmol) in water (30 mL) and concentrated HCl (30 mL) a solution of NaNO₂ (998 mg, 14.5 mmol) in water (5 mL) was added. The mixture was stirred 1 h at 0° C. Then a solution of tin(II) chloride dihydrate (4.35 g, 19.3 mmol) dissolved in concentrated HCl (10.0 mL) was added and the mixture was stirred 3 h at rt. The mixture was basified by addition of aq NaOH until pH reached 11. The mixture was extracted with EtOAc (2×100 mL). The combined organic phases were dried over Na₂SO₄, evaporated and purified by column chromatography (PE/EtOAc=10/1˜1/1, silica-CS 40 g, 50 m/min, silica gel, UV 254 nm) to afford the product (1.10 g, 51%). ESI-MS m/z calcd for [C₅H₅BrClN₃] [M+H]⁺: 221.9; found: 222.2. ¹H NMR (400 MHz, Chloroform-d) δ 7.73 (d, J=2.4 Hz, 1H), 7.48 (d, J=2.0 Hz, 1H), 5.82 (s, 1H), 3.71 (s, 2H).

### 3-(Dimethylamino)-2-(3,4,5-trifluorophenyl)prop-2-enal

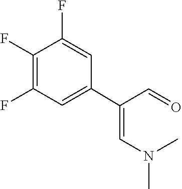 [SMILES: CN(C)/C=C(\C=O)c1cc(F)c(F)c(F)c1]

To a cooled (0° C.) solution of N,N-dimethylformamide (913 mg, 12.5 mmol) and phosphoryl trichloride (1724 mg, 11.2 mmol) 2-(3,4,5-trifluorophenyl)acetic acid (475 mg, 2.50 mmol) was added and the mixture was stirred overnight at 60-70° C. After cooling to rt, the mixture was slowly added to a mixture of ice and water with external cooling and ice intermittently to keep the temperature below 10° C. K₂CO₃ was added slowly until pH=11 was achieved. Small quantities of ethanol were added to control frothing. To the alkaline mixture toluene (10 mL) was added, and the mixture was refluxed for 1.5 h and cooled to rt. The aqueous phase was extracted with additional toluene (20 mL). The combined organic phases were washed with water, dried over Na₂SO₄ and evaporated. The obtained solid was recrystallized from hexane to give the product (320 mg, 56%). ESI-MS m/z calcd for [C₁₁H₁₀F₃NO] [M+H]⁺: 230.1; found: 230.1. ¹H NMR (400 MHz, Chloroform-d) δ 9.05 (s, 1H), 6.91 (s, 1H), 6.82 (dd, J=8.2, 6.6 Hz, 2H), 2.91 (s, 6H).

### 3-[5-(3-Azido-4,6-O-benzylidene-3-deoxy-β-D-galactopyranosyl)-3-methyl-1H-1,2,4-triazol-1-yl]-2-bromo-5-chloropyridine

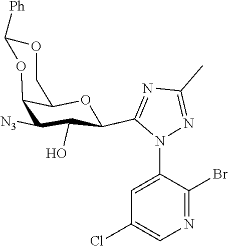

To a solution of 2,6-anhydro-4-azido-5,7-O-benzylidene-4-deoxy-D-glycero-L-manno-N-[1-(dimethylamino)ethylidene]heptonamide (1.60 g, 4.11 mmol) in acetic acid (5 mL) (2-bromo-5-chloro-3-pyridyl)hydrazine (1.00 g, 4.49 mmol) was added and the mixture was stirred 2 h at 60° C. The mixture was concentrated and purified by column chromatography (EtOAc/PE=1/10˜1/1, Silica-CS 20 g, 25 mL/min, silica gel, UV 254) to give the product (960 mg, 43%). ESI-MS m/z calcd for [C₂₁H₁₉BrClN₇O₄][M+H]⁺: 548.0; found: 547.8. ¹H NMR (400 MHz, Chloroform-d) δ 8.34 (s, 1H), 7.83 (d, J=2.0 Hz, 1H), 7.46-7.31 (m, 5H), 5.48 (s, 1H), 4.66 (d, J=9.2 Hz, 1H), 4.45 (t, J=9.2 Hz, 1H), 4.22 (d, J=2.8 Hz, 1H), 3.91 (dd, J=12.8, 1.2 Hz, 1H), 3.66-3.57 (m, 1H), 3.48 (dd, J=10.0, 3.2 Hz, 1H), 3.40 (s, 1H), 2.48 (s, 3H), 2.14 (s, 1H).

### 3-[5-(3-Amino-4,6-O-benzylidene-3-deoxy-β-D-galactopyranosyl)-3-methyl-1H-1,2,4-triazol-1-yl]-2-bromo-5-chloropyridine

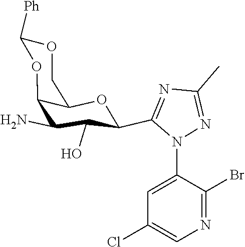

To a solution of 3-[5-(3-azido-4,6-O-benzylidene-3-deoxy-β-D-galactopyranosyl)-3-methyl-1H-1,2,4-triazol-1-yl]-2-bromo-5-chloropyridine (960 mg, 1.75 mmol) in THF (20 mL) and water (4 mL) triphenylphosphine (2.29 g, 8.75 mmol) was added and the mixture was stirred overnight at 50° C. The mixture was concentrated and purified by column chromatography (DCM/MeOH=1/0˜10/1, Silica-CS 40 g, 50 mL/min, silica gel, UV 254) to afford the product (620 mg, 68%). ESI-MS m/z calcd for [C₂IH₂IBrClN₅O₄] [M+H]⁺: 522.0; found: 521.8. ¹H NMR (400 MHz, Chloroform-d) δ 8.37 (s, 1H), 7.82 (s, 1H), 7.42-7.27 (m, 5H), 5.45 (s, 1H), 4.48 (d, J=8.8 Hz, 1H), 4.18 (s, 1H), 4.01 (t, J=9.6 Hz, 1H), 3.90 (d, J=12.4, 1H), 3.61 (d, J=12.0 Hz, 1H), 3.38 (s, 1H), 2.83 (s, 1H), 2.43 (s, 3H).

### 3-{5-{4,6-O-Benzylidene-3-[(2-tert-butoxycarbonyl)hydrazinyl]-3-deoxy-β-D-galactopyranosyl}-3-methyl-1H-1,2,4-triazol-1-yl}-2-bromo-5-chloropyridine

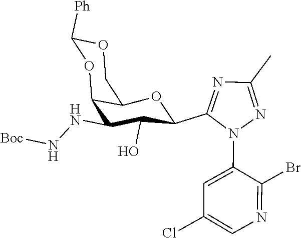

To a solution of 3-[5-(3-amino-4,6-O-benzylidene-3-deoxy-β-D-galactopyranosyl)-3-methyl-1H-1,2,4-triazol-1-yl]-2-bromo-5-chloropyridine (320 mg, 0.61 mmol) in DCM (5 mL) N-tert-butyloxycarbonyl-3-(4-cyanophenyl)oxaziridine (181 mg, 0.74 mmol) was added and the mixture was stirred overnight at rt. The mixture was concentrated and purified by column chromatography (MeOH/DCM=0˜1/10, Silica-CS 12 g, 20 m/min, silica gel, UV 254) to afford the product (260 mg, 67%). ESI-MS m/z calcd for [C₂₆H₃₀BrClN₆O₆] [M+H]⁺: 637.1; found: 636.8.

### 2-Bromo-5-chloro-3-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}pyridine

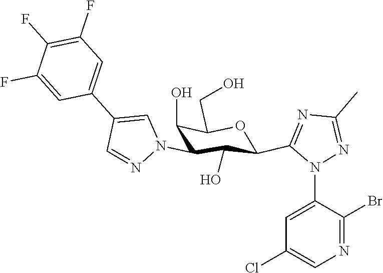

To a solution of 3-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-2-bromo-5-chloropyridine (260 mg, 0.41 mmol) in EtOH (5 mL) 3-(dimethylamino)-2-(3,4,5-trifluorophenyl)prop-2-enal (121 mg, 0.53 mmol) and concentrated HCl (0.2 mL) were added and the mixture was stirred 2 h at 80° C. The mixture was concentrated and purified by column chromatography (MeOH/DCM=0˜1/10, Silica-CS 12 g, 20 m/min, silica gel, UV 254) to give the product (80 mg, 32%). ESI-MS m/z calcd for [C₂₃H₁₉BrClF₃N₆O₄] [M+H]⁺: 615.0; found: 614.8. ¹H NMR (400 MHz, Methanol-d₄) δ 8.62 (d, J=2.4 Hz, 1H), 8.20-8.13 (m, 2H), 7.89 (s, 1H), 7.35 (dd, J=9.2, 6.4 Hz, 2H), 4.49-4.36 (m, 2H), 4.66 (d, J=2.4 Hz, 1H), 3.73-3.54 (m, 1H), 3.13-3.01 (m, 3H), 2.43 (s, 3H).

### Intermediate 22

### (2,5-Dibromo-3-pyridyl)hydrazine

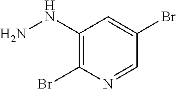

To a cooled (0° C.) solution of 2,5-dibromopyridin-3-amine (1.50 g, 5.95 mmol) in water (15 mL) and concentrated HCl (15 mL) a solution of NaNO₂ (493 mg, 7.15 mmol) in water (3 mL) was added and the mixture was stirred 1 h at 0° C. Tin(II) chloride dihydrate (3.36 g, 14.9 mmol) dissolved in concentrated HCl (5.0 mL) was added and the mixture was stirred 2 h at rt. The mixture was basified by addition of aq NaOH until pH reached 10. The mixture was extracted with DCM (3×50 mL). The combined organic phases were dried over Na₂SO₄, evaporated and purified by column chromatography (PE/EtOAc=10/1˜3/1, silica-CS 40 g, 40 mL/min, silica gel, UV 254 nm) to afford the product (700 mg, 66%). ESI-MS m/z calcd for [C₅H₅Br₂N₃] [M+H]⁺: 265.9; found: 265.8. ¹H NMR (400 MHz, DMSO-d₆) δ 7.68 (d, J=2.4 Hz, 1H), 7.54 (d, J=2.0 Hz, 1H), 7.02 (br s, 1H), 4.38 (br s, 2H).

### 3-[5-(3-Azido-4,6-O-benzylidene-3-deoxy-β-D-galactopyranosyl)-3-methyl-1H-1,2,4-triazol-1-yl]-2,5-dibromopyridine

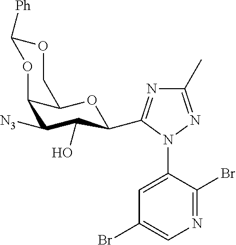

To a solution of 2,6-anhydro-4-azido-5,7-O-benzylidene-4-deoxy-D-glycero-L-manno-N-[1-(dimethylamino)ethylidene]heptonamide (1.30 g, 3.34 mmol) in 1,4-dioxane (15 mL) and acetic acid (5 mL) (2,5-dibromo-3-pyridyl)hydrazine (1.07 g, 4.01 mmol) was added and the mixture was stirred 3 h at 60° C. The mixture was concentrated and basified by addition of aq NaHCO₃ until pH reached 8. The mixture was extracted with DCM (3×50 mL). The combined organic phases were dried over Na₂SO₄, evaporated and purified by column chromatography (EtOAc/PE=1/10˜1/1, Silica-CS 40 g, 40 m/min, silica gel, UV 254) to give the product (800 mg, 40%). ESI-MS m/z calcd for [C₂₁H₁₉Br₂N₇O₄] [M+H]⁺: 592.0; found: 591.7. ¹H NMR (400 MHz, Chloroform-d) δ 8.46 (s, 1H), 7.95 (d, J=1.6 Hz, 1H), 7.43-7.41 (m, 2H), 7.37-7.32 (m, 3H), 5.48 (s, 1H), 4.60 (d, J=9.2 Hz, 1H), 4.46 (t, J=10.0 Hz, 1H), 4.22 (d, J=2.8 Hz, 1H), 3.89 (d, J=11.6 Hz, 1H), 3.62-3.58 (m, 1H), 3.47 (dd, J=10.0, 3.2 Hz, 1H), 3.38 (s, 1H), 2.46 (s, 3H).

### 3-[5-(3-Amino-4,6-O-benzylidene-3-deoxy-β-D-galactopyranosyl)-3-methyl-1H-1,2,4-triazol-1-yl]-2,5-dibromopyridine

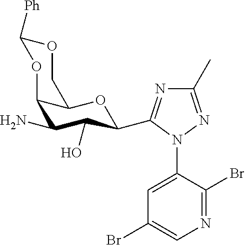

To a solution of 3-[5-(3-azido-4,6-O-benzylidene-3-deoxy-β-D-galactopyranosyl)-3-methyl-1H-1,2,4-triazol-1-yl]-2,5-dibromopyridine (754 mg, 1.27 mmol) in THF (20 mL) and water (1 mL) triphenylphosphine (1.67 g, 6.36 mmol) was added and the mixture was stirred overnight at 50° C. The mixture was concentrated and purified by column chromatography (DCM/MeOH=1/0˜10/1, Silica-CS 40 g, 50 mL/min, silica gel, UV 254) to afford the product (620 mg, 86%). ESI-MS m/z calcd for [C₂₁H₂₁Br₂N₅O₄] [M+H]⁺: 568.0; found: 567.7. ¹H NMR (400 MHz, DMSO-d₆) δ 8.60 (s, 1H), 8.28 (s, 1H), 7.43-7.36 (m, 5H), 5.52 (s, 1H), 5.18 (d, J=5.6 Hz, 1H), 4.20 (d, J=9.2 Hz, 1H), 4.05 (d, J=2.8 Hz, 1H), 4.01 (d, J=12.4 Hz, 1H), 3.91 (d, J=12.0 Hz, 1H), 3.64-3.58 (m, 1H), 3.55 (s, 1H), 2.66 (dd, J=9.6, 3.2 Hz, 1H), 2.34 (s, 3H), 1.74 (br s, 2H).

### 3-{5-{4,6-O-Benzylidene-3-[(2-tert-butoxycarbonyl)hydrazinyl]-3-deoxy-β-D-galactopyranosyl}-3-methyl-1H-1,2,4-triazol-1-yl}-2,5-dibromopyridine

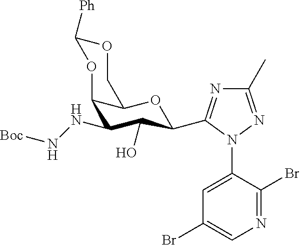

To a solution of 3-[5-(3-amino-4,6-O-benzylidene-3-deoxy-β-D-galactopyranosyl)-3-methyl-1H-1,2,4-triazol-1-yl]-2,5-dibromopyridine (620 mg, 1.09 mmol) in DCM (10 mL) N-tert-butyloxycarbonyl-3-(4-cyanophenyl)oxaziridine (323 mg, 1.31 mmol) was added and the mixture was stirred overnight at rt. The mixture was concentrated and purified by column chromatography (DCM/EtOAc=1/0˜0/1, Silica-CS 20 g, 40 mL/min, silica gel, UV 254) to afford the product (480 mg, 64%). ESI-MS m/z calcd for [C₂₆H₃₀Br₂N₆O₆] [M+H]⁺: 681.1; found: 680.8. ¹H NMR (400 MHz, DMSO-d₆) δ 8.62 (s, 1H), 8.36 (s, 1H), 8.27 (s, 1H), 7.41-7.37 (m, 5H), 5.48 (s, 1H), 5.19 (d, J=5.6 Hz, 1H), 4.56 (s, 1H), 4.29 (d, J=9.6 Hz, 1H), 4.14 (d, J=2.4 Hz, 1H), 4.05-3.99 (m, 1H), 3.90 (d, J=12.0 Hz, 1H), 3.87-3.81 (m, 1H), 3.54 (s, 1H), 2.90 (d, J=10.0 Hz, 1H), 2.34 (s, 3H), 1.39 (s, 9H).

### 2,5-Dibromo-3-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}pyridine

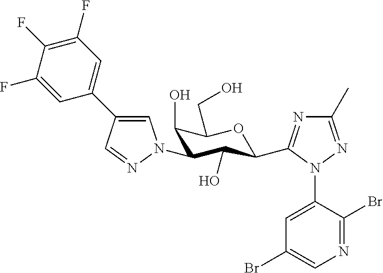

To a solution of 3-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-2,5-dibromopyridine (240 mg, 0.35 mmol) in EtOH (5 mL) 3-(dimethylamino)-2-(3,4,5-trifluorophenyl)prop-2-enal (105 mg, 0.46 mmol) and concentrated HCl (0.2 mL) were added and the mixture was stirred 2 h at 80° C. The mixture was basified by addition of NaHCO₃ to pH=8. The mixture was concentrated and purified by column chromatography (MeOH/DCM=0/1˜1/10, Silica-CS 12 g, 20 mL/min, silica gel, UV 254) to give the product (80 mg, 32%). ESI-MS m/z calcd for [C₂₃H₁₉Br₂F₃N₆O₄][M+H]⁺: 661.0; found: 660.7. ¹H NMR (400 MHz, Chloroform-d) δ 8.51 (d, J=2.0 Hz, 1H), 7.92 (d, J=2.4 Hz, 1H), 7.82 (s, 1H), 7.69 (s, 1H), 7.00 (dd, J=8.0, 6.4 Hz, 2H), 4.62-4.53 (m, 2H), 4.40 (s, 1H), 4.17 (d, J=9.2 Hz, 1H), 3.59-3.52 (m, 3H), 2.36 (s, 3H).

### Intermediate 23

### (4-Bromo-5-chloro-3-pyridyl)hydrazine

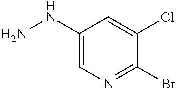

To a cooled (−5° C.) solution of 6-bromo-5-chloropyridin-3-amine (1.00 g, 4.82 mmol) in water (15 mL) and concentrated HCl (15 mL) a cooled (−5° C.) solution of NaNO₂ (399 mg, 5.78 mmol) in water (3 mL) was added dropwise. The mixture was stirred 1 h at −5° C. Tin(II) chloride dihydrate (2.18 g, 9.64 mmol) dissolved in concentrated HCl (5 mL) was added dropwise and the mixture was stirred 2 h at −5° C. The mixture was basified by addition of aq NaOH until pH reached 10. Water (50 mL) and DCM (100 mL) were added and the aqueous phase was extracted with DCM (2×50 mL). The combined organic phases were dried over Na₂SO₄, evaporated and purified by column chromatography (PE/EtOAc=1/0˜0/1, silica-CS 40 g, 40 mL/min, silica gel, UV 254 nm) to afford the product (300 mg, 28%). ESI-MS m/z calcd for [C₅H₅BrClN₃][M+H]⁺: 221.9; found: 221.9. ¹H NMR (400 MHz, Chloroform-d) δ 7.81 (s, 1H), 7.30 (s, 1H), 5.28 (br s, 1H), 3.60 (br s, 2H).

### 5-[5-(3-Azido-4,6-O-benzylidene-3-deoxy-β-D-galactopyranosyl)-3-methyl-1H-1,2,4-triazol-1-yl]-2-bromo-3-chloropyridine

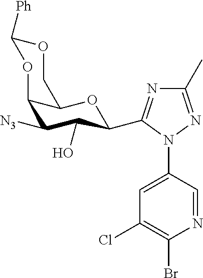

To a solution of 2,6-anhydro-4-azido-5,7-O-benzylidene-4-deoxy-D-glycero-L-manno-N-[1-(dimethylamino)ethylidene]heptonamide (500 mg, 1.28 mmol) in 1,4-dioxane (10 mL) and acetic acid (3 mL) (4-bromo-5-chloro-3-pyridyl)hydrazine (285 mg, 1.28 mmol) was added and the mixture was stirred 2 h at 60° C. The mixture was concentrated and purified by column chromatography (EtOAc/PE=1/10˜1/0, Silica-CS 20 g, 25 mL/min, silica gel, UV 254) to give the product (380 mg, 54%). ESI-MS m/z calcd for [C₂₁H₁₉BrClN₇₀₄] [M+H]⁺: 548.0; found: 547.8. ¹H NMR (400 MHz, Chloroform-d) δ 8.47 (d, J=2.0 Hz, 1H), 8.01 (d, J=2.4 Hz, 1H), 7.43-7.40 (m, 2H), 7.32-7.27 (m, 3H), 5.53 (s, 1H), 4.74 (t, J=9.6 Hz, 1H), 4.35 (d, J=9.2 Hz, 1H), 4.27 (d, J=3.2 Hz, 1H), 4.07-3.98 (m, 2H), 3.50 (s, 1H), 3.42 (dd, J=10.0, 3.2 Hz, 1H), 2.39 (s, 3H).

### 5-[5-(3-Amino-4,6-O-benzylidene-3-deoxy-β-D-galactopyranosyl)-3-methyl-1H-1,2,4-triazol-1-yl]-2-bromo-3-chloropyridine

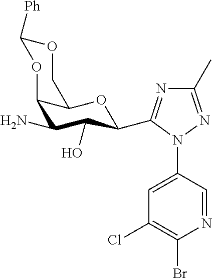

To a solution of 5-[5-(3-azido-4,6-O-benzylidene-3-deoxy-β-D-galactopyranosyl)-3-methyl-1H-1,2,4-triazol-1-yl]-2-bromo-3-chloropyridine (380 mg, 0.69 mmol) in THF (10 mL) and water (0.5 mL) triphenylphosphine (908 mg, 3.46 mmol) was added and the mixture was stirred overnight at 50° C. The mixture was concentrated and purified by column chromatography (DCM/MeOH=1/0˜10/1, Silica-CS 20 g, 40 mL/min, silica gel, UV 254) to afford the product (250 mg, 69%). ESI-MS m/z calcd for [C₂₁H₂₁BrClN₅O₄] [M+H]⁺: 522.0; found: 521.8. ¹H NMR (400 MHz, DMSO-d₆) δ 8.69 (d, J=2.0 Hz, 1H), 8.38 (d, J=2.4 Hz, 1H), 7.48-7.45 (m, 2H), 7.43-7.37 (m, 3H), 5.60 (s, 1H), 5.28 (dd, J=5.2, 2.4 Hz, 1H), 4.43 (d, J=9.6 Hz, 1H), 4.14 (d, J=3.2 Hz, 1H), 4.11-4.06 (m, 2H), 3.81-3.75 (m, 1H), 3.73 (s, 1H), 2.73 (dd, J=10.0, 3.2 Hz, 1H), 2.36 (s, 3H), 1.68 (br s, 2H).

### 5-{5-{4,6-O-Benzylidene-3-[(2-tert-butoxycarbonyl)hydrazinyl]-3-deoxy-β-D-galactopyranosyl}-3-methyl-1H-1,2,4-triazol-1-yl}-2-bromo-3-chloropyridine

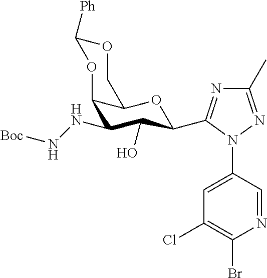

To a solution of 5-[5-(3-amino-4,6-O-benzylidene-3-deoxy-β-D-galactopyranosyl)-3-methyl-1H-1,2,4-triazol-1-yl]-2-bromo-3-chloropyridine (250 mg, 0.48 mmol) in DCM (10 mL) N-tert-butyloxycarbonyl-3-(4-cyanophenyl)oxaziridine (141 mg, 0.57 mmol) was added and the mixture was stirred overnight at rt. The mixture was concentrated and purified by column chromatography (DCM/EtOAc=1/0˜0/1, Silica-CS 20 g, 40 m/min, silica gel, UV 254) to afford the product (180 mg, 59%). ESI-MS m/z calcd for [C₂₆H₃₀BrClN₆O₆] [M+H]⁺: 637.1; found: 636.8. ¹H NMR (400 MHz, Chloroform-d) δ 8.53 (d, J=2.4 Hz, 1H), 8.02 (d, J=2.0 Hz, 1H), 7.41-7.40 (m, 2H), 7.32-7.28 (m, 3H), 6.67 (br s, 1H), 5.49 (s, 1H), 4.54-4.38 (m, 3H), 4.09 (d, J=12.4 Hz, 1H), 4.01 (d, J=13.4 Hz, 1H), 3.52 (s, 1H), 3.17-3.15 (m, 1H), 2.37 (s, 3H), 1.38 (s, 9H).

### 2-Bromo-3-chloro-5-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}pyridine

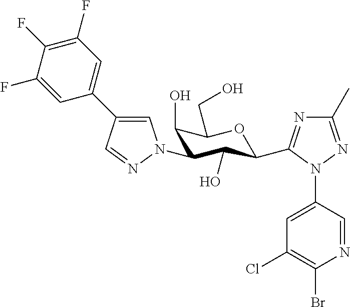

To a solution of 5-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-2-bromo-3-chloropyridine (180 mg, 0.28 mmol) in EtOH (5 mL) 3-(dimethylamino)-2-(3,4,5-trifluorophenyl)prop-2-enal (77.6 mg, 0.34 mmol) and concentrated HCl (0.2 mL) were added and the mixture was stirred 2 h at 80° C. The mixture was basified by addition of NaHCO₃ to pH=8. The mixture was concentrated and purified by column chromatography (MeOH/DCM=0/1˜1/10, Silica-CS 20 g, 30 mL/min, silica gel, UV 254) to give the product (140 mg, 81%). ESI-MS m/z calcd for [C₂₃H₁₉BrClF₃N₆O₄][M+H]⁺: 615.0; found: 614.8. ¹H NMR (400 MHz, DMSO-d₆) δ 8.72 (d, J=2.4 Hz, 1H), 8.45 (d, J=2.4 Hz, 1H), 8.38 (s, 1H), 8.02 (s, 1H), 7.64 (dd, J=9.6, 6.8 Hz, 2H), 5.34 (dd, J=6.8, 3.6 Hz, 1H), 5.16 (d, J=5.6 Hz, 1H), 4.85-4.78 (m, 2H), 4.56 (d, J=9.2 Hz, 1H), 4.48 (dd, J=10.8, 1.2 Hz, 1H), 3.95 (dd, J=5.6, 2.8 Hz, 1H), 3.86-3.83 (m, 1H), 3.57-3.45 (m, 2H), 2.40 (s, 3H).

### Intermediate 24

### 5-Bromo-2-(trifluoromethyl)aniline

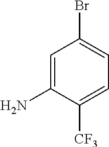 [SMILES: Nc1cc(Br)ccc1C(F)(F)F]

To a solution of 4-bromo-2-nitro-1-(trifluoromethyl)benzene (2.0 g, 7.41 mmol) in EtOH/H₂O (30 mL, 3:1) iron (2.07 g, 37.0 mmol) and NH₄Cl (1.98 g, 37.0 mmol) were added and the mixture was stirred 2 h at 85° C. The mixture was concentrated and purified by column chromatography (PE/EtOAc=1/0˜10/1, Silica-CS 20 g, 30 mL/min, silica gel, UV 254) to give the product (1.4 g, 79%). ESI-MS m/z calcd for [C₇H₅BrF₃N] [M+H]⁺: 240.0; found: 239.8. ¹H NMR (400 MHz, Chloroform-d) δ 7.19 (d, J=8.4 Hz, 1H), 6.86-6.79 (m, 2H), 3.51 (br s, 2H).

### [5-Bromo-2(trifluoromethyl)phenyl]hydrazine

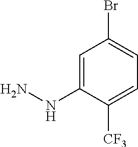 [SMILES: NNc1cc(Br)ccc1C(F)(F)F]

To a cooled (−10° C.) solution of 5-bromo-2-(trifluoromethyl)aniline (400 mg, 1.67 mmol) in concentrated HCl (10 mL) a solution of NaNO₂ (138 mg, 2.00 mmol) in water (1 mL) was added dropwise and the mixture was stirred 30 min at −10° C. Tin(II) chloride dihydrate (1.13 g, 5.00 mmol) dissolved in concentrated HCl (3.0 mL) was added dropwise at −10° C. The resulting mixture was slowly warmed to rt and then stirred 2 h. The mixture was poured into saturated aq NaHCO₃ and extracted with EtOAc (3×20 mL). The combined organic phases were washed with brine, dried over Na₂SO₄ and evaporated to afford the product (300 mg, 71%). ESI-MS m/z calcd for [C₇H₆BrF₃N₂] [M+H]⁺: 255.0; found: 254.9. ¹H NMR (400 MHz, Chloroform-d) δ 7.47 (d, J=0.8 Hz, 1H), 7.20 (d, J=8.4 Hz, 1H), 6.84 (dd, J=8.0, 0.8 Hz, 1H), 5.80 (s, 1H), 3.56 (br s, 2H).

### 1-[5-(3-Azido-4,6-O-benzylidene-3-deoxy-β-D-galactopyranosyl)-3-methyl-1H-1,2,4-triazol-1-yl]-5-bromo-2-(trifluoromethyl)benzene

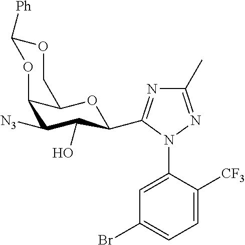

To a solution of 2,6-anhydro-4-azido-5,7-O-benzylidene-4-deoxy-D-glycero-L-manno-heptonamide (247 mg, 0.77 mmol) in 1,4-dioxane (9.0 mL) N,N-dimethylacetamide dimethyl acetal (113 mg, 0.85 mmol) was added and the mixture was stirred 3 h at 50° C. The mixture was evaporated, and the residue was dissolved in 1,4-dioxane (9.0 mL) and acetic acid (3 mL). To this solution [5-bromo-2(trifluoromethyl)phenyl]hydrazine (236 mg, 0.93 mmol) was added and the mixture was stirred 2 h at 60° C. The mixture was evaporated and partitioned between water (20 mL) and DCM (20 mL). The aqueous phase was extracted with DCM (2×20 mL). The combined organic phases were washed with water (20 mL) and brine (20 mL), dried over Na₂SO₄, evaporated and purified by column chromatography (PE/EtOAc=5/1˜1/2, Silica-CS 20 g, 30 m/min, silica gel, UV 254) to afford the product (300 mg, 67%). ESI-MS m/z calcd for [C₂₃H₂₀BrF₃N₆O₄] [M+H]⁺: 581.1; found: 580.8. ¹H NMR (400 MHz, Chloroform-d) δ 7.71-7.62 (m, 2H), 7.53 (d, J=8.4 Hz, 1H), 7.41-7.36 (m, 2H), 7.35-7.25 (m, 3H), 5.41 (s, 1H), 4.45 (t, J=9.6 Hz, 1H), 4.34-4.24 (m, 1H), 4.13 (d, J=3.2 Hz, 1H), 3.81 (dd, J=12.4, 1.2 Hz, 1H), 3.64-3.54 (m, 1H), 3.36 (dd, J=10.0, 3.2 Hz, 1H), 3.21 (s, 1H), 2.38 (s, 3H).

### 1-[5-(3-Amino-4,6-O-benzylidene-3-deoxy-β-D-galactopyranosyl)-3-methyl-1H-1,2,4-triazol-1-yl]-5-bromo-2-(trifluoromethyl)benzene

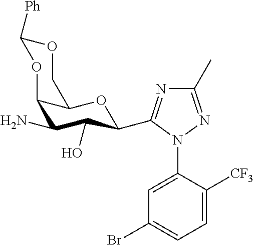

To a solution of 1-[5-(3-azido-4,6-O-benzylidene-3-deoxy-β-D-galactopyranosyl)-3-methyl-1H-1,2,4-triazol-1-yl]-5-bromo-2-(trifluoromethyl)benzene (300 mg, 0.52 mmol) in THF/water (12 mL, 5:1) triphenylphosphine (474 mg, 1.81 mmol) was added and the mixture was stirred 6 h at 50° C. The mixture was concentrated and purified by column chromatography (DCM/MeOH=1/0˜10/1, Silica-CS 20 g, 30 mL/min, silica gel, UV 254) to give the product (200 mg, 70%). ESI-MS m/z calcd for [C₂₃H₂₂BrF₃N₄O₄] [M+H]⁺: 555.1; found: 554.9. ¹H NMR (400 MHz, DMSO-d₆) δ 7.97 (d, J=8.4 Hz, 1H), 7.89 (d, J=8.4 Hz, 1H), 7.84 (s, 1H), 7.46-7.36 (m, 5H), 5.54 (s, 1H), 5.22 (d, J=3.6 Hz, 1H), 4.11-3.97 (m, 3H), 3.88 (d, J=11.6 Hz, 1H), 3.78-3.67 (m, 1H), 3.48 (s, 1H), 2.64 (dd, J=9.2, 3.2 Hz, 1H), 2.32 (s, 3H), 1.81 (br s, 2H).

### 1-{5-{4,6-O-Benzylidene-3-[(2-tert-butoxycarbonyl)hydrazinyl]-3-deoxy-β-D-galactopyranosyl}-3-methyl-1H-1,2,4-triazol-1-yl}-5-bromo-2-(trifluoromethyl)benzene

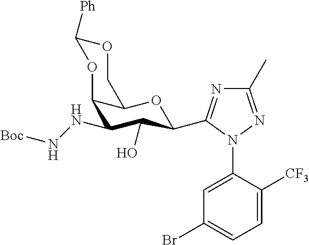

To a solution of 1-[5-(3-amino-4,6-O-benzylidene-3-deoxy-β-D-galactopyranosyl)-3-methyl-1H-1,2,4-triazol-1-yl]-5-bromo-2-(trifluoromethyl)benzene (200 mg, 0.36 mmol) in DCM (10 mL) N-tert-butyloxycarbonyl-3-(4-cyanophenyl)oxaziridine (97.6 mg, 0.40 mmol) was added and the mixture was stirred overnight at rt. The mixture was concentrated and purified by column chromatography (PE/EtOAc=4/1˜0/1, Silica-CS 20 g, 30 mL/min, silica gel, UV 254) to afford the product (150 mg, 62%). ESI-MS m/z calcd for [C₂₅H₃₁BrF₃N₅O₆] [M+H]⁺: 670.1; found: 669.9. ¹H NMR (400 MHz, DMSO-d₆) δ 8.34 (br s, 1H), 7.98 (d, J=8.0 Hz, 1H), 7.89 (d, J=8.8 Hz, 1H), 7.82 (s, 1H), 7.49-7.34 (m, 5H), 5.50 (s, 1H), 5.22 (d, J=4.0 Hz, 1H), 4.56 (br s, 1H), 4.19-4.12 (m, 2H), 4.04-3.92 (m, 2H), 3.88 (d, J=12.0 Hz, 1H), 3.48 (s, 1H), 2.94-2.83 (m, 1H), 2.32 (s, 3H), 1.38 (s, 9H).

### Intermediate 25

### 5-Bromo-3-nitro-2-(trifluoromethyl)pyridine

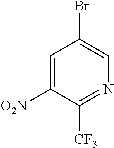 [SMILES: O=[N+]([O-])c1cc(Br)cnc1C(F)(F)F]

A solution of 2,5-dibromo-3-nitropyridine (2.00 g, 7.09 mmol), methyl 2,2-difluoro-2-(fluorosulfonyl)acetate (2.05 g, 10.6 mmol) and CuI (1.62 g, 8.51 mmol) in DMF (20.0 mL) was stirred 2 h at 100° C. After cooling to rt, the mixture was poured into water (50 mL) and extracted with EtOAc (3×50 mL). The combined organic phases were washed with water (50 mL) and brine (50 mL), dried over Na₂SO₄, evaporated, and purified by column chromatography (EtOAc/PE=0/1˜1/4, Silica-CS 20 g, 20 mL/min, silica gel, UV 254) to give the product (1.8 g, 94%). ESI-MS m/z calcd for [C₆H₂BrF₃N₂O₂] [M]: 269.9; found: 270.0. ¹H NMR (400 MHz, Chloroform-d) δ 8.90 (d, J=1.6 Hz, 1H), 8.30 (d, J=1.6 Hz, 1H).

### 5-Bromo-2-(trifluoromethyl)pyridin-3-amine

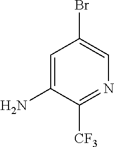 [SMILES: Nc1cc(Br)cnc1C(F)(F)F]

To a solution of 5-bromo-3-nitro-2-(trifluoromethyl)pyridine (1.8 g, 6.64 mmol) in EtOH/H₂O (22 mL, 10:1) iron (1.86 g, 33.2 mmol) and NH₄Cl (2.13 g, 39.9 mmol) were added and the mixture was stirred 6 h at 85° C. The mixture was concentrated and purified by column chromatography (PE/EtOAc=1/0˜4/1, Silica-CS 20 g, 20 mL/min, silica gel, UV 254) to give the product (1.45 g, 91%). ESI-MS m/z calcd for [C₆H₄BrF₃N₂] [M+H]⁺: 241.0; found: 240.9. ¹H NMR (400 MHz, Chloroform-d) δ 8.01 (d, J=1.6 Hz, 1H), 7.21 (d, J=1.6 Hz, 1H), 4.22 (br s, 2H).

### [5-Bromo-2(trifluoromethyl)pyrid-3-yl]hydrazine

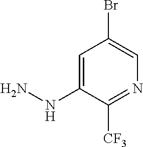 [SMILES: NNc1cc(Br)cnc1C(F)(F)F]

To a cooled (−5° C.) solution of 5-bromo-2-(trifluoromethyl)pyridin-3-amine (600 mg, 2.49 mmol) in acetic acid (3.0 mL) concentrated HCl (9.0 mL) was added followed by dropwise addition of a solution of NaNO₂ (223 mg, 3.24 mmol) in water (1 mL). The resulting mixture was stirred 45 min at −5° C. Tin(II) chloride dihydrate (1.69 g, 7.47 mmol) dissolved in concentrated HCl (3.0 mL) was added dropwise at 0° C. The resulting mixture was stirred 2 h at −5° C. Water (30 mL) was added and the pH was adjusted to 8 by dropwise addition of aq NaOH (5 M). The aqueous phase was extracted with EtOAc (2×50 mL). The combined organic phases were washed with water (50 mL) and brine (50 mL), dried over Na₂SO₄, evaporated, and purified by column chromatography (EtOAc/PE=0/1˜1/4, Silica-CS 20 g, 20 m/min, silica gel, UV 254) to afford the product (350 mg, 55%). ESI-MS m/z calcd for [C₆H₅BrF₃N₃] [M+H]⁺: 256.0; found: 256.1. ¹H NMR (400 MHz, Chloroform-d) δ 7.98 (s, 2H), 5.93 (s, 1H), 3.64 (br s, 2H).

### 1-[5-(3-Azido-4,6-O-benzylidene-3-deoxy-β-D-galactopyranosyl)-3-methyl-1H-1,2,4-triazol-1-yl]-5-bromo-2-(trifluoromethyl)pyridine

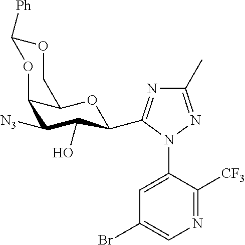

To a solution of 2,6-anhydro-4-azido-5,7-O-benzylidene-4-deoxy-D-glycero-L-manno-heptonamide (340 mg, 1.06 mmol) in 1,4-dioxane (15.0 mL) N,N-dimethylacetamide dimethyl acetal (156 mg, 1.17 mmol) was added and the mixture was stirred 3 h at 50° C. The mixture was evaporated, and the residue was dissolved in acetic acid (10 mL). To this solution [5-bromo-2(trifluoromethyl)pyrid-3-yl]hydrazine (326 mg, 1.27 mmol) was added and the mixture was stirred 2 h at 70° C. The mixture was evaporated and partitioned between water (20 mL) and DCM (20 mL). The aqueous phase was extracted with DCM (2×20 mL). The combined organic phases were washed with water (20 mL) and brine (20 mL), dried over Na₂SO₄, evaporated, and purified by column chromatography (PE/EtOAc=5/1˜1/2, Silica-CS 20 g, 30 m/min, silica gel, UV 254). The obtained material was purified by reversed-phase chromatography (MeCN/H₂O=1/20˜1/1, C-18 column, 20 m/min, UV 254) to afford the product (190 mg, 31%). ESI-MS m/z calcd for [C₂₂H₁₉BrF₃N₇₀₄] [M+H]⁺: 582.1; found: 581.8. ¹H NMR (400 MHz, Chloroform-d) δ 8.74 (d, J=2.0 Hz, 1H), 7.99 (d, J=2.0 Hz, 1H), 7.40-7.25 (m, 5H), 5.40 (s, 1H), 4.62 (s, 1H), 4.39 (d, J=8.8 Hz, 1H), 4.32 (t, J=9.2 Hz, 1H), 4.13 (d, J=2.8 Hz, 1H), 3.80 (dd, J=12.4, 1.6 Hz, 1H), 3.44 (dd, J=12.4, 1.2 Hz, 1H), 3.37 (dd, J=10.0, 3.2 Hz, 1H), 3.26 (d, J=0.8 Hz, 1H), 2.37 (s, 3H).

### 1-[5-(3-Amino-4,6-O-benzylidene-3-deoxy-β-D-galactopyranosyl)-3-methyl-1H-1,2,4-triazol-1-yl]-5-bromo-2-(trifluoromethyl)pyridine

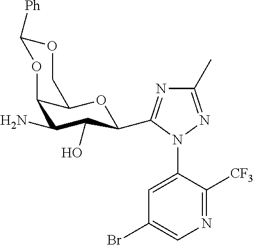

To a solution of 1-[5-(3-azido-4,6-O-benzylidene-3-deoxy-β-D-galactopyranosyl)-3-methyl-1H-1,2,4-triazol-1-yl]-5-bromo-2-(trifluoromethyl)pyridine (190 mg, 0.33 mmol) in THF/water (12 mL, 5:1) triphenylphosphine (428 mg, 1.63 mmol) was added and the mixture was stirred 6 h at 50° C. The mixture was concentrated and purified by column chromatography (DCM/MeOH=1/0˜10/1, Silica-CS 20 g, 30 mL/min, silica gel, UV 254) to give the product (175 mg, 96%). ESI-MS m/z calcd for [C₂₂H₂₁BrF₃N₅O₄] [M+H]⁺: 556.1; found: 555.8. ¹H NMR (400 MHz, Chloroform-d) δ 8.74 (s, 1H), 8.02 (s, 1H), 7.38-7.25 (m, 5H), 5.37 (s, 1H), 4.27 (d, J=9.2 Hz, 1H), 4.11-4.00 (m, 1H), 3.89-3.76 (m, 2H), 3.46 (d, J=12.8 Hz, 1H), 3.26 (s, 1H), 2.79 (d, J=8.4 Hz, 1H), 2.37 (s, 3H), 1.71 (br s, 2H), 0.87-0.71 (m, 1H).

### 1-{5-{4,6-O-Benzylidene-3-[(2-tert-butoxycarbonyl)hydrazinyl]-3-deoxy-β-D-galactopyranosyl}-3-methyl-1H-1,2,4-triazol-1-yl}-5-bromo-2-(trifluoromethyl)pyridine

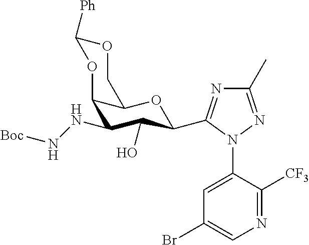

To a solution of 1-[5-(3-amino-4,6-O-benzylidene-3-deoxy-β-D-galactopyranosyl)-3-methyl-1H-1,2,4-triazol-1-yl]-5-bromo-2-(trifluoromethyl)pyridine (175 mg, 0.32 mmol) in DCM (10 mL) N-tert-butyloxycarbonyl-3-(4-cyanophenyl)oxaziridine (93 mg, 0.38 mmol) was added and the mixture was stirred overnight at rt. The mixture was concentrated and purified by column chromatography (PE/EtOAc=4/1˜0/1, Silica-CS 20 g, 30 mL/min, silica gel, UV 254) to afford the product (85 mg, 40%). ESI-MS m/z calcd for [C₂₇H₃₀BrF₃N₆O₆] [M+H]⁺: 671.1; found: 670.8. ¹H NMR (400 MHz, Chloroform-d) δ 8.67 (d, J=1.2 Hz, 1H), 8.00 (d, J=2.0 Hz, 1H), 7.35-7.25 (m, 5H), 6.39 (br s, 1H), 5.36 (s, 1H), 4.63 (br s, 1H), 4.46 (br s, 1H), 4.35 (d, J=9.2 Hz, 1H), 4.22 (d, J=2.8 Hz, 1H), 4.13-4.07 (m, 1H), 3.82 (dd, J=12.8, 1.6 Hz, 1H), 3.55 (d, J=13.2 Hz, 1H), 3.25 (s, 1H), 2.99 (dd, J=9.6, 2.4 Hz, 1H), 2.37 (s, 3H), 1.38 (s, 9H).

### Intermediate 26

### 4-Bromo-2-hydrazinylbenzonitrile

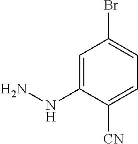 [SMILES: N#Cc1ccc(Br)cc1NN]

To a solution of 4-bromo-2-fluorobenzonitrile (2.0 g, 10.0 mmol) in THF (30 mL), hydrazine monohydrate (1.46 mL, 30.0 mmol) was added, and the mixture was stirred overnight at rt. The mixture was evaporated, DCM (100 mL) was added, and the mixture was stirred 30 min at rt. The precipitate was filtered off and dried to afford the product (1.0 g, 47%). ESI-MS m/z calcd for [C₇H₆BrN₃] [M+H]⁺: 212.0; found: 212.0. ¹H NMR (400 MHz, DMSO-d₆) δ 7.70 (br s, 1H), 7.37 (d, J=2.0 Hz, 1H), 7.36 (d, J=8.4 Hz, 1H), 6.77 (dd, J=8.4, 2.0 Hz, 1H), 4.36 (br s, 2H).

### 1-[5-(3-Azido-4,6-O-benzylidene-3-deoxy-β-D-galactopyranosyl)-3-methyl-1H-1,2,4-triazol-1-yl]-5-bromo-2-cyanobenzene

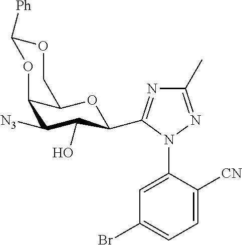

To a solution of 2,6-anhydro-4-azido-5,7-O-benzylidene-4-deoxy-D-glycero-L-manno-N-[1-(dimethylamino)ethylidene]heptonamide (500 mg, 1.28 mmol) in 1,4-dioxane (10 mL) and acetic acid (0.3 mL) 4-bromo-2-hydrazinylbenzonitrile (817 mg, 3.85 mmol) was added and the mixture was stirred overnight at rt. The mixture was concentrated and DCM (100 mL) was added. The solution was washed with saturated aq NaHCO₃ and brine. The organic phase was dried over Na₂SO₄, evaporated and purified by column chromatography (EtOAc/PE=0/1˜1/0, Silica-CS 40 g, 40 m/min, silica gel, UV 254) to give the product (100 mg, 15%). ESI-MS m/z calcd for [C₂₃H₂₀BrN₇O₄][M+H]⁺: 538.1; found: 537.8. ¹H NMR (400 MHz, Chloroform-d) δ 7.76 (d, J=1.6 Hz, 1H), 7.53 (dd, J=8.0, 1.6 Hz, 1H), 7.43 (d, J=8.4 Hz, 1H), 7.37-7.33 (m, 2H), 7.31-7.24 (m, 3H), 5.42 (s, 1H), 4.60 (d, J=8.8 Hz, 1H), 4.43 (t, J=9.6 Hz, 1H), 4.17 (d, J=3.2 Hz, 1H), 3.85 (dd, J=12.4, 1.2 Hz, 1H), 3.63 (dd, J=12.8, 0.8 Hz, 1H), 3.42 (dd, J=10.0, 3.2 Hz, 1H), 3.89 (s, 1H), 2.39 (s, 3H).

### 1-[5-(3-Amino-4,6-O-benzylidene-3-deoxy-β-D-galactopyranosyl)-3-methyl-1H-1,2,4-triazol-1-yl]-5-bromo-2-cyanobenzene

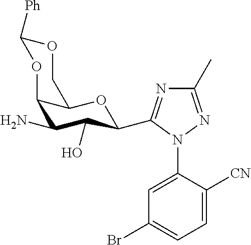

To a solution of 1-[5-(3-azido-4,6-O-benzylidene-3-deoxy-β-D-galactopyranosyl)-3-methyl-1H-1,2,4-triazol-1-yl]-5-bromo-2-cyanobenzene (100 mg, 0.19 mmol) in THF (10 mL) and water (0.5 mL) triphenylphosphine (244 mg, 0.93 mmol) was added and the mixture was stirred overnight at 50° C. The mixture was concentrated and purified by column chromatography (DCM/MeOH=1/0˜10/1, Silica-CS 12 g, 40 mL/min, silica gel, UV 254) to give the product (50 mg, 53%). ESI-MS m/z calcd for [C₂₃H₂₂BrN₅O₄] [M+H]⁺: 512.1; found: 511.9. ¹H NMR (400 MHz, Chloroform-d) δ 7.78 (d, J=2.0 Hz, 1H), 7.50 (dd, J=8.4, 1.6 Hz, 1H), 7.43 (d, J=8.4 Hz, 1H), 7.37-7.33 (m, 2H), 7.31-7.25 (m, 3H), 5.39 (s, 1H), 4.47 (d, J=9.2 Hz, 1H), 4.13 (d, J=2.8 Hz, 1H), 3.98 (t, J=9.6 Hz, 1H), 3.87 (dd, J=12.8, 1.6 Hz, 1H), 3.67 (d, J=12.0 Hz, 1H), 3.39 (s, 1H), 2.96 (d, J=8.0 Hz, 1H), 2.35 (s, 3H).

### 1-{5-{4,6-O-Benzylidene-3-[(2-tert-butoxycarbonyl)hydrazinyl]-3-deoxy-β-D-galactopyranosyl}-3-methyl-1H-1,2,4-triazol-1-yl}-5-bromo-2-cyanobenzene

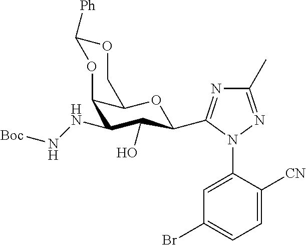

To a solution of 1-[5-(3-amino-4,6-O-benzylidene-3-deoxy-β-D-galactopyranosyl)-3-methyl-1H-1,2,4-triazol-1-yl]-5-bromo-2-cyanobenzene (50 mg, 0.098 mmol) in DCM (10 mL) N-tert-butyloxycarbonyl-3-(4-cyanophenyl)oxaziridine (28.8 mg, 0.12 mmol) was added and the mixture was stirred overnight at rt. The mixture was concentrated and purified by column chromatography (DCM/EtOAc=1/0˜0/1, Silica-CS 12 g, 40 m/min, silica gel, UV 254) to afford the product (35 mg, 57%). ESI-MS m/z calcd for [C₂₈H₃IBrN₆O₆] [M+H]⁺: 627.1; found: 626.8. ¹H NMR (400 MHz, DMSO-d₆) δ 8.34 (br s, 1H), 7.97 (d, J=1.2 Hz, 1H), 7.93 (d, J=8.4 Hz, 1H), 7.79 (dd, J=8.4, 2.0 Hz, 1H), 7.43-7.37 (m, 5H), 5.50 (s, 1H), 5.28 (d, J=5.2 Hz, 1H), 4.57 (br s, 1H), 4.39 (d, J=9.6 Hz, 1H), 4.17 (d, J=2.8 Hz, 1H), 4.07 (d, J=11.2 Hz, 1H), 3.96 (d, J=12.8 Hz, 1H), 3.93-3.88 (m, 1H), 3.60 (s, 1H), 2.92 (d, J=10.0 Hz, 1H), 2.36 (s, 3H), 1.39 (s, 9H).

### Intermediate 27

### (2-Bromo-5-chlorophenyl)hydrazine

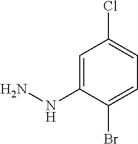

To a solution of 1-bromo-4-chloro-2-fluorobenzene (3.00 g, 14.3 mmol) in DMSO (5 mL), hydrazine monohydrate (2.78 mL, 57.3 mmol) was added, and the mixture was stirred 2 h at 70° C. The mixture was cooled to rt, and water (50 mL) was added. The precipitate was filtered off and dried to afford the product (1.40 g, 44%). ESI-MS m/z calcd for [C₇H₆BrN₃] [M+H]⁺: 212.0; found: 212.0. ¹H NMR (400 MHz, DMSO-d₆) δ 7.35 (d, J=8.0 Hz, 1H), 7.14 (d, J=2.4 Hz, 1H), 6.62 (s, 1H), 6.55 (dd, J=8.0, 2.4 Hz, 1H), 4.25 (s, 2H).

### 1-[5-(3-Azido-4,6-O-benzylidene-3-deoxy-β-D-galactopyranosyl)-3-methyl-1H-1,2,4-triazol-1-yl]-2-bromo-5-chlorobenzene

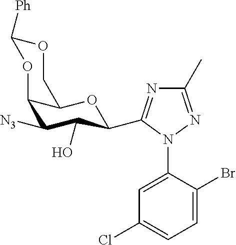

To a solution of 2,6-anhydro-4-azido-5,7-O-benzylidene-4-deoxy-D-glycero-L-manno-N-[1-(dimethylamino)ethylidene]heptonamide (430 mg, 1.10 mmol) in 1,4-dioxane (6 mL) and acetic acid (2 mL) (2-bromo-5-chlorophenyl)hydrazine (254 mg, 1.14 mmol) was added and the mixture was stirred 3 h at 60° C. The mixture was concentrated and DCM (10 mL) was added. The solution was washed with saturated aq NaHCO₃ and brine. The organic phase was dried over Na₂SO₄, evaporated and purified by column chromatography (EtOAc/PE=0/1˜3/1, Silica-CS 12 g, 20 m/min, silica gel, UV 254) to give the product (290 mg, 38%). ESI-MS m/z calcd for [C₂₂H₂₀BrClN₆O₄] [M+H]⁺: 547.0; found: 546.8.

### 1-[5-(3-Amino-4,6-O-benzylidene-3-deoxy-β-D-galactopyranosyl)-3-methyl-1H-1,2,4-triazol-1-yl]-2-bromo-5-chlorobenzene

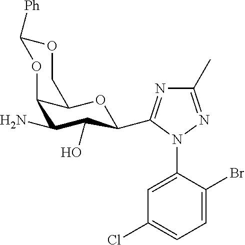

To a solution of 1-[5-(3-azido-4,6-O-benzylidene-3-deoxy-β-D-galactopyranosyl)-3-methyl-1H-1,2,4-triazol-1-yl]-2-bromo-5-chlorobenzene (290 mg, 0.42 mmol) in THF (5 mL) and water (1 mL) triphenylphosphine (555 mg, 2.12 mmol) was added and the mixture was stirred 16 h at 50° C. The mixture was concentrated and purified by column chromatography (DCM/MeOH=1/0˜10/1, Silica-CS 12 g, 20 mL/min, silica gel, UV 254) to give the product (149 mg, 67%). ESI-MS m/z calcd for [C₂₂H₂₂BrClN₄O₄][M+H]⁺: 521.1; found: 520.9. ¹H NMR (400 MHz, Chloroform-d) δ 7.59-7.52 (m, 2H), 7.48-7.33 (m, 6H), 5.46 (s, 1H), 4.35 (d, J=8.8 Hz, 1H), 4.24-4.17 (m, 2H), 3.89 (d, J=12.4 Hz, 1H), 3.76 (d, J=12.4 Hz, 1H), 3.30 (s, 1H), 3.10-3.08 (m, 1H), 2.38 (s, 3H).

### 1-{5-{4,6-O-Benzylidene-3-[(2-tert-butoxycarbonyl)hydrazinyl]-3-deoxy-β-D-galactopyranosyl}-3-methyl-1H-1,2,4-triazol-1-yl}-2-bromo-5-chlorobenzene

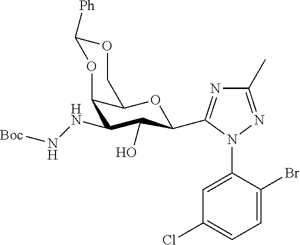

To a solution of 1-[5-(3-amino-4,6-O-benzylidene-3-deoxy-β-D-galactopyranosyl)-3-methyl-1H-1,2,4-triazol-1-yl]-2-bromo-5-chlorobenzene (149 mg, 0.29 mmol) in DCM (3.0 mL) N-tert-butyloxycarbonyl-3-(4-cyanophenyl)oxaziridine (77.4 mg, 0.31 mmol) was added and the mixture was stirred overnight at rt. The mixture was concentrated and purified by column chromatography (PE/EtOAc=4/1˜0/1, Silica-CS 12 g, 20 mL/min, silica gel, UV 254) to afford the product (106 mg, 58%). ESI-MS m/z calcd for [C₂₇H₃₁BrClN₅O₆] [M+H]⁺: 636.1; found: 635.8. ¹H NMR (400 MHz, Chloroform-d) δ 7.48-7.41 (m, 2H), 7.36-7.33 (m, 2H), 7.30-7.25 (m, 3H), 7.17 (dd, J=8.8, 2.0 Hz, 1H), 6.58 (s, 1H), 5.38 (s, 1H), 4.27-4.22 (m, 3H), 3.84 (dd, J=12.4, 1.6 Hz, 1H), 3.21 (s, 1H), 2.99-2.98 (m, 1H), 2.35 (s, 3H), 1.37 (s, 9H).

### 2-Bromo-5-chloro-1-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}benzene

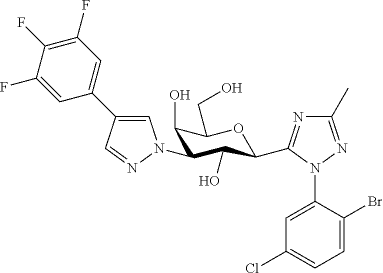

To a solution of 1-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-2-bromo-5-chlorobenzene (106 mg, 0.17 mmol) in EtOH (5.0 mL) 3-(dimethylamino)-2-(3,4,5-trifluorophenyl)prop-2-enal (42 mg, 0.18 mmol) was added followed by concentrated HCl (0.25 mL) and the mixture was stirred 2 h at 80° C. The mixture was concentrated and purified by column chromatography (DCM/MeOH=1/0˜15/1, Silica-CS 12 g, 20 m/min, silica gel, UV 254) to afford the product (77 mg, 75%). ESI-MS m/z calcd for [C₂₄H₂₀BrClF₃N₅O₄] [M+H]⁺: 614.0; found: 613.8. ¹H NMR (400 MHz, Chloroform-d) δ 7.82 (s, 1H), 7.59 (s, 1H), 7.56 (d, J=8.4 Hz, 1H), 7.41 (d, J=2.0 Hz, 1H), 7.30 (dd, J=8.8, 2.4 Hz, 1H), 6.94 (dd, J=8.4, 6.4 Hz, 2H), 4.67 (t, J=9.6 Hz, 1H), 4.38 (d, J=7.2 Hz, 1H), 4.30 (d, J=1.6 Hz, 1H), 4.15 (dd, J=10.4, 2.0 Hz, 1H), 3.55-3.49 (m, 3H), 2.29 (s, 3H).

### Intermediate 29

### 5-Chloro-1-{5-[3-deoxy-3-(3-ethoxycarbonyl-1H-1,2-pyrazol-1-yl)-β-D-galactopyranosyl]-3-methyl-1H-1,2,4-triazol-1-yl}-2-(trifluoromethyl)benzene

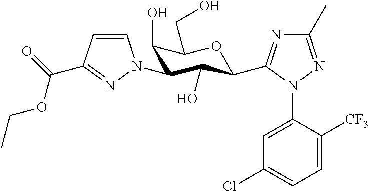

To a solution of 1-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-5-chloro-2-(trifluoromethyl)benzene (346 mg, 0.55 mmol) in DCM (10 mL) TFA (1.0 mL) was added and the mixture was stirred overnight at rt. The mixture was concentrated, and the residue was dissolved in EtOH (5.0 mL). Ethyl 4-(dimethylamino)-2-oxobut-3-enoate (284 mg, 1.66 mmol) was added and the mixture was stirred 2 h at 60° C. The mixture was concentrated and partitioned between EtOAc (20 mL) and diluted aq Na₂CO₃ (10 mL). The organic phase was dried over anhydrous Na₂SO₄, evaporated and purified by prep HPLC [MeOH/H₂O (10 mmol/L, 0.05% TFA), X-Select 10 μm 19*250 mm, 20 m/min, UV 254] to afford the product (60 mg, 30%). ESI-MS m/z calcd for [C₂₂H₂₃ClF₃N₅O₆] [M+H]⁺: 546.1; found: 546.0. ¹H NMR (400 MHz, Methanol-d₄) δ 7.94 (d, J=8.4 Hz, 1H), 7.86-7.83 (m, 2H), 7.75 (s, 1H), 6.81 (d, J=2.4 Hz, 1H), 4.74-4.72 (m, 1H), 4.44 (dd, J=10.8, 2.4 Hz, 1H), 4.33 (q, J=7.2 Hz, 2H), 4.27 (d, J=9.2 Hz, 1H), 4.06 (d, J=2.4 Hz, 1H), 3.65-3.58 (m, 3H), 2.43 (s, 3H), 1.35 (t, J=7.2 Hz, 3H).

### 5-Chloro-1-{5-[3-deoxy-3-(3-ethoxycarbonyl-4-iodo-1H-1,2-pyrazol-1-yl)-β-D-galactopyranosyl]-3-methyl-1H-1,2,4-triazol-1-yl}-2-(trifluoromethyl)benzene

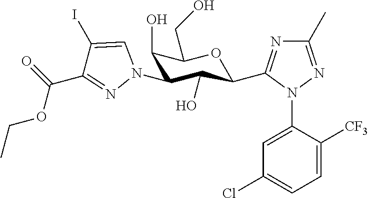

To a solution of 5-chloro-1-{5-[3-deoxy-3-(3-ethoxycarbonyl-1H-1,2-pyrazol-1-yl)-β-D-galactopyranosyl]-3-methyl-1H-1,2,4-triazol-1-yl}-2-(trifluoromethyl)benzene (90 mg, 0.17 mmol) in DMF (3 mL) N-iodosuccinimde (742 mg, 3.30 mmol) was added and the mixture was stirred 2 days at 45° C. The mixture was purified by prep HPLC [MeOH/H₂O (10 mmol/L, 0.05% TFA), X-Select 10 μm 19*250 mm, 20 mL/min, UV 254] to afford the product (54 mg, 49%). ESI-MS m/z calcd for [C₂₂H₂₂ClF₃IN₅O₆][M+H]⁺: 672.0; found: 671.7. ¹H NMR (400 MHz, Chloroform-d) δ 7.87 (s, 1H), 7.75 (d, J=8.8 Hz, 1H), 7.64 (dd, J=8.8, 1.2 Hz, 1H), 7.56 (s, 1H), 4.73-4.67 (m, 1H), 4.56-4.36 (m, 7H), 4.30 (dd, J=10.8, 2.8 Hz, 1H), 3.58-3.49 (m, 3H), 2.40 (s, 3H), 1.39 (d, J=7.2 Hz, 3H).

### Intermediate 33

### (5-Chlorothiazol-2-yl)hydrazine

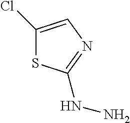

To a cooled (−5° C.) solution of 5-chlorothiazol-2-amine hydrochloride (513 mg, 3.00 mmol) in concentrated HCl (6 mL) a solution of NaNO₂ (310 mg, 4.50 mmol) in water (2 mL) was added and the mixture was stirred 1 h at 0° C. Tin(II) chloride dihydrate (1.69 g, 7.50 mmol) dissolved in concentrated HCl (5 mL) was added dropwise at 0° C. The resulting mixture was slowly warmed to rt and then stirred 3 h. The mixture was concentrated and purified by reversed phase column chromatography (MeCN/H₂O (10 mmol/L TFA), C-18 40 g, 50 m/min, UV 254) to afford the product (400 mg, 89%). ESI-MS m/z calcd for [C₃H₄ClN₃S] [M+NH₄]⁺: 150.0; found: 150.1. ¹H NMR (400 MHz, Deuterium oxide) δ 7.09 (s, 1H).

### 2-[5-(3-Azido-4,6-O-benzylidene-3-deoxy-β-D-galactopyranosyl)-3-methyl-1H-1,2,4-triazol-1-yl]-5-chlorothiazole

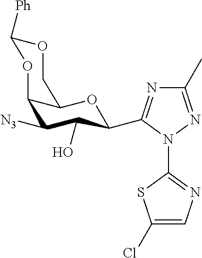

To a solution of 2,6-anhydro-4-azido-5,7-O-benzylidene-4-deoxy-D-glycero-L-manno-N-[1-(dimethylamino)ethylidene]heptonamide (310 mg, 0.80 mmol) in 1,4-dioxane (6 mL) and acetic acid (3 mL) (5-chlorothiazol-2-yl)hydrazine (357 mg, 2.39 mmol) was added and the mixture was stirred 2 h at 60° C. The organic phase was concentrated and purified by column chromatography (EtOAc/DCM=0˜1/1, Silica-CS 20 g, 25 mL/min, silica gel, UV 254) to give the product (185 mg, 49%). ESI-MS m/z calcd for [C₁₉H₁₈C₁N₇O₄S] [M+H]⁺: 476.1; found: 475.9. ¹H NMR (400 MHz, Chloroform-d) δ 7.52-7.48 (m, 2H), 7.37-7.32 (m, 4H), 5.60 (s, 1H), 5.12 (d, J=9.2 Hz, 1H), 4.87 (t, J=10.0 Hz, 1H), 4.37 (d, J=2.8 Hz, 1H), 4.16 (d, J=12.4 Hz, 1H), 4.06 (d, J=12.8 Hz, 1H), 3.68 (s, 1H), 3.55 (dd, J=10.0, 3.2 Hz, 1H), 2.43 (s, 3H).

### 2-[5-(3-Amino-4,6-O-benzylidene-3-deoxy-β-D-galactopyranosyl)-3-methyl-1H-1,2,4-triazol-1-yl]-5-chlorothiazole

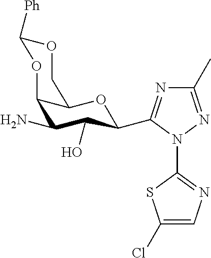

To a solution of 2-[5-(3-azido-4,6-O-benzylidene-3-deoxy-β-D-galactopyranosyl)-3-methyl-1H-1,2,4-triazol-1-yl]-5-chlorothiazole (180 mg, 0.38 mmol) in THF (20 mL) and water (2 mL) triphenylphosphine (471 mg, 1.80 mmol) was added and the mixture was stirred overnight at 50° C. The mixture was concentrated and purified by column chromatography (DCM/MeOH=1/0˜10/1, Silica-CS 20 g, 25 mL/min, silica gel, UV 254) to afford the product (143 mg, 84%). ESI-MS m/z calcd for [C₁₉H₂₀C₁N₅O₄S][M+H]⁺: 450.1; found: 450.0. ¹H NMR (400 MHz, Chloroform-d) δ 7.50-7.47 (m, 2H), 7.39-7.33 (m, 4H), 5.56 (s, 1H), 5.08 (d, J=9.2 Hz, 1H), 4.37 (t, J=9.2 Hz, 1H), 4.30 (s, 1H), 4.15 (d, J=12.4 Hz, 1H), 4.05 (d, J=12.4 Hz, 1H), 3.68 (s, 1H), 3.05 (br s, 1H), 2.38 (s, 3H).

### 2-{5-{4,6-O-Benzylidene-3-[(2-tert-butoxycarbonyl)hydrazinyl]-3-deoxy-β-D-galactopyranosyl}-3-methyl-1H-1,2,4-triazol-1-yl}-5-chlorothiazole

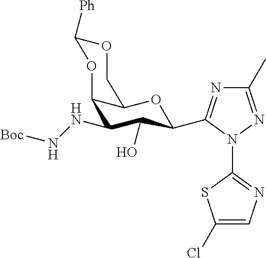

To a solution of 2-[5-(3-amino-4,6-O-benzylidene-3-deoxy-β-D-galactopyranosyl)-3-methyl-1H-1,2,4-triazol-1-yl]-5-chlorothiazole (123 mg, 0.27 mmol) in DCM (5 mL) N-tert-butyloxycarbonyl-3-(4-cyanophenyl)oxaziridine (101 mg, 0.41 mmol) was added and the mixture was stirred overnight at rt. The mixture was concentrated and purified by column chromatography (MeOH/DCM=0/1˜1/10, Silica-CS 12 g, 20 mL/min, silica gel, UV 254) to afford the product (59 mg, 38%). ESI-MS m/z calcd for [C₂₄H₂₉ClN₆O₆S] [M+H]⁺: 565.2; found: 565.2. ¹H NMR (400 MHz, Chloroform-d) δ 7.50-7.47 (m, 2H), 7.39-7.33 (m, 4H), 6.78 (br s, 1H), 5.57 (s, 1H), 5.23 (dd, J=10.0, 6.0 Hz, 1H), 4.65 (t, J=9.2 Hz, 1H), 4.49-4.48 (m, 1H), 4.17 (d, J=12.8 Hz, 1H), 4.05 (d, J=11.6 Hz, 1H), 3.67 (s, 1H), 3.31 (d, J=9.6 Hz, 1H), 2.41 (s, 3H), 1.45 (s, 9H).

### Intermediate 34

### 1,3-Benzothiazol-6-ylhydrazine

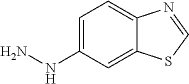 [SMILES: NNc1ccc2ncsc2c1]

To a cooled (−5° C.) solution of 1,3-benzothiazol-6-amine (2.00 g, 13.3 mmol) in water (30 mL) HCl (30 mL, 33% w/w) was added. A solution of NaNO₂ (919 mg, 13.3 mmol) in water (6 mL) was added dropwise at −5° C. and the mixture was stirred 1 h at −5° C. Tin(II) chloride dihydrate (6.01 g, 26.6 mmol) dissolved in concentrated HCl (10 mL) was added dropwise at −5° C. and the resulting mixture was stirred 2 h at −5° C. NaOH (100 mL, 10% w/w) was added dropwise at 0° C. to adjust pH to 10. Water (50 mL) and DCM (100 mL) were added. The aqueous phase was extracted with DCM (2×50 mL). The combined organic phases were washed with water (50 mL) and brine (2×50 mL), dried over Na₂SO₄, evaporated and purified by column chromatography (PE/EtOAc=1/0˜0/1, silica-CS 40 g 40 mL/min, silica gel, UV 254 nm) to afford the product (600 mg, 27%). ESI-MS m/z calcd for [C₇H₇N₃S] [M+H]⁺: 166.0; found: 166.1. ¹H NMR (400 MHz, DMSO-d₆) δ 8.92 (s, 1H), 7.75 (d, J=8.8 Hz, 1H), 7.39 (d, J=2.0 Hz, 1H), 7.07 (br s, 1H), 6.93 (dd, J=8.8, 2.0 Hz, 1H), 4.15 (br s, 2H).

### 6-[5-(3-Azido-4,6-O-benzylidene-3-deoxy-β-D-galactopyranosyl)-3-methyl-1H-1,2,4-triazol-1-yl]benzothiazole

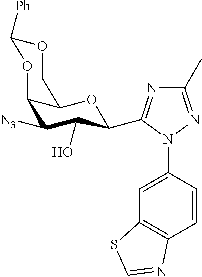

To a solution of 2,6-anhydro-4-azido-5,7-O-benzylidene-4-deoxy-D-glycero-L-manno-N-[1-(dimethylamino)ethylidene]heptonamide (1.0 g, 2.57 mmol) in 1,4-dioxane (15 mL) and acetic acid (5 mL) 1,3-benzothiazol-6-ylhydrazine (420 mg, 2.54 mmol) was added and the mixture was stirred 3 h at 60° C. The mixture was concentrated and the pH was adjusted to 8 by addition of aq NaHCO₃. The mixture was extracted with DCM (3×50 mL), and the combined organic phases were dried over Na₂SO₄ and evaporated. The residue was purified by column chromatography (EtOAc/PE=1/10˜1/0, Silica-CS 40 g, 40 m/min, silica gel, UV 254) to afford the product (720 mg, 58%). ESI-MS m/z calcd for [C₂₃H₂₁N₇O₄S] [M+H]⁺: 492.1; found: 492.1. ¹H NMR (400 MHz, Chloroform-d) δ 9.03 (s, 1H), 8.13 (d, J=2.0 Hz, 1H), 8.10 (d, J=8.8 Hz, 1H), 7.63 (dd, J=8.8, 2.0 Hz, 1H), 7.43-7.40 (m, 2H), 7.30-7.27 (m, 3H), 5.50 (s, 1H), 4.76 (t, J=9.6 Hz, 1H), 4.37 (d, J=9.2 Hz, 1H), 4.22 (d, J=2.8 Hz, 1H), 3.94 (s, 2H), 3.40-3.36 (m, 2H), 2.41 (s, 3H).

### 6-[5-(3-Amino-4,6-O-benzylidene-3-deoxy-β-D-galactopyranosyl)-3-methyl-1H-1,2,4-triazol-1-yl]benzothiazole

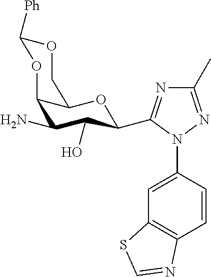

To a solution of 6-[5-(3-azido-4,6-O-benzylidene-3-deoxy-β-D-galactopyranosyl)-3-methyl-1H-1,2,4-triazol-1-yl]benzothiazole (500 mg, 1.02 mmol) in THF (20 mL) and water (1 mL) triphenylphosphine (1.33 g, 5.09 mmol) was added and the mixture was stirred overnight at 50° C. The mixture was concentrated and purified by column chromatography (DCM/MeOH=1/0˜10/1, Silica-CS 40 g, 50 mL/min, silica gel, UV 254) to give the product (320 mg, 68%). ESI-MS m/z calcd for [C₂₃H₂₃N₅O₄S] [M+H]⁺: 466.1; found: 466.0. ¹H NMR (400 MHz, DMSO-d₆) δ 9.52 (s, 1H), 8.41 (d, J=2.0 Hz, 1H), 8.14 (d, J=8.8 Hz, 1H), 7.77 (dd, J=8.8, 2.0 Hz, 1H), 7.48-7.45 (m, 2H), 7.43-7.39 (m, 3H), 5.62 (s, 1H), 5.19 (d, J=5.6 Hz, 1H), 4.31 (d, J=9.6 Hz, 1H), 4.13-4.05 (m, 3H), 3.91-3.85 (m, 1H), 3.66 (s, 1H), 2.69 (dd, J=9.6, 3.2 Hz, 1H), 2.36 (s, 3H), 1.72 (br s, 2H).

### 6-{5-{4,6-O-Benzylidene-3-[(2-tert-butoxycarbonyl)hydrazinyl]-3-deoxy-β-D-galactopyranosyl}-3-methyl-1H-1,2,4-triazol-1-yl}benzothiazole

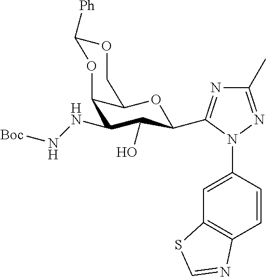

To a solution of 6-[5-(3-amino-4,6-O-benzylidene-3-deoxy-β-D-galactopyranosyl)-3-methyl-1H-1,2,4-triazol-1-yl]benzothiazole (300 mg, 0.64 mmol) in DCM (10 mL) N-tert-butyloxycarbonyl-3-(4-cyanophenyl)oxaziridine (190 mg, 0.77 mmol) was added and the mixture was stirred overnight at rt. The mixture was concentrated and purified by column chromatography (DCM/EtOAc=10/1˜0/1, Silica-CS 20 g, 40 mL/min, silica gel, UV 254) to afford the product (240 mg, 64%). ESI-MS m/z calcd for [C₂₈H₃₄N₆O₆S] [M+H]⁺: 581.2; found: 580.9. ¹H NMR (400 MHz, DMSO-d₆) δ 9.51 (s, 1H), 8.40 (d, J=2.0 Hz, 1H), 8.36 (br s, 1H), 8.12 (d, J=8.8 Hz, 1H), 7.76 (dd, J=8.4, 2.0 Hz, 1H), 7.53-7.51 (m, 2H), 7.42-7.38 (m, 3H), 5.57 (s, 1H), 5.21 (d, J=5.2 Hz, 1H), 4.58 (br s, 1H), 4.42 (d, J=9.2 Hz, 1H), 4.21 (d, J=2.8 Hz, 1H), 4.12-4.06 (m, 3H), 3.67 (s, 1H), 2.91 (d, J=10.0 Hz, 1H), 2.36 (s, 3H), 1.37 (s, 9H).

### Intermediate 35

### 2-Ethyl-1,3-benzothiazole

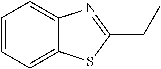

To a solution of 2-aminobenzenethiol (3.00 g, 24.0 mmol) in DCM (10 mL) 1,1,1-triethoxypropane (5.07 g, 28.8 mmol) and boron trifluoride diethyl etherate (0.30 mL, 2.40 mmol) were added and the mixture was stirred overnight at rt. The mixture was washed with aq NaHCO₃ and brine. The organic phase was dried over Na₂SO₄, concentrated, and purified by column chromatography (PE/EtOAc=1/0˜3/1, silica-CS 40 g, 40 m/min, silica gel, UV 254 nm) to afford the product (2.30 g, 59%). ESI-MS m/z calcd for [C₉H₉NS] [M+H]⁺: 164.0; found: 164.2. ¹H NMR (400 MHz, Chloroform-d) δ 7.90 (d, J=8.4 Hz, 1H), 7.77 (d, J=8.0 Hz, 1H), 7.40-7.36 (m, 1H), 7.30-7.26 (m, 1H), 3.09 (q, J=7.6 Hz, 2H), 1.41 (t, J=7.6 Hz, 3H).

### 2-Ethyl-6-nitro-1,3-benzothiazole

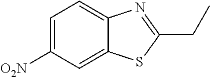

To a cooled (0° C.) solution of 2-ethyl-1,3-benzothiazole (2.30 g, 14.1 mmol) in concentrated H₂SO₄ (10 mL) fuming HNO₃ (4.44 g, 70.4 mmol) was added dropwise, and the mixture was stirred 2 h at 0° C. The mixture was concentrated and poured into ice-water. DCM (100 mL) was added, and the phases were separated. The organic phase was washed with aq NaHCO₃ and brine, dried over Na₂SO₄ and evaporated. The residue was purified by column chromatography (PE/EtOAc=1/0˜0/1, silica-CS 40 g, 40 mL/min, silica gel, UV 254 nm) to afford the product (2.00 g, 68%). ESI-MS m/z calcd for [C₉HsN₂O₂S] [M+H]⁺: 209.0; found: 209.1. ¹H NMR (400 MHz, Chloroform-d) δ 8.75 (d, J=2.4 Hz, 1H), 8.17 (dd, J=8.8, 2.0 Hz, 1H), 7.90 (d, J=8.8 Hz, 1H), 3.14 (q, J=7.6 Hz, 2H), 1.44 (t, J=7.6 Hz, 3H). 2-Ethyl-1,3-benzothiazol-6-amine

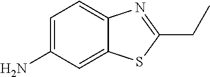

To a solution of 2-ethyl-6-nitro-1,3-benzothiazole (2.00 g, 9.60 mmol) in MeOH (30 mL), Pd/C (5% wt Pd, 50% wt H₂O, 102 mg, 0.024 mmol) was added and the mixture was stirred overnight at rt under a hydrogen atmosphere. The mixture was filtered, concentrated, and purified by column chromatography (PE/EtOAc=1/0˜1/1, Silica-CS 40 g, 20 mL/min, silica gel, UV 254) to afford the product (1.30 g, 76%). ESI-MS m/z calcd for [C₉H₁₀N₂S] [M+H]⁺: 179.1; found: 179.1. ¹H NMR (400 MHz, DMSO-d₆) δ 7.54 (d, J=8.4 Hz, 1H), 7.02 (d, J=2.4 Hz, 1H), 6.72 (dd, J=8.4, 2.0 Hz, 1H), 5.29 (br s, 2H). 2.98 (q, J=7.6 Hz, 2H), 1.31 (t, J=7.6 Hz, 3H).

### (2-Ethyl-1,3-benzothiazol-6-yl)hydrazine

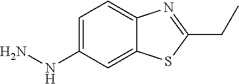

To a cooled (−5° C.) solution of 2-ethyl-1,3-benzothiazol-6-amine (700 mg, 3.93 mmol) in water (10 mL) HCl (10 mL, 33% w/w) and acetic acid (10 mL) were added. A solution of NaNO₂ (325 mg, 4.71 mmol) in water (2 mL) was added dropwise at −5° C. and the resulting mixture was stirred 1 h at −5° C. Tin(II) chloride dihydrate (1.77 g, 7.85 mmol) dissolved in concentrated HCl (5 mL) was added dropwise at −5° C. and the resulting mixture was stirred 2 h at −5° C. NaOH (100 mL, 10% w/w) was added dropwise at 0° C. to adjust pH to 10. Water (50 mL) and DCM (100 mL) were added. The aqueous phase was extracted with DCM (2×50 mL), and the combined organic phases were washed with water (50 mL) and brine (2×50 mL), dried over Na₂SO₄ and evaporated. The residue was purified by column chromatography (DCM/EtOAc=1/0˜0/1, silica-CS 40 g 20 m/min, silica gel, UV 254 nm) to afford the product (260 mg, 34%). ESI-MS m/z calcd for [C₉H₁₁N₃S] [M+H]⁺: 194.1; found: 194.1. ¹H NMR (400 MHz, DMSO-d₆) δ 7.60 (d, J=8.8 Hz, 1H), 7.29 (d, J=2.0 Hz, 1H), 6.95 (br s, 1H), 6.86 (dd, J=8.8, 2.0 Hz, 1H), 4.06 (br s, 2H), 3.00 (q, J=7.6 Hz, 2H), 1.32 (t, J=7.6 Hz, 3H).

### 6-[5-(3-Azido-4,6-O-benzylidene-3-deoxy-β-D-galactopyranosyl)-3-methyl-1H-1,2,4-triazol-1-yl]-2-ethylbenzothiazole

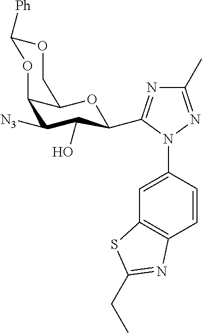

To a solution of 2,6-anhydro-4-azido-5,7-O-benzylidene-4-deoxy-D-glycero-L-manno-N-[1-(dimethylamino)ethylidene]heptonamide (380 mg, 0.98 mmol) in 1,4-dioxane (10 mL) and acetic acid (3 mL) (2-ethyl-1,3-benzothiazol-6-yl)hydrazine (207 mg, 1.07 mmol) was added and the mixture was stirred 2 h at 60° C. The mixture was concentrated, DCM (100 mL) was added and the mixture was washed with aq NaHCO₃ (5%) and brine. The organic phase was dried over Na₂SO₄, evaporated, and purified by column chromatography (EtOAc/PE=0/1˜1/0, Silica-CS 40 g, 40 mL/min, silica gel, UV 254) to afford the product (380 mg, 75%). ESI-MS m/z calcd for [C₂₅H₂₅N₇O₄S] [M+H]⁺: 520.2; found: 520.0. ¹H NMR (400 MHz, Chloroform-d) δ 8.00 (d, J=1.6 Hz, 1H), 7.93 (d, J=8.8 Hz, 1H), 7.54 (dd, J=8.8, 2.0 Hz, 1H), 7.45-7.40 (m, 2H), 7.32-7.28 (m, 3H), 5.50 (s, 1H), 4.77 (t, J=9.2 Hz, 1H), 4.36 (d, J=9.6 Hz, 1H), 4.22 (d, J=2.8 Hz, 1H), 3.98-3.91 (m, 2H), 3.39-3.35 (m, 2H), 3.12 (q, J=7.6 Hz, 2H), 2.42 (s, 3H), 1.43 (t, J=7.6 Hz, 3H).

### 6-[5-(3-Amino-4,6-O-benzylidene-3-deoxy-β-D-galactopyranosyl)-3-methyl-1H-1,2,4-triazol-1-yl]-2-ethylbenzothiazole

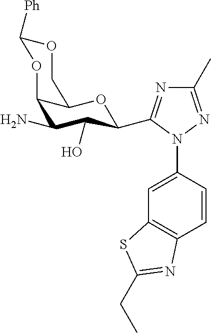

To a solution of 6-[5-(3-azido-4,6-O-benzylidene-3-deoxy-β-D-galactopyranosyl)-3-methyl-1H-1,2,4-triazol-1-yl]-2-ethylbenzothiazole (380 mg, 0.73 mmol) in MeOH (10 mL), Pd/C (5% wt Pd, 50% wt H₂O, 38.9 mg, 0.009 mmol) was added and the mixture was stirred overnight at rt under a hydrogen atmosphere. The mixture was filtered through a celite pad, concentrated, and purified by column chromatography (DCM/MeOH=1/0˜10/1, Silica-CS 20 g, 20 mL/min, silica gel, UV 254) to afford the product (250 mg, 69%). ESI-MS m/z calcd for [C₂₅H₂₇N₅O₄S] [M+H]⁺: 494.2; found: 494.0. ¹H NMR (400 MHz, DMSO-d₆) δ 8.28 (d, J=2.4 Hz, 1H), 8.00 (d, J=8.8 Hz, 1H), 7.68 (dd, J=8.8, 2.4 Hz, 1H), 7.47-7.45 (m, 2H), 7.43-7.39 (m, 3H), 5.61 (s, 1H), 5.19 (d, J=5.2 Hz, 1H), 4.29 (d, J=9.6 Hz, 1H), 4.12-4.04 (m, 3H), 3.91-3.85 (m, 1H), 3.64 (s, 1H), 3.16 (q, J=7.6 Hz, 2H), 2.69 (dd, J=9.6, 3.2 Hz, 1H), 2.35 (s, 3H), 1.83 (br s, 2H), 1.39 (t, J=7.6 Hz, 3H).

### 6-{5-{4,6-O-Benzylidene-3-[(2-tert-butoxycarbonyl)hydrazinyl]-3-deoxy-β-D-galactopyranosyl}-3-methyl-1H-1,2,4-triazol-1-yl}-2-ethylbenzothiazole

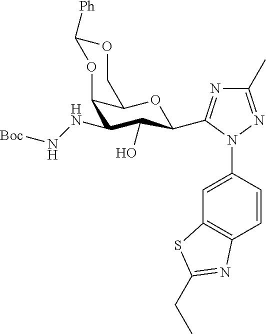

To a solution of 6-[5-(3-amino-4,6-O-benzylidene-3-deoxy-β-D-galactopyranosyl)-3-methyl-1H-1,2,4-triazol-1-yl]-2-ethylbenzothiazole (150 mg, 0.30 mmol) in DCM (10 mL) N-tert-butyloxycarbonyl-3-(4-cyanophenyl)oxaziridine (89.8 mg, 0.37 mmol) was added and the mixture was stirred overnight at rt. The mixture was concentrated and purified by column chromatography (DCM/EtOAc=1/0˜0/1, Silica-CS 20 g, 30 m/min, silica gel, UV 254) to afford the product (100 mg, 54%). ESI-MS m/z calcd for [C₃₀H₃₆N₆O₆S] [M+H]⁺: 609.2; found: 609.0. ¹H NMR (400 MHz, DMSO-d₆) δ 8.42 (br s, 1H), 8.28 (d, J=2.0 Hz, 1H), 7.98 (d, J=8.8 Hz, 1H), 7.67 (dd, J=8.4, 2.0 Hz, 1H), 7.53-7.50 (m, 2H), 7.42-7.38 (m, 3H), 5.57 (s, 1H), 5.25 (br s, 1H), 4.39 (d, J=9.6 Hz, 1H), 4.21 (d, J=2.8 Hz, 1H), 4.12-4.08 (m, 3H), 3.65 (s, 1H), 3.16 (q, J=7.6 Hz, 2H), 2.92 (dd, J=10.4, 2.8 Hz, 1H), 2.36 (s, 3H), 1.39 (t, J=7.6 Hz, 3H), 1.38 (s, 9H).

### Intermediate 36

### [2-(Trifluoromethyl)-1,3-benzothiazol-6-yl]hydrazine

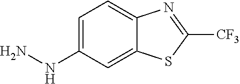 [SMILES: NNc1ccc2nc(C(F)(F)F)sc2c1]

To a solution of 2-(trifluoromethyl)-1,3-benzothiazol-6-amine (200 mg, 0.92 mmol) in water (1 mL) HCl (3 mL, 33% w/w) and acetic acid (1 mL) were added. A solution of NaNO₂ (76 mg, 1.10 mmol) in water (2 mL) was added dropwise at −10° C. and the resulting mixture was stirred 2 h at −10° C. Tin(II) chloride dihydrate (414 mg, 1.83 mmol) dissolved in concentrated HCl (2 mL, 33% w/w) was added dropwise at −10° C. and the resulting mixture was stirred 2 h at −10° C. Aqueous NaHCO₃ was added at 0° C. to adjust pH to 7. Water (20 mL) and DCM (20 mL) were added. The aqueous phase was extracted with DCM (2×10 mL), and the combined organic phases were washed with brine (5 mL), dried over Na₂SO₄ and evaporated. The residue was purified by column chromatography (PE/EtOAc=1/0˜5/1, silica-CS 12 g, 12 m/min, silica gel, UV 254 nm) to afford the product (98 mg, 46%). ESI-MS m/z calcd for [C₈H₆F₃N₃S][M+H]⁺: 234.0; found: 234.0. ¹H NMR (400 MHz, Chloroform-d) δ 7.96 (d, J=8.0 Hz, 1H), 7.42 (s, 1H), 6.99 (d, J=7.6 Hz, 1H).

### 6-[5-(3-Azido-4,6-O-benzylidene-3-deoxy-β-D-galactopyranosyl)-3-methyl-1H-1,2,4-triazol-1-yl]-2-(trifluoromethyl)benzothiazole

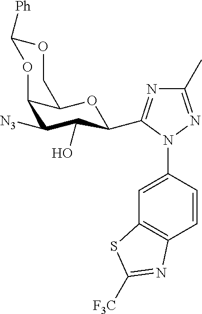

To a solution of 2,6-anhydro-4-azido-5,7-O-benzylidene-4-deoxy-D-glycero-L-manno-N-[1-(dimethylamino)ethylidene]heptonamide (150 mg, 0.39 mmol) in 1,4-dioxane (3 mL) and acetic acid (1 mL) [2-(trifluoromethyl)-1,3-benzothiazol-6-yl]hydrazine (98 mg, 0.42 mmol) was added and the mixture was stirred 3 h at 60° C. The mixture was concentrated, DCM (10 mL) was added and the mixture was washed with aq NaHCO₃ (10 mL) and brine (10 mL). The organic phase was dried over Na₂SO₄, evaporated, and purified by column chromatography (PE/EtOAc=4/1˜0/1, Silica-CS 12 g, 15 mL/min, silica gel, UV 254) to afford the product (115 mg, 54%). ESI-MS m/z calcd for [C₂₄H₂₀F₃N₇O₄S] [M+H]⁺: 560.1; found: 559.9. ¹H NMR (400 MHz, Chloroform-d) δ 8.14 (d, J=1.6 Hz, 1H), 8.09 (d, J=8.8 Hz, 1H), 7.69 (dd, J=8.8, 1.6 Hz, 1H), 7.37-7.34 (m, 2H), 7.25-7.22 (m, 3H), 5.44 (s, 1H), 4.61 (t, J=9.6 Hz, 1H), 4.37 (d, J=9.2 Hz, 1H), 4.18-4.10 (m, 2H), 3.98-3.95 (m, 1H), 3.89-3.86 (m, 1H), 3.32-3.28 (m, 2H), 2.33 (s, 3H).

### 6-[5-(3-Amino-4,6-O-benzylidene-3-deoxy-β-D-galactopyranosyl)-3-methyl-1H-1,2,4-triazol-1-yl]-2-(trifluoromethyl)benzothiazole

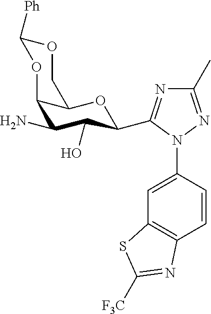

To a solution of 6-[5-(3-azido-4,6-O-benzylidene-3-deoxy-β-D-galactopyranosyl)-3-methyl-1H-1,2,4-triazol-1-yl]-2-(trifluoromethyl)benzothiazole (480 mg, 0.89 mmol) in THF/water (6 mL, 5:1) triphenylphosphine (270 mg, 1.03 mmol) was added and the mixture was stirred overnight at 50° C. The mixture was concentrated and purified by column chromatography (DCM/MeOH=1/0˜10/1, Silica-CS 12 g, 40 mL/min, silica gel, UV 254) to afford the product (105 mg, 85%). ESI-MS m/z calcd for [C₂₄H₂₂F₃N₅O₄S] [M+H]⁺: 534.1; found: 534.0. ¹H NMR (400 MHz, Chloroform-d) δ 8.27-8.23 (m, 2H), 7.83 (dd, J=8.4, 1.6 Hz, 1H), 7.46-7.44 (m, 2H), 7.34-7.33 (m, 3H), 5.53 (s, 1H), 4.55-4.32 (m, 3H), 4.07-3.99 (m, 3H), 3.49 (s, 1H), 3.08-3.07 (m, 1H), 2.37 (s, 3H).

### 6-{5-{4,6-O-Benzylidene-3-[(2-tert-butoxycarbonyl)hydrazinyl]-3-deoxy-β-D-galactopyranosyl}-3-methyl-1H-1,2,4-triazol-1-yl}-2-(trifluoromethyl)benzothiazole

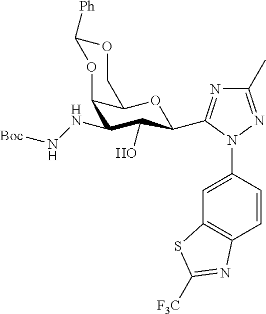

To a solution of 6-[5-(3-amino-4,6-O-benzylidene-3-deoxy-β-D-galactopyranosyl)-3-methyl-1H-1,2,4-triazol-1-yl]-2-(trifluoromethyl)benzothiazole (97 mg, 0.18 mmol) in DCM (5 mL) N-tert-butyloxycarbonyl-3-(4-cyanophenyl)oxaziridine (49.3 mg, 0.20 mmol) was added and the mixture was stirred overnight at rt. The mixture was concentrated and purified by column chromatography (PE/EtOAc=4/1˜0/1, Silica-CS 20 g, 30 mL/min, silica gel, UV 254) to afford the product (65 mg, 55%). ESI-MS m/z calcd for [C₂₉H₃₁F₃N₆O₆S] [M+H]⁺: 649.2; found: 648.8. ¹H NMR (400 MHz, Chloroform-d) δ 8.22 (d, J=2.0 Hz, 1H), 8.09 (d, J=8.8 Hz, 1H), 7.79 (dd, J=8.8, 2.0 Hz, 1H), 7.35-7.32 (m, 2H), 7.29-7.23 (m, 3H), 6.59 (s, 1H), 5.47 (s, 1H), 4.45 (d, J=9.2 Hz, 1H), 4.35 (t, J=9.6 Hz, 1H), 4.29 (d, J=2.4 Hz, 1H), 4.09 (d, J=12.8 Hz, 1H), 3.97 (dd, J=12.8, 1.2 Hz, 1H), 3.41 (s, 1H), 2.97-2.95 (m, 1H), 2.34 (s, 3H), 1.31 (s, 9H).

### Intermediate 37

### 5-Fluoro-2-methyl-6-nitro-1,3-benzothiazole

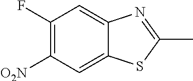

To a cooled (0° C.) solution of 5-fluoro-2-methyl-1,3-benzothiazole (2.37 g, 14.2 mmol) in H₂SO₄ (10.0 mL) KNO₃ (1.58 g, 15.6 mmol) was added slowly and the mixture was stirred 2 h at 0° C. The mixture was poured into cooled (0° C.) aq NaOH (10 M) and extracted with EtOAc (2×100 mL). The combined organic phases were dried over Na₂SO₄ and concentrated to give crude product (3 g), which was used without purification in the next step. ESI-MS m/z calcd for [C₈H₅FN₂O₂S] [M+H]⁺: 213.0; found: 213.0.

### 5-Fluoro-2-methyl-1,3-benzothiazol-6-amine

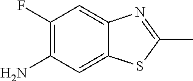

To a solution of crude 5-fluoro-2-methyl-6-nitro-1,3-benzothiazole (3 g) in EtOH/H₂O (120 mL, v/v 2:1) iron (1.74 g, 31.1 mmol) and NH₄Cl (7.56 g, 141 mmol) were added, and the mixture was stirred 1 h at 80° C. The mixture was filtered through a short silica column and washed by EtOH. The filtrate was concentrated, poured into water (100 mL) and extracted with EtOAc (2×100 mL). The combined organic phases were washed with brine (50 mL), dried over Na₂SO₄, concentrated, and purified by column chromatography (PE/EtOAc=1/˜3/1, Silica-CS 40 g, 50 m/min, silica gel, UV 254) to give the product (1.5 g, 58%). ESI-MS m/z calcd for [C₈H₇FN₂S] [M+H]⁺: 183.0; found: 183.1. ¹H NMR (400 MHz, DMSO-d₆) δ 7.55 (d, J=12.0 Hz, 1H), 7.22 (d, J=8.4 Hz, 1H), 5.36 (s, 2H), 2.68 (s, 3H).

### (5-Fluoro-2-methyl-1,3-benzothiazol-6-yl)hydrazine

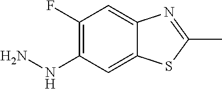

To a cooled (−5° C.) solution of 5-fluoro-2-methyl-1,3-benzothiazol-6-amine (1.40 g, 7.68 mmol) in concentrated HCl (20 mL) and water (20 mL) a solution of NaNO₂ (795 mg, 11.5 mmol) in water (5 mL) was added dropwise. The resulting mixture was stirred 1 h at 0° C. Tin(II) chloride dihydrate (3.81 g, 16.9 mmol) dissolved in concentrated HCl (8 mL) was added dropwise at 0° C. The resulting mixture was allowed to reach rt and was then stirred 3 h. The pH of the mixture was adjusted to 11 by addition of NaOH (1 M). The mixture was extracted with EtOAc (2×100 mL). The combined organic phases were dried over Na₂SO₄, evaporated, and purified by column chromatography (EtOAc/DCM=0˜1/10, Silica-CS 40 g, 50 mL/min, silica gel, UV 254) to afford the product (510 mg, 34%). ESI-MS m/z calcd for [C₈H₈FN₃S] [M+H]⁺: 198.0; found: 198.2. ¹H NMR (400 MHz, Methanol-d₄) δ 7.56 (d, J=8.0 Hz, 1H), 7.45 (d, J=12.0 Hz, 1H), 2.74 (s, 3H).

### 6-[5-(3-Azido-4,6-O-benzylidene-3-deoxy-β-D-galactopyranosyl)-3-methyl-1H-1,2,4-triazol-1-yl]-5-fluoro-2-methylbenzothiazole

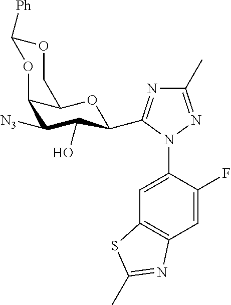

To a solution of 2,6-anhydro-4-azido-5,7-O-benzylidene-4-deoxy-D-glycero-L-manno-N-[1-(dimethylamino)ethylidene]heptonamide (660 mg, 1.69 mmol) in 1,4-dioxane (10 mL) and acetic acid (5 mL) (5-fluoro-2-methyl-1,3-benzothiazol-6-yl)hydrazine (368 mg, 1.86 mmol) was added and the mixture was stirred 2 h at 60° C. The mixture was concentrated and purified by column chromatography (EtOAc/PE=0/1˜1/1, Silica-CS 20 g, 25 mL/min, silica gel, UV 254) to afford the product (290 mg, 33%). ESI-MS m/z calcd for [C₂₄H₂₂FN₇O₄S] [M+H]⁺: 524.1; found: 524.0. ¹H NMR (400 MHz, Chloroform-d) δ 7.98 (d, J=6.8 Hz, 1H), 7.72 (d, J=10.8 Hz, 1H), 7.42-7.36 (m, 2H), 7.35-7.31 (m, 3H), 5.46 (s, 1H), 4.67-4.55 (m, 2H), 4.22 (d, J=2.8 Hz, 1H), 3.84 (dd, J=12.6, 1.4 Hz, 1H), 3.56-3.47 (m, 2H), 3.37 (s, 1H), 2.86 (s, 3H), 2.48 (s, 3H).

### 6-[5-(3-Amino-4,6-O-benzylidene-3-deoxy-β-D-galactopyranosyl)-3-methyl-1H-1,2,4-triazol-1-yl]-5-fluoro-2-methylbenzothiazole

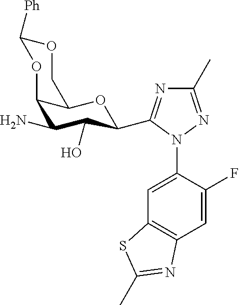

To a solution of 6-[5-(3-azido-4,6-O-benzylidene-3-deoxy-β-D-galactopyranosyl)-3-methyl-1H-1,2,4-triazol-1-yl]-5-fluoro-2-methylbenzothiazole (290 mg, 0.55 mmol) in THF/water (12 mL, 5:1) triphenylphosphine (726 mg, 2.77 mmol) was added and the mixture was stirred overnight at 50° C. The mixture was concentrated and purified by column chromatography (DCM/MeOH=1/0˜10/1, Silica-CS 40 g, 50 mL/min, silica gel, UV 254) to give the product (147 mg, 53%). ESI-MS m/z calcd for [C₂₄H₂₄FN₅O₄S] [M+H]⁺: 498.2; found: 498.0. ¹H NMR (400 MHz, Chloroform-d) δ 7.99 (d, J=6.8 Hz, 1H), 7.74 (d, J=10.8 Hz, 1H), 7.42-7.27 (m, 5H), 5.45 (s, 1H), 4.42 (d, J=9.2 Hz, 1H), 4.21-4.10 (m, 2H), 3.86 (d, J=12.8 Hz, 1H), 3.61 (d, J=12.4 Hz, 1H), 3.35 (s, 1H), 2.95 (d, J=8.4 Hz, 1H), 2.86 (s, 3H), 2.39 (s, 3H).

### 6-{5-{4,6-O-Benzylidene-3-[(2-tert-butoxycarbonyl)hydrazinyl]-3-deoxy-β-D-galactopyranosyl}-3-methyl-1H-1,2,4-triazol-1-yl}-5-fluoro-2-methylbenzothiazole

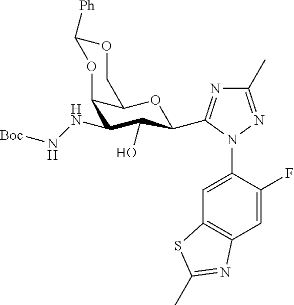

To a solution of 6-[5-(3-amino-4,6-O-benzylidene-3-deoxy-β-D-galactopyranosyl)-3-methyl-1H-1,2,4-triazol-1-yl]-5-fluoro-2-methylbenzothiazole (147 mg, 0.30 mmol) in DCM (5 mL) N-tert-butyloxycarbonyl-3-(4-cyanophenyl)oxaziridine (87.3 mg, 0.36 mmol) was added and the mixture was stirred overnight at rt. The mixture was concentrated and purified by column chromatography (MeOH/DCM=0˜1/10, Silica-CS 12 g, 20 m/min, silica gel, UV 254) to afford the product (115 mg, 64%). ESI-MS m/z calcd for [C₂₉H₃₃FN₆O₆S] [M+H]⁺: 613.2; found: 613.0. ¹H NMR (400 MHz, Chloroform-d) δ 8.00 (d, J=6.8 Hz, 1H), 7.72 (d, J=10.8 Hz, 1H), 7.39-7.26 (m, 5H), 6.72 (br s, 1H), 5.43 (s, 1H), 4.59 (d, J=8.8 Hz, 1H), 4.41-4.32 (m, 2H), 3.87 (d, J=12.4 Hz, 1H), 3.66 (d, J=12.4 Hz, 1H), 3.38 (s, 1H), 3.20 (d, J=8.4 Hz, 1H), 2.84 (s, 3H), 2.48 (s, 3H), 1.45 (s, 9H).

### Intermediate 38

### N-(2-Bromo-3-fluorophenyl)acetamide

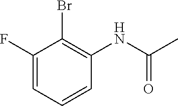

To a solution of 2-bromo-3-fluoroaniline (10.0 g, 52.6 mmol) in DMF (50 mL) acetic anhydride (10.0 mL) was added and the mixture was stirred overnight at rt. The mixture was concentrated and partitioned between EtOAc (40 mL) and aq NaHCO₃ (30 mL). The organic phase was dried over Na₂SO₄, evaporated, and purified by column chromatography (PE/EtOAc=8/1, Silica-CS 80 g, 40 m/min, silica gel, UV 254) to afford the product (10.5 g, 86%). ¹H NMR (400 MHz, Chloroform-d) δ 8.18 (d, J=8.0 Hz, 1H), 7.65 (s, 1H), 7.32-7.26 (m, 1H), 6.92-6.87 (m, 1H), 2.26 (s, 3H).

### N-(2-Bromo-3-fluorophenyl)thioacetamide

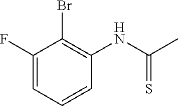

To a solution of N-(2-bromo-3-fluorophenyl)acetamide (10.5 g, 45.2 mmol) in THF (50 mL) Lawesson's reagent (12.8 g, 31.7 mmol) was added and the mixture was stirred 24 h at rt. The mixture was concentrated and purified by column chromatography (EtOAc/PE=1/8, Silica-CS 80 g, 40 mL/min, silica gel, UV 254) to give the product (9.6 g, 86%). ¹H NMR (400 MHz, Chloroform-d) δ 8.74 (s, 1H), 8.34 (d, J=8.0 Hz, 1H), 7.39-7.34 (m, 1H), 7.09-7.05 (m, 1H), 2.81 (s, 3H).

### 7-Fluoro-2-methyl-1,3-benzothiazole

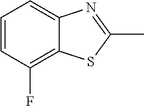

A nitrogen purged solution of N-(2-bromo-3-fluorophenyl)thioacetamide (6.0 g, 24.2 mmol) potassium tert-butoxide (4.07 g, 36.3 mmol), (2-biphenyl)di-tert-butylphosphine (397 mg, 1.33 mmol) and Pd₂(dba)₃ in 1,4-dioxane (100 mL) was stirred overnight at 80° C. The mixture was cooled to rt, filtered over celite and concentrated. The residue was purified by column chromatography (EtOAc/PE=1/10, Silica-CS 40 g, 40 m/min, silica gel, UV 254) to afford the product (3.10 g, 77%). ¹H NMR (400 MHz, Chloroform-d) δ 7.77 (d, J=8.4 Hz, 1H), 7.43-7.38 (m, 1H), 7.07 (t, J=8.4 Hz, 1H), 2.86 (s, 3H).

### 7-Fluoro-2-methyl-1,3-benzothiazol-6-amine

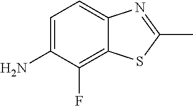

To a solution of 7-fluoro-2-methyl-1,3-benzothiazole (3.20 g, 19 mmol) in concentrated H₂SO₄ (25 mL) fuming nitric acid (1.5 mL) was added dropwise over several minutes at 45° C. with vigorous stirring. The mixture was stirred 30 min at 45° C. The mixture was poured into ice-water and extracted with DCM (3×20 mL). The combined organic phases were washed with aq NaHCO₃ (20 mL) and brine (20 mL), dried over Na₂SO₄ and evaporated. The residue was purified by column chromatography (EtOAc/PE=1/10, Silica-CS 40 g, 30 m/min, silica gel, UV 254). The obtained material was dissolved in EtOH/water (10 mL, 3:1). Iron (758 mg, 13.6 mmol) and NH₄Cl (726 mg, 13.6 mmol) were added and the mixture was stirred 2 h at 85° C. The mixture was filtered and extracted with DCM (3×30 mL). The combined organic phases were dried over MgSO₄, evaporated, and purified by purified by column chromatography (PE/EtOAc=10/1˜8/1, Silica-CS 20 g, 25 mL/min, silica gel, UV 254) to afford the product (355 mg, 10%). ESI-MS m/z calcd for [C₈H₇FN₂S] [M+H]⁺: 183.0; found: 183.1. ¹H NMR (400 MHz, Chloroform-d) δ 7.55 (d, J=8.8 Hz, 1H), 6.92 (t, J=8.8 Hz, 1H), 2.79 (s, 3H).

### (7-Fluoro-2-methyl-1,3-benzothiazol-6-yl)hydrazine

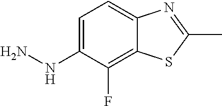

To a cooled (−5° C.) solution of 7-fluoro-2-methyl-1,3-benzothiazol-6-amine (200 mg, 1.10 mmol) in water (4 mL) HCl (4 mL, 33% wt) was added followed by dropwise addition of a solution of NaNO₂ (90.9 mg, 1.32 mmol) in water (1 mL). The mixture was stirred 1 h at −5° C. Tin(II) chloride dihydrate (416 mg, 2.20 mmol) dissolved in concentrated HCl (2 mL) was added dropwise at −5° C. The resulting mixture was stirred 2 h at −5° C. before aqueous NaOH (10% wt) was added to adjust pH to 9. Water (10 mL) and DCM (10 mL) were added. The aqueous phase was extracted with EtOAc (2×10 mL). The combined organic phases were washed with brine (10 mL), dried over Na₂SO₄, evaporated, and purified by column chromatography (PE/EtOAc=1/0˜3/1, Silica-CS 20 g, 20 m/min, silica gel, UV 254) to afford the product (105 mg, 49%). ESI-MS m/z calcd for [C₈H₈FN₃S] [M+H]⁺: 198.0; found: 198.1. ¹H NMR (400 MHz, DMSO-d₆) δ 7.58 (d, J=8.8 Hz, 1H), 7.38 (t, J=8.8 Hz, 1H), 6.86 (s, 1H), 4.16 (s, 2H), 2.72 (s, 3H).

### 6-[5-(3-Azido-4,6-O-benzylidene-3-deoxy-β-D-galactopyranosyl)-3-methyl-1H-1,2,4-triazol-1-yl]-7-fluoro-2-methylbenzothiazole

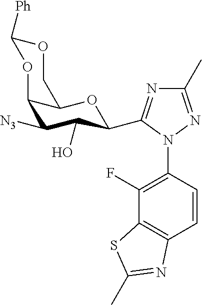

To a solution of 2,6-anhydro-4-azido-5,7-O-benzylidene-4-deoxy-D-glycero-L-manno-N-[1-(dimethylamino)ethylidene]heptonamide (200 mg, 0.51 mmol) in 1,4-dioxane (3 mL) and acetic acid (1 mL) (7-fluoro-2-methyl-1,3-benzothiazol-6-yl)hydrazine (105 mg, 0.53 mmol) was added and the mixture was stirred 3 h at 60° C. The mixture was concentrated and DCM (10 mL) was added. The mixture was washed with aq NaHCO₃ and brine, dried over Na₂SO₄, evaporated, and purified by column chromatography (PE/EtOAc=1/0˜1/3, Silica-CS 12 g, 20 mL/min, silica gel, UV 254) to afford the product (146 mg, 43%). ESI-MS m/z calcd for [C₂₄H₂₂FN₇O₄S] [M+H]⁺: 524.1; found: 523.8.

### 6-[5-(3-Amino-4,6-O-benzylidene-3-deoxy-β-D-galactopyranosyl)-3-methyl-1H-1,2,4-triazol-1-yl]-7-fluoro-2-methylbenzothiazole

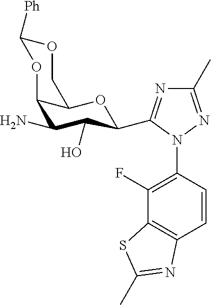

To a solution of 6-[5-(3-azido-4,6-O-benzylidene-3-deoxy-β-D-galactopyranosyl)-3-methyl-1H-1,2,4-triazol-1-yl]-7-fluoro-2-methylbenzothiazole (146 mg, 0.22 mmol) in THF/water (6 mL, 5:1) triphenylphosphine (293 mg, 1.12 mmol) was added and the mixture was stirred 16 h at 50° C. The mixture was concentrated and purified by column chromatography (DCM/MeOH=1/0˜10/1, Silica-CS 12 g, 20 mL/min, silica gel, UV 254) to give the product (97 mg, 87%). ESI-MS m/z calcd for [C₂₄H₂₄FN₅O₄S] [M+H]⁺: 498.2; found: 498.0. ¹H NMR (400 MHz, Chloroform-d) δ 7.81 (d, J=8.4 Hz, 1H), 7.59 (dd, J=8.4, 7.2 Hz, 1H), 7.44-7.40 (m, 2H), 7.38-7.26 (m, 3H), 5.46 (s, 1H), 4.35 (d, J=9.2 Hz, 1H), 4.28-4.21 (m, 2H), 3.87 (dd, J=12.4, 1.6 Hz, 1H), 3.71 (d, J=12.4 Hz, 1H), 3.32 (s, 1H), 2.94 (dd, J=9.6, 3.6 Hz, 1H), 2.89 (s, 3H), 2.39 (s, 3H).

### 6-{5-{4,6-O-Benzylidene-3-[(2-tert-butoxycarbonyl)hydrazinyl]-3-deoxy-β-D-galactopyranosyl}-3-methyl-1H-1,2,4-triazol-1-yl}-7-fluoro-2-methylbenzothiazole

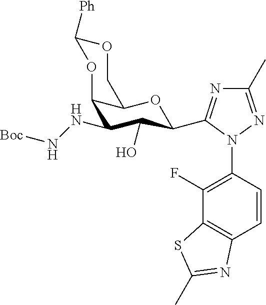

To a solution of 6-[5-(3-amino-4,6-O-benzylidene-3-deoxy-β-D-galactopyranosyl)-3-methyl-1H-1,2,4-triazol-1-yl]-7-fluoro-2-methylbenzothiazole (97 mg, 0.20 mmol) in DCM (2.0 mL) N-tert-butyloxycarbonyl-3-(4-cyanophenyl)oxaziridine (52.8 mg, 0.21 mmol) was added and the mixture was stirred overnight at rt. The mixture was concentrated and purified by column chromatography (PE/EtOAc=4/1˜0/1, Silica-CS 12 g, 20 mL/min, silica gel, UV 254) to afford the product (63 mg, 53%). ESI-MS m/z calcd for [C₂₉H₃₃FN₆O₆S] [M+H]⁺: 613.2; found: 613.0. ¹H NMR (400 MHz, Methanol-d₄) δ 7.74 (d, J=8.8 Hz, 1H), 7.65-7.61 (m, 1H), 7.34-7.29 (m, 5H), 5.48 (s, 1H), 4.40 (d, J=9.6 Hz, 1H), 4.26 (s, 1H), 4.07-4.02 (m, 3H), 3.47 (s, 1H), 2.88 (d, J=10.0 Hz, 1H), 2.83 (s, 3H), 2.42 (s, 3H), 1.41 (s, 9H).

### Intermediate 39

### 4-Fluoro-6-nitro-1,3-benzothiazol-2-amine

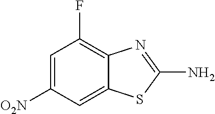 [SMILES: Nc1nc2c(F)cc([N+](=O)[O-])cc2s1]

To a cooled (−50° C.) solution of 4-fluoro-1,3-benzothiazol-2-amine (1.0 g, 5.95 mmol) in H₂SO₄ (10.0 mL) fuming HNO₃ (0.3 mL, 6.54 mmol) was added dropwise. The mixture was allowed to warm to rt and was stirred 1 h. The mixture was poured into ice cold water (50 mL) and extracted with EtOAc (2×30 mL). The combined organic phases were dried and concentrated to give the product (1.2 g, 95%). ESI-MS m/z calcd for [C₇H₄FN₃O₂S] [M+H]⁺: 214.0; found: 214.0. ¹H NMR (400 MHz, DMSO-d₆) δ 8.61 (d, J=2.0 Hz, 1H), 8.48 (s, 2H), 8.00 (dd, J=10.4, 2.0 Hz, 1H).

### 2-Bromo-4-fluoro-6-nitro-1,3-benzothiazole

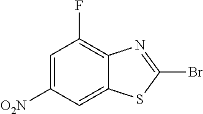 [SMILES: O=[N+]([O-])c1cc(F)c2nc(Br)sc2c1]

To a cooled (0° C.) solution of 4-fluoro-6-nitro-1,3-benzothiazol-2-amine (500 mg, 2.35 mmol) and copper(II) bromide (786 mg, 3.52 mmol) in MeCN (10.0 mL) tert-butyl nitrite (363 mg, 3.52 mmol) was added dropwise. The mixture was stirred 1 h at 0° C. and then 1 h at rt. Water (20 mL) was added, and the pH was adjusted to 1 using 2N HCl. The mixture was extracted with DCM (3×30 mL), the combined organic phases were dried and concentrated. The residue was purified by column chromatography (PE/EtOAc=10/1˜3/1, Silica-CS 20 g, 30 m/min, silica gel, UV 254) to afford the product (260 mg, 40%). ¹H NMR (400 MHz, DMSO-d₆) δ 9.07 (d, J=2.0 Hz, 1H), 8.32 (dd, J=10.4, 2.0 Hz, 1H).

### 2-Bromo-4-fluoro-1,3-benzothiazol-6-amine

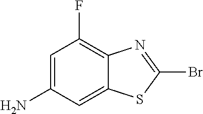 [SMILES: Nc1cc(F)c2nc(Br)sc2c1]

To a solution of 2-bromo-4-fluoro-6-nitro-1,3-benzothiazole (260 mg, 0.94 mmol) in EtOH/H₂O (8.0 mL, v/v 3:1) iron (262 mg, 4.69 mmol) and NH₄Cl (251 mg, 4.69 mmol) were added and the mixture was stirred 2 h at 50° C. The mixture was concentrated and purified by column chromatography (PE/EtOAc=10/1˜3/1, Silica-CS 20 g, 30 m/min, silica gel, UV 254) to give the product (210 mg, 91%). ESI-MS m/z calcd for [C₇H₄BrFN₂S] [M+H]⁺: 246.9; found: 247.1. ¹H NMR (400 MHz, DMSO-d₆) δ 6.87 (d, J=2.0 Hz, 1H), 6.58 (dd, J=13.2, 2.0 Hz, 1H), 5.82 (s, 2H).

### 4-Fluoro-2-methyl-1,3-benzothiazol-6-amine

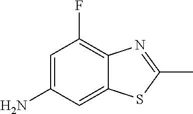

To a solution of 2-bromo-4-fluoro-1,3-benzothiazol-6-amine (500 mg, 2.02 mmol) and 2,4,6-trimethyl-1,3,5,2,4,6-trioxatriborinane (508 mg, 4.05 mmol) in DMF/H₂O (6.0 mL, 5:1) Pd(dppf)Cl₂ (148 mg, 0.20 mmol) and K₂CO₃ (839 mg, 6.07 mmol) were added. The mixture was purged three times with nitrogen and stirred 3 h at 70° C. Water (20 mL) was added, and the mixture was extracted with EtOAc (20 mL). The organic phase was dried, concentrated and purified by column chromatography (PE/EtOAc=10/1˜3/1, Silica-CS 20 g, 30 m/min, silica gel, UV 254) to give the product (180 mg, 49%). ESI-MS m/z calcd for [C₈H₇FN₂S] [M+H]⁺: 183.0; found: 183.1. ¹H NMR (400 MHz, DMSO-d₆) δ 6.82 (d, J=1.6 Hz, 1H), 6.52 (dd, J=13.2, 1.6 Hz, 1H), 5.54 (s, 2H), 2.67 (s, 3H).

### (4-Fluoro-2-methyl-1,3-benzothiazol-6-yl)hydrazine

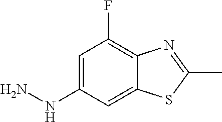

To a cooled (−10° C.) solution of 4-fluoro-2-methyl-1,3-benzothiazol-6-amine (180 mg, 0.99 mmol) in concentrated HCl (10 mL) a solution of NaNO₂ (81.8 mg, 1.19 mmol) in water (1.0 mL) was added dropwise at −10° C. The resulting mixture was stirred 30 min at −10° C. Tin(II) chloride dihydrate (669 mg, 2.96 mmol) dissolved in concentrated HCl (3.0 mL) was added dropwise at −10° C. The resulting mixture was allowed to reach rt and was then stirred 2 h. The mixture was poured into saturated aq NaHCO₃ and extracted with EtOAc (3×20 mL). The combined organic phases were washed with brine, dried over Na₂SO₄ and evaporated to afford the product (170 mg, 65%). ESI-MS m/z calcd for [C₈H₈FN₃S] [M+H]⁺: 198.0; found: 198.1. ¹H NMR (400 MHz, DMSO-d₆) δ 7.17 (s, 1H), 7.07 (d, J=1.6 Hz, 1H), 6.70 (dd, J=13.6, 2.0 Hz, 1H), 4.20 (br s, 2H), 2.68 (s, 3H).

### 6-[5-(3-Azido-4,6-O-benzylidene-3-deoxy-β-D-galactopyranosyl)-3-methyl-1H-1,2,4-triazol-1-yl]-4-fluoro-2-methylbenzothiazole

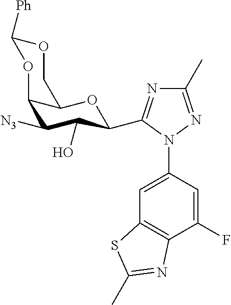

To a solution of 2,6-anhydro-4-azido-5,7-O-benzylidene-4-deoxy-D-glycero-L-manno-heptonamide (240 mg, 0.75 mmol) in 1,4-dioxane (9.0 mL) N,N-dimethylacetamide dimethyl acetal (110 mg, 0.82 mmol) was added and the mixture was stirred 3 h at 50° C. The mixture was evaporated, and the residue was dissolved in 1,4-dioxane (9.0 mL) and acetic acid (10 mL). To this solution (4-fluoro-2-methyl-1,3-benzothiazol-6-yl)hydrazine (170 mg, 0.86 mmol) was added and the mixture was stirred 2 h at 60° C. The mixture was evaporated and partitioned between water (20 mL) and DCM (20 mL). The aqueous phase was extracted with DCM (2×20 mL). The combined organic phases were washed with water (20 mL) and brine (20 mL), dried over Na₂SO₄ and evaporated. The residue was purified by column chromatography (EtOAc/PE=1/5˜2/1, Silica-CS 20 g, 30 mL/min, silica gel, UV 254) to afford the product (280 mg, 71%). ESI-MS m/z calcd for [C₂₄H₂₂FN₇O₄S] [M+H]⁺: 524.1; found: 524.0. ¹H NMR (400 MHz, Chloroform-d) δ 7.81 (s, 1H), 7.45-7.36 (m, 3H), 7.34-7.25 (m, 3H), 5.52 (s, 1H), 4.78 (t, J=10.0 Hz, 1H), 4.35 (d, J=9.2 Hz, 1H), 4.25 (d, J=3.2 Hz, 1H), 4.03-3.94 (m, 2H), 3.43 (s, 1H), 3.39 (dd, J=10.4, 3.2 Hz, 1H), 2.83 (s, 3H), 2.40 (s, 3H).

### 6-[5-(3-Amino-4,6-O-benzylidene-3-deoxy-β-D-galactopyranosyl)-3-methyl-1H-1,2,4-triazol-1-yl]-4-fluoro-2-methylbenzothiazole

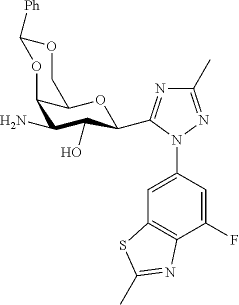

To a solution of 6-[5-(3-azido-4,6-O-benzylidene-3-deoxy-β-D-galactopyranosyl)-3-methyl-1H-1,2,4-triazol-1-yl]-4-fluoro-2-methylbenzothiazole (280 mg, 0.54 mmol) in THF/water (12 mL, 5:1) triphenylphosphine (701 mg, 2.67 mmol) was added and the mixture was stirred 6 h at 50° C. The mixture was concentrated and purified by column chromatography (DCM/MeOH=1/0˜10/1, Silica-CS 12 g, 40 mL/min, silica gel, UV 254) to afford the product (210 mg, 79%). ESI-MS m/z calcd for [C₂₄H₂₄FN₅O₄S][M+H]⁺: 498.2; found: 498.0. ¹H NMR (400 MHz, DMSO-d₆) δ 8.16 (d, J=1.6 Hz, 1H), 7.58 (dd, J=11.2, 2.0 Hz, 1H), 7.47-7.36 (m, 5H), 5.60 (s, 1H), 5.23 (br s, 1H), 4.36 (d, J=9.2 Hz, 1H), 4.17-4.05 (m, 3H), 3.87 (t, J=9.6 Hz, 1H), 3.67 (s, 1H), 2.86 (s, 3H), 2.72 (dd, J=10.0, 3.6 Hz, 1H), 2.35 (s, 3H).

### 6-{5-{4,6-O-Benzylidene-3-[(2-tert-butoxycarbonyl)hydrazinyl]-3-deoxy-β-D-galactopyranosyl}-3-methyl-1H-1,2,4-triazol-1-yl}-4-fluoro-2-methylbenzothiazole

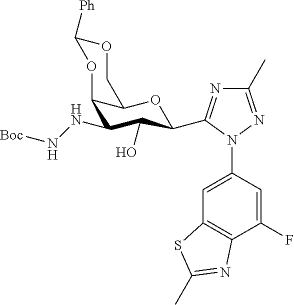

To a solution of 6-[5-(3-amino-4,6-O-benzylidene-3-deoxy-β-D-galactopyranosyl)-3-methyl-1H-1,2,4-triazol-1-yl]-4-fluoro-2-methylbenzothiazole (210 mg, 0.42 mmol) in DCM (5 mL) N-tert-butyloxycarbonyl-3-(4-cyanophenyl)oxaziridine (114 mg, 0.46 mmol) was added and the mixture was stirred overnight at rt. The mixture was concentrated and purified by column chromatography (PE/EtOAc=4/1˜0/1, Silica-CS 20 g, 30 mL/min, silica gel, UV 254) to afford the product (140 mg, 54%). ESI-MS m/z calcd for [C₂₉H₃₃FN₆O₆S] [M+H]⁺: 613.2; found: 613.0. ¹H NMR (400 MHz, DMSO-d₆) δ 8.35 (br s, 1H), 8.15 (d, J=1.6 Hz, 1H), 7.60-7.54 (m, 1H), 7.51-7.46 (m, 2H), 7.40-7.34 (m, 3H), 5.56 (s, 1H), 5.24 (d, J=5.2 Hz, 1H), 4.65-4.56 (m, 1H), 4.46 (d, J=9.6 Hz, 1H), 4.22 (d, J=3.2 Hz, 1H), 4.15-4.05 (m, 2H), 3.68 (s, 1H), 2.99-2.90 (m, 1H), 2.86 (s, 3H), 2.35 (s, 3H), 1.38 (s, 9H).

### Intermediate 40

### 7-Chloro-2-methyl-1,3-benzothiazol-6-amine

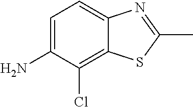

To a solution of 2-methyl-1,3-benzothiazol-6-amine (2.00 g, 12.2 mmol) in propan-2-ol (30 mL) N-chlorosuccinimide (1.79 g, 13.4 mmol) was added and the mixture was stirred 50 min at 65° C. The mixture was cooled to rt and poured into saurated. aq NaHCO₃. The aqueous mixture was extracted with DCM (2×30 mL). The combined organic phases were washed with brine, dried over Na₂SO₄, evaporated, and purified by column chromatography (PE/EtOAc=4/1˜1/1, Silica-CS 20 g, 30 mL/min, silica gel, UV 254) to afford the product (1.48 g, 61%). ESI-MS m/z calcd for [C₈H₇ClN₂S][M+H]⁺: 199.0; found: 199.1. ¹H NMR (400 MHz, Chloroform-d) δ 7.56 (d, J=8.4 Hz, 1H), 6.82 (d, J=8.4 Hz, 1H), 4.04 (br s, 2H), 2.70 (s, 3H).

### (7-Chloro-2-methyl-1,3-benzothiazol-6-yl)hydrazine

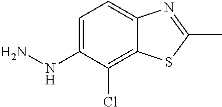

To a cooled (−10° C.) solution of 7-chloro-2-methyl-1,3-benzothiazol-6-amine (720 mg, 3.62 mmol) in concentrated HCl (10 mL) a solution of NaNO₂ (300 mg, 4.35 mmol) in water (1.5 mL) was added dropwise at −10° C. The resulting mixture was stirred 30 min at −10° C. Tin(II) chloride dihydrate (2.45 g, 10.9 mmol) dissolved in concentrated HCl (3.0 mL) was added dropwise at −10° C. The resulting mixture was allowed to reach rt and was then stirred 2 h. The mixture was poured into saturated aq NaHCO₃ and extracted with EtOAc (3×30 mL). The combined organic phases were washed with brine, dried over Na₂SO₄ and evaporated to afford the product (700 mg, 77%). ESI-MS m/z calcd for [C₈H₈ClN₃S] [M+H]⁺: 214.0; found: 214.0. ¹H NMR (400 MHz, DMSO-d₆) δ 7.70 (d, J=8.8 Hz, 1H), 7.40 (d, J=8.8 Hz, 1H), 6.78 (s, 1H), 4.31 (s, 2H), 2.71 (s, 3H).

### 6-[5-(3-Azido-4,6-O-benzylidene-3-deoxy-β-D-galactopyranosyl)-3-methyl-1H-1,2,4-triazol-1-yl]-7-chloro-2-methylbenzothiazole

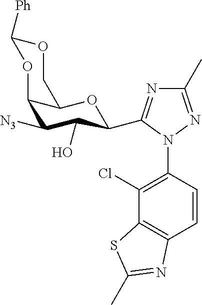

To a solution of 2,6-anhydro-4-azido-5,7-O-benzylidene-4-deoxy-D-glycero-L-manno-heptonamide (750 mg, 2.34 mmol) in 1,4-dioxane (15.0 mL) N,N-dimethylacetamide dimethyl acetal (343 mg, 2.58 mmol) was added and the mixture was stirred 3 h at 50° C. The mixture was evaporated, and the residue was dissolved in 1,4-dioxane (15.0 mL) and acetic acid (5.0 mL). To this solution (7-chloro-2-methyl-1,3-benzothiazol-6-yl)hydrazine (600 mg, 2.81 mmol) was added and the mixture was stirred 2 h at 60° C. The mixture was evaporated and partitioned between water (20 mL) and DCM (20 mL). The aqueous phase was extracted with DCM (2×20 mL). The combined organic phases were washed with water (20 mL) and brine (20 mL), dried over Na₂SO₄ and evaporated. The residue was purified by column chromatography (EtOAc/PE=1/5˜2/1, Silica-CS 20 g, 30 mL/min, silica gel, UV 254) to afford the product (970 mg, 77%). ESI-MS m/z calcd for [C₂₄H₂₂C₁N₇O₄S] [M+H]⁺: 540.1; found: 539.9. ¹H NMR (400 MHz, DMSO-d₆) δ 8.04 (d, J=8.8 Hz, 1H), 7.63 (d, J=8.4 Hz, 1H), 7.43-7.27 (m, 5H), 5.85-5.79 (m, 1H), 5.60 (s, 1H), 4.28 (d, J=2.8 Hz, 1H), 4.26-4.20 (m, 2H), 4.02-3.94 (m, 2H), 3.57-3.46 (m, 2H), 2.88 (s, 3H), 2.37 (s, 3H).

### 6-[5-(3-Amino-4,6-O-benzylidene-3-deoxy-β-D-galactopyranosyl)-3-methyl-1H-1,2,4-triazol-1-yl]-7-chloro-2-methylbenzothiazole

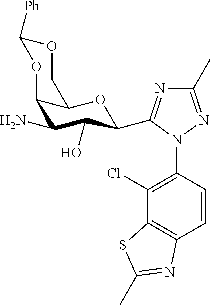

To a solution of 6-[5-(3-azido-4,6-O-benzylidene-3-deoxy-β-D-galactopyranosyl)-3-methyl-1H-1,2,4-triazol-1-yl]-7-chloro-2-methylbenzothiazole (480 mg, 0.89 mmol) in THF/water (12 mL, 5:1) triphenylphosphine (1.17 g, 4.44 mmol) was added and the mixture was stirred overnight at 50° C. The mixture was concentrated and purified by column chromatography (DCM/MeOH=1/0˜10/1, Silica-CS 12 g, 40 mL/min, silica gel, UV 254) to afford the product (300 mg, 66%). ESI-MS m/z calcd for [C₂₄H₂₄C₁N₅O₄S] [M+H]⁺: 514.1; found: 514.0. ¹H NMR (400 MHz, DMSO-d₆) δ 8.01 (d, J=8.4 Hz, 1H), 7.62 (d, J=8.4 Hz, 1H), 7.44-7.28 (m, 5H), 5.51 (s, 1H), 5.15 (d, J=3.6 Hz, 1H), 4.05-3.90 (m, 4H), 3.79-3.67 (m, 1H), 3.47 (s, 1H), 2.88 (s, 3H), 2.60 (dd, J=9.6, 3.2 Hz, 1H), 2.35 (s, 3H), 1.76 (br s, 2H).

### 6-{5-{4,6-O-Benzylidene-3-[(2-tert-butoxycarbonyl)hydrazinyl]-3-deoxy-β-D-galactopyranosyl}-3-methyl-1H-1,2,4-triazol-1-yl}-7-chloro-2-methylbenzothiazole

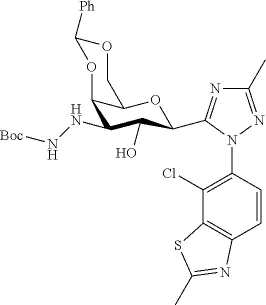

To a solution of 6-[5-(3-amino-4,6-O-benzylidene-3-deoxy-β-D-galactopyranosyl)-3-methyl-1H-1,2,4-triazol-1-yl]-7-chloro-2-methylbenzothiazole (300 mg, 0.58 mmol) in DCM (10 mL) N-tert-butyloxycarbonyl-3-(4-cyanophenyl)oxaziridine (216 mg, 0.88 mmol) was added and the mixture was stirred overnight at rt. The mixture was concentrated and purified by column chromatography (PE/EtOAc=4/1˜0/1, Silica-CS 20 g, 30 mL/min, silica gel, UV 254) to afford the product (230 mg, 63%). ESI-MS m/z calcd for [C₂₉H₃₃C₁N₆O₆S] [M+H]⁺: 629.2; found: 628.9. ¹H NMR (400 MHz, DMSO-d₆) δ 8.31 (br s, 1H), 8.00 (d, J=8.4 Hz, 1H), 7.61 (d, J=8.4 Hz, 1H), 7.45-7.32 (m, 5H), 5.48 (s, 1H), 5.20 (s, 1H), 4.53 (br s, 1H), 4.16 (d, J=9.6 Hz, 1H), 4.12 (d, J=3.2 Hz, 1H), 4.02-3.90 (m, 3H), 3.46 (s, 1H), 2.88 (s, 3H), 2.82 (dd, J=10.0, 2.8 Hz, 1H), 2.35 (s, 3H), 1.36 (s, 9H).

### Intermediate 41

### 4-Chloro-6-nitro-1,3-benzothiazol-2-amine

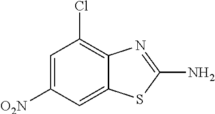 [SMILES: Nc1nc2c(Cl)cc([N+](=O)[O-])cc2s1]

To a cooled (−40° C.) solution of 4-chloro-1,3-benzothiazol-2-amine (4.00 g, 21.7 mmol) in H₂SO₄ (25 mL) fuming HNO₃ (2 mL) was added dropwise. The mixture was allowed to warm to rt and was stirred 1 h. The mixture was poured into ice cold water and the precipitate was collected by filtration. The obtained solid was dried under vacuum to afford the product (3.6 g, 72%). ¹H NMR (400 MHz, DMSO-d₆) δ 8.70 (d, J=2.4 Hz, 1H), 8.61 (s, 2H), 8.16 (d, J=2.4 Hz, 1H).

### 2-Bromo-4-chloro-6-nitro-1,3-benzothiazole

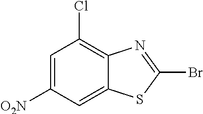 [SMILES: O=[N+]([O-])c1cc(Cl)c2nc(Br)sc2c1]

To a cooled (0° C.) solution of 4-chloro-6-nitro-1,3-benzothiazol-2-amine (2.00 g, 8.71 mmol) and copper(II) bromide (2.91 g, 13.1 mmol) in MeCN (40 mL) tert-butyl nitrite (1.35 g, 13.1 mmol) was added dropwise. The mixture was stirred 1 h at 0° C. and then 1 h at rt. Water (20 mL) was added, and the pH was adjusted to 1 using HCl (12N). The mixture was extracted with DCM (3×30 mL) and the combined organic phases were dried and concentrated. The residue was purified by column chromatography (PE/EtOAc=1/0˜0/1, Silica-CS 40 g, 40 mL/min, silica gel, UV 254) to afford the product (1.30 g, 51%). ¹H NMR (400 MHz, DMSO-d₆) δ 9.17 (d, J=2.0 Hz, 1H), 8.46 (d, J=2.0 Hz, 1H).

### 2-Bromo-4-chloro-1,3-benzothiazol-6-amine

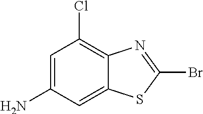 [SMILES: Nc1cc(Cl)c2nc(Br)sc2c1]

To a solution of 2-bromo-4-chloro-6-nitro-1,3-benzothiazole (300 mg, 1.02 mmol) in EtOH/H₂O (11 mL, v/v 10:1) iron (286 mg, 5.11 mmol) and NH₄Cl (276 mg, 5.11 mmol) were added and the mixture was stirred 3 h at 70° C. The mixture was filtered, and brine was added to the filtrate. The mixture was extracted with DCM (3×30 mL). The combined organic phases were dried, concentrated and purified by column chromatography (PE/EtOAc=1/0˜1/1, Silica-CS 12 g, 20 mL/min, silica gel, UV 254) to afford the product (156 mg, 58%). ESI-MS m/z calcd for [C₇H₄BrClN₂S] [M+H]⁺: 262.9; found: 262.9. ¹H NMR (400 MHz, DMSO-d₆) δ 7.02 (d, J=2.0 Hz, 1H), 6.85 (d, J=2.0 Hz, 1H), 5.79 (s, 2H).

### 4-Chloro-2-methyl-1,3-benzothiazol-6-amine

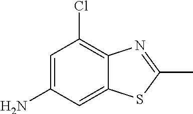

To a solution of 2-bromo-4-chloro-1,3-benzothiazol-6-amine (156 mg, 0.59 mmol) in DMF/H₂O (3.5 mL, 6:1) 2,4,6-trimethyl-1,3,5,2,4,6-trioxatriborinane (149 mg, 1.18 mmol), Pd(dppf)Cl₂ (43.3 mg, 0.059 mmol) and K₂CO₃ (247 mg, 1.78 mmol) were added and the mixture was stirred 3 h at 70° C. The mixture was filtered, concentrated, and purified by column chromatography (PE/EtOAc=1/0˜1/3, Silica-CS 12 g, 20 m/min, silica gel, UV 254) to give the product (98 mg, 83%). ESI-MS m/z calcd for [C₈H₇ClN₂S] [M+H]⁺: 199.0; found: 199.0. ¹H NMR (400 MHz, Chloroform-d) δ 6.88 (d, J=2.0 Hz, 1H), 6.77 (d, J=2.4 Hz, 1H), 3.73 (s, 2H), 2.72 (s, 3H).

### (4-Chloro-2-methyl-1,3-benzothiazol-6-yl)hydrazine

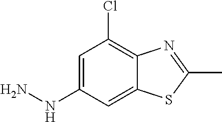

To a cooled (−10° C.) solution of 4-chloro-2-methyl-1,3-benzothiazol-6-amine (98 mg, 0.60 mmol) in HCl (2 mL, 33% wt) and acetic acid (0.6 mL) a solution of NaNO₂ (49.4 mg, 0.72 mmol) in water (2 mL) was added dropwise at −10° C. The resulting mixture was stirred 2 h at −10° C. Tin(II) chloride dihydrate (269 mg, 1.19 mmol) was added in small portions at −10° C. The resulting mixture was stirred 2 h at −10° C. before aq NaHCO₃ was added to adjust pH to 7. Water (20 mL) and DCM (20 mL) was added and the aqueous phase was extracted with DCM (2×10 mL). The combined organic phases were washed with water (5 mL) and brine (5 mL), dried over Na₂SO₄ and evaporated to afford the product (112 mg, 88%). ESI-MS m/z calcd for [C₈H₈ClN₃S][M+H]⁺: 214.0; found: 214.0.

### 6-[5-(3-Azido-4,6-O-benzylidene-3-deoxy-β-D-galactopyranosyl)-3-methyl-1H-1,2,4-triazol-1-yl]-4-chloro-2-methylbenzothiazole

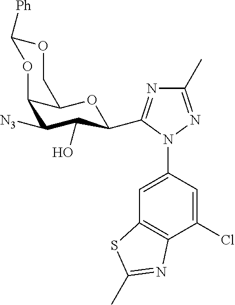

To a solution of 2,6-anhydro-4-azido-5,7-O-benzylidene-4-deoxy-D-glycero-L-manno-N-[1-(dimethylamino)ethylidene]heptonamide (130 mg, 0.33 mmol) in 1,4-dioxane (3 mL) and acetic acid (1 mL) (4-chloro-2-methyl-1,3-benzothiazol-6-yl)hydrazine (112 mg, 0.52 mmol) was added and the mixture was stirred 3 h at 60° C. The mixture was concentrated, DCM (10 mL) was added and the mixture was washed with aq NaHCO₃ and brine. The organic phase was dried over Na₂SO₄, evaporated, and purified by column chromatography (PE/EtOAc=1/0˜1/3, Silica-CS 12 g, 20 m/min, silica gel, UV 254) to afford the product (78 mg, 43%). ESI-MS m/z calcd for [C₂₄H₂₂C₁N₇O₄S][M+H]⁺: 540.1; found: 539.8. ¹H NMR (400 MHz, Chloroform-d) δ 7.92 (d, J=2.0 Hz, 1H), 7.72 (d, J=2.0 Hz, 1H), 7.42-7.40 (m, 2H), 7.30-7.26 (m, 3H), 5.52 (s, 1H), 4.73 (t, J=9.6 Hz, 1H), 4.33 (d, J=9.2 Hz, 1H), 4.24 (d, J=2.8 Hz, 1H), 4.12 (dd, J=12.4, 1.2 Hz, 1H), 4.02-3.98 (m, 1H), 3.43 (d, J=0.8 Hz, 1H), 3.36 (dd, J=10, 3.2 Hz, 1H), 2.84 (s, 3H), 2.39 (s, 3H).

### 6-[5-(3-Amino-4,6-O-benzylidene-3-deoxy-β-D-galactopyranosyl)-3-methyl-1H-1,2,4-triazol-1-yl]-4-chloro-2-methylbenzothiazole

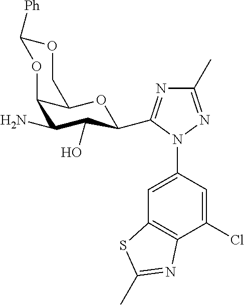

To a solution of 6-[5-(3-azido-4,6-O-benzylidene-3-deoxy-β-D-galactopyranosyl)-3-methyl-1H-1,2,4-triazol-1-yl]-4-chloro-2-methylbenzothiazole (130 mg, 0.24 mmol) in THF/water (6 mL, 5:1) triphenylphosphine (316 mg, 1.20 mmol) was added and the mixture was stirred 16 h at 50° C. The mixture was concentrated and purified by column chromatography (DCM/MeOH=1/0˜10/1, Silica-CS 12 g, 20 mL/min, silica gel, UV 254) to give the product (105 mg, 85%). ESI-MS m/z calcd for [C₂₄H₂₄C₁N₅O₄S][M+H]⁺: 514.1; found: 514.0. ¹H NMR (400 MHz, Chloroform-d) δ 7.94 (d, J=2.0 Hz, 1H), 7.66 (d, J=2.0 Hz, 1H), 7.42-7.39 (m, 2H), 7.30-7.28 (m, 3H), 5.49 (s, 1H), 4.31 (t, J=9.6 Hz, 1H), 4.21 (d, J=9.2 Hz, 1H), 4.18 (d, J=2.8 Hz, 1H), 4.12-4.36 (m, 3H), 3.46 (s, 1H), 2.85 (m, 3H), 2.34 (s, 3H).

### 6-{5-{4,6-O-Benzylidene-3-[(2-tert-butoxycarbonyl)hydrazinyl]-3-deoxy-β-D-galactopyranosyl}-3-methyl-1H-1,2,4-triazol-1-yl}-4-chloro-2-methylbenzothiazole

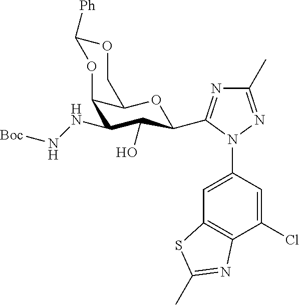

To a solution of 6-[5-(3-amino-4,6-O-benzylidene-3-deoxy-β-D-galactopyranosyl)-3-methyl-1H-1,2,4-triazol-1-yl]-4-chloro-2-methylbenzothiazole (105 mg, 0.20 mmol) in DCM (5 mL) N-tert-butyloxycarbonyl-3-(4-cyanophenyl)oxaziridine (55.3 mg, 0.23 mmol) was added and the mixture was stirred overnight at rt. The mixture was concentrated and purified by column chromatography (PE/EtOAc=4/1˜0/1, Silica-CS 12 g, 20 mL/min, silica gel, UV 254) to afford the product (102 mg, 79%). ESI-MS m/z calcd for [C₂₉H₃₃C₁N₆O₆S] [M+H]⁺: 629.2; found: 628.9. ¹H NMR (400 MHz, Chloroform-d) δ 7.93 (d, J=2.0 Hz, 1H), 7.72 (d, J=1.6 Hz, 1H), 7.38-7.36 (m, 2H), 7.26-7.24 (m, 3H), 6.61 (s, 1H), 5.48 (s, 1H), 4.48 (t, J=9.6 Hz, 1H), 4.33-4.31 (m, 2H), 4.14 (d, J=12.4 Hz, 1H), 4.00 (d, J=12.0 Hz, 1H), 3.42 (s, 1H), 2.99 (d, J=10.0 Hz, 1H), 2.81 (s, 3H), 2.31 (s, 3H), 1.35 (s, 9H).

### Intermediate 42

### N-(2,6-Dibromo-4-nitrophenyl)acetamide

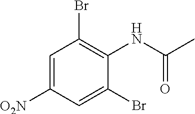

To a solution of 2,6-dibromo-4-nitroaniline (3.5 g, 11.8 mmol) in DMF (40 mL) acetyl chloride (1.3 g, 16.6 mmol) was added and the mixture was stirred overnight at 55° C. The mixture was poured into crushed ice-water (300 mL). The white solid was filtered, washed with water, and dried to afford the product (3.2 g, 80%). ESI-MS m/z calcd for [C₈H₆Br₂N₂O₃] [M+H]⁺: 338.9; found: 338.9. ¹H NMR (400 MHz, DMSO-d₆) δ 10.22 (s, 1H), 8.51 (s, 2H), 2.09 (s, 3H).

### N-(2,6-Dibromo-4-nitrophenyl)thioacetamide

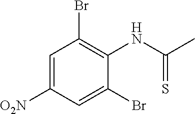

To a solution of N-(2,6-dibromo-4-nitrophenyl)acetamide (3.2 g, 9.47 mmol) in toluene (100 mL) Lawesson's reagent (3.83 g, 9.47 mmol) was added and the mixture was stirred overnight at 110° C. The mixture was concentrated and purified by column chromatography (EtOAc/PE=0/1˜1/1, Silica-CS 40 g, 50 m/min, silica gel, UV 254) to give the product (3.1 g, 93%). ESI-MS m/z calcd for [C₈H₆Br₂N₂O₂S] [M+H]⁺: 355.0; found: 354.9.

### 4-Bromo-2-methyl-6-nitro-1,3-benzothiazole

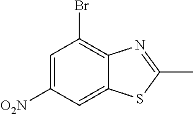

To a solution of N-(2,6-dibromo-4-nitrophenyl)thioacetamide (3.1 g, 8.76 mmol) in 1,2-dimethoxyethane (40 mL) CuI (83.4 mg, 0.44 mmol), 1,10-phenanthroline (158 mg, 0.88 mmol) and Cs₂CO₃ (4.28 g, 13.1 mmol) were added and the mixture was stirred overnight at 80° C. The mixture was cooled to rt, filtered over celite and concentrated. The residue was purified by column chromatography (EtOAc/PE=0/1˜2/1, Silica-CS 40 g, 50 mL/min, silica gel, UV 254) to afford the product (1.8 g, 75%). ESI-MS m/z calcd for [C₈H₅BrN₂O₂S] [M+H]⁺: 272.9; found: 273.0. ¹H NMR (400 MHz, DMSO-d₆) δ 9.16 (d, J=2.0 Hz, 1H), 8.51 (d, J=2.4 Hz, 1H), 2.92 (s, 3H).

### 4-Bromo-2-methyl-1,3-benzothiazol-6-amine

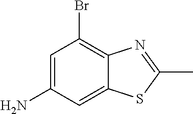

To a solution of 4-bromo-2-methyl-6-nitro-1,3-benzothiazole (2.0 g, 7.32 mmol) in EtOH (80 mL) iron (1.22 g, 22.0 mmol), NH₄Cl (3.92 g, 73.2 mmol) and water (40 mL) were added and the mixture was stirred 1 h at 80° C. The mixture was cooled, filtered over celite and concentrated. The residue was purified by column chromatography (PE/EtOAc=1/0˜1/1, Silica-CS 40 g, 40 mL/min, silica gel, UV 254) to afford the product (1.7 g, 96%). ESI-MS m/z calcd for [C₈H₇BrN₂S] [M+H]⁺: 243.0; found: 243.0. ¹H NMR (400 MHz, DMSO-d₆) δ 7.00 (d, J=2.0 Hz, 1H), 6.97 (d, J=2.0 Hz, 1H), 5.50 (s, 2H), 2.67 (s, 3H).

### (4-Bromo-2-methyl-1,3-benzothiazol-6-yl)hydrazine

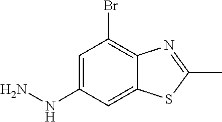

To a cooled (0° C.) solution of 4-bromo-2-methyl-1,3-benzothiazol-6-amine (800 mg, 3.29 mmol) in HCl (16 mL), water (8 mL) and acetic acid (8 mL) a solution of NaNO₂ (199 mg, 2.88 mmol) in water (4 mL) was added over 10 min. The mixture was stirred 1 h at 0° C. Tin(II) chloride (1.17 g, 6.17 mmol) dissolved in concentrated HCl (10 mL) was added dropwise at 0° C. The resulting mixture was warmed to rt and stirred 3 h before aq NaOH (5 M) was added to adjust pH to 8. The mixture was extracted with EtOAc (2×250 mL). The combined organic phases were dried over Na₂SO₄, evaporated, and purified by column chromatography (EtOAc/DCM=0/1˜1/1, Silica-CS 20 g, 25 mL/min, silica gel, UV 254) to afford the product (330 mg, 62%). ESI-MS m/z calcd for [C₈H₈BrN₃S] [M+H]⁺: 258.0; found: 258.0. ¹H NMR (400 MHz, DMSO-d₆) δ 7.25 (d, J=2.0 Hz, 1H), 7.14-7.12 (m, 2H), 4.14 (s, 2H), 2.69 (s, 3H).

### 6-[5-(3-Azido-4,6-O-benzylidene-3-deoxy-β-D-galactopyranosyl)-3-methyl-1H-1,2,4-triazol-1-yl]-4-bromo-2-methylbenzothiazole

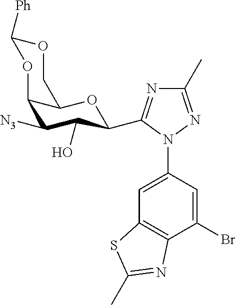

To a solution of 2,6-anhydro-4-azido-5,7-O-benzylidene-4-deoxy-D-glycero-L-manno-N-[1-(dimethylamino)ethylidene]heptonamide (200 mg, 0.51 mmol) in 1,4-dioxane (6 mL) and acetic acid (3 mL) (4-bromo-2-methyl-1,3-benzothiazol-6-yl)hydrazine (199 mg, 0.77 mmol) was added and the mixture was stirred 2 h at 60° C. The mixture was concentrated and purified by column chromatography (DCM/EtOAc=1/0˜1/1, Silica-CS 20 g, 25 m/min, silica gel, UV 254) to afford the product (210 mg, 70%). ESI-MS m/z calcd for [C₂₄H₂₂BrN₇O₄S] [M+H]⁺: 584.1; found: 583.8. ¹H NMR (400 MHz, Chloroform-d) δ 8.04 (br s, 1H), 7.98 (s, 1H), 7.52-7.48 (m, 2H), 7.37-7.34 (m, 3H), 5.60 (s, 1H), 4.90-4.84 (m, 1H), 4.42-4.32 (m, 2H), 4.16-4.07 (m, 2H), 3.51-3.44 (m, 2H), 2.91 (s, 3H), 2.48 (s, 3H).

### 6-[5-(3-Amino-4,6-O-benzylidene-3-deoxy-β-D-galactopyranosyl)-3-methyl-1H-1,2,4-triazol-1-yl]-4-bromo-2-methylbenzothiazole

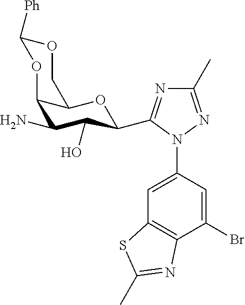

To a solution of 6-[5-(3-azido-4,6-O-benzylidene-3-deoxy-β-D-galactopyranosyl)-3-methyl-1H-1,2,4-triazol-1-yl]-4-bromo-2-methylbenzothiazole (210 mg, 0.36 mmol) in THF/water (22 mL, 10:1) triphenylphosphine (471 mg, 1.80 mmol) was added and the mixture was stirred overnight at 50° C. The mixture was concentrated and purified by column chromatography (DCM/MeOH=1/0˜10/1, Silica-CS 20 g, 25 mL/min, silica gel, UV 254) to afford the product (130 mg, 65%). ESI-MS m/z calcd for [C₂₄H₂₄BrN₅O₄S] [M+H]⁺: 558.1; found: 557.9. ¹H NMR (400 MHz, Chloroform-d) δ 7.98 (d, J=2.0 Hz, 1H), 7.95 (d, J=2.0 Hz, 1H), 7.43-7.39 (m, 2H), 7.31-7.28 (m, 3H), 5.50 (s, 1H), 4.29 (t, J=9.6 Hz, 1H), 4.21-4.16 (m, 2H), 4.15-4.11 (m, 1H), 4.03-3.99 (m, 1H), 3.46 (s, 1H), 2.84 (s, 3H), 2.83-2.81 (m, 1H), 2.36 (s, 3H).

### 6-{5-{4,6-O-Benzylidene-3-[(2-tert-butoxycarbonyl)hydrazinyl]-3-deoxy-β-D-galactopyranosyl}-3-methyl-1H-1,2,4-triazol-1-yl}-4-bromo-2-methylbenzothiazole

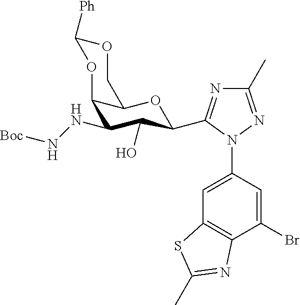

To a solution of 6-[5-(3-amino-4,6-O-benzylidene-3-deoxy-β-D-galactopyranosyl)-3-methyl-1H-1,2,4-triazol-1-yl]-4-bromo-2-methylbenzothiazole (130 mg, 0.23 mmol) in DCM (5 mL) N-tert-butyloxycarbonyl-3-(4-cyanophenyl)oxaziridine (68.8 mg, 0.28 mmol) was added and the mixture was stirred overnight at rt. The mixture was concentrated and purified by column chromatography (DCM/MeOH=1/0˜10/1, Silica-CS 12 g, 20 m/min, silica gel, UV 254) to afford the product (115 mg, 73%). ESI-MS m/z calcd for [C₂₉H₃₃BrN₆O₆S] [M+H]⁺: 673.1; found: 672.8. ¹H NMR (400 MHz, Chloroform-d) δ 8.08 (d, J=1.6 Hz, 1H), 7.97 (d, J=1.6 Hz, 1H), 7.45-7.41 (m, 2H), 7.34-7.29 (m, 3H), 5.56 (s, 1H), 4.62-4.55 (m, 1H), 4.49-4.41 (m, 2H), 4.21 (d, J=12.4 Hz, 1H), 4.09-4.05 (m, 1H), 3.53 (br s, 1H), 3.19-3.13 (m, 1H), 2.88 (s, 3H), 2.46 (s, 3H), 1.43 (s, 9H).

### 4-Bromo-6-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-methylbenzothiazole

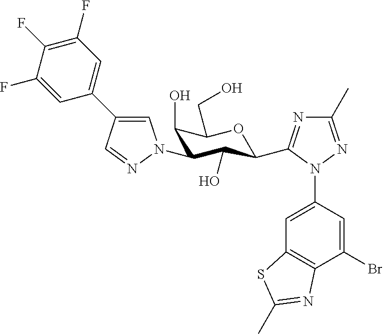

To a solution of 6-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-4-bromo-2-methylbenzothiazole (115 mg, 0.18 mmol) in EtOH (5 mL) 3-(dimethylamino)-2-(3,4,5-trifluorophenyl)prop-2-enal (60.7 mg, 0.27 mmol) and concentrated HCl (0.2 mL) were added and the mixture was stirred 2 h at 80° C. The mixture was concentrated and purified by column chromatography (DCM/MeOH=1/0˜10/1, Silica-CS 12 g, 20 m/min, silica gel, UV 254) to afford the product (45 mg, 41%). ESI-MS m/z calcd for [C₂₆H₂₂BrF₃N₆O₄S] [M+H]⁺: 651.1; found: 650.8. ¹H NMR (400 MHz, DMSO-d₆) δ Methanol-d₄) δ 8.40 (d, J=2.0 Hz, 1H), 8.18 (s, 1H), 8.02 (d, J=2.0 Hz, 1H), 7.89 (s, 1H), 7.39-7.32 (m, 2H), 4.91-4.89 (m, 1H), 4.51 (d, J=9.6 Hz, 1H), 4.44 (dd, J=10.8, 2.8 Hz, 1H), 4.12-4.10 (m, 1H), 3.84-3.81 (m, 2H), 3.75-3.70 (m, 1H), 2.91 (s, 3H), 2.47 (s, 3H).

### Intermediate 43

### 5-Bromo-2-methyl-6-nitro-1,3-benzothiazole

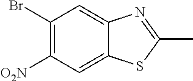

To a cooled (0° C.) solution of 5-bromo-2-methyl-1,3-benzothiazole (1.85 g, 8.11 mmol) in H₂SO₄ (10 mL) KNO₃ (902 mg, 8.92 mmol) was added in small portions. The mixture was stirred 2 h at 0° C. The mixture was poured into ice cold water and extracted with DCM (2×50 mL). The combined organic phases were dried over Na₂SO₄, evaporated, and purified by column chromatography (PE/EtOAc=1/0˜1/1, silica-CS 40 g, 40 mL/min, silica gel, UV 254 nm) to give the product (1.2 g, 54%). ESI-MS m/z calcd for [C₈H₅BrN₂O₂S] [M+H]⁺: 272.9; found: 273.0. ¹H NMR (400 MHz, Chloroform-d) δ 8.33 (s, 1H), 8.21 (s, 1H), 2.84 (s, 3H).

### 5-Bromo-2-methyl-1,3-benzothiazol-6-amine

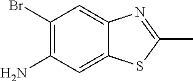

To a solution of 5-bromo-2-methyl-6-nitro-1,3-benzothiazole (1.2 g, 4.39 mmol) in EtOH (15 mL) iron (1.23 g, 22.0 mmol) and NH₄Cl (1.18 g, 22.0 mmol) were added and the mixture was stirred 2 h at 80° C. The mixture was filtered, poured into ice cold water and extracted with DCM (3×50 mL). The combined organic phases were washed with water and brine, dried over Na₂SO₄, concentrated, and purified by column chromatography (PE/EtOAc=1/0˜1/1, Silica-CS 40 g, 40 mL/min, silica gel, UV 254) to afford the product (900 mg, 84%). ESI-MS m/z calcd for [C₈H₇BrN₂S] [M+H]⁺: 243.0; found: 243.0. ¹H NMR (400 MHz, DMSO-d₆) δ 7.91 (s, 1H), 7.28 (s, 1H), 5.46 (br s, 2H), 2.68 (s, 3H).

### (5-Bromo-2-methyl-1,3-benzothiazol-6-yl)hydrazine

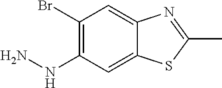

To a cooled (−5° C.) solution of 5-bromo-2-methyl-1,3-benzothiazol-6-amine (800 mg, 3.29 mmol) in water (15 mL) HCl (15 mL, 33% wt) was added followed by dropwise addition of a solution of NaNO₂ (272 mg, 3.95 mmol) in water (2 mL). The mixture was stirred 1 h at −5° C. Tin(II) chloride dihydrate (1782 mg, 7.90 mmol) dissolved in concentrated HCl (5 mL) was added dropwise at −5° C. The resulting mixture was stirred 2 h at −5° C. before NaOH (100 mL, 10% wt) was added to adjust pH to 7. Water (50 mL) and DCM (100 mL) was added and the aqueous phase was extracted with DCM (2×50 mL). The combined organic phases were washed with water (50 mL) and brine (2×50 mL), dried over Na₂SO₄ and evaporated to afford the product (350 mg, 41%). ESI-MS m/z calcd for [C₈H₈BrN₃S] [M+H]⁺: 258.0; found: 257.8. ¹H NMR (400 MHz, DMSO-d₆) δ 7.93 (s, 1H), 7.65 (s, 1H), 6.48 (br s, 1H), 4.25 (br s, 2H), 2.70 (s, 3H).

### 6-[5-(3-Azido-4,6-O-benzylidene-3-deoxy-β-D-galactopyranosyl)-3-methyl-1H-1,2,4-triazol-1-yl]-5-bromo-2-methylbenzothiazole

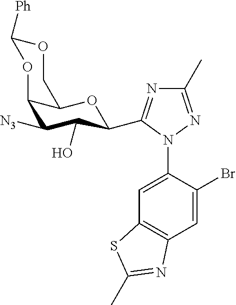

To a solution of 2,6-anhydro-4-azido-5,7-O-benzylidene-4-deoxy-D-glycero-L-manno-N-[1-(dimethylamino)ethylidene]heptonamide (500 mg, 1.28 mmol) in 1,4-dioxane (10 mL) and acetic acid (3.0 mL) (5-bromo-2-methyl-1,3-benzothiazol-6-yl)hydrazine (348 mg, 1.35 mmol) was added and the mixture was stirred 3 h at 60° C. The mixture was concentrated, DCM (10 mL) was added and the mixture was washed with aq NaHCO₃ and brine. The organic phase was dried over Na₂SO₄, evaporated, and purified by column chromatography (PE/EtOAc=1/0˜0/1, Silica-CS 40 g, 40 m/min, silica gel, UV 254) to afford the product (360 mg, 48%). ESI-MS m/z calcd for [C₂₄H₂₂BrN₇O₄S][M+H]⁺: 584.1; found: 583.8. ¹H NMR (400 MHz, Chloroform-d) δ 8.15 (s, 1H), 7.93 (s, 1H), 7.37-7.26 (m, 5H), 5.37 (s, 1H), 4.56-4.52 (m, 1H), 4.43 (d, J=8.0 Hz, 1H), 4.10 (d, J=2.8 Hz, 1H), 3.72 (d, J=11.6 Hz, 1H), 3.43-3.40 (m, 2H), 3.17 (s, 1H), 2.79 (s, 3H), 2.42 (s, 3H).

### 6-[5-(3-Amino-4,6-O-benzylidene-3-deoxy-β-D-galactopyranosyl)-3-methyl-1H-1,2,4-triazol-1-yl]-5-bromo-2-methylbenzothiazole

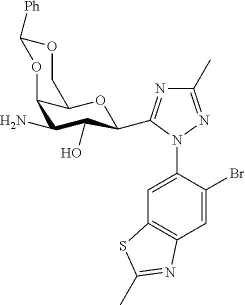

To a solution of 6-[5-(3-azido-4,6-O-benzylidene-3-deoxy-β-D-galactopyranosyl)-3-methyl-1H-1,2,4-triazol-1-yl]-5-bromo-2-methylbenzothiazole (360 mg, 0.62 mmol) in THF/water (10.5 mL, 20:1) triphenylphosphine (808 mg, 3.08 mmol) was added and the mixture was stirred overnight at 50° C. The mixture was concentrated and purified by column chromatography (DCM/MeOH=1/0˜10/1, Silica-CS 20 g, 40 mL/min, silica gel, UV 254) to afford the product (300 mg, 87%). ESI-MS m/z calcd for [C₂₄H₂₄BrN₅O₄S] [M+H]⁺: 558.1; found: 557.8. ¹H NMR (400 MHz, DMSO-d₆) δ 8.38 (s, 1H), 8.32-8.20 (m, 1H), 7.36 (s, 5H), 5.53 (s, 1H), 5.23 (d, J=4.0 Hz, 1H), 4.06-4.00 (m, 2H), 3.97 (s, 2H), 3.78-3.72 (m, 1H), 3.46-3.42 (m, 1H), 2.86 (s, 3H), 2.72-2.68 (m, 1H), 2.35 (s, 3H).

### 6-{5-{4,6-O-Benzylidene-3-[(2-tert-butoxycarbonyl)hydrazinyl]-3-deoxy-β-D-galactopyranosyl}-3-methyl-1H-1,2,4-triazol-1-yl}-5-bromo-2-methylbenzothiazole

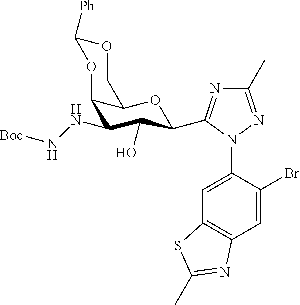

To a solution of 6-[5-(3-amino-4,6-O-benzylidene-3-deoxy-β-D-galactopyranosyl)-3-methyl-1H-1,2,4-triazol-1-yl]-5-bromo-2-methylbenzothiazole (200 mg, 0.36 mmol) in DCM (10 mL) N-tert-butyloxycarbonyl-3-(4-cyanophenyl)oxaziridine (106 mg, 0.43 mmol) was added and the mixture was stirred overnight at rt. The mixture was concentrated and purified by column chromatography (DCM/EtOAc=1/0˜0/1, Silica-CS 20 g, 40 m/min, silica gel, UV 254) to afford the product (115 mg, 48%). ESI-MS m/z calcd for [C₂₉H₃₃BrN₆O₆S] [M+H]⁺: 673.1; found: 672.8. ¹H NMR (400 MHz, DMSO-d₆) δ 8.37 (s, 1H), 8.31 (br s, 1H), 8.24-8.20 (m, 1H), 7.42-7.36 (m, 5H), 5.48 (s, 1H), 5.16 (d, J=4.0 Hz, 1H), 4.54 (br s, 1H), 4.10-4.06 (m, 2H), 3.97-3.91 (m, 3H), 3.45-3.40 (m, 1H), 2.85 (s, 3H), 2.82-2.79 (m, 1H), 2.35 (s, 3H), 1.36 (s, 9H).

### Intermediate 45

### N-Acetyl-N-(3-bromo-5-nitropyridin-2-yl)acetamide

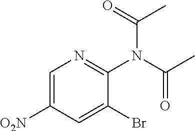 [SMILES: CC(=O)N(C(C)=O)c1ncc([N+](=O)[O-])cc1Br]

A suspension of 3-bromo-5-nitropyridin-2-amine (4.00 g, 18.3 mmol) in acetic anhydride (60 mL) was stirred 2 h at 100° C. The mixture was cooled to rt, poured into aq NaHCO₃ (500 mL) and extracted with EtOAc (2×200 mL). The combined organic phases were washed with brine (250 mL, dried over Na₂SO₄, evaporated, and purified by column chromatography (EtOAc/PE=1/3˜1/2, Silica-CS 40 g, 50 mL/min, silica gel, UV 254) to afford the product (4.5 g, 81%). ESI-MS m/z calcd for [C₉H₈BrN₃O₄][M+H]⁺: 301.0; found: 300.9. ¹H NMR (400 MHz, Chloroform-d) δ 9.29 (d, J=2.4 Hz, 1H), 8.77 (d, J=2.4 Hz, 1H), 2.24 (s, 6H).

### N-(3-Bromo-5-nitro-pyridin-2-yl)thioacetamide

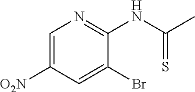

To a solution of N-acetyl-N-(3-bromo-5-nitropyridin-2-yl)acetamide (5.20 g, 17.2 mmol) in toluene (100 mL) Lawesson's reagent (4.87 g, 12.0 mmol) was added and the mixture was stirred 2 h at 110° C. The mixture was concentrated and purified by column chromatography (EtOAc/PE=1/10˜1/1, Silica-CS 40 g, 50 mL/min, silica gel, UV 254) to afford the product (3.2 g, 67%). ESI-MS m/z calcd for [C₇H₆BrN₃O₂S] [M+H]⁺: 275.9; found: 275.9. ¹H NMR (400 MHz, Chloroform-d) δ 9.85 (br s, 1H), 9.09 (d, J=2.4 Hz, 1H), 8.63 (d, J=2.0 Hz, 1H), 3.07 (s, 3H).

### 2-Methyl-6-nitrothiazolo[4,5-b]pyridine

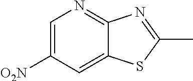

To a solution of N-(3-bromo-5-nitro-pyridin-2-yl)thioacetamide (2.80 g, 10.1 mmol) in DMF (30 mL) sodium carbonate (2.15 g, 20.3 mmol) was added and the mixture was stirred 3 h at 90° C. The mixture was cooled to rt, poured into water (150 mL) and extracted with EtOAc (3×150 mL). The combined organic phases were washed with water (150 mL) and brine (150 mL), dried over Na₂SO₄, evaporated, and purified by column chromatography (EtOAc/PE=0/1˜1/1, Silica-CS 40 g, 50 m/min, silica gel, UV 254) to afford the product (1.1 g, 56%). ESI-MS m/z calcd for [C₇H₅N₃O₂S][M+H]⁺: 196.0; found: 196.1. ¹H NMR (400 MHz, Chloroform-d) δ 9.55 (d, J=2.8 Hz, 1H), 9.04 (d, J=2.4 Hz, 1H), 3.01 (s, 3H).

### 2-Methylthiazolo[4,5-b]pyridin-6-amine

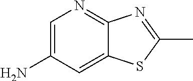

To a solution of 2-methyl-6-nitrothiazolo[4,5-b]pyridine (1.10 g, 5.64 mmol) in EtOH (55 mL) iron (944 mg, 16.9 mmol), NH₄Cl (3.01 g, 56.3 mmol) and water (25 mL) were added and the mixture was stirred 2 h at 85° C. The mixture was filtered, concentrated, and purified by column chromatography (DCM/MeOH=1/0˜10/1, Silica-CS 20 g, 20 m/min, silica gel, UV 254) to afford the product (550 mg, 59%). ESI-MS m/z calcd for [C₇H₇N₃S] [M+H]⁺: 166.0; found: 166.1. ¹H NMR (400 MHz, Chloroform-d) δ 8.16 (d, J=2.8 Hz, 1H), 7.41 (d, J=2.8 Hz, 1H), 2.81 (s, 3H).

### (2-Methylthiazolo[4,5-b]pyridin-6-yl)hydrazine

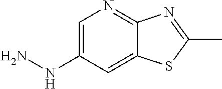

To a cooled (−5° C.) solution of 2-methylthiazolo[4,5-b]pyridin-6-amine (285 mg, 1.73 mmol) in concentrated HCl (9.0 mL) a solution of NaNO₂ (179 mg, 2.59 mmol) in water (1.0 mL) was added over 5 min. The mixture was stirred 45 min at −5° C. Tin(II) chloride dihydrate (1.17 g, 5.19 mmol) dissolved in concentrated HCl (3.0 mL) was added dropwise over 10 min at 0° C. The resulting mixture was stirred 2 h at −5° C. before water (30 mL) was added and pH was adjusted to 8 by dropwise addition of aqueous NaOH (5 M) at −5° C. The mixture was extracted with DCM (4×50 mL). The combined organic phases were washed with brine (50 mL), dried over Na₂SO₄, evaporated, and purified by column chromatography (MeOH/DCM=0/1˜1/10, Silica-CS 20 g, 20 m/min, silica gel, UV 254) to afford the product (221 mg, 71%). ESI-MS m/z calcd for [C₇HsN₄S] [M+H]⁺: 181.1; found: 181.2. ¹H NMR (400 MHz, Chloroform-d) δ 8.23 (d, J=2.4 Hz, 1H), 7.71 (d, J=2.8 Hz, 1H), 5.44 (br s, 1H), 3.72 (br s, 2H), 2.82 (s, 3H).

### 6-[5-(3-Azido-4,6-O-benzylidene-3-deoxy-β-D-galactopyranosyl)-3-methyl-1H-1,2,4-triazol-1-yl]-2-methylthiazolo[4,5-b]pyridine

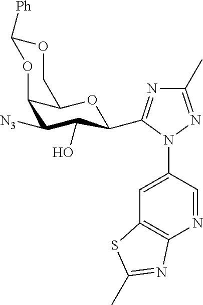

To a solution of 2,6-anhydro-4-azido-5,7-O-benzylidene-4-deoxy-D-glycero-L-manno-N-[1-(dimethylamino)ethylidene]heptonamide (285 mg, 0.57 mmol) in 1,4-dioxane (6 mL) and acetic acid (3 mL) (2-methylthiazolo[4,5-b]pyridin-6-yl)hydrazine (112 mg, 0.62 mmol) was added and the mixture was stirred 2 h at 60° C. The mixture was concentrated and partitioned between water (20 mL) and DCM (20 mL). The aqueous phase was extracted with DCM (2×20 mL). The combined organic phases were washed with water (20 mL) and brine (20 mL), dried over Na₂SO₄, evaporated, and purified by column chromatography (PE/EtOAc=5/1˜1/2, Silica-CS 20 g, 30 mL/min, silica gel, UV 254) to afford the product (167 mg, 58%). ESI-MS m/z calcd for [C₂₃H₂₂NsO₄S] [M+H]⁺: 507.2; found: 507.2. ¹H NMR (400 MHz, Chloroform-d) δ 8.84 (d, J=2.4 Hz, 1H), 8.41 (d, J=2.0 Hz, 1H), 7.48-7.45 (m, 2H), 7.36-7.33 (m, 3H), 5.56 (s, 1H), 4.78 (t, J=9.6 Hz, 1H), 4.50 (d, J=9.2 Hz, 1H), 4.30 (d, J=3.2 Hz, 1H), 3.96-3.95 (m, 2H), 3.50-3.46 (m, 2H), 2.94 (s, 3H), 2.47 (s, 3H).

### 6-[5-(3-Amino-4,6-O-benzylidene-3-deoxy-β-D-galactopyranosyl)-3-methyl-1H-1,2,4-triazol-1-yl]-2-methylthiazolo[4,5-b]pyridine

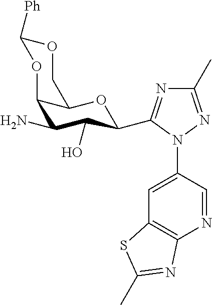

To a solution of 6-[5-(3-azido-4,6-O-benzylidene-3-deoxy-β-D-galactopyranosyl)-3-methyl-1H-1,2,4-triazol-1-yl]-2-methylthiazolo[4,5-b]pyridine (167 mg, 0.33 mmol) in THF/water (12 mL, 5:1) triphenylphosphine (432 mg, 1.65 mmol) was added and the mixture was stirred 16 h at 50° C. The mixture was concentrated and purified by column chromatography (DCM/MeOH=1/0˜10/1, Silica-CS 12 g, 20 mL/min, silica gel, UV 254) to afford the product (139 mg, 88%). ESI-MS m/z calcd for [C₂₃H₂₄N₆O₄S][M+H]⁺: 481.2; found: 481.0. ¹H NMR (400 MHz, Chloroform-d) δ 8.77 (br s, 1H), 8.32 (br s, 1H), 7.38-7.19 (m, 5H), 5.43 (s, 1H), 4.51-4.27 (m, 3H), 3.95-3.88 (m, 2H), 3.55-3.18 (m, 2H), 2.78 (s, 3H), 2.30 (s, 3H).

### 6-{5-{4,6-O-Benzylidene-3-[(2-tert-butoxycarbonyl)hydrazinyl]-3-deoxy-β-D-galactopyranosyl}-3-methyl-1H-1,2,4-triazol-1-yl}-2-methylthiazolo[4,5-b]pyridine

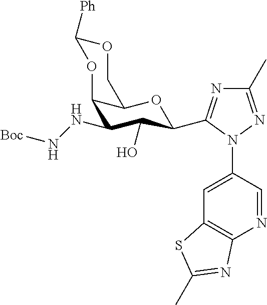

To a solution of 6-[5-(3-amino-4,6-O-benzylidene-3-deoxy-β-D-galactopyranosyl)-3-methyl-1H-1,2,4-triazol-1-yl]-2-methylthiazolo[4,5-b]pyridine (139 mg, 0.29 mmol) in DCM (5.0 mL) N-tert-butyloxycarbonyl-3-(4-cyanophenyl)oxaziridine (78.4 mg, 0.32 mmol) was added and the mixture was stirred overnight at rt. The mixture was concentrated and purified by column chromatography (PE/EtOAc=4/1˜0/1, Silica-CS 20 g, 30 mL/min, silica gel, UV 254) to afford the product (109 mg, 63%). ESI-MS m/z calcd for [C₂₈H₃₃N₇O₆S] [M+H]⁺: 596.2; found: 595.9. ¹H NMR (400 MHz, Chloroform-d) δ 8.86 (d, J=2.4 Hz, 1H), 8.44 (d, J=2.4 Hz, 1H), 7.42-7.39 (m, 2H), 7.33-7.30 (m, 3H), 6.36 (br s, 1H), 5.51 (s, 1H), 4.49-4.46 (m, 2H), 4.36 (d, J=2.4 Hz, 1H), 4.03-3.98 (m, 2H), 3.50 (br s, 1H), 3.07-3.04 (m, 1H), 2.83 (s, 3H), 2.40 (s, 3H), 1.43 (s, 9H).

### Intermediate 47

### (E)-1,2,3-Trifluoro-5-(2-nitrovinyl)benzene

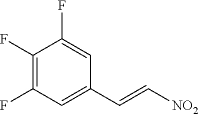 [SMILES: O=[N+]([O-])/C=C/c1cc(F)c(F)c(F)c1]

Ammonium acetate (1.16 g, 15 mmol) was added to a solution of 3,4,5-trifluorobenzaldehyde (2.0 g, 12.5 mmol) and nitromethane (2.71 mL) in acetic acid (8.0 mL). The mixture was stirred 2 h at 100° C. and was then cooled to rt. The mixture was concentrated and partitioned between EtOAc (30 mL) and saturated aq NaHCO₃. The organic phase was dried over Na₂SO₄, evaporated, and purified by column chromatography (EtOAc/PE=0/1˜1/10, Silica-CS 20 g, 20 m/min, silica gel, UV 254) to afford the product (1.01 g, 40%). ¹H NMR (400 MHz, Chloroform-d) δ 7.85 (d, J=13.6 Hz, 1H), 7.49 (d, J=13.6 Hz, 1H), 7.22-7.18 (m, 2H).

### 5-Chloro-1-{5-{3-deoxy-3-[2-(phenylmethylene)hydrazinyl]-β-D-galactopyranosyl}-3-methyl-1H-1,2,4-triazol-1-yl}-2-(trifluoromethyl)benzene

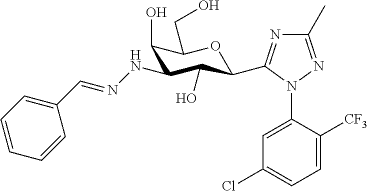

To a solution of 1-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-5-chloro-2-(trifluoromethyl)benzene (200 mg, 0.32 mmol) in EtOH (10 mL) HCl (2.0 mL, 4 M) was added and the mixture was stirred overnight at rt. The mixture was concentrated and purified by reversed-phase chromatography (MeCN/H₂O=1/20˜1/1, (10 mmol/L NH₄HCO₃), C-18 column, 20 mL/min, UV 214) to afford the product (70 mg, 42%). ESI-MS m/z calcd for [C₂₃H₂₃ClF₃N₅O₄] [M+H]⁺: 526.1; found: 525.9. ¹H NMR (400 MHz, Methanol-d₄) δ 7.93 (d, J=8.4 Hz, 1H), 7.87-7.74 (m, 3H), 7.55-7.48 (m, 2H), 7.35-7.20 (m, 3H), 4.21-4.12 (m, 2H), 4.05 (d, J=2.4 Hz, 1H), 3.64-3.62 (m, 2H), 3.52 (t, J=6.0 Hz, 1H), 3.34-3.30 (m, 1H), 2.41 (s, 3H).

### Intermediate 48

### 1-[5-(3-Amino-3-deoxy-β-D-galactopyranosyl)-3-methyl-1H-1,2,4-triazol-1-yl]-5-chloro-2-(trifluoromethyl)benzene

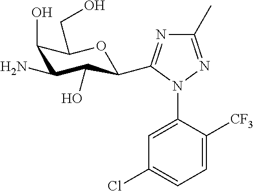

To a solution of 1-[5-(3-amino-4,6-O-benzylidene-3-deoxy-β-D-galactopyranosyl)-3-methyl-1H-1,2,4-triazol-1-yl]-5-chloro-2-(trifluoromethyl)benzene (200 mg, 0.39 mmol) in DCM (10 mL) TFA (1.0 mL) was added and the mixture was stirred overnight at rt. The mixture was concentrated and purified by reversed-phase chromatography (MeCN/H₂O (10 mmol/L NH₄HCO₃)=1/20˜1/2, C-18 column, 20 mL/min, UV 214) to afford the product (140 mg, 85%). ESI-MS m/z calcd for [C₁₆H₁₈ClF₃N₄O₄] [M+H]⁺: 423.1; found: 423.0. ¹H NMR (400 MHz, Methanol-d₄) δ 7.92 (d, J=8.8 Hz, 1H), 7.83 (dd, J=8.8, 1.2 Hz, 1H), 7.75 (s, 1H), 4.03 (d, J=9.6 Hz, 1H), 3.89 (br s, 1H), 3.81 (d, J=2.4 Hz, 1H), 3.62-3.60 (m, 2H), 3.45 (t, J=6.4 Hz, 1H), 2.60 (dd, J=9.6, 1.6 Hz, 1H), 2.42 (s, 3H).

### 1-{5-{3-[(2-tert-Butoxycarbonyl)hydrazinyl]-3-deoxy-β-D-galactopyranosyl}-3-methyl-1H-1,2,4-triazol-1-yl}-5-chloro-2-(trifluoromethyl)benzene

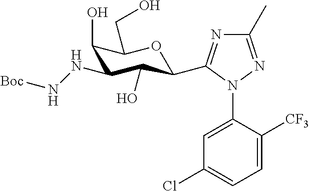

To a solution of 1-[5-(3-amino-3-deoxy-β-D-galactopyranosyl)-3-methyl-1H-1,2,4-triazol-1-yl]-5-chloro-2-(trifluoromethyl)benzene (130 mg, 0.31 mmol) in DCM (10 mL) N-tert-butyloxycarbonyl-3-(4-cyanophenyl)oxaziridine (114 mg, 0.46 mmol) was added and the mixture was stirred overnight at rt. The mixture was concentrated and purified by reversed-phase chromatography (MeCN/H₂O (10 mmol/L NH₄HCO₃)=1/20˜1/2, C-18 column, 20 mL/min, UV 214) to afford the product (30 mg, 18%). ESI-MS m/z calcd for [C₂₁H₂₇ClF₃N₅O₆] [M+H]⁺: 538.2; found: 538.0. ¹H NMR (400 MHz, DMSO-d₆) δ 8.12 (s, 1H), 7.99 (d, J=8.4 Hz, 1H), 7.90 (dd, J=8.4, 1.2 Hz, 1H), 7.73 (s, 1H), 5.15 (d, J=5.2 Hz, 1H), 4.64-4.50 (m, 2H), 4.10-3.98 (m, 2H), 3.87 (br s, 1H), 3.65 (s, 1H), 3.51-3.40 (m, 1H), 3.40-3.35 (m, 1H), 3.32-3.25 (m, 1H), 2.65 (d, J=9.6 Hz, 1H), 2.32 (s, 3H), 1.38 (s, 9H).

### 5-Chloro-1-{5-{3-deoxy-3-{2-[(2-fluorophenyl)methylene]hydrazinyl}-β-D-galactopyranosyl}-3-methyl-1H-1,2,4-triazol-1-yl}-2-(trifluoromethyl)benzene

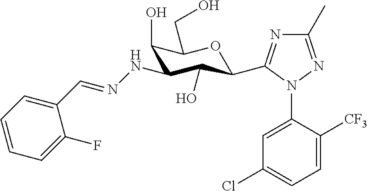

To a solution of 1-{5-{3-[(2-tert-butoxycarbonyl)hydrazinyl]-3-deoxy-β-D-galactopyranosyl}-3-methyl-1H-1,2,4-triazol-1-yl}-5-chloro-2-(trifluoromethyl)benzene (160 mg, 0.30 mmol) in EtOH (4.0 mL) 2-fluorobenzaldehyde (111 mg, 0.89 mmol) was added followed by HCl (2.0 mL, 4 M in 1,4-dioxane) and the mixture was stirred overnight at rt. The mixture was concentrated and purified by reversed-phase chromatography (MeCN/H₂O (10 mmol/L NH₄HCO₃)=1/20˜1/1, C-18 column, 20 mL/min, UV 214) to afford the product (55 mg, 34%). ESI-MS m/z calcd for [C₂₃H₂₂ClF₄N₅O₄] [M+H]⁺: 544.1; found: 543.9. ¹H NMR (400 MHz, DMSO-d₆) δ 8.01 (d, J=8.4 Hz, 1H), 7.96-7.88 (m, 2H), 7.75 (s, 1H), 7.69 (td, J=7.6, 1.2 Hz, 1H), 7.36 (d, J=7.2 Hz, 1H), 7.31-7.22 (m, 1H), 7.18-7.12 (m, 2H), 5.01 (s, 1H), 4.72 (d, J=4.4 Hz, 1H), 4.60-4.50 (m, 1H), 4.17-3.97 (m, 2H), 3.92-3.77 (m, 1H), 3.50-3.35 (m, 4H), 2.33 (s, 3H).

### Intermediate 50

### 1-Phenylpropan-2-one

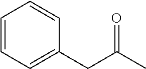

To a solution of 1-phenylpropan-2-ol (2.0 g, 14.7 mmol) in DCM (30 mL) Dess-Martin Periodinane (7.47 g, 17.6 mmol) was added at 0° C. and the mixture was stirred 1 h. The precipitate was removed by filtration and the filtrate was concentrated. The residue was purified by column chromatography (EtOAc/PE=0/1˜1/4, Silica-CS 20 g, 20 mL/min, silica gel, UV 254) to afford the product (1.8 g, 91%). ESI-MS m/z calcd for [C₉H₁₀O][M]: 134.1; found: 134.0. ¹H NMR (400 MHz, Chloroform-d) δ 7.35-7.08 (m, 5H), 3.62 (s, 2H), 2.08 (s, 3H).

### (E)-4-Hydroxy-1-phenylbut-3-en-2-one

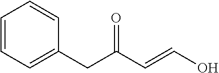 [SMILES: O=C(/C=C/O)Cc1ccccc1]

A solution 1-phenylpropan-2-one (500 mg, 3.73 mmol) in anhydrous diethyl ether (25 mL) containing sodium (85.7 mg, 3.73 mmol) cut in small pieces was prepared. To the solution ethyl formate (414 mg, 5.59 mmol) was added slowly with stirring and ice-cooling under nitrogen atmosphere. The mixture was stirred overnight at rt. Water (20 mL) and EtOAc (20 mL) were added. The aqueous phase was separated, and the pH was adjusted to 3-4 using 2N HCl. The aqueous phase was extracted with EtOAc (3×20 mL). The combined organic phases were dried and concentrated to afford the product (300 mg, 50%). ESI-MS m/z calcd for [C₁₀H₁₀O₂] [M+H]⁺: 163.1; found: 163.2. ¹H NMR (400 MHz, Chloroform-d) δ 7.77 (d, J=4.4 Hz, 1H), 7.35-7.10 (m, 5H), 5.42 (d, J=4.4 Hz, 1H), 3.58 (s, 2H).

### (E)-4-(Dimethylamino)-1-phenylbut-3-en-2-one

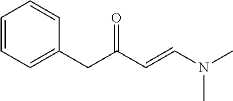

To a solution of (E)-4-hydroxy-1-phenylbut-3-en-2-one (300 mg, 1.85 mmol) in THF (10 mL) K₂CO₃ (1.53 g, 11.1 mmol) was added followed by dimethylamine hydrochloride (453 mg, 5.55 mmol), and the mixture was stirred overnight at rt. The mixture was evaporated and purified by reversed-phase chromatography (MeCN/H₂O=1/20˜1/1, C-18 column, 20 mL/min, UV 214) to afford the product (160 mg, 46%). ESI-MS m/z calcd for [C₁₂H₁₅NO] [M+H]⁺: 190.1; found: 190.2. ¹H NMR (400 MHz, Methanol-d₄) δ 7.67 (d, J=12.4 Hz, 1H), 7.33-7.12 (m, 5H), 5.12 (d, J=12.4 Hz, 1H), 3.62 (s, 2H), 3.11 (s, 3H), 2.82 (s, 3H).

### 1-{5-[3-(3-Benzyl-1H-1,2-pyrazol-1-yl)-3-deoxy-β-D-galactopyranosyl]-3-methyl-1H-1,2,4-triazol-1-yl}-5-chloro-2-(trifluoromethyl)benzene

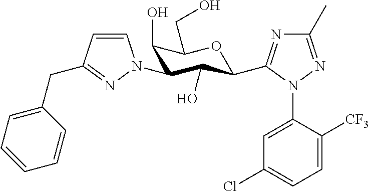

To a solution of 1-{5-{3-[(2-tert-butoxycarbonyl)hydrazinyl]-3-deoxy-3-D-galactopyranosyl}-3-methyl-1H-1,2,4-triazol-1-yl}-5-chloro-2-(trifluoromethyl)benzene (100 mg, 0.19 mmol) and (E)-4-(dimethylamino)-1-phenylbut-3-en-2-one (70.4 mg, 0.37 mol) in EtOH (5.0 mL) concentrated HCl (0.25 mL) was added and the mixture was stirred 1 h at 80° C. The mixture was concentrated and first purified by reversed-phase chromatography (MeCN/H₂O=1/20˜1/1, C-18 column, 20 mL/min, UV 214) and then prep TLC (MeOH/DCM=1/20) to afford the product (26 mg, 25%). ESI-MS m/z calcd for [C₂₆H₂₅ClF₃N₅O₄] [M+H]⁺: 564.2; found: 563.9. ¹H NMR (400 MHz, Methanol-d₄) δ 7.94 (d, J=8.8 Hz, 1H), 7.84 (dd, J=8.8, 0.8 Hz, 1H), 7.76 (s, 1H), 7.66 (d, J=2.4 Hz, 1H), 7.28-7.20 (m, 4H), 7.19-7.12 (m, 1H), 6.07 (d, J=2.0 Hz, 1H), 4.72-4.59 (m, 1H), 4.28 (d, J=2.8 Hz, 1H), 4.24 (d, J=8.8 Hz, 1H), 4.04 (d, J=2.8 Hz, 1H), 3.93 (s, 2H), 3.67-3.59 (m, 3H), 2.42 (s, 3H).

### 1-{5-[3-(3-Benzyl-4-iodo-1H-1,2-pyrazol-1-yl)-3-deoxy-β-D-galactopyranosyl]-3-methyl-1H-1,2,4-triazol-1-yl}-5-chloro-2-(trifluoromethyl)benzene

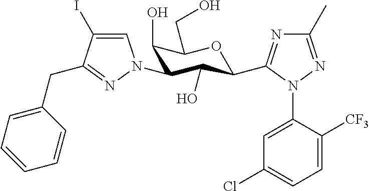

To a solution of 1-{5-[3-(3-benzyl-1H-1,2-pyrazol-1-yl)-3-deoxy-β-D-galactopyranosyl]-3-methyl-1H-1,2,4-triazol-1-yl}-5-chloro-2-(trifluoromethyl)benzene (24 mg, 0.043 mmol) in THF (5.0 mL) N-iodosuccinimide (239 mg, 1.06 mmol) was added and the mixture was stirred 3 h at rt. The mixture was concentrated and purified by reversed-phase chromatography (MeCN/H₂O=1/20˜1/1, C-18 column, 20 mL/min, UV 214) to afford the product (28 mg, 95%). ESI-MS m/z calcd for [C₂₆H₂₄ClF₃IN₅O₄] [M+H]⁺: 690.1; found: 690.1. ¹H NMR (400 MHz, Methanol-d₄) δ 7.94 (d, J=8.8 Hz, 1H), 7.84 (dd, J=8.8, 1.2 Hz, 1H), 7.81 (s, 1H), 7.75 (s, 1H), 7.26-7.11 (m, 5H), 4.72-4.59 (m, 1H), 4.31 (dd, J=10.4, 2.4 Hz, 1H), 4.24 (d, J=9.2 Hz, 1H), 4.02 (d, J=2.4 Hz, 1H), 3.93 (s, 2H), 3.65-3.57 (m, 3H), 2.42 (s, 3H).

### Intermediate 51

### 5-Chloro-1-{5-{3-deoxy-3-[4-(3,4,5-trifluorophenyl)-3-ethoxycarbonyl-1H-1,2-pyrazol-1-yl]-β-D-galactopyranosyl}-3-methyl-1H-1,2,4-triazol-1-yl}-2-(trifluoromethyl)benzene

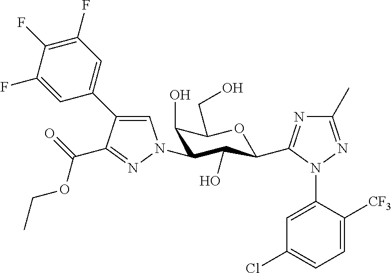

A nitrogen purged solution of 5-chloro-1-{5-[3-deoxy-3-(3-ethoxycarbonyl-4-iodo-1H-1,2-pyrazol-1-yl)-β-D-galactopyranosyl]-3-methyl-1H-1,2,4-triazol-1-yl}-2-(trifluoromethyl)benzene (65 mg, 0.097 mmol), (3,4,5-trifluorophenyl)boronic acid (34.0 mg, 0.19 mmol), K₂CO₃ (40.3 mg, 0.29 mmol) and Pd(dppf)Cl₂ (7.07 mg, 0.0097 mmol) in 1,4-dioxane (2 mL) and water (0.5 mL) was stirred 2 h at 60° C. The mixture was filtered, concentrated, and purified by prep HPLC (MeCN/H₂O (10 mmol/L NH₄CO₃), X-Select10 μm 19*250 mm, 20 mL/min, UV 254) to afford the product (42 mg, 64%). ESI-MS m/z calcd for [C₂₈H₂₄ClF₆N₅O₆] [M+H]⁺: 676.1; found: 675.7. ¹H NMR (400 MHz, Methanol-d₄) δ 8.04 (s, 1H), 7.95 (d, J=8.8 Hz, 1H), 7.85 (d, J=8.4 Hz, 1H), 7.77 (s, 1H), 7.35-7.29 (m, 2H), 4.81-4.76 (m, 1H), 4.47 (dd, J=10.8, 2.8 Hz, 1H), 4.36-4.28 (m, 3H), 4.11 (d, J=2.8 Hz, 1H), 3.67-3.63 (m, 3H), 2.44 (s, 3H), 1.29 (t, J=7.2 Hz, 3H).

### Intermediate 52

### 5-Chloro-1-{5-{3-deoxy-3-{2-[(2-methoxyphenyl)methylene]hydrazinyl}-β-D-galactopyranosyl}-3-methyl-1H-1,2,4-triazol-1-yl}-2-(trifluoromethyl)benzene

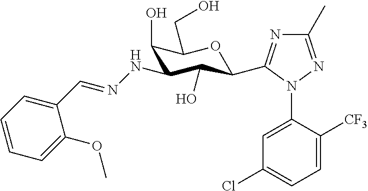

To a solution of 1-{5-{3-[(2-tert-butoxycarbonyl)hydrazinyl]-3-deoxy-β-D-galactopyranosyl}-3-methyl-1H-1,2,4-triazol-1-yl}-5-chloro-2-(trifluoromethyl)benzene (100 mg, 0.19 mmol) in EtOH (4.0 mL) 2-methoxybenzaldehyde (75.9 mg, 0.56 mmol) was added followed by HCl (2.0 mL, 4 M in 1,4-dioxane) and the mixture was stirred overnight at rt. The mixture was concentrated and purified by reversed-phase chromatography (MeCN/H₂O (10 mmol/L NH₄HCO₃)=1/20˜1/1, C-18 column, 20 mL/min, UV 214) to afford the product (48 mg, 46%). ESI-MS m/z calcd for [C₂₄H₂₅ClF₃N₅O₅] [M+H]⁺: 556.1; found: 555.9. ¹H NMR (400 MHz, Methanol-d₄) δ 8.20 (s, 1H), 7.93 (d, J=8.8 Hz, 1H), 7.84 (dd, J=8.8, 1.2 Hz, 1H), 7.78 (s, 1H), 7.66 (dd, J=8.0, 1.6 Hz, 1H), 7.31-7.22 (m, 1H), 6.97 (d, J=7.6 Hz, 1H), 6.89 (t, J=7.6 Hz, 1H), 4.22-4.12 (m, 2H), 4.06 (d, J=2.0 Hz, 1H), 3.84 (s, 3H), 3.65-3.63 (m, 2H), 3.52 (t, J=6.0 Hz, 1H), 3.29-3.26 (m, 1H), 2.42 (s, 3H).

### Intermediate 53

### (E)-3-(Dimethylamino)-1-(2-methylphenyl)-2-propen-1-one

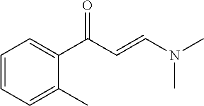

To a solution of 2′-methylacetophenone (500 mg, 3.73 mmol) in toluene (8.0 mL) 1,1-dimethoxy-N,N-dimethylmethanamine (888 mg, 7.45 mmol) was added and the mixture was stirred 5 h at 110° C. The mixture was concentrated and purified by column chromatography (PE/EtOAc=5/1˜0/1, Silica-CS 40 g, 50 mL/min, silica gel, UV 254) to afford the product (205 mg, 29%). ESI-MS m/z calcd for [C₁₂H₁₅NO] [M+H]⁺: 190.1; found: 190.3. ¹H NMR (400 MHz, Chloroform-d) δ 7.27-7.07 (m, 5H), 5.27 (d, J=12.8 Hz, 1H), 2.99 (s, 3H), 2.79 (s, 3H), 2.32 (s, 3H).

### 5-Chloro-1-{5-{3-deoxy-3-[3-(2-methylphenyl)-1H-1,2-pyrazol-1-yl]-β-D-galactopyranosyl}-3-methyl-1H-1,2,4-triazol-1-yl}-2-(trifluoromethyl)benzene

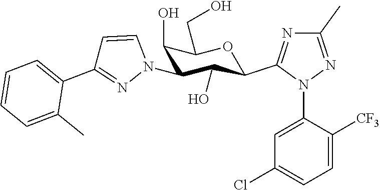

To a solution of 1-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-5-chloro-2-(trifluoromethyl)benzene (120 mg, 0.22 mmol) and (E)-3-(dimethylamino)-1-(2-methylphenyl)-2-propen-1-one (63.3 mg, 0.34 mmol) in EtOH (5.0 mL) concentrated HCl (0.25 mL) was added and the mixture was stirred 1 h at 90° C. The mixture was concentrated and purified by reversed-phase chromatography (MeCN/H₂O=1/20˜1/1, C-18 column, 20 mL/min, UV 214) to afford the product (34 mg, 27%). ESI-MS m/z calcd for [C₂₆H₂₅ClF₃N₅O₄] [M+H]⁺: 564.1; found: 564.2. ¹H NMR (400 MHz, Methanol-d₄) δ 7.95 (d, J=8.8 Hz, 1H), 7.85 (dd, J=8.4, 1.2 Hz, 1H), 7.81 (d, J=2.4 Hz, 1H), 7.78 (s, 1H), 7.50-7.46 (m, 1H), 7.26-7.17 (m, 3H), 6.49 (d, J=2.4 Hz, 1H), 4.81-4.68 (m, 1H), 4.38 (dd, J=10.8, 2.8 Hz, 1H), 4.29 (d, J=9.2 Hz, 1H), 4.14 (d, J=2.4 Hz, 1H), 3.69-3.62 (m, 3H), 2.46-2.41 (m, 6H).

### 5-Chloro-1-{5-{3-deoxy-3-[4-iodo-3-(2-methylphenyl)-1H-1,2-pyrazol-1-yl]-β-D-galactopyranosyl}-3-methyl-1H-1,2,4-triazol-1-yl}-2-(trifluoromethyl)benzene

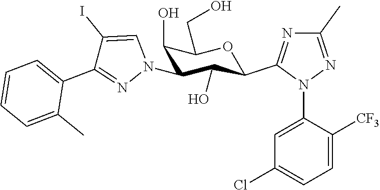

To a solution of 5-chloro-1-{5-{3-deoxy-3-[3-(2-methylphenyl)-1H-1,2-pyrazol-1-yl]-β-D-galactopyranosyl}-3-methyl-1H-1,2,4-triazol-1-yl}-2-(trifluoromethyl)benzene (34 mg, 0.060 mmol) in THF (5.0 mL) N-iodosuccinimide (239 mg, 1.06 mmol) was added and the mixture was stirred overnight at rt. The mixture was concentrated and purified by reversed-phase chromatography (MeCN/H₂O=1/20˜1/1, C-18 column, 20 m/min, UV 214) to afford the product (35 mg, 84%). ESI-MS m/z calcd for [C₂₆H₂₄ClF₃IN₅O₄] [M+H]⁺: 690.1; found: 690.1. ¹H NMR (400 MHz, Methanol-d₄) δ 7.96-7.93 (m, 2H), 7.85 (d, J=8.8 Hz, 1H), 7.76 (s, 1H), 7.33-7.18 (m, 4H), 4.79-4.66 (m, 1H), 4.39 (dd, J=10.8, 2.8 Hz, 1H), 4.27 (d, J=9.6 Hz, 1H), 4.12-4.07 (m, 1H), 3.69-3.61 (m, 3H), 2.44 (s, 3H), 2.22 (s, 3H).

### Intermediate 54

### (E)-3-(Dimethylamino)-1-(3-fluorophenyl)-2-propen-1-one

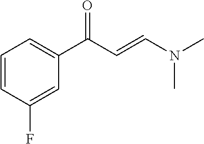

To a solution of 1-(3-fluorophenyl)ethanone (500 mg, 3.62 mmol) in toluene (10.0 mL) 1,1-dimethoxy-N,N-dimethylmethanamine (863 mg, 7.24 mmol) was added and the mixture was stirred 5 h at 110° C. The mixture was concentrated and purified by column chromatography (PE/EtOAc=5/1˜0/1, Silica-CS 40 g, 50 mL/min, silica gel, UV 254) to afford the product (265 mg, 38%). ESI-MS m/z calcd for [C_{II}H₁₂FNO] [M+H]⁺: 194.1; found: 194.2. ¹H NMR (400 MHz, Chloroform-d) δ 8.03 (d, J=10.8 Hz, 1H), 7.67 (dd, J=7, 6, 1.2 Hz, 1H), 7.61-7.56 (m, 1H), 7.41-7.35 (m, 1H), 7.18-7.14 (m, 1H), 5.69 (d, J=12.0 Hz, 1H), 3.21 (s, 3H), 2.98 (s, 3H).

### 5-Chloro-1-{5-{3-deoxy-3-[3-(3-fluorophenyl)-1H-1,2-pyrazol-1-yl]-β-D-galactopyranosyl}-3-methyl-1H-1,2,4-triazol-1-yl}-2-(trifluoromethyl)benzene

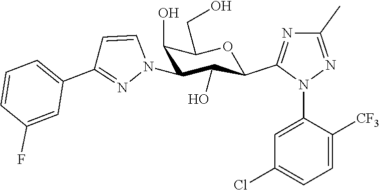

To a solution of 1-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-5-chloro-2-(trifluoromethyl)benzene (115 mg, 0.21 mmol) and (E)-3-(dimethylamino)-1-(3-fluorophenyl)-2-propen-1-one (74.4 mg, 0.39 mmol) in EtOH (5.0 mL) concentrated HCl (0.25 mL) was added and the mixture was stirred 1 h at 90° C. The mixture was concentrated and purified by column chromatography (EtOAc/PE=1/2˜5/1, Silica-CS 12 g, 20 mL/min, silica gel, UV 254) to give the product (35 mg, 29%). ESI-MS m/z calcd for [C₂₅H₂₂ClF₄N₅O₄] [M+H]⁺: 568.1; found: 567.9. ¹H NMR (400 MHz, Methanol-d₄) δ 7.77 (d, J=8.4 Hz, 1H), 7.71 (d, J=2.4 Hz, 1H), 7.68-7.60 (m, 2H), 7.57-7.52 (m, 1H), 7.49-7.44 (m, 1H), 7.41-7.32 (m, 1H), 7.04-6.99 (m, 1H), 6.61 (d, J=2.4 Hz, 1H), 4.74 (t, J=9.6 Hz, 1H), 4.46 (d, J=9.2, 1H), 4.43 (d, J=2.0 Hz, 1H), 4.22 (dd, J=10.4, 2.0 Hz, 1H), 3.65-3.35 (m, 3H), 2.43 (s, 3H).

### 5-Chloro-1-{5-{3-deoxy-3-[3-(3-fluorophenyl)-4-iodo-1H-1,2-pyrazol-1-yl]-β-D-galactopyranosyl}-3-methyl-1H-1,2,4-triazol-1-yl}-2-(trifluoromethyl)benzene

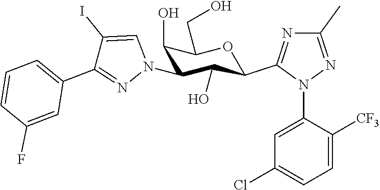

To a solution of 5-chloro-1-{5-{3-deoxy-3-[3-(3-fluorophenyl)-1H-1,2-pyrazol-1-yl]-β-D-galactopyranosyl}-3-methyl-1H-1,2,4-triazol-1-yl}-2-(trifluoromethyl)benzene (33 mg, 0.058 mmol) in THF (5.0 mL) N-iodosuccinimide (288 mg, 1.28 mmol) was added and the mixture was stirred overnight at rt. The mixture was concentrated and purified by reversed-phase chromatography (MeCN/H₂O=1/20˜1/1, C-18 column, 20 m/min, UV 214) to afford the product (26 mg, 65%). ESI-MS m/z calcd for [C₂₅H₂₁ClF₄IN₅O₄] [M+H]⁺: 694.0; found: 693.7. ¹H NMR (400 MHz, Methanol-d₄) δ 7.96-7.93 (m, 2H), 7.87-7.83 (m, 1H), 7.78-7.74 (m, 1H), 7.67-7.65 (m, 1H), 7.58-7.53 (m, 1H), 7.46-7.42 (m, 1H), 7.12-7.07 (m, 1H), 4.82-4.71 (m, 1H), 4.42 (dd, J=10.8, 2.8 Hz, 1H), 4.28 (d, J=9.2 Hz, 1H), 4.09 (d, J=2.4 Hz, 1H), 3.67-3.62 (m, 3H), 2.44 (s, 3H).

### Intermediate 55

### (E)-3-(Dimethylamino)-1-(4-fluorophenyl)-2-propen-1-one

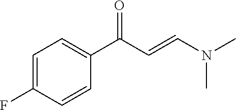

To a solution of 1-(4-fluorophenyl)ethanone (200 mg, 1.45 mmol) in toluene (5.0 mL) 1,1-dimethoxy-N,N-dimethylmethanamine (345 mg, 2.90 mmol) was added and the mixture was stirred 5 h at 110° C. The mixture was concentrated and purified by column chromatography (PE/EtOAc=5/1˜0/1, Silica-CS 40 g, 50 mL/min, silica gel, UV 254) to afford the product (75 mg, 27%). ESI-MS m/z calcd for [C_{II}H₁₂FNO] [M+H]⁺: 194.1; found: 194.3. ¹H NMR (400 MHz, Chloroform-d) δ 7.90-7.81 (m, 2H), 7.78 (d, J=12.0 Hz, 1H), 7.05-6.97 (m, 2H), 5.61 (d, J=12.0 Hz, 1H), 3.10 (br s, 3H), 2.86 (br s, 3H).

### 5-Chloro-1-{5-{3-deoxy-3-[3-(4-fluorophenyl)-1H-1,2-pyrazol-1-yl]-β-D-galactopyranosyl}-3-methyl-1H-1,2,4-triazol-1-yl}-2-(trifluoromethyl)benzene

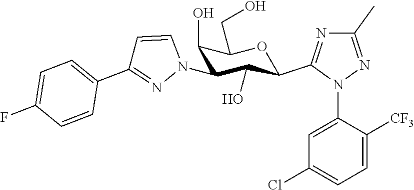

To a solution of 1-{5-{4,6-O-benzylidene-3-[(2-tert-butoxycarbonyl)hydrazinyl]-3-deoxy-β-D-galactopyranosyl}-3-methyl-1H-1,2,4-triazol-1-yl}-5-chloro-2-(trifluoromethyl)benzene (120 mg, 0.22 mmol) and (E)-3-(dimethylamino)-1-(4-fluorophenyl)-2-propen-1-one (64.7 mg, 0.34 mmol) in EtOH (5.0 mL) concentrated HCl (0.25 mL) was added and the mixture was stirred 1 h at 90° C. The mixture was concentrated and purified by reversed-phase chromatography (MeCN/H₂O=1/20˜1/1, C-18 column, 20 m/min, UV 214). The obtained material was further purified by preparative TLC (PE/EtOAc=1/3) to afford the product (35 mg, 28%). ESI-MS m/z calcd for [C₂₅H₂₂ClF₄N₅O₄] [M+H]⁺: 568.1; found: 568.2. ¹H NMR (400 MHz, Methanol-d₄) δ 7.95 (d, J=8.8 Hz, 1H), 7.85 (d, J=8.8 Hz, 1H), 7.83-7.74 (m, 4H), 7.16-7.07 (m, 2H), 6.65 (d, J=2.4 Hz, 1H), 4.81-4.68 (m, 1H), 4.38 (dd, J=10.8, 2.8 Hz, 1H), 4.29 (d, J=9.2 Hz, 1H), 4.13 (d, J=2.8 Hz, 1H), 3.69-3.62 (m, 3H), 2.43 (s, 3H).

### 5-Chloro-1-{5-{3-deoxy-3-[3-(4-fluorophenyl)-4-iodo-1H-1,2-pyrazol-1-yl]-β-D-galactopyranosyl}-3-methyl-1H-1,2,4-triazol-1-yl}-2-(trifluoromethyl)benzene

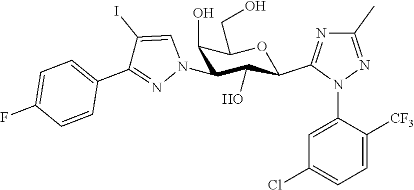

To a solution of 5-chloro-1-{5-{3-deoxy-3-[3-(4-fluorophenyl)-1H-1,2-pyrazol-1-yl]-β-D-galactopyranosyl}-3-methyl-1H-1,2,4-triazol-1-yl}-2-(trifluoromethyl)benzene (35 mg, 0.062 mmol) in THF (5.0 mL) N-iodosuccinimide (208 mg, 0.92 mmol) was added and the mixture was stirred overnight at rt. The mixture was concentrated and purified by reversed-phase chromatography (MeCN/H₂O=1/20˜1/1, C-18 column, 20 m/min, UV 214) to afford the product (32 mg, 75%). ESI-MS m/z calcd for [C₂₅H₂₁ClF₄IN₅O₄] [M+H]⁺: 694.0; found: 694.0. ¹H NMR (400 MHz, Methanol-d₄) δ 7.89-7.83 (m, 2H), 7.79-7.64 (m, 4H), 7.11-7.01 (m, 2H), 4.71-4.59 (m, 1H), 4.31 (dd, J=10.4, 2.4 Hz, 1H), 4.18 (d, J=9.2 Hz, 1H), 3.99 (d, J=2.8 Hz, 1H), 3.61-3.51 (m, 3H), 2.34 (s, 3H).

### Intermediate 56

### 3-(Dimethylamino)-2-[4-(trifluoromethyl)phenyl]prop-2-enal

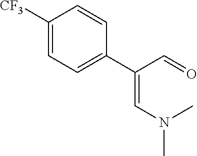 [SMILES: CN(C)/C=C(\C=O)c1ccc(C(F)(F)F)cc1]

To a cooled (0° C.) solution of N,N-dimethylformamide (1.07 g, 14.7 mmol) and phosphoryl trichloride (2.25 g, 14.7 mmol) 2-[4-(trifluoromethyl)phenyl]acetic acid (300 mg, 1.47 mmol) was added and the mixture was stirred 3 h at 65° C. After cooling to rt, the mixture was slowly added to a mixture of ice and water. K₂CO₃ was added slowly until pH reached 11. Small quantities of ethanol were added to control frothing. To the mixture toluene (30 mL) was added, and the mixture was refluxed for 1.5 h and then cooled to rt. The aqueous phase was extracted with additional toluene (30 mL). The combined organic phases were washed with water, dried over Na₂SO₄ and evaporated. The obtained solid was recrystallized from hexane to afford the product (180 mg, 50%). ESI-MS m/z calcd for [C₁₂H₁₂F₃NO] [M+H]⁺: 244.1; found: 244.1. ¹H NMR (400 MHz, Chloroform-d) δ 9.03 (d, J=1.6 Hz, 1H), 7.52 (d, J=7.2 Hz, 2H), 7.24 (d, J=7.6 Hz, 2H), 6.79 (br s, 1H), 2.79 (br s, 6H).

### Intermediate 57

### 3-(Dimethylamino)-2-(3-fluoro-4-methylphenyl)prop-2-enal

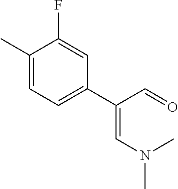

To a cooled (0° C.) solution of N,N-dimethylformamide (652 mg, 8.92 mmol) and phosphoryl trichloride (1277 mg, 8.33 mmol) 2-(3-fluoro-4-methylphenyl)acetic acid (100 mg, 0.60 mmol) was added and the mixture was stirred overnight at 60-70° C. After cooling to rt, the mixture was slowly added to a mixture of ice and water with external cooling and ice intermittently to keep the temperature below 10° C. K₂CO₃ was added slowly until pH reached 11. Small quantities of ethanol were added to control frothing. To the alkaline mixture toluene (20 mL) was added, and the mixture was refluxed for 1.5 h and then cooled to rt. The aqueous phase was extracted with additional toluene (40 mL). The combined organic phases were washed with water, dried over Na₂SO₄, evaporated, and purified by column chromatography (PE/EtOAc=1/1˜0/1, Silica-CS 12 g, 20 mL/min, silica gel, UV 254) to afford the product (245 mg, 45%). ESI-MS m/z calcd for [C₁₂H₁₄FNO] [M+H]⁺: 208.1; found: 208.2. ¹H NMR (400 MHz, Chloroform-d) δ 9.07 (s, 1H), 7.15-7.11 (m, 1H), 6.87-6.75 (m, 3H), 2.88 (br s, 6H), 2.25 (s, 3H).

### Intermediate 58

### 3-(Dimethylamino)-2-(3-chlorophenyl)prop-2-enal

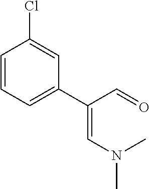

To a cooled (0° C.) solution of N,N-dimethylformamide (1.29 g, 17.6 mmol) and phosphoryl trichloride (2.70 g, 17.6 mmol) 2-(3-chlorophenyl)acetic acid (300 mg, 1.76 mmol) was added and the mixture was stirred 3 h at 65° C. After cooling to rt, the mixture was slowly added to a mixture of ice and water. K₂CO₃ was added slowly until pH reached 11. Small quantities of ethanol were added to control frothing. To the alkaline mixture toluene (30 mL) was added, and the mixture was refluxed for 1.5 h and then cooled to rt. The aqueous phase was extracted with EtOAc (30 mL). The combined organic phases were washed with brine, dried Na₂SO₄ and evaporated. The obtained solid was recrystallized from hexane to afford the product (145 mg, 39%).

ESI-MS m/z calcd for [C₁₁H₁₂ClNO] [M+H]⁺: 210.1; found: 210.4. ¹H NMR (400 MHz, Chloroform-d) δ 9.08 (s, 1H), 7.29-7.22 (m, 2H), 7.18 (s, 1H), 7.08 (d, J=7.2 Hz, 1H), 6.80 (br s, 1H), 2.86 (br s, 6H).

### Intermediate 59

### 3-(Dimethylamino)-2-(4-chlorophenyl)prop-2-enal

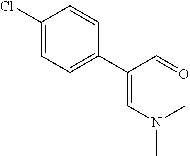

To a cooled (0° C.) solution of N,N-dimethylformamide (2 mL) and phosphoryl trichloride (2 mL) 2-(4-chlorophenyl)acetic acid (341 mg, 2.00 mmol) was added and the mixture was stirred overnight at 60-70° C. After cooling to rt, the mixture was slowly added to a mixture of ice and water with external cooling and ice intermittently to keep the temperature below 10° C. K₂CO₃ was added slowly until pH reached 11. Small quantities of ethanol were added to control frothing. To the alkaline mixture toluene (20 mL) was added, and the mixture was refluxed for 1.5 h and then cooled to rt. The aqueous phase was extracted with additional toluene (40 mL). The combined organic phases were washed with water, dried Na₂SO₄ and evaporated. The obtained solid was washed with PE/EtOAc (3 mL, 10:1) to afford the product (223 mg, 53%). ESI-MS m/z calcd for [C₁₁H₁₂ClNO] [M+H]⁺: 210.1; found: 210.1. H NMR (400 MHz, Chloroform-d) δ 9.08 (s, 1H), 7.31 (d, J=8.4 Hz, 2H), 7.12 (d, J=8.4 Hz, 2H), 6.85 (br s, 1H), 2.88 (br s, 6H).

### REFERENCES

Bennett, D.; Bargagli, E.; Bianchi, N.; Landi, C.; Fossi, A.; Fui, A.; Sestini, P.;
Refini, R. M.; Rottoli, P. Elevated Level of Galectin-1 in Bronchoalveolar Lavage of Patients with Idiopathic Pulmonary Fibrosis. Respir Physiol Neurobiol 2019, 273, 103323. https://doi.org/10.1016/j.resp.2019.103323.
Blanchard, H.; Yu, X.; Collins, P. M.; Burn-Erdene, K. Galectin-3 Inhibitors: A Patent Review (2008-Present). Expert Opin. Ther. Patents 2014, 24 (10), 1053-1065. https://doi.org/10.1517/13543776.2014.947961.
Blanchard, H.; Burn-Erdene, K.; Bohari, M. H.; Yu, X. Galectin-1 Inhibitors and Their Potential Therapeutic Applications: A Patent Review. Expert Opin. Ther. Patents 2016, 26 (5), 537-554. https://doi.org/10.1517/13543776.2016.1163338.
Daley, D.; Mani, V. R.; Mohan, N.; Akkad, N.; Ochi, A.; Heindel, D. W.; Lee, K. B.; Zambirinis, C. P.; Pandian, G. S. D. B.; Savadkar, S.; Torres-Hernandez, A.; Nayak, S.; Wang, D.; Hundeyin, M.; Diskin, B.; Aykut, B.; Werba, G.; Barilla, R. M.; Rodriguez, R.; Chang, S.; Gardner, L.; Mahal, L. K.; Ueberheide, B.; Miller, G. Dectin-1 Activation on Macrophages by Galectin-9 Promotes Pancreatic Carcinoma and Peritumoral Immune-Tolerance. Nat Med 2017, 23 (5), 556-567. https://doi.org/10.1038/nm.4314.
Dings, R. P. M., Miller, M. C., Griffin, R. J. & Mayo, K. H. Galectins as Molecular Targets for Therapeutic Intervention. IJMS 19, (2018).
Dubd-Delarosbil, C.; St-Pierre, Y. The Emerging Role of Galectins in High-Fatality Cancers. Cell. Mol. Life Sci. 2017, 75 (7), 1215-1226. https://doi.org/10.1007/s00018-017-2708-5.
Drake, I.; Fryk, E.; Strindberg, L.; Lundqvist, A.; Rosengren, A. H.; Groop, L.; Ahlqvist, E.; Bordn, J.; Orho-Melander, M.; Jansson, P.-A. The Role of Circulating Galectin-1 in Type 2 Diabetes and Chronic Kidney Disease: Evidence from Cross-Sectional, Longitudinal and Mendelian Randomisation Analyses. Diabetologia 2022, 65 (1), 128-139. https://doi.org/10.1007/s00125-021-05594-1.
Hsu, Y.-A.; Chang, C.-Y.; Lan, J.-L.; Li, J.-P.; Lin, H.-J.; Chen, C.-S.; Wan, L.; Liu, F.-T. Amelioration of Bleomycin-Induced Pulmonary Fibrosis via TGF-β-Induced Smad and Non-Smad Signaling Pathways in Galectin-9-Deficient Mice and Fibroblast Cells. J Biomed Sci 2020, 27 (1), 24. https://doi.org/10.1186/s12929-020-0616-8.
Johannes, L.; Jacob, R.; Leffler, H. Galectins at a Glance. J Cell Sci 2018, 131 (9), jcs208884. https://doi.org/10.1242/jcs.208884.
Kathiriya, J. J.; Nakra, N.; Nixon, J.; Patel, P. S.; Vaghasiya, V.; Alhassani, A.; Tian, Z.; Allen-Gipson, D.; Dav, V. Galectin-1 Inhibition Attenuates Profibrotic Signaling in Hypoxia-Induced Pulmonary Fibrosis. Cell Death Discovery 2017, 3, 17010-17013. https://doi.org/10.1038/cddiscovery.2017.10.
Li, P.; Liu, S.; Lu, M.; Bandyopadhyay, G.; Oh, D.; Imamura, T.; Johnson, A. M. F.; Sears, D.; Shen, Z.; Cui, B.; Kong, L.; Hou, S.; Liang, X.; Iovino, S.; Watkins, S. M.; Ying, W.; Osborn, O.; Wollam, J.; Brenner, M.; Olefsky, J. M. Hematopoietic-Derived Galectin-3 Causes Cellular and Systemic Insulin Resistance. Cell 2016, 167 (4), 973-984.e12. https://doi.org/10.1016/j.cell.2016.10.025.
Sethi, A.; Sanam, S.; Alvala, R.; Alvala, M. An Updated Patent Review of Galectin-1 and Galectin-3 Inhibitors and Their Potential Therapeutic Applications (2016-Present). Expert Opin Ther Pat 2021, 31 (8), 1-13. https://doi.org/10.1080/13543776.2021.1903430.
Slack, R. J.; Mills, R.; Mackinnon, A. C. The Therapeutic Potential of Galectin-3 Inhibition in Fibrotic Disease. Int J Biochem Cell Biology 2020, 105881. https://doi.org/10.1016/j.biocel.2020.105881.
Sundblad, V., Morosi, L. G., Geffner, J. R. & Rabinovich, G. A. Galectin-1: A Jack-of-All-Trades in the Resolution of Acute and Chronic Inflammation. J. Immunol. 199, 3721-3730 (2017).
Wolf, Y.; Anderson, A. C.; Kuchroo, V. K. TIM3 Comes of Age as an Inhibitory Receptor. Nat Rev Immunol 2020, 20 (3), 173-185. https://doi.org/10.1038/s41577-019-0224-6.
Wu, D.; Kanda, A.; Liu, Y.; Kase, S.; Noda, K.; Ishida, S. Galectin-1 Promotes Choroidal Neovascularization and Subretinal Fibrosis Mediated via Epithelial-Mesenchymal Transition. FASEB J. 2019, 33 (2), 2498-2513. https://doi.org/10.1096/fj.201801227r.
Yang, R.; Sun, L.; Li, C.-F.; Wang, Y.-H.; Yao, J.; Li, H.; Yan, M.; Chang, W.-C.; Hsu, J.-M.; Cha, J.-H.; Hsu, J. L.; Chou, C.-W.; Sun, X.; Deng, Y.; Chou, C.-K.; Yu, D.; Hung, M.-C. Galectin-9 Interacts with PD-1 and TIM-3 to Regulate T Cell Death and Is a Target for Cancer Immunotherapy. Nat Commun 2021, 12 (1), 832. https://doi.org/10.1038/s41467-021-21099-2.

1-15. (canceled)

16. A D-galactopyranose compound of formula (I) 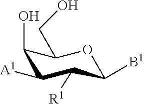
wherein
the pyranose ring is β-D-galactopyranose,
A1 is 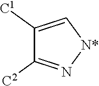
wherein the asterix * indicates the nitrogen atom of the triazole ring that is covalently attached to the galactopyranose;
C¹ is selected from a) an aryl, optionally substituted with a group selected from a halogen; CN; a C₂₋₄ alkenyl; an ethynyl; a spiro heterocycle; —COOH; —CONR²R³, wherein R² and R³ are independently selected from H, C₁₋₃ alkyl, and cyclopropyl, or R² and R³ together with the nitrogen may form a heterocycloalkyl; C₁₋₃ alkyl, optionally substituted with a F; C₁₋₃ alkyl, optionally substituted with an ethynyl; cyclopropyl, optionally substituted with a F; OC₁₋₃ alkyl, optionally substituted with a F; O-cyclopropyl, optionally substituted with a F; NR⁴R⁵, wherein R⁴ and R⁵ are independently selected from H, C₁₋₃ alkyl and cyclopropyl; C(═O)—R¹¹, wherein R^{11 a} is selected from H and C₁₋₃ alkyl OH; and R⁶—CONH— wherein R⁶ is selected from C₁₋₃ alkyl and cyclopropyl; or b) a heterocycle, optionally substituted with a group selected from a halogen; a spiro heterocycle; CN; —COOH; —CONR⁷R⁸, wherein R⁷ and R⁸ are independently selected from H, C₁₋₃ alkyl, and cyclopropyl, or R⁷ and R⁸ together with the nitrogen may form a heterocycloalkyl; C₁₋₃ alkyl, optionally substituted with a F; cyclopropyl, optionally substituted with a F; OC₁₋₃ alkyl, optionally substituted with a F; O-cyclopropyl, optionally substituted with a F; NR⁹R¹⁰, wherein R⁹ and R¹⁰ are independently selected from H, C₁₋₃ alkyl, and cyclopropyl; C(═O)—R¹¹, wherein R¹¹ is selected from H and C₁₋₃ alkyl; OH; and R¹²—CONH— wherein R¹² is selected from C₁₋₃ alkyl and cyclopropyl;
C² is selected from the group consisting of
a) H; C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{4a}R^{5a}, wherein R^{4a} and R^{5a} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{2a}R^{3a}, wherein R^{2a} and R^{3a} are independently selected from H, C₁₋₃ alkyl optionally substituted with a halogen, and cyclopropyl optionally substituted with a halogen; and S(O₂)NR^{6a}R^{7a} wherein R^{6a} and R^{7a} are independently selected from H, C₁₋₆ alkyl optionally substituted with a halogen, and cyclopropyl optionally substituted with a halogen;
b) phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{4b}R^{5b}, wherein R^{4b} and R^{5b} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{2b}R^{3b}, wherein R^{2b} and R^{3b} are independently selected from H, C₁₋₃ alkyl optionally substituted with a halogen, and cyclopropyl optionally substituted with a halogen; and S(O₂)NR^{6b}R^{7b} wherein R^{6b} and R^{7b} are independently selected from H, C₁₋₆ alkyl optionally substituted with a halogen, and cyclopropyl optionally substituted with a halogen;
c) Y—Z wherein
Y is linked to the pyrazole of A¹ and is selected from the group consisting of S, Se, SO, SO₂, O, C═O, and CR^{2c}R^{3c} wherein R^{2c} and R^{3c} are independently selected from hydrogen, OH, or halogen; and
Z is selected from the group consisting of phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of C₁₋₆ alkyl optionally substituted with a halogen; halogen; CN; C₂-alkynyl; OH; OC₁₋₆ alkyl optionally substituted with a halogen; C₃₋₆ cycloalkyl optionally substituted with a halogen; SH; SC₁₋₆ alkyl optionally substituted with a halogen; NR^{4c}R^{5c}, wherein R^{4c} and R^{5c} are independently selected from H, C₁₋₆ alkyl optionally substituted with a halogen, C₃₋₆ cycloalkyl optionally substituted with a halogen, C(O)C₁₋₆ alkyl optionally substituted with a halogen, and S(O₂)C₁₋₆ alkyl optionally substituted with a halogen; C(O)C₃₋₆ cycloalkyl optionally substituted with a halogen; S(O₂)C₃₋₆ cycloalkyl optionally substituted with a halogen; C₁₋₆ alkenyl optionally substituted with a halogen; C(O)C₁₋₆ alkyl optionally substituted with a halogen; C(O)C₃₋₆ cycloalkyl optionally substituted with a halogen; COOH; C(O)OC₁₋₆ alkyl optionally substituted with a halogen; C(O)OC₃₋₆ cycloalkyl optionally substituted with a halogen; C(O)NR^{2d}R^{3d}, wherein R^{2d} and R^{3d} are independently selected from H, C₁₋₃ alkyl optionally substituted with a halogen, and cyclopropyl optionally substituted with a halogen; and S(O₂)NR^{6c}R^{7c} wherein R^{6c} and R^{7c} are independently selected from H, C₁₋₆ alkyl optionally substituted with a halogen, and cyclopropyl optionally substituted with a halogen;
B¹ is selected from the group consisting of a heteroaryl, an aryl and a heterocycloalkyl, optionally substituted with one or more groups selected from a) C₁₋₆ alkyl optionally substituted with one or more of C₁₋₆ alkyl, amino, CN, halogen, hydroxy, C₁₋₆ alkoxy, carboxy, alkoxycarbonyl, H₂NCO, b) R¹³—C₁₋₆ alkyl, c) C₃₋₆ cycloalkyl optionally substituted with one or more of C₁₋₆ alkyl, amino, CN, halogen, or hydroxy, d) C₁₋₆ alkoxy, e) C₁₋₆ alkylthio, f) C₁₋₆ alkylsulfonyl, g) carbonyl substituted with any one of hydroxy, C₁₋₆ alkoxy, C₁₋₆ alkylNH, ((R¹⁴)(R¹⁵)N)C₁₋₆ alkylNH, or (pyridinyl)C₁₋₆ alkylNH, h) (R¹⁶)(R¹⁷)N, i) C₂-alkynyl, j) R¹³, and k) C₁₋₆ alkenyl, wherein R¹³ is selected from any one of a) phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thiazolo[4,5-b]pyridinyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of cyano, nitro, OH, C₂-alkynyl, halogen, C₁₋₆ alkyl, halo-C₁₋₆ alkyl, C₃₋₆ cycloalkyl, C₁₋₆ alkoxy, halo-C₁₋₆ alkoxy, C₁₋₆ alkylthio, carboxy, C₁₋₆ alkoxycarbonyl, CONH₂, and (R¹⁸)(R¹⁹)N; or b) (C₁₋₆ alkyl-SO₂)phenyl, (C₁₋₆ alkyl SO₂)(halo)phenyl, (aminoSO₂)phenyl, (di-C₁₋₆ alkylaminoSO₂)phenyl, ((C₁₋₆ alkyl-NHSO₂)—C₁₋₆ alkyl)phenyl, (pyrrolyl)phenyl, (imidazolyl)phenyl, (oxazolyl)phenyl, (tetrazolyl)phenyl, ((pyridinyl)methyl)phenyl, phenoxyphenyl, (benzyloxy)phenyl, ((methyl)thiazolyl)-phenyl, (thiazolyl)-benzenesulfamido, ((methyl)thiadiazolyl)benzenesulfamido, (methyl)-benzothiazolonyl, or fluoropyrazolopyrimidinyl;
wherein
R¹⁴ is hydrogen or C₁₋₆ alkyl;
R¹⁵ is hydrogen or C₁₋₆ alkyl; or
(R¹⁴)(R¹⁵)N taken together is any one of azetidinyl, pyrrolidinyl, piperidinyl, piperazinyl, or morpholinyl, optionally substituted with one or more substituents selected from halogen, C₁₋₆ alkyl, and hydroxy;
R¹⁶ is hydrogen, C₁₋₆ alkyl, C₁₋₆ alkylcarbonyl, or C₁₋₆ alkylsulfonyl;
R¹⁷ is hydrogen or C₁₋₆ alkyl; or
(R¹⁶)(R¹⁷)N taken together is any one of azetidinyl, pyrrolidinyl, piperidinyl, piperazinyl, or morpholinyl, optionally substituted with one or more substituents selected from halogen, C₁₋₆ alkyl, and C₁₋₆ alkylcarbonyl;
R¹⁸ is hydrogen, C₁₋₆ alkyl, C₁₋₆ alkylcarbonyl, or C₁₋₆ alkylsulfonyl;
R¹⁹ is hydrogen or C₁₋₆ alkyl; or
(R¹⁸)(R¹⁹)N taken together is any one of azetidinyl, pyrrolidinyl, piperidinyl, piperazinyl, or morpholinyl, optionally substituted with one or more substituents selected from halogen, C₁₋₆ alkyl, and C₁₋₆ alkylcarbonyl;
R¹ is selected from the group consisting of a) H, b) OH, c) OC₁₋₆ alkyl optionally substituted with one or more halogen, phenyl, phenyl substituted with one or more groups selected form OH and halogen, CN, OR²⁰, NR²¹R²², and CONH₂, wherein R²⁰ is selected from the group consisting of H, CN, a halogen, methyl optionally substituted with a F, OCH₃ optionally substituted with a F, OCH₂CH₃ optionally substituted with a F, OH, and R²³—CONH— wherein R²³ is selected from C₁₋₃ alkyl and cyclopropyl, R²¹ is selected from the group consisting of H, CN, a halogen, methyl optionally substituted with a F, OCH₃ optionally substituted with a F, OCH₂CH₃ optionally substituted with a F, OH, and R²⁴—CONH— wherein R²⁴ is selected from C₁₋₃ alkyl and cyclopropyl, and R²² is selected from the group consisting of H, CN, a halogen, methyl optionally substituted with a F, OCH₃ optionally substituted with a F, OCH₂CH₃ optionally substituted with a F, OH, and R²⁵—CONH— wherein R²⁵ is selected from C₁₋₃ alkyl and cyclopropyl, d) branched OC₃₋₆ alkyl optionally substituted with one or more halogen, CN, OR²⁶, NR²⁷R²⁸, and CONH₂, wherein R²⁶ is selected from the group consisting of H, CN, a halogen, methyl optionally substituted with a F, OCH₃ optionally substituted with a F, OCH₂CH₃ optionally substituted with a F, OH, and R²⁹—CONH— wherein R²⁹ is selected from C₁₋₃ alkyl and cyclopropyl, R²⁷ is selected from the group consisting of H, CN, a halogen, methyl optionally substituted with a F, OCH₃ optionally substituted with a F, OCH₂CH₃ optionally substituted with a F, OH, and R³⁰—CONH— wherein R^{3c} is selected from C₁₋₃ alkyl and cyclopropyl, and R²⁸ is selected from the group consisting of H, CN, a halogen, methyl optionally substituted with a F, OCH₃ optionally substituted with a F, OCH₂CH₃ optionally substituted with a F, OH, and R³¹—CONH— wherein R³¹ is selected from C₁₋₃ alkyl and cyclopropyl, e) cyclic OC₃₋₆ alkyl optionally substituted with one or more halogen, CN, OR³², NR³³R³⁴, and CONH₂, wherein R³² is selected from the group consisting of H, CN, a halogen, methyl optionally substituted with a F, OCH₃ optionally substituted with a F, OCH₂CH₃ optionally substituted with a F, OH, and R³⁵—CONH— wherein R³⁵ is selected from C₁₋₃ alkyl and cyclopropyl, R³³ is selected from the group consisting of H, CN, a halogen, methyl optionally substituted with a F, OCH₃ optionally substituted with a F, OCH₂CH₃ optionally substituted with a F, OH, and R³⁶—CONH— wherein R³⁶ is selected from C₁₋₃ alkyl and cyclopropyl, and R³⁴ is selected from the group consisting of H, CN, a halogen, methyl optionally substituted with a F, OCH₃ optionally substituted with a F, OCH₂CH₃ optionally substituted with a F, OH, and R³⁷—CONH— wherein R³⁷ is selected from C₁₋₃ alkyl and cyclopropyl, and f) a fluorine (F); or a pharmaceutically acceptable salt or solvate thereof.

17. The compound of claim 16 wherein C¹ is a phenyl, optionally substituted with a group selected from a halogen; CN; —COOH; —CONR²R³, wherein R² and R³ are independently selected from H, C₁₋₃ alkyl, cyclopropyl, and iso-propyl, or R² and R³ together with the nitrogen may form a heterocycloalkyl; C₂₋₄ alkenyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR⁴R⁵, wherein R⁴ and R⁵ are independently selected from H, C₁₋₃ alkyl and isopropyl; OH; and R⁶—CONH— wherein R⁶ is selected from C₁₋₃ alkyl and cyclopropyl; or b) a heterocycle, optionally substituted with a group selected from a halogen; a spiro heterocycle; CN; —COOH; —CONR⁷R⁸, wherein R⁷ and R⁸ are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR⁹R¹⁰, wherein R⁹ and R¹⁰ are independently selected from H, C₁₋₃ alkyl, cyclopropyl and isopropyl, C(═O)—R¹¹, wherein R¹¹ is selected from H and C₁₋₃ alkyl; OH; and R¹²—CONH— wherein R¹² is selected from C₁₋₃ alkyl and cyclopropyl.

18. The compound of claim 16 wherein C¹ is a phenyl substituted with one to three selected from halogen, C₂₋₄ alkenyl, ethynyl, OC₁₋₃ alkyl optionally substituted with a F, and C₁₋₃ alkyl optionally substituted with a F.

19. The compound of claim 16 wherein C¹ is a heteroaryl, optionally substituted with a group selected from a halogen; a spiro heterocycle; CN; —COOH; —CONR⁷R⁸, wherein R⁷ and R⁸ are independently selected from H, C₁₋₃ alkyl, cyclopropyl, and iso-propyl; C₁₋₃ alkyl, optionally substituted with a F; cyclopropyl, optionally substituted with a F; isopropyl, optionally substituted with a F; OC₁₋₃ alkyl, optionally substituted with a F; O-cyclopropyl, optionally substituted with a F; O-isopropyl, optionally substituted with a F; NR⁹R^{1c}, wherein R⁹ and R¹⁰ are independently selected from H, C₁₋₃ alkyl, cyclopropyl and isopropyl, C(═O)—R¹¹, wherein R¹¹ is selected from H and C₁₋₃ alkyl; OH; and R¹²—CONH— wherein R¹² is selected from C₁₋₃ alkyl and cyclopropyl.

20. The compound of claim 16 wherein C¹ is a pyridinyl optionally substituted with one to three selected from halogen, and C₁₋₃ alkyl.

21. The compound of claim 16 wherein C² is selected from a group consisting of H; C₁₋₄ alkyl optionally substituted with a halogen; CN; phenyl optionally substituted with a group selected from halogen, OC₁₋₃ alkyl substituted with a halogen, and C₁₋₃ alkyl; a group Y—Z wherein
Y is linked to the pyrazole of A¹ and is CR^{2c}R^{3c} wherein R^{2c} and R^{3c} are both hydrogen, and Z is a phenyl;
C(O)OC₁₋₃ alkyl; COOH; and C(O)NH₂.

22. The compound of claim 16 wherein B¹ is selected from the group consisting of pyrrolyl, furanyl, thienyl, pyrazolyl, isoxazolyl, isothiazolyl, imidazolyl, oxazolyl, thiazolyl, triazolyl, oxadiazolyl, thiadiazolyl, phenyl, or indolyl; optionally substituted with a group selected from a) C₁₋₆ alkyl optionally substituted with one or more of C₁₋₆ alkyl, halogen, hydroxy, C₁₋₆ alkoxy, carboxy, alkoxycarbonyl, H₂NCO, b) R¹³—C₁₋₆ alkyl, c) C₃₋₆ cycloalkyl optionally substituted with one or more of C₁₋₆ alkyl, halogen, or hydroxy, d) C₁₋₆ alkoxy, e) C₁₋₆ alkylthio, f) C₁₋₆ alkylsulfonyl, g) carbonyl substituted with any one of hydroxy, C₁₋₆ alkoxy, C₁₋₆ alkylNH, ((R¹⁴)(R¹⁵)N)C₁₋₆ alkylNH, or (pyridinyl)C₁₋₆ alkylNH, h) (R¹⁶)(R¹⁷)N, j) R¹³, and k) C₁₋₆ alkenyl, wherein R¹³, R¹⁴, R¹⁵, R¹⁶ and R¹⁷ are as defined in claim 1.

23. The compound of claim 16 wherein B¹ is triazolyl substituted with a group selected from a) C₁₋₆ alkyl optionally substituted with one or more of halogen, and j) R¹³; wherein R¹³ is selected from the group consisting of phenyl, naphthalinyl, biphenyl, pyridinyl, pyridazinyl, pyrimidinyl, pyrazinyl, quinolinyl, isoquinolinyl, quinoxainyl, indolyl, indazolyl, benzimidazolyl, benzisoxazolyl, benzisothiazolyl, benzoxazolyl, benzothiazolyl, benzodioxolyl, dihydrobenzodioxinyl, dihydroquinolinonyl, dihydrobenzothiophene-2,2-dioxide, pyrrolyl, furanyl, thiazolo[4,5-b]pyridinyl, thienyl, pyrazolyl, isoxazolyl, isothiazolyl, imidazolyl, oxazolyl, thiazolyl, triazolyl, oxadiazolyl, or thiadiazolyl; optionally substituted with one or more substituents selected from the group consisting of cyano, nitro, OH, C₂-alkynyl, halogen, C₁₋₆ alkyl, halo-C₁₋₆ alkyl, C₃₋₆ cycloalkyl, C₁₋₆ alkoxy, halo-C₁₋₆ alkoxy, C₁₋₆ alkylthio, carboxy, C₁₋₆ alkoxycarbonyl, and CONH₂.

24. The compound of claim 16 wherein R¹ is selected from H, OH, OC₁₋₄ alkyl, or OC₁₋₄ alkyl substituted with at least one from the group consisting of phenyl and phenyl substituted with one or more groups selected from OH and halogen.

25. The compound of claim 16 wherein R¹ is OH.

26. The compound of claim 16 selected from any one of the group consisting of:
3-{5-{3-[4-(4-Chloro-3,5-difluorophenyl)-1H-1,2-pyrazol-1-yl]-3-deoxy-β-D-galactopyranosyl}-3-methyl-1H-1,2,4-triazol-1-yl}-5-chloro-2-cyclopropylpyridine,
6-{3-{3-Deoxy-3-[4-(3,4,5-trifluorophenyl)-1H-1,2-pyrazol-1-yl]-β-D-galactopyranosyl}-4H-1,2,4-triazol-4-yl}-2-methylbenzothiazole,
5-Chloro-1-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-(trifluoromethyl)benzene,
1-{5-{3-Deoxy-3-[4-(3,4,5-trifluorophenyl)-1H-1,2-pyrazol-1-yl]-β-D-galactopyranosyl}-3-methyl-1H-1,2,4-triazol-1-yl}-2-(trifluoromethyl)-2-(3,4,5-trifluorophenyl)benzene,
1-{5-{3-[4-(4-Chloro-3,5-difluorophenyl)-1H-1,2-pyrazol-1-yl]-3-deoxy-β-D-galactopyranosyl}-3-methyl-1H-1,2,4-triazol-1-yl}-5-chloro-2-(trifluoromethyl)benzene,
5-Chloro-1-{5-{3-deoxy-3-[4-(6-fluoropyridin-2-yl)-1H-1,2-pyrazol-1-yl]-β-D-galactopyranosyl}-3-methyl-1H-1,2,4-triazol-1-yl}-2-(trifluoromethyl)benzene,
5-Chloro-1-{5-{3-deoxy-3-[4-(4-fluoropyridin-2-yl)-1H-1,2-pyrazol-1-yl]-β-D-galactopyranosyl}-3-methyl-1H-1,2,4-triazol-1-yl}-2-(trifluoromethyl)benzene,
6-{5-{3-Deoxy-3-[4-(3,4,5-trifluorophenyl)-1H-1,2-pyrazol-1-yl]-β-D-galactopyranosyl}-3-methyl-1H-1,2,4-triazol-1-yl}-2-methylbenzothiazole,
6-{5-{3-[4-(4-Chloro-3,5-difluorophenyl)-1H-1,2-pyrazol-1-yl]-3-deoxy-β-D-galactopyranosyl}-3-methyl-1H-1,2,4-triazol-1-yl}-2-methylbenzothiazole,
1-{3-{3-[4-(4-Chloro-3,5-difluorophenyl)-1H-1,2-pyrazol-1-yl]-3-deoxy-β-D-galactopyranosyl}-5-methyl-4H-1,2,4-triazol-4-yl}-5-chloro-2-(trifluoromethyl)benzene,
1-{3-{3-[4-(4-Chloro-3,5-difluorophenyl)-1H-1,2-pyrazol-1-yl]-3-deoxy-β-D-galactopyranosyl}-5-methyl-4H-1,2,4-triazol-4-yl}-5-chloro-2-(trifluoromethyl)benzene,
1-{5-{3-[4-(4-Chloro-3,5-difluorophenyl)-3-methyl-1H-1,2-pyrazol-1-yl]-3-deoxy-β-D-galactopyranosyl}-3-methyl-1H-1,2,4-triazol-1-yl}-5-chloro-2-(trifluoromethyl)benzene,
5-Chloro-3-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-cyclopropylpyridine,
1-{5-{3-[4-(5-Chloro-4-fluoropyridin-2-yl)-1H-1,2-pyrazol-1-yl]-3-deoxy-β-D-galactopyranosyl}-3-methyl-1H-1,2,4-triazol-1-yl}-5-chloro-2-(trifluoromethyl)benzene,
5-Chloro-1-{5-{3-deoxy-3-[4-(4-fluoro-5-methylpyridin-2-yl)-1H-1,2-pyrazol-1-yl]-β-D-galactopyranosyl}-3-methyl-1H-1,2,4-triazol-1-yl}-2-(trifluoromethyl)benzene,
1-{5-{3-[4-(5-Chloro-4-methylpyridin-2-yl)-1H-1,2-pyrazol-1-yl]-3-deoxy-β-D-galactopyranosyl}-3-methyl-1H-1,2,4-triazol-1-yl}-5-chloro-2-(trifluoromethyl)benzene,
5-Chloro-1-{5-{3-deoxy-3-[4-(4-ethynyl-3,5-difluorophenyl)-1H-1,2-pyrazol-1-yl]-β-D-galactopyranosyl}-3-methyl-1H-1,2,4-triazol-1-yl}-2-(trifluoromethyl)benzene,
5-Chloro-1-{5-{3-deoxy-3-{4-[3,5-difluoro-4-(2-propyn-1-yl)phenyl]-1H-1,2-pyrazol-1-yl}-β-D-galactopyranosyl}-3-methyl-1H-1,2,4-triazol-1-yl}-2-(trifluoromethyl)benzene,
5-Bromo-3-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}pyridine,
5-Chloro-3-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-(trifluoromethyl)pyridine,
5-Chloro-2-cyano-3-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}pyridine,
5-Bromo-2-cyano-3-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}pyridine,
3-Chloro-2-cyano-5-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}pyridine,
5-Bromo-1-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-(trifluoromethyl)benzene,
5-Bromo-3-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-(trifluoromethyl)pyridine,
5-Bromo-2-cyano-1-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}benzene,
5-Chloro-2-cyano-1-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}benzene,
3-{5-{3-[4-(4-Chloro-3,5-difluorophenyl)-1H-1,2-pyrazol-1-yl]-3-deoxy-β-D-galactopyranosyl}-3-methyl-1H-1,2,4-triazol-1-yl}-5-chloro-2-(trifluoromethyl)pyridine,
1-{5-{3-[4-(4-Chloro-3,5-difluorophenyl)-3-ethoxycarbonyl-1H-1,2-pyrazol-1-yl]-3-deoxy-β-D-galactopyranosyl}-3-methyl-1H-1,2,4-triazol-1-yl}-5-chloro-2-(trifluoromethyl)benzene,
1-{5-{3-[3-Carboxy-4-(4-chloro-3,5-difluorophenyl)-1H-1,2-pyrazol-1-yl]-3-deoxy-β-D-galactopyranosyl}-3-methyl-1H-1,2,4-triazol-1-yl}-5-chloro-2-(trifluoromethyl)benzene,
1-{5-{3-[4-(4-Chloro-3,5-difluorophenyl)-3-cyano-1H-1,2-pyrazol-1-yl]-3-deoxy-β-D-galactopyranosyl}-3-methyl-1H-1,2,4-triazol-1-yl}-5-chloro-2-(trifluoromethyl)benzene,
1-{5-{3-[3-Aminocarbonyl-4-(4-chloro-3,5-difluorophenyl)-1H-1,2-pyrazol-1-yl]-3-deoxy-β-D-galactopyranosyl}-3-methyl-1H-1,2,4-triazol-1-yl}-5-chloro-2-(trifluoromethyl)benzene,
5-Chloro-2-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-thiazole,
6-{5-{3-Deoxy-3-[4-(3,4,5-trifluorophenyl)-1H-1,2-pyrazol-1-yl]-β-D-galactopyranosyl}-3-methyl-1H-1,2,4-triazol-1-yl}benzothiazole,
6-{5-{3-Deoxy-3-[4-(3,4,5-trifluorophenyl)-1H-1,2-pyrazol-1-yl]-β-D-galactopyranosyl}-3-methyl-1H-1,2,4-triazol-1-yl}-2-ethylbenzothiazole,
6-{5-{3-Deoxy-3-[4-(3,4,5-trifluorophenyl)-1H-1,2-pyrazol-1-yl]-β-D-galactopyranosyl}-3-methyl-1H-1,2,4-triazol-1-yl}-2-(trifluoromethyl)benzothiazole,
6-{5-{3-Deoxy-3-[4-(3,4,5-trifluorophenyl)-1H-1,2-pyrazol-1-yl]-β-D-galactopyranosyl}-3-methyl-1H-1,2,4-triazol-1-yl}-5-fluoro-2-methylbenzothiazole,
6-{5-{3-Deoxy-3-[4-(3,4,5-trifluorophenyl)-1H-1,2-pyrazol-1-yl]-β-D-galactopyranosyl}-3-methyl-1H-1,2,4-triazol-1-yl}-7-fluoro-2-methylbenzothiazole,
6-{5-{3-Deoxy-3-[4-(3,4,5-trifluorophenyl)-1H-1,2-pyrazol-1-yl]-β-D-galactopyranosyl}-3-methyl-1H-1,2,4-triazol-1-yl}-4-fluoro-2-methylbenzothiazole,
7-Chloro-6-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-methylbenzothiazole,
4-Chloro-6-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-methylbenzothiazole,
4-Cyano-6-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-methylbenzothiazole,
5-Bromo-6-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-methylbenzothiazole,
5-Cyano-6-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-methylbenzothiazole,
6-{5-{3-Deoxy-3-[4-(3,4,5-trifluorophenyl)-1H-1,2-pyrazol-1-yl]-β-D-galactopyranosyl}-3-methyl-1H-1,2,4-triazol-1-yl}-2-methylthiazolo[4,5-b]pyridine,
5-Cyano-1-{5-{3-deoxy-3-[4-(3,4,5-trifluorophenyl)-1H-1,2-pyrazol-1-yl]-β-D-galactopyranosyl}-3-methyl-1H-1,2,4-triazol-1-yl}-2-(trifluoromethyl)benzene,
5-Chloro-1-{5-{3-deoxy-3-[4-(3,4,5-trifluorophenyl)-3-phenyl-1H-1,2-pyrazol-1-yl]-β-D-galactopyranosyl}-3-methyl-1H-1,2,4-triazol-1-yl}-2-(trifluoromethyl)benzene,
5-Chloro-1-{5-{3-deoxy-3-[4-(3,4,5-trifluorophenyl)-3-(2-fluorophenyl)-1H-1,2-pyrazol-1-yl]-β-D-galactopyranosyl}-3-methyl-1H-1,2,4-triazol-1-yl}-2-(trifluoromethyl)benzene,
5-Chloro-1-{5-{3-deoxy-3-[4-(3,4,5-trifluorophenyl)-3-methyl-1H-1,2-pyrazol-1-yl]-β-D-galactopyranosyl}-3-methyl-1H-1,2,4-triazol-1-yl}-2-(trifluoromethyl)benzene,
1-{5-{3-[3-Benzyl-4-(3,4,5-trifluorophenyl)-1H-1,2-pyrazol-1-yl]-3-deoxy-β-D-galactopyranosyl}-3-methyl-1H-1,2,4-triazol-1-yl}-5-chloro-2-(trifluoromethyl)benzene,
5-Chloro-1-{5-{3-[3-cyano-4-(3,4,5-trifluorophenyl)-1H-1,2-pyrazol-1-yl]-3-deoxy-β-D-galactopyranosyl}-3-methyl-1H-1,2,4-triazol-1-yl}-2-(trifluoromethyl)benzene,
5-Chloro-1-{5-{3-deoxy-3-[4-(3,4,5-trifluorophenyl)-3-(2-methoxyphenyl)-1H-1,2-pyrazol-1-yl]-β-D-galactopyranosyl}-3-methyl-1H-1,2,4-triazol-1-yl}-2-(trifluoromethyl)benzene,
5-Chloro-1-{5-{3-deoxy-3-[4-(3,4,5-trifluorophenyl)-3-(2-methylphenyl)-1H-1,2-pyrazol-1-yl]-β-D-galactopyranosyl}-3-methyl-1H-1,2,4-triazol-1-yl}-2-(trifluoromethyl)benzene,
5-Chloro-1-{5-{3-deoxy-3-[4-(3,4,5-trifluorophenyl)-3-(3-fluorophenyl)-1H-1,2-pyrazol-1-yl]-β-D-galactopyranosyl}-3-methyl-1H-1,2,4-triazol-1-yl}-2-(trifluoromethyl)benzene,
5-Chloro-1-{5-{3-deoxy-3-[4-(3,4,5-trifluorophenyl)-3-(4-fluorophenyl)-1H-1,2-pyrazol-1-yl]-β-D-galactopyranosyl}-3-methyl-1H-1,2,4-triazol-1-yl}-2-(trifluoromethyl)benzene,
5-Chloro-1-{5-{3-deoxy-3-{4-[4-(trifluoromethyl)phenyl]-1H-1,2-pyrazol-1-yl}-β-D-galactopyranosyl}-3-methyl-1H-1,2,4-triazol-1-yl}-2-(trifluoromethyl)benzene,
5-Chloro-1-{5-{3-deoxy-3-[4-(3-fluoro-4-methylphenyl)-1H-1,2-pyrazol-1-yl]-β-D-galactopyranosyl}-3-methyl-1H-1,2,4-triazol-1-yl}-2-(trifluoromethyl)benzene,
1-{5-{3-[4-(3-Chlorophenyl)-1H-1,2-pyrazol-1-yl]-3-deoxy-β-D-galactopyranosyl}-3-methyl-1H-1,2,4-triazol-1-yl}-5-chloro-2-(trifluoromethyl)benzene, and
1-{5-{3-[4-(4-Chlorophenyl)-1H-1,2-pyrazol-1-yl]-3-deoxy-β-D-galactopyranosyl}-3-methyl-1H-1,2,4-triazol-1-yl}-5-chloro-2-(trifluoromethyl)benzene; or a pharmaceutically acceptable salt or solvat thereof.

27. A pharmaceutical composition comprising the compound of a claim 16 and optionally a pharmaceutically acceptable additive.

28. A method for treating a disorder relating to the binding of a galectin-3 to a ligand in a mammal, wherein said disorder is selected from the group consisting of inflammation, acute post myocardial infarctions (MI), acute coronary syndrome, acute stent occlusion, acute myocardial reperfusion injury, acute pneumonitidies, acute lung injury (ALI), acute kidney injury (AKI), acute hepatitis, acute on chronic liver failure, acute alcohol hepatitis, acute pancreatitis, acute uveitis, acute pancreatitis related liponecrosis, acute retinitis, acute nephritis, acute myocarditis, chronic autoimmune diseases in all organs, (e.g. lung, liver, kidney, heart, skin, muscle, gut), chronic bacterial infections, chronic viral related inflammation; fibrosis, pulmonary fibrosis, liver fibrosis, kidney fibrosis, ophthalmological fibrosis and fibrosis of the skin and heart, acute post-surgical ocular fibrosis, acute transplantation rejection of the kidney, heart, lung, liver, and pancreas, acute post explosion/improvised explosive devices, acute post toxic dust, acute chemical exposure, chronic lung fibrosis, interstitial lung fibrosis (IPF), Interstitial Lung Disease (ILD), Childhood ILD (ChILD); chronic liver fibrosis, chronic alcohol fibrosis, chronic viral fibrosis, chronic diabetic fibrosis, diabetic nephropathy, chronic glomerulonephritis, renal artery stenosis, endometriosis; scarring; keloid formation; aberrant scar formation; surgical adhesions; scleroderma; systemic sclerosis; septic shock; cancers, carcinomas, sarcomas, leukemias and lymphomas, T-cell lymphomas; metastasising cancers; autoimmune diseases, psoriasis, rheumatoid arthritis, Crohn's disease, ulcerative colitis, intestinal fibrosis, ankylosing spondylitis, systemic lupus erythematosus; metabolic disorders; coagulopathies, thrombosis proneness idiopathic (thrombophilia), autoimmune based thrombophilia, microthrombosis at multiorgan failure, COVID-19 related coagulopathy, thrombophilia in cancer disease; cardiovascular disorders, cardiac fibrosis, cardiac failure, left and right atrial fibrillation, atheromatosis, arterial inflammation, arterial calcification, aortic stenosis; heart disease; heart failure; aortic stenosis, atherosclerosis, pathological angiogenesis, ocular angiogenesis or a disease or condition associated with ocular angiogenesis, e.g. neovascularization related to cancer; and eye diseases, age-related macular degeneration and corneal neovascularization; atherosclerosis; endocrine disorders, Addison, autoimmune hypophysitis; metabolic diseases, diabetes; type 2 diabetes; insulin resistance; obesity; Diastolic HF; atrophic diseases in the brain, Alzheimer's and Parkinson's, atrophic diseases in the cerebellum, cerebellar atrophy, atrophic spinal diseases, ALS; disorders related to transplantation in organs, anti-rejection prophylaxis, anti-acute rejection, anti-chronic rejection; acute burn; acute inflammatory reaction; chronic acute skin graft rejection; chronic scarring; asthma and other interstitial lung diseases, including Hermansky-Pudlak syndrome, pulmonary arterial hypertension, Rheumatoid disease associated interstitial lung disease RA-ILD, Systemic Sclerosis SSc-ILD, lung disease with fibrosis, COPD (Chronic Obstructive Pulmonary Disease) and asthma; Otosclerosis, mesothelioma; post-surgery disorders, anti-keloid, anti-stricture, anti-adhesion, anti-thrombosis, fibrosis/scar reduction following cosmetic procedures; toxin exposure disorders, toxic hepatitis, cholera toxin related, mushroom toxin based acute renal failure, pertussis toxin, Aeromonas hydrophila enterotoxin, cadmium induced cardiac toxicity, helicobacter O-antigen related toxicity, LPS based toxicity, Streptozotocin toxicity, asbestos exposure, Nephrogenic Systemic Fibrosis (Post Contrast Agents); Tissue injury, Spinal cord injury, Peripheral nerve repair; congenital hepatic fibrosis; hereditary fibrosing poikiloderma with tendon contractures, myopathy, and pulmonary fibrosis; liver disorders, non-alcoholic steatohepatitis (NASH) or non-alcoholic fatty liver disease, liver cirrhosis of various origins, alcoholic and non-alcoholic, autoimmune cirrhosis primary biliary cirrhosis and sclerosing cholangitis, virally induced cirrhosis, cirrhosis induced by genetic disease; Liver cancer, cholangiocarcinoma, biliary tract cancer; neurodegenerative disorders, Parkinsons disease, Alzheimers disease, cognitive impairment, cerebrovascular diseases, stroke, traumatic brain injury, Huntington's disease, amyotrophic lateral sclerosis (ALS), multiple sclerosis (MS), peripheral nephropathy,
Comprising administering a therapeutically effective amount of the compound of claim 16.
